(19) United States
(12) Patent Application Publication
(10) Pub. No.: US 2024/0041765 A1
(43) Pub. Date: Feb. 8, 2024

(54) ORAL LIQUID SUSPENSION OF PAN-RAF KINASE INHIBITOR

(71) Applicant: DAY ONE BIOPHARMACEUTICALS, INC., Brisbane, CA (US)

(72) Inventors: Donald T. CORSON, Erie, CO (US); Michael PREIGH, Boulder, CO (US); Dan SMITHEY, Bend, OR (US); Randy WALD, Bend, OR (US); Erica B. SCHLESINGER, Sisters, OR (US)

(21) Appl. No.: 18/332,906

(22) Filed: Jun. 12, 2023

Related U.S. Application Data
(63) Continuation of application No. PCT/US23/22430, filed on May 16, 2023.
(60) Provisional application No. 63/342,533, filed on May 16, 2022.

(51) Int. Cl. A61K 9/10; A61K 47/32; A61K 47/38; A61K 47/26; A61K 47/20; A61K 47/34; A61K 47/02; A61K 31/506; A61K 9/00; A61K 9/19

(52) U.S. Cl. CPC A61K 9/10; A61K 47/32; A61K 47/38; A61K 47/26; A61K 47/20; A61K 47/34; A61K 47/02; A61K 31/506; A61K 9/0053; A61K 9/19

(57) ABSTRACT

Disclosed herein are solid formulations of (R)-2-(1-(6-amino-5-chloropyrimidine-4-carboxamido)ethyl)-N-(5-chloro-4-(trifluoromethyl)pyridin-2-yl)thiazole-5-carboxamide (Compound A) or a pharmaceutically acceptable salt thereof for preparing liquid suspensions. In another aspect, the instant application provides methods of preparation of liquid suspensions and kits of solid formulations comprising Compound A. In another aspect, the instant application provides methods of treating cancer with formulations of Compound A. Additionally, the instant disclosure provides methods of treating pediatric cancer patients by the administration of formulations of Compound A.

### CROSS-REFERENCE

This application is a continuation of International Patent Application PCT/US2023/022430 filed on May 16, 2023, which claims the benefit of U.S. Provisional Patent Application No. 63/342,533 filed on May 16, 2022, each of which is incorporated herein by reference in its entirety.

### BACKGROUND

In 2018, there were 18.1 million new cases and 9.5 million cancer-related deaths worldwide. By 2040, the number of new cancer cases per year is expected to rise to 29.5 million and the number of cancer-related deaths to 16.4 million. In 2020, there was an estimated 1.8 million new cancer cases diagnosed and 606,520 cancer deaths in the US. Cancer remains the second most common cause of death in the US, accounting for nearly 1 of every 24 deaths.

Each year, approximately 15,500 children under the age of 18 in the United States and 300,000 globally are diagnosed with cancer. Moreover, cancer remains the most common cause of death by disease for children in the United States, accounting for over 1,700 deaths per year. Despite the need for safer and more effective therapies for childhood cancers, new drugs for pediatric patients are rare. (R)-2-(1-(6-amino-5-chloropyrimidine-4-carboxamido)ethyl)-N-(5-chloro-4-(trifluoro methyl) pyridin-2-yl) thiazole-5-carboxamide (“Compound A”) (also known as tovorafenib, DAY101, DOT101, TAK-580, BIIB024, or MLN 2480) is a class II pan Raf kinase inhibitor useful for the treatment of Raf-mediated diseases such as cancer. Compound A is currently administered in the form of oral tablets. Thus, there is an unmet need to develop new and useful formulations of Compound A as an effective treatment for pediatric cancer patients.

### SUMMARY

In certain aspects, the present disclosure provides a pharmaceutical powder comprising:
(a) an amorphous solid dispersion that comprises (R)-2-(1-(6-amino-5-chloropyrimidine-4-carboxamido)ethyl)-N-(5-chloro-4-(trifluoromethyl)pyridin-2-yl)thiazole-5-carboxamide (Compound A) or a pharmaceutically acceptable salt thereof; and
(b) one or more pharmaceutically acceptable excipients, wherein the excipient comprises a flow-aid and a surfactant.

In some embodiments, the one or more pharmaceutically acceptable excipients stabilize Compound A in the amorphous state and/or aid in its solubilization.

In some embodiments, the pharmaceutical powder is reconstituted into an oral liquid suspension. In some embodiments, the amorphous solid dispersion comprises one or more polymers. In some embodiments, the one or more polymers comprise polyvinylpyrrolidone, vinylpyrrolidone-vinyl acetate copolymer (PVP-VA), cross linked polyvinyl N-pyrrolidone, polyvinyl alcohol (PVA), polysaccharide, hydroxypropyl methylcellulose (HPMC or Hypromellose), hydroxyethyl cellulose (HEC), hydroxypropyl cellulose (HPC), polyethylene oxide, hydroxypropyl-β-cyclodextrin (HP-3-CD), sulfobutylether-β-cyclodextrin (Captisol), γ-cyclodextrin, hydroxypropyl methylcellulose acetate succinate (HPMCAS), polyethylene glycol (PEG), polyvinyl caprolactam-polyvinyl acetate-polyethylene glycol graft copolymer (PVAc-PVCap-PEG), polysaccharide, poly(methacrylic acid-co-methyl methacrylate) (Eudragit), poloxamers, silica gel, aluminosilicate, or a combination thereof. In some embodiments, the one or more polymers comprise PVP-VA or HPMCAS.

In some embodiments, the amorphous solid dispersion comprises about 10 wt % to about 90 wt % of the one or more polymers. In some embodiments, the amorphous solid dispersion comprises about 10 wt % to about 90 wt % of the Compound A or a pharmaceutically acceptable salt thereof. In some embodiments, a weight ratio of the Compound A or a pharmaceutically acceptable salt thereof to the one or more polymers is from 2:1 to 1:2. In some embodiments, the amorphous solid dispersion comprises Compound A. In some embodiments, the amorphous solid dispersion is a hot melt extrudate (HME). In some embodiments, the amorphous solid dispersion is prepared by dissolving the Compound A or a pharmaceutically acceptable salt thereof in a solvent and then removing at least a part of the solvent. In some embodiments, Compound A or a pharmaceutically acceptable salt thereof is amorphous. In some embodiments, the amorphous solid dispersion has a D50 value of about 10 μm to about 500 μm. In some embodiments, the pharmaceutical powder comprises about 10 wt % to about 80 wt % of the amorphous solid dispersion. In some embodiments, the pharmaceutical powder comprises about 20 wt % to about 60 wt % of the amorphous solid dispersion. In some embodiments, the one or more pharmaceutically acceptable excipients are in a mixture with the amorphous solid dispersion in the pharmaceutical powder. In some embodiments, the pharmaceutical powder comprises about 5 wt % to about 80 wt % of the one or more pharmaceutically acceptable excipients. In some embodiments, the one or more pharmaceutically acceptable excipients comprise an antifoam, a filler, a colorant, a preservative, a flavoring agent, a sweetener, or a combination thereof. In some embodiments, the antifoam comprises simethicone. In some embodiments, the antifoam is present in the pharmaceutical powder from about 0.1 wt % to about 10 wt %. In some embodiments, the filler comprises a polysaccharide, a sugar or a derivative thereof, or both. In some embodiments, the filler comprises cellulose, starch, a synthetic soluble fiber, a sugar alcohol, or a combination thereof. In some embodiments, the filler comprises mannitol. In some embodiments, the filler comprises microcrystalline cellulose, polydextrose, sodium carboxymethyl cellulose (sodium CMC), or a combination thereof. In some embodiments the pharmaceutical powder comprises about 5 wt % to about 80 wt % of the filler. In some embodiments, the flow-aid is selected from silicon dioxide, magnesium stearate, talc, starch, magnesium silicate, hydrated sodium sulfoaluminate, and a combination thereof. In some embodiments, the silicon dioxide comprises fumed silica, colloidal silicon dioxide (CSD), or both. In some embodiments, the pharmaceutical powder comprises about 0.25 wt % to about 10 wt % of the flow-aid. In some embodiments, the surfactant comprises cationic surfactant, anionic surfactant, non-ionic surfactant, or a combination thereof. In some embodiments, the surfactant comprises sodium lauryl sulfate (SLS). In some embodiments, the surfactant comprises poloxamer. In some embodiments, the pharmaceutical powder comprises about 0.01 wt % to about 10 wt % of the surfactant. In some embodiments, the pharmaceutical powder comprises:
a) about 10 wt % to about 60 wt % of an amorphous solid dispersion comprising (i) about 40 wt % to about 60 wt % of Compound A and (ii) about 40 wt % to about 60 wt % of PVP-VA, wherein the amorphous solid dispersion is a hot melt extrudate;
b) about 30 wt % to about 70 wt % of a filler, wherein the filler comprises microcrystalline cellulose and mannitol;
c) about 0.1 wt % to 5 wt % of a surfactant, wherein the surfactant is SLS;
d) about 0.25 wt % to 6 wt % of a flow-aid, wherein the flow-aid is CSD; and
e) about 0.5 wt % to 5 wt % of an antifoam, wherein the antifoam is simethicone or dimethicone.

In certain aspects the present disclosure provides an oral liquid suspension comprising, a solid formulation, e.g., powder formulation as described herein, in a kit as disclosed herein, and an aqueous solution. In some embodiments, an oral liquid suspension produced by contacting the solid formulation, e.g., powder formulation as described herein, in a kit as disclosed herein with an aqueous solution. In some embodiments, the oral liquid suspension comprises, a pharmaceutical powder, e.g., powder formulation as described herein, and an aqueous solution. In some embodiments, an oral liquid suspension produced by contacting the pharmaceutical powder, e.g., powder formulation as described herein, as disclosed herein with an aqueous solution. In some embodiments, the powder will be reconstituted with water and the resulting suspension will be administered orally, or enterally via a nasal or gastric feeding tube with a dosing syringe. In certain aspects the present disclosure provides an oral liquid suspension comprising: (a) (R)-2-(1-(6-amino-5-chloropyrimidine-4-carboxamido)ethyl)-N-(5-chloro-4-(trifluoromethyl)pyridin-2-yl)thiazole-5-carboxamide (Compound A) or a pharmaceutically acceptable salt thereof; (b) one or more pharmaceutically acceptable excipients; and (c) water. In some embodiments, Compound A or a pharmaceutically acceptable salt thereof is in a form of an amorphous solid dispersion. In some embodiments, the amorphous solid dispersion comprises one or more polymers. In some embodiments, the one or more polymers comprise polyvinylpyrrolidone, vinylpyrrolidone-vinyl acetate copolymer (PVP-VA), cross linked polyvinyl N-pyrrolidone, polyvinyl alcohol (PVA), polysaccharide, hydroxypropyl methylcellulose (HPMC or Hypromellose), hydroxyethyl cellulose (HEC), hydroxypropyl cellulose (HPC), polyethylene oxide, hydroxypropyl-β-cyclodextrin (HP-β-CD), sulfobutylether-β-cyclodextrin (Captisol), cyclodextrin (e.g., -cyclodextrin), hydroxypropyl methylcellulose acetate succinate (HPMCAS), polyethylene glycol (PEG), polyvinyl caprolactam-polyvinyl acetate-polyethylene glycol graft copolymer (PVAc-PVCap-PEG), polysaccharide, poly(methacrylic acid-co-methyl methacrylate) (Eudragit), poloxamers, silica gel, aluminosilicate, or a combination thereof. In some embodiments, the one or more pharmaceutically acceptable excipients are selected from an antifoam, a flow-aid, a surfactant, a filler, a colorant, a preservative, a flavoring agent, a sweetener, and a combination thereof. In some embodiments, the oral liquid suspension is prepared by adding water to a powder formulation of Compound A. In some embodiments, a concentration of Compound A or a pharmaceutically acceptable salt thereof is about 10 mg/mL to about 125 mg/mL in the oral liquid suspension. In some embodiments, a concentration of Compound A or a pharmaceutically acceptable salt thereof is about 10 to about 50 mg/mL in the oral liquid suspension. In some embodiments, a concentration of Compound A or a pharmaceutically acceptable salt thereof is about 25 to about 50 mg/mL in the oral liquid suspension. In some embodiments, the aqueous solution is water. In some embodiments, the pH value of the liquid suspension is about 2 to 7. In some embodiments, the suspension is dosed via an oral syringe for at least 30 minutes. In some embodiments, the suspension retains syringeability for at least 15 minutes following contacting the powder formulation with the aqueous solution. In some embodiments, the suspension retains syringeable for at least 30 minutes following contacting the powder formulation with the aqueous solution. The term syringable refers to the ability to pull the oral suspension into the barrel of a syringe for administration, e.g., oral administration, to a subject, e.g., a child or infant. In some embodiments, a concentration of the one or more polymers in the suspension is about 10 mg/mL to about 200 mg/mL. In some embodiments, the concentration of the one or more polymers in the suspension is about 30 mg/mL to about 50 mg/mL. In some embodiments, the concentration of the one or more polymers in the suspension is about 60 mg/mL to about 80 mg/mL. In some embodiments, a concentration of the filler in the suspension is about 30 mg/mL to about 500 mg/mL. In some embodiments, the concentration of the filler in the suspension is about 50 mg/mL to about 300 mg/mL. In some embodiments, the concentration of the filler in the suspension is about 125 mg/mL to about 200 mg/mL. In some embodiments, a concentration of the surfactant in the suspension is about 0.5 mg/mL to about 10 mg/mL. In some embodiments, the concentration of the surfactant in the suspension is about 1 mg/mL to about 2.5 mg/mL. In some embodiments, a concentration of the flow-aid in the suspension is about 3 mg/mL to about 30 mg/mL. In some embodiments, the concentration of the flow-aid in the suspension is about 5 mg/mL to about 15 mg/mL. In some embodiments, the concentration of the flow-aid in the suspension is about 10 mg/mL to about 15 mg/mL. In some embodiments, a concentration of the antifoam in the suspension is about 1 mg/mL to about 30 mg/mL. In some embodiments, the concentration of the antifoam in the suspension is about 1 mg/mL to about 10 mg/mL. In some embodiments, the concentration of the antifoam in the suspension is about 3 mg/mL to about 8 mg/mL. In some embodiments, the suspension comprises, based on the weight of the solids:
(a) about 10 wt % to about 50 wt % of an amorphous solid dispersion comprising (i) about 40 wt % to about 60 wt % of Compound A and (ii) about 40 wt % to about 60 wt % of PVP-VA, wherein the amorphous solid dispersion is a hot melt extrudate;
(b) about 40 wt % to about 70 wt % of a filler, wherein the filler comprises microcrystalline cellulose and mannitol;
(c) about 0.25 wt % to about 1 wt % of a surfactant, wherein the surfactant is SLS; (d) about 1 wt % to about 6 wt % of a flow-aid, wherein the flow-aid is colloidal silicon dioxide (CSD);
(e) about 1 wt % to about 5 wt % of an antifoam, wherein the antifoam is simethicone; and
(f) optionally a preservative, a flavoring agent, a sweetener, or a combination thereof.

In some embodiments, the suspension comprises, based on the weight of the solids:
(a) about 10 wt % of an amorphous solid dispersion comprising (i) about 40 wt % of Compound A and (ii) about 60 wt % of copovidone, wherein the amorphous solid dispersion is a hot melt extrudate;
(b) about 30 wt % to 32 wt % mannitol;
(c) about 30 wt % to 32 wt % microcrystalline cellulose;
(d) about 0.5 wt % to 1 wt % SLS;
(e) about 4 wt % to 5 wt % CSD;
(f) about 2 wt % to 3 wt % simethicone; and
(g) optionally a preservative, a flavoring agent, a sweetener, or a combination thereof.

In some embodiments, the oral liquid suspension is bioequivalent to a tablet formulation of Compound A, wherein the tablet composition comprises an amorphous solid dispersion comprising (i) about 40 wt % of Compound A and (ii) about 60 wt % of copovidone, wherein the amorphous solid dispersion is a hot melt extrudate; and one or more pharmaceutically acceptable excipients. In some embodiments, the oral liquid suspension, when administered to a human subject in an amount equivalent to about 100 mg of Compound A, is sufficient to achieve in the subject a maximum observed blood plasma concentration (Cmax) of Compound A of at least about 100 ng/mL. In some embodiments, the oral liquid suspension, when administered to a human subject in an amount equivalent to about 100 mg of Compound A, is sufficient to achieve in the subject a maximum observed blood plasma concentration (Cmax) of Compound A of at least about 100 ng/mL.

In certain aspects the present disclosure provides methods of treating cancer comprising administering to the subject an oral liquid suspension comprising (R)-2-(1-(6-amino-5-chloropyrimidine-4-carboxamido)ethyl)-N-(5-chloro-4-(trifluoromethyl)pyridin-2-yl)thiazole-5-carboxamide (Compound A), or a pharmaceutically acceptable salt thereof in an amount sufficient to achieve in the subject a maximum observed blood plasma concentration (Cmax) of Compound A of at least 2000 ng/mL. In some aspects, the present disclosure provides methods of treating cancer in a subject comprising administering to the subject a pharmaceutical powder as disclosed herein or a reconstituted oral liquid formulation as disclosed herein. In some aspects the present disclosure provides methods of treating cancer in a subject comprising reconstituting a solid formulation in a kit as disclosed herein and administering the reconstituted formulation to the subject. In some embodiments, the subject is 18 years old or younger. In some embodiments, the cancer is low grade glioma. In some aspects, the present disclosure provides methods of treating a subject with pediatric low grade glioma (pLGG), comprising reconstituting an amorphous solid dispersion of Compound A or a salt thereof in an aqueous solution and administering a pharmaceutically acceptable dosage of the reconstituted Compound A or a salt thereof to the subject in need thereof. In some embodiments, the method is administered with food, before consuming food, or after consuming food.

In certain aspects the present disclosure provides a kit comprising: (a) a solid formulation of an amorphous solid dispersion of (R)-2-(1-(6-amino-5-chloropyrimidine-4-carboxamido)ethyl)-N-(5-chloro-4-(trifluoromethyl)pyridin-2-yl)thiazole-5-carboxamide (Compound A) or a pharmaceutically acceptable salt thereof, wherein the solid formulation comprises one or more pharmaceutically acceptable excipients; and (b) instructions for aqueous reconstitution of the solid formulation. In some embodiments, the solid formulation is in a powder, granular, or pellet form. In some embodiments, the solid formulation is in a powder form. In some embodiments, the amorphous solid dispersion comprises one or more polymers. In some embodiments, the one or more polymers comprise polyvinylpyrrolidone, vinylpyrrolidone-vinyl acetate copolymer (PVP-VA), cross linked polyvinyl N-pyrrolidone, polyvinyl alcohol (PVA), polysaccharide, hydroxypropyl methylcellulose (HPMC or Hypromellose), hydroxyethyl cellulose (HEC), hydroxypropyl cellulose (HPC), polyethylene oxide, hydroxypropyl-β-cyclodextrin (HP-β-CD), sulfobutylether-β-cyclodextrin (Captisol), cyclodextrin (e.g., γ-cyclodextrin), hydroxypropyl methylcellulose acetate succinate (HPMCAS), polyethylene glycol (PEG), polyvinyl caprolactam-polyvinyl acetate-polyethylene glycol graft copolymer (PVAc-PVCap-PEG), polysaccharide, poly(methacrylic acid-co-methyl methacrylate) (Eudragit), poloxamers, silica gel, aluminosilicate, or a combination thereof. In some embodiments, the one or more polymers comprise PVP-VA or HPMCAS. In some embodiments, the one or more polymers comprise HPMCAS. In some embodiments, the one or more polymers comprise PVP-VA. In some embodiments, the one or more polymers comprise copovidone. In some embodiments, the amorphous solid dispersion comprises about 10 wt % to about 90 wt % of the one or more polymers. In some embodiments, the amorphous solid dispersion comprises about 40 wt % to about 80 wt % of the one or more polymers. In some embodiments, the amorphous solid dispersion comprises about 55 wt % to about 65 wt % of the one or more polymers. In some embodiments, the amorphous solid dispersion comprises about 10 wt % to about 90 wt % of the Compound A or a pharmaceutically acceptable salt thereof. In some embodiments, the amorphous solid dispersion comprises about 20 wt % to about 60 wt % of the Compound A or a pharmaceutically acceptable salt thereof. In some embodiments, the amorphous solid dispersion comprises about 35 wt % to about 45 wt % of the Compound A or a pharmaceutically acceptable salt thereof. In some embodiments, a weight ratio of the Compound A or a pharmaceutically acceptable salt thereof to the one or more polymers is 2:1 to 1:2. In some embodiments, a weight ratio of the Compound A or a pharmaceutically acceptable salt thereof to the one or more polymers is about 1:1 to 1:2. In some embodiments, the amorphous solid dispersion comprises Compound A. In some embodiments, the amorphous solid dispersion is a hot melt extrudate (HME). In some embodiments, the amorphous solid dispersion is prepared by dissolving the Compound A or a pharmaceutically acceptable salt thereof in a solvent and then removing at least a part of the solvent. In some embodiments, the amorphous solid dispersion is prepared by dissolving the Compound A or a pharmaceutically acceptable salt thereof in a solvent and then removing at least a part of the solvent. In some embodiments, the particles in the amorphous solid dispersion have a D50 value of about 10 μm to about 500 μm. In some embodiments, the particles in the amorphous solid dispersion have a D50 value of about 100 μm to about 400 μm. In some embodiments, the solid formulation comprises about 10 wt % to about 80 wt % of the amorphous solid dispersion. In some embodiments, the solid formulation comprises about 40 wt % to about 60 wt % of the amorphous solid dispersion. In some embodiments, the solid formulation comprises about 15 wt % to about 35 wt % of the amorphous solid dispersion. In some embodiments, the one or more pharmaceutically acceptable excipients are in a mixture with the amorphous solid dispersion in the solid formulation. In some embodiments, the amorphous solid dispersion comprises one or more pharmaceutically acceptable excipients. In some embodiments, the solid formulation comprises about 5 wt % to about 80 wt % of the one or more pharmaceutically acceptable excipients. In some embodiments, the solid formulation comprises about 40 wt % to about 60 wt % of the one or more pharmaceutically acceptable excipients. In some embodiments, the one or more pharmaceutically acceptable excipients are selected from an antifoam, a flow-aid, a surfactant, a filler, a colorant, a preservative, a flavoring agent, a sweetener, and a combination thereof. In some embodiments, the one or more pharmaceutically acceptable excipients are selected from an antifoam, a flow-aid, a surfactant, a filler, and a combination thereof. In some embodiments, the antifoam comprises simethicone. In some embodiments, the solid formulation comprises about 0.1 wt % to about 15 wt % of the antifoam. In some embodiments, the solid formulation comprises about 1 wt % to about 8 wt % of the antifoam. In some embodiments, the solid formulation comprises about 3 wt % to about 8 wt % of the antifoam. In some embodiments, the solid formulation comprises about 0.5 wt % to about 3 wt % of simethicone. In some embodiments, the flow-aid is selected from silicon dioxide, magnesium stearate, talc, starch, magnesium silicate, hydrated sodium sulfoaluminate, and a combination thereof. In some embodiments, the silicon dioxide comprises fumed silica, colloidal silicon dioxide (CSD), or both. In some embodiments, the solid formulation comprises about 0.25 wt % to about 10 wt % of the flow-aid. In some embodiments, the solid formulation comprises about 1 wt % to about 5 wt % of the flow-aid. In some embodiments, the surfactant comprises cationic surfactant, anionic surfactant, non-ionic surfactant, or a combination thereof. In some embodiments, the surfactant comprises sodium lauryl sulfate (SLS). In some embodiments, the solid formulation comprises about 0.01 wt % to about 10 wt % of the surfactant. In some embodiments, the solid formulation comprises about 0.1 wt % to about 2 wt % of the surfactant. In some embodiments, the filler comprises a polysaccharide, a sugar or a derivative thereof, or both. In some embodiments, the filler comprises cellulose, starch, a synthetic soluble fiber, a sugar alcohol, or a combination thereof. In some embodiments, the filler comprises mannitol. In some embodiments, the filler comprises microcrystalline cellulose, polydextrose, sodium carboxymethyl cellulose (sodium CMC), or a combination thereof. In some embodiments, the solid formulation comprises about 5 wt % to about 80 wt % of the filler. In some embodiments, the solid formulation comprises about 30 wt % to about 75 wt % of the filler. In some embodiments, the instructions for aqueous reconstitution are in the form of a reference that refers to the instructions.

In some embodiments, the solid formulation in the kit comprises:
(a) about 10 wt % to about 60 wt % of an amorphous solid dispersion comprising (i) about 40 wt % to about 60 wt % of Compound A and (ii) about 40 wt % to about 60 wt % of PVP-VA, wherein the amorphous solid dispersion is a hot melt extrudate;
(b) about 30 wt % to 70 wt % of a filler, wherein the filler comprises microcrystalline cellulose and mannitol;
(c) about 0.1 wt % to 5 wt % of a surfactant, wherein the surfactant is SLS;
(d) about 0.25 wt % to 6 wt % of a flow-aid, wherein the flow-aid is CSD; and
(e) about 0.5 wt % to 5 wt % of an antifoam, wherein the antifoam is simethicone.

In some embodiments, the kit comprises one or more doses of compound A or a pharmaceutically acceptable salt thereof. In some embodiments, the kit comprises 1 to 5 unit doses of compound A or a pharmaceutically acceptable salt thereof. In some embodiments, the kit comprises a single unit dose of compound A or a pharmaceutically acceptable salt thereof. In some embodiments, the dose is a pediatric dose. In some embodiments, each dose comprises about 50 mg to about 800 mg of compound A or a pharmaceutically acceptable salt thereof. In some embodiments, each dose comprises about 300 mg or about 600 mg of compound A. In some embodiments, the kit further comprises one or more containers that optionally comprise markings denoting the measurement of volume. In certain aspects the present disclosure provides a kit comprising a pharmaceutical powder as disclosed herein for reconstitution.

In certain aspects, the present disclosure provides a unit dose composition for a powder formulation of Compound A, wherein the powder formulation comprises: Compound A, and optionally one or more of the following: one or more polymers, one or more fillers, a surfactant, an anti-foam, a flow-aid, a sweetener, and a natural flavor. In certain embodiments, the present disclosure provides a unit dose composition for a powder formulation of Compound A, wherein the powder formulation comprises: Compound A, one or more polymers, one or more fillers, a surfactant, an anti-foam, a flow-aid, a sweetener, and a natural flavor. In some embodiments, the one or more polymers can be a polymer as described herein. In some embodiments, the one or more polymers is copovidone. In some embodiments, the one or more fillers can be a filler as described herein. In some embodiments, the one or more fillers are microcrystalline cellulose, mannitol, maltodextrin, or a combination thereof. In some embodiments, the surfactant can be a surfactant as described herein. In some embodiments, the surfactant is sodium lauryl sulfate. In some embodiments, the anti-foam can be an anti-foam as described herein. In some embodiments, the anti-foam is simethicone. In some embodiments, the flow-aid can be a flow-aid as described herein. In some embodiments, the flow-aid is colloidal silicon dioxide. In some embodiments, the sweetener can be a sweetener as described herein. In some embodiments, the sweetener is sucralose powder. In some embodiments, the natural flavor can be a natural flavor as described herein. In some embodiments, the natural flavor is artificial strawberry powder.

In some embodiments, the powder formulation of Formula A comprises: about 5% to about 15% w/w of Compound A, and optionally one or more of the following: one or more polymers, one or more fillers, a surfactant, an anti-foam, a flow-aid, a sweetener, and a natural flavor. In certain embodiments, the powder formulation of Formula A comprises about 5% to about 20% w/w of one or more polymers, about 50% to about 70% w/w of one or more fillers, about 0.1% to about 1% w/w of surfactant, about 0.5% to about 2.5% w/w of anti-foam, about 1% to about 5% w/w of flow-aid, about 0.10% to about 1% w/w of sweetener, and about 0.5% to 1.5% w/w of natural flavor. In some embodiments, the powder formulation of Formula A comprises: about 9% to about 15% w/w of Compound A, about 10% to about 20% w/w of one or more polymers, about 60% to about 70% w/w of one or more fillers, about 0.5% to about 1% w/w of surfactant, about 1.5% to about 2.5% w/w of anti-foam, about 4% to about 5% w/w of flow-aid, about 0.5% to about 1% w/w of sweetener, and about 1.0% to 1.5% w/w of natural flavor. In some embodiments, the powder formulation of Formula A comprises: about 14% to about 15% w/w of Compound A, about 14% to about 15% w/w of one or more polymers, about 66% to about 67% w/w of one or more fillers, about 0.7% to about 0.8% w/w of surfactant, about 2.2% to about 2.3% w/w of anti-foam, about 4.4% to about 4.5% w/w of flow-aid, about 0.5% to about 0.6% w/w of sweetener, and about 1.5% to 1.6% w/w of natural flavor.

In some embodiments, the powder formulation of Formula A comprises: about 5% to about 15% w/w of Compound A, about 5% to about 20% w/w copovidone, about 50% to about 70% w/w of fillers including microcrystalline cellulose, mannitol, and maltodextrin, about 0.1% to about 1% w/w of sodium lauryl sulfate, about 0.5% to about 2.5% w/w of simethicone, about 1% to about 5% w/w of colloidal silicon dioxide, about 0.1% to about 1% w/w of sucralose powder, and about 0.5% to 1.5% w/w of artificial strawberry flavor. In some embodiments, the powder formulation of Formula A comprises: about 9% to about 15% w/w of Compound A, about 10% to about 20% w/w of copovidone, about 60% to about 70% w/w of microcrystalline cellulose, mannitol, and maltodextrin, about 0.5% to about 1% w/w of sodium lauryl sulfate, about 1.5% to about 2.5% w/w of simethicone, about 4% to about 5% w/w of colloidal silicon dioxide, about 0.5% to about 1% w/w of sucralose powder, and about 1.0% to 1.5% w/w of artificial strawberry flavor. In some embodiments, the powder formulation of Formula A comprises: about 14% to about 15% w/w of Compound A, about 14% to about 15% w/w of copovidone, about 66% to about 67% w/w of microcrystalline cellulose, mannitol, and maltodextrin, about 0.7% to about 0.8% w/w of sodium lauryl sulfate, about 2.2% to about 2.3% w/w of simethicone, about 4.4% to about 4.5% w/w of colloidal silicon dioxide, about 0.5% to about 0.6% w/w of sucralose powder, and about 1.5% to 1.6% w/w of artificial flavor, e.g., strawberry flavor.

In some embodiments, the powder formulation of Formula A comprises: about 5% to about 15% w/w of Compound A, about 5% to about 20% w/w copovidone, about 25% to about 35% w/w of microcrystalline cellulose, about 25% to about 35% w/w of mannitol, and about 2% to about 8% w/w of maltodextrin, about 0.1% to about 1% w/w of sodium lauryl sulfate, about 0.5% to about 2.5% w/w of simethicone, about 1% to about 5% w/w of colloidal silicon dioxide, about 0.1% to about 1% w/w of sucralose powder, and about 0.5% to 1.5% w/w of artificial strawberry flavor. In some embodiments, the powder formulation of Formula A comprises: about 9% to about 15% w/w of Compound A, about 10% to about 20% w/w of copovidone, about 30% to about 35% w/w of microcrystalline cellulose, about 30% to about 35% w/w of mannitol, and about 4% to about 6% w/w of maltodextrin, about 0.5% to about 1% w/w of sodium lauryl sulfate, about 1.5% to about 2.5% w/w of simethicone, about 4% to about 5% w/w of colloidal silicon dioxide, about 0.5% to about 1% w/w of sucralose powder, and about 1.0% to 1.5% w/w of artificial flavor, e.g., strawberry flavor. In some embodiments, the powder formulation of Formula A comprises: about 14% to about 15% w/w of Compound A, about 14% to about 15% w/w of copovidone, about 30% to about 31% w/w of microcrystalline cellulose, about 30% to about 31% w/w of mannitol, and about 5% to about 6% w/w of maltodextrin, about 0.7% to about 0.8% w/w of sodium lauryl sulfate, about 2.2% to about 2.3% w/w of simethicone, about 4.4% to about 4.5% w/w of colloidal silicon dioxide, about 0.5% to about 0.6% w/w of sucralose powder, and about 1.5% to 1.6% w/w of artificial flavor, e.g., strawberry flavor.

In some embodiments, the powder formulation of Formula A comprises: about 350 mg to about 450 mg of Compound A, about 600 mg to about 700 mg of one or more polymers, about 2500 mg to about 3500 mg of one or more fillers, about 25 mg to about 50 mg of surfactant, about 75 mg to about 150 mg of anti-foam, about 150 mg to about 250 mg of flow-aid, about 10 mg to about 75 mg of sweetener, and about 50 mg to 100 mg of natural flavor. In some embodiments, the powder formulation of Formula A comprises: about 400 mg to about 450 mg of Compound A, about 600 mg to about 650 mg of one or more polymers, about 2500 mg to about 3000 mg of one or more fillers, about 30 mg to about 40 mg of surfactant, about 75 mg to about 100 mg of anti-foam, about 150 mg to about 200 mg of flow-aid, about 10 mg to about 30 mg of sweetener, and about 50 mg to 75 mg of natural flavor. In some embodiments, the powder formulation of Formula A comprises: about 420 mg to about 430 mg of Compound A, about 640 mg to about 650 mg of one or more polymers, about 2850 mg to about 3000 mg of one or more fillers, about 30 mg to about 35 mg of surfactant, about 90 mg to about 100 mg of anti-foam, about 190 mg to about 200 mg of flow-aid, about 20 mg to about 30 mg of sweetener, and about 60 mg to 70 mg of natural or artificial flavor.

In some embodiments, the powder formulation of Formula A comprises: about 350 mg to about 450 mg of Compound A, about 600 mg to about 700 mg of copovidone, about 2500 mg to about 3500 mg of microcrystalline cellulose, mannitol, and maltodextrin, about 25 mg to about 50 mg of sodium lauryl sulfate, about 75 mg to about 150 mg of simethicone, about 150 mg to about 250 mg of colloidal silicon dioxide, about 10 mg to about 75 mg of sucralose powder, and about 50 mg to 100 mg of artificial strawberry flavor. In some embodiments, the powder formulation of Formula A comprises: about 400 mg to about 450 mg of Compound A, about 600 mg to about 650 mg of copovidone, about 2500 mg to about 3000 mg of microcrystalline cellulose, mannitol, and maltodextrin, about 30 mg to about 40 mg of sodium lauryl sulfate, about 75 mg to about 100 mg of simethicone, about 150 mg to about 200 mg of colloidal silicon dioxide, about 10 mg to about 30 mg of sucralose powder, and about 50 mg to 75 mg of artificial strawberry flavor. In some embodiments, the powder formulation of Formula A comprises: about 420 mg to about 430 mg of Compound A, about 640 mg to about 650 mg of copovidone, about 2850 mg to about 3000 mg of microcrystalline cellulose, mannitol, and maltodextrin, about 30 mg to about 35 mg of sodium lauryl sulfate, about 90 mg to about 100 mg of simethicone, about 190 mg to about 200 mg of colloidal silicon dioxide, about 20 mg to about 30 mg of sucralose powder, and about 60 mg to 70 mg of artificial flavor, e.g., strawberry flavor.

In some embodiments, the powder formulation of Formula A comprises: about 350 mg to about 450 mg of Compound A, about 600 mg to about 700 mg of copovidone, about 1000 mg to about 1500 mg of microcrystalline cellulose, about 1000 mg to about 1500 of mannitol, about 175 mg to about 250 of maltodextrin, about 25 mg to about 50 mg of sodium lauryl sulfate, about 75 mg to about 150 mg of simethicone, about 150 mg to about 250 mg of colloidal silicon dioxide, about 10 mg to about 75 mg of sucralose powder, and about 50 mg to 100 mg of artificial strawberry flavor. In some embodiments, the powder formulation of Formula A comprises: about 400 mg to about 450 mg of Compound A, about 600 mg to about 650 mg of copovidone, about 1200 mg to about 1500 mg of microcrystalline cellulose, about 1200 mg to about 1500 of mannitol, about 200 mg to about 250 of maltodextrin, about 30 mg to about 40 mg of sodium lauryl sulfate, about 75 mg to about 100 mg of simethicone, about 150 mg to about 200 mg of colloidal silicon dioxide, about 10 mg to about 30 mg of sucralose powder, and about 50 mg to 75 mg of artificial strawberry flavor. In some embodiments, the powder formulation of Formula A comprises: about 420 mg to about 430 mg of Compound A, about 640 mg to about 650 mg of copovidone, about 1300 mg to about 1400 mg of microcrystalline cellulose, about 1300 mg to about 1400 of mannitol, about 200 mg to about 240 of maltodextrin, about 30 mg to about 35 mg of sodium lauryl sulfate, about 90 mg to about 100 mg of simethicone, about 190 mg to about 200 mg of colloidal silicon dioxide, about 20 mg to about 30 mg of sucralose powder, and about 60 mg to 70 mg of artificial flavor, e.g., strawberry flavor.

In certain aspects the present disclosure provides methods of preparing an aqueous formulation comprising: (a) providing a solid formulation of an amorphous solid dispersion, wherein the amorphous solid dispersion comprises: (i) (R)-2-(1-(6-amino-5-chloropyrimidine-4-carboxamido)ethyl)-N-(5-chloro-4-(trifluoromethyl)pyridin-2-yl)thiazole-5-carboxamide (Compound A) or a pharmaceutically acceptable salt thereof and (ii) one or more polymers; and (b) contacting the solid formulation with an aqueous solution. In some embodiments, the contacting comprises mixing the solid formulation with the aqueous solution. In some embodiments, the mixing comprises shaking the solid formulation and the aqueous solution. In certain aspects the present disclosure provides a method of preparing an oral liquid suspension of Compound A or a salt thereof, comprising reconstituting the solid formulation in a kit as disclosed herein in an aqueous solution. In certain aspects the present disclosure provides a method of preparing an oral liquid suspension of Compound A or a salt thereof, comprising reconstituting a pharmaceutical powder as disclosed herein in an aqueous solution. In some embodiments, the kit comprises a pharmaceutical powder as disclosed herein for reconstitution.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates exemplary admixture instructions (Steps 1 through 12).

FIG. 2 illustrates a plot of mean plasma concentrations (Cₘₐₓ) (ng/mL) vs. time (h), following PO administration of DAY101 to male CD-1 mice.

FIGS. 3A-3D illustrate relative bioavailability of HME tablet and PfR (powder for reconstitution) by suspension geometric mean plasma concentration (ng/mL) vs. nominal time post-dose (hr), following administration of 300 mg DAY101 in Regimen G1 and Regimen H1 (FIGS. 3A-B) and 100 mg DAY101 in Regimen G2 and Regimen H2 (FIGS. 3C-3D).

FIG. 4A-4D illustrate relative bioavailability comparing the fed state and fasted state by geometric mean plasma concentration (ng/mL) vs. nominal time post-dose (h), following administration of 300 mg DAY101 in Regimen G1 and Regimen I1 (FIGS. 4A-4B) and 100 mg DAY101 in Regimen G2 and Regimen I2 (FIGS. 4C-4D).

FIG. 5 illustrates the overall taste/palatability assessments on a stacked bar chart: taste/palatability analysis set (Part 1). (Subjects taste 5 mL Day101 HME PfR oral suspension, 6 different formulations (Regimen A-F=Formulation 1-6); Key for grade: 1=Dislike extremely, 2=Dislike very much, 3=Dislike moderately, 4=Dislike slightly, 5=Neither like nor dislike, 6=like slightly, 7=Like moderately, 8=Like very much, 9=Like extremely; Results: Dislike (Grades 1-3), Neutral (Grades 4-6), Like (Grades 7-9))

FIG. 6 illustrates geometric mean (×/÷ geometric SD) plasma concentrations of DAY101 (ng/mL) following single oral doses of 300 mg DAY101 to healthy male and female subjects administered as tablet and suspension formulations in the fasted state: Regimen H1 and Regimen G1 (Log₁₀/Linear).

FIG. 7 illustrates geometric mean (×/÷ geometric SD) plasma concentrations of DAY101 (ng/mL) following single oral doses of 100 mg DAY101 to healthy male and female subjects administered as tablet and suspension formulations in the fasted state: Regimen H2 and Regimen G2 (Log 10/Linear).

FIG. 8 illustrates geometric mean (×/÷ geometric SD) plasma concentrations of DAY101 (ng/mL) following single oral doses of 300 mg DAY101 to healthy male and female subjects administered as tablet formulations in the fed and fasted states: Regimen I1 and Regimen G1 (Log 10/Linear).

FIG. 9 illustrates geometric mean (×/÷ geometric SD) plasma concentrations of DAY101 (ng/mL) following single oral doses of 100 mg DAY101 to healthy male and female subjects administered as tablet formulations in the fed and fasted states: Regimen I2 and Regimen G2 (Log 10/Linear).

FIG. 10 illustrates exemplary admixture instructions (Section A: Steps 1 through 17).

FIG. 11 illustrates exemplary instructions of giving a dose of the disclosed liquid suspension through a feeding tube (Section B: Steps 19-23).

### DETAILED DESCRIPTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Definitions

Unless defined otherwise, all technical and scientific terms used herein have the same meaning as is commonly understood by one of skill in the art to which this invention belongs. All patents and publications referred to herein are incorporated by reference.

As used in the specification and claims, the singular form “a”, “an” and “the” includes plural references unless the context clearly dictates otherwise.

The term “solid dispersion” or “solid formulation” as used herein refers to an amorphous dispersion comprising Compound A or a pharmaceutically acceptable salt thereof a solid state that is optionally prepared by hot melt extrusion.

The term “amorphous” as used herein refers to the solid dispersion or solid formulation comprising a solid form of Compound A or a salt thereof, that lacks the long-range order characteristic of a crystal, i.e., the solid is non-crystalline.

The term “micron” or “μm” as used herein refer to “micrometer,” which is 1×10⁻⁶ meter.

The term “in vivo” is used to describe an event that takes place in a subject's body.

The term “ex vivo” is used to describe an event that takes place outside of a subject's body. An ex vivo assay is not performed on a subject. Rather, it is performed upon a sample separate from a subject. An example of an ex vivo assay performed on a sample is an “in vitro” assay.

The term “in vitro” is used to describe an event that takes place in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

The terms “subject,” “individual,” and “patient” may be used interchangeably and refer to humans, the as well as non-human mammals (e.g., non-human primates, canines, equines, felines, porcine, bovines, ungulates, lagomorphs, and the like). In various embodiments, the subject can be a human (e.g., adult male, adult female, adolescent male, adolescent female, male child, female child) under the care of a physician or other health worker in a hospital, as an outpatient, or other clinical context. In certain embodiments, the subject may not be under the care or prescription of a physician or other health worker.

As used herein, the phrase “a subject in need thereof” refers to a subject, as described infra, that suffers from, or is at risk for, a pathology to be prophylactically or therapeutically treated with a compound or salt described herein.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “administer”, “administered”, “administers” and “administering” are defined as providing a composition to a subject via a route known in the art, including but not limited to intravenous, intraarterial, oral, parenteral, enteral, nasal, buccal, topical, transdermal, rectal, intramuscular, subcutaneous, intraosseous, transmucosal, or intraperitoneal routes of administration. In certain embodiments, oral routes of administering a composition can be used.

The terms “administer”, “administered”, “administers” and “administering” a compound should be understood to mean providing a compound of the disclosure or a prodrug of a compound of the disclosure to the individual in need.

The term “effective amount” or “therapeutically effective amount” refers to that amount of a solid dispersion, liquid formulation or solid formulation, comprising Compound A or a salt thereof, sufficient to treat one or more symptoms of cancer, or cause regression of the cancer. For example, in one embodiment, a therapeutically effective amount will refer to the amount of Compound A that decreases the rate of tumor growth, decreases tumor mass, decreases the number of metastases, increases time to tumor progression, or increases survival time by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%.

The term “pharmaceutically acceptable salt” as used herein refers to those salts suitable for use in contact with the tissues of humans without undue toxicity, irritation, allergic response and the like, and are commensurate with a reasonable benefit/risk ratio. Pharmaceutically acceptable salts are well known in the art. See Berge et al., J. Pharmaceutical Sciences, 1977, 66, 1-19.

The term “pharmaceutically acceptable excipient” or “excipient” as used herein refers to any ingredient in a Composition of the Disclosure other than the solid dispersion of Compound A or a salt thereof. An excipient is typically an inert substance added to a composition to facilitate processing, handling, administration, etc. of the composition. Useful excipients include, but are not limited to, adjuvants, anti-adherents, binders, carriers, disintegrants, fillers, flavors, colors, diluents, lubricants, glidants, preservatives, sorbents, solvents, surfactants, and sweeteners.

Conventional pharmaceutical excipients are well known to those of skill in the art. In particular, one of skill in the art will recognize that a wide variety of pharmaceutically acceptable excipients can be used in admixture with the solid dispersion of Compound 1 and vinylpyrrolidone-vinyl acetate copolymer, including those listed in the Handbook of Pharmaceutical Excipients, Pharmaceutical Press 4th Ed. (2003), and Remington: The Science and Practice of Pharmacy, Lippincott Williams & Wilkins, 21st ed. (2005).

As used herein, “treatment” or “treating” refers to an approach for obtaining beneficial or desired results with respect to a disease, disorder, or medical condition including, but not limited to, a therapeutic benefit. In certain embodiments, treatment or treating involves administering a compound or composition disclosed herein to a subject. A therapeutic benefit may include the eradication or amelioration of the underlying disorder being treated. Also, a therapeutic benefit may be achieved with the eradication or amelioration of one or more of the physiological symptoms associated with the underlying disorder, such as observing an improvement in the subject, notwithstanding that the subject may still be afflicted with the underlying disorder. Treating can include, for example, reducing, delaying or alleviating the severity of one or more symptoms of the disease or condition, or it can include reducing the frequency with which symptoms of a disease, defect, disorder, or adverse condition, and the like, are experienced by a patient. Treating can be used herein to refer to a method that results in some level of treatment or amelioration of the disease or condition, and can contemplate a range of results directed to that end.

In certain embodiments, the term “prevent” or “preventing” as related to a disease or disorder may refer to a compound that, in a statistical sample, reduces the occurrence of the disorder or condition in the treated sample relative to an untreated control sample, or delays the onset or reduces the severity of one or more symptoms of the disorder or condition relative to the untreated control sample. In certain embodiments, “prevent” or “preventing” includes administering the compounds or compositions to a subject at risk of developing a particular disease, or to a subject reporting one or more of the physiological symptoms of a disease, even though a diagnosis of this disease may not have been made.

The term “vinylpyrrolidone-vinyl acetate copolymer” as used herein refers a polymer comprising vinylpyrrolidone and vinyl acetate. Names and abbreviations for vinylpyrrolidone-vinyl acetate copolymer include, but are not limited to, copovidone, copovidonum, copolyvidone, copovidon, PVP-VAc-Copolymer. Copovidone is a vinylpyrrolidone-vinyl acetate copolymer comprised of 6 parts of vinylpyrrolidone and 4 parts of vinyl acetate e.g., CAS 25086-89-9. Examples of copovidone commercial products are Kollidon® VA 64 and Kollidon® 64 Fine. Another example is “Plasdone S-630,” a 60:40 random copolymer of N-vinyl pyrrolidinone and vinyl acetate.

“HPMCAS” refers to Hypromellose acetate succinate, a polymer containing acetyl and succinoyl groups. There are different types and grades of HPMCAS (e.g., HPMCAS-LG, HPMCAS-MG, HPMCAS-HG), which dissolve at different pHs due to different composition and ratio of its functional groups (e.g., acetyl, succinoyl). HPMCAS comes in L, M, and H grades dependent on content of acetyl and succinoyl wt %. The particle sizes for HPMCAS are fine (F) and granular (G).

“HPMCP” refers to a hydroxypropyl methylcellulose phthalate polymer. There are different types and grades of HPMCP (e.g., HP-55s, HP-50, HP-55), which dissolve at different pHs due to different composition and ratio of its functional groups (e.g., phthalyl).

“HPC” refers to hydroxypropyl cellulose. There are different types and grades of HPC (e.g., HPC-SSL, HPC-SL, HOC-SLT).

The term “w/w” means by weight. For example, 50% w/w means that the mass of the substance is 50% of the total mass of the solution or mixture.

As used herein, the term “mass median diameter” or “D₅₀” describes the diameter where 50 mass-% of the particles in a powder dispersion have a larger equivalent diameter, and the other 50 mass-% have a smaller equivalent diameter as determined by laser diffraction in Malvern Master Sizer Microplus equipment or its equivalent, or other suitable techniques. For example, if the D₅₀ of a powder dispersion is 105 μm, then 50% of the particles are larger than 105 μm, and 50% of the particles are smaller than 105 μm. Likewise, the term “D₉₀” describes the diameter where 90 mass-% of the particles in a powder dispersion have a smaller equivalent diameter, and the other 10 mass-% have a larger equivalent diameter. The term “D₁₀” describes the diameter where 10 mass-% of the particles in a powder dispersion have a smaller equivalent diameter, and the other 90 mass-% have a larger equivalent diameter

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “at most”, “less than,” or “less than or equal to” precedes the first numerical value in a series of two or more numerical values, the term “no more than,” “at most”, “less than,” or “less than or equal to” applies to each of the numerical values in that series of numerical values. For example, less than or equal to 3, 2, or 1 is equivalent to less than or equal to 3, less than or equal to 2, or less than or equal to 1.

The term “about,” as used herein, includes the recited number ±10%. Thus, “about 10” means 9 to 11. The term “about” can also refer to values within an acceptable error range for the particular value as determined by one of ordinary skill in the art, which will depend in part on how the value is measured or determined, e.g., the limitations of the measurement system. In an example, “about” can mean within 1 standard deviation, per the practice in the given value.

The section headings used herein are for organizational purposes only and are not to be construed as limiting the subject matter described.

Any aspect or embodiment described herein can be combined with any other aspect or embodiment as disclosed herein.

### Pharmaceutical Powder and Other Solid Form Pharmaceutical Compositions, Liquid Formulations

In certain aspects, the present disclosure provides a solid form pharmaceutical composition that comprises (R)-2-(1-(6-amino-5-chloropyrimidine-4-carboxamido)ethyl)-N-(5-chloro-4-(trifluoromethyl) pyridin-2-yl) thiazole-5-carboxamide (Compound A). In some embodiments, the solid form pharmaceutical composition is in a powder, granule, flake, or pellet form. In some embodiments, the solid form pharmaceutical composition is in a tablet or capsule form.

In certain aspects, the present disclosure provides a pharmaceutical powder. In some embodiments, the pharmaceutical powder comprises an amorphous solid dispersion of (R)-2-(1-(6-amino-5-chloropyrimidine-4-carboxamido)ethyl)-N-(5-chloro-4-(trifluoromethyl) pyridin-2-yl) thiazole-5-carboxamide (Compound A) or a pharmaceutically acceptable salt thereof, wherein the solid formulation comprises one or more pharmaceutically acceptable excipients. In some embodiments, the pharmaceutical powder comprises (a) an amorphous solid dispersion that comprises (R)-2-(1-(6-amino-5-chloropyrimidine-4-carboxamido)ethyl)-N-(5-chloro-4-(trifluoromethyl) pyridin-2-yl)thiazole-5-carboxamide (Compound A) or a pharmaceutically acceptable salt thereof; and (b) one or more pharmaceutically acceptable excipients, wherein the excipients comprises a flow-aid and a surfactant. In some embodiments, the pharmaceutical powder is configured to be reconstituted into an oral liquid suspension. In some embodiments, the powder for oral suspension contains 25 mg/mL tovorafenib after reconstitution with water and the following inactive ingredients: microcrystalline cellulose, copovidone, colloidal silicon dioxide, mannitol, sodium lauryl sulfate, simethicone, maltodextrin, sucralose, and strawberry flavor.

In some embodiments, each tablet of Compound A contains 100 mg tovorafemb and the following inactive ingredients: microcrystalline cellulose, copovidone, croscarmellose sodium, colloidal silicon dioxide, magnesium stearate, and OPADRY® Orange.

In certain aspects the present disclosure provides an oral liquid suspension. In some embodiments, the oral liquid suspension comprises, a pharmaceutical powder or other solid formulation as disclosed herein, and an aqueous solution. In some embodiments, an oral liquid suspension produced by contacting the pharmaceutical powder as disclosed herein with an aqueous solution. In some embodiments, the aqueous solution is water. In some embodiments, the oral liquid suspension is reconstituted. In some embodiments, the oral liquid suspension comprises, a solid formulation in a kit as disclosed herein, and an aqueous solution. In some embodiments, an oral liquid suspension produced by contacting the solid formulation in a kit as disclosed herein with an aqueous solution. In some embodiments, the tablets and powder for oral suspension may be used interchangeably.

In some embodiments, a solid formulation described herein such as a pharmaceutical powder comprises about 10 wt % to about 80 wt % of the amorphous solid dispersion that comprises Compound A or a pharmaceutically acceptable salt thereof. In some embodiments, a solid formulation described herein such as a pharmaceutical powder comprises about 15 wt % to about 80 wt % of the amorphous solid dispersion. In some embodiments, the solid formulation comprises about 20 wt % to about 80 wt % of the amorphous solid dispersion. In some embodiments, the solid formulation comprises about 25 wt % to about 80 wt % of the amorphous solid dispersion. In some embodiments, the solid formulation comprises about 30 wt % to about 80 wt % of the amorphous solid dispersion. In some embodiments, the solid formulation comprises about 35 wt % to about 80 wt % of the amorphous solid dispersion. In some embodiments, the solid formulation comprises about 40 wt % to about 80 wt % of the amorphous solid dispersion. In some embodiments, the solid formulation comprises about 45 wt % to about 80 wt % of the amorphous solid dispersion. In some embodiments, the solid formulation comprises about 50 wt % to about 80 wt % of the amorphous solid dispersion. In some embodiments, the solid formulation comprises about 55 wt % to about 80 wt % of the amorphous solid dispersion. In some embodiments, the solid formulation comprises about 60 wt % to about 80 wt % of the amorphous solid dispersion. In some embodiments, the solid formulation comprises about 65 wt % to about 80 wt % of the amorphous solid dispersion. In some embodiments, the solid formulation comprises about 70 wt % to about 80 wt % of the amorphous solid dispersion. In some embodiments, the solid formulation comprises about 75 wt % to about 80 wt % of the amorphous solid dispersion. In some embodiments, the solid formulation comprises about 20 wt % to about 75 wt % of the amorphous solid dispersion. In some embodiments, the solid formulation comprises about 20 wt % to about 70 wt % of the amorphous solid dispersion. In some embodiments, the solid formulation comprises about 20 wt % to about 65 wt % of the amorphous solid dispersion. In some embodiments, the solid formulation comprises about 20 wt % to about 60 wt % of the amorphous solid dispersion. In some embodiments, the solid formulation comprises about 20 wt % to about 55 wt % of the amorphous solid dispersion. In some embodiments, the solid formulation comprises about 20 wt % to about 50 wt % of the amorphous solid dispersion. In some embodiments, the solid formulation comprises about 20 wt % to about 45 wt % of the amorphous solid dispersion. In some embodiments, the solid formulation comprises about 20 wt % to about 40 wt % of the amorphous solid dispersion. In some embodiments, the solid formulation comprises about 20 wt % to about 35 wt % of the amorphous solid dispersion. In some embodiments, the solid formulation comprises about 20 wt % to about 30 wt % of the amorphous solid dispersion. In some embodiments, the solid formulation comprises about 20 wt % to about 25 wt % of the amorphous solid dispersion. In some embodiments, the solid formulation comprises about 10 wt % to about 75 wt % of the amorphous solid dispersion. In some embodiments, the solid formulation comprises about 10 wt % to about 70 wt % of the amorphous solid dispersion. In some embodiments, the solid formulation comprises about 10 wt % to about 65 wt % of the amorphous solid dispersion. In some embodiments, the solid formulation comprises about 10 wt % to about 60 wt % of the amorphous solid dispersion. In some embodiments, the solid formulation comprises about 10 wt % to about 55 wt % of the amorphous solid dispersion. In some embodiments, the solid formulation comprises about 10 wt % to about 50 wt % of the amorphous solid dispersion. In some embodiments, the solid formulation comprises about 10 wt % to about 45 wt % of the amorphous solid dispersion. In some embodiments, the solid formulation comprises about 10 wt % to about 40 wt % of the amorphous solid dispersion. In some embodiments, the solid formulation comprises about 10 wt % to about 35 wt % of the amorphous solid dispersion. In some embodiments, the solid formulation comprises about 10 wt % to about 30 wt % of the amorphous solid dispersion. In some embodiments, the solid formulation comprises about 10 wt % to about 25 wt % of the amorphous solid dispersion. In some embodiments, the solid formulation is a powder. In some embodiments, the solid formulation is in a form of granules or pellets.

In some embodiments, a solid formulation described herein such as a pharmaceutical powder comprises about 5 wt %, about 10 wt %, about 15 wt %, about 20 wt %, about 25 wt %, about 30 wt %, about 35 wt %, about 40 wt %, about 45 wt %, about 50 wt %, about 55 wt %, about 60 wt %, about 65 wt %, about 70 wt %, about 75 wt %, about 80 wt %, about 85 wt %, about 90 wt %, or about 95 wt % of the amorphous solid dispersion. In some embodiments, the solid formulation comprises at least 5 wt %, at least 10 wt %, at least 15 wt %, at least 20 wt %, at least 25 wt %, at least 30 wt %, at least 35 wt %, at least 40 wt %, at least 45 wt %, at least 50 wt %, at least 55 wt %, at least 60 wt %, at least 65 wt %, at least 70 wt %, at least 75 wt %, at least 80 wt %, at least 85 wt %, at least 90 wt %, or at least 95 wt % of the amorphous solid dispersion. In some embodiments, the solid formulation comprises at most at most 5 wt %, at most 10 wt %, at most 15 wt %, at most 20 wt %, at most 25 wt %, at most 30 wt %, at most 35 wt %, at most 40 wt %, at most 45 wt %, at most 50 wt %, at most 55 wt %, at most 60 wt %, at most 65 wt %, at most 70 wt %, at most 75 wt %, at most 80 wt %, at most 85 wt %, at most 90 wt %, or at most 95 wt % of the amorphous solid dispersion. In some embodiments, the solid formulation is a powder. In some embodiments, the solid formulation is in a form of granules or pellets.

### Amorphous Solid Dispersion

In one aspect, described herein is an amorphous solid dispersion that comprises Compound A or a pharmaceutically acceptable salt thereof. In some embodiments, the amorphous solid dispersion has a D50 value of about 10 μm to about 500 μm. In some embodiments, the amorphous solid dispersion has a D50 value of about 50 μm to about 500 μm. In some embodiments, the amorphous solid dispersion has a D50 value of about 100 μm to about 500 μm. In some embodiments, the amorphous solid dispersion has a D50 value of about 150 μm to about 500 μm. In some embodiments, the amorphous solid dispersion has a D50 value of about 200 μm to about 500 μm. In some embodiments, the amorphous solid dispersion has a D50 value of about 250 μm to about 500 μm. In some embodiments, the amorphous solid dispersion has a D50 value of about 300 μm to about 500 μm. In some embodiments, the amorphous solid dispersion has a D50 value of about 350 μm to about 500 μm. In some embodiments, the amorphous solid dispersion has a D50 value of about 400 μm to about 500 μm. In some embodiments, the amorphous solid dispersion has a D50 value of about 450 μm to about 500 μm. In some embodiments, In some embodiments, the amorphous solid dispersion has a D50 value of about 50 μm to about 450 μm. In some embodiments, In some embodiments, the amorphous solid dispersion has a D50 value of about 50 μm to about 400 μm. In some embodiments, In some embodiments, the amorphous solid dispersion has a D50 value of about 50 μm to about 350 μm. In some embodiments, In some embodiments, the amorphous solid dispersion has a D50 value of about 50 μm to about 300 μm. In some embodiments, In some embodiments, the amorphous solid dispersion has a D50 value of about 50 μm to about 250 μm. In some embodiments, In some embodiments, the amorphous solid dispersion has a D50 value of about 50 μm to about 200 μm. In some embodiments, In some embodiments, the amorphous solid dispersion has a D50 value of about 50 μm to about 150 μm. In some embodiments, In some embodiments, the amorphous solid dispersion has a D50 value of about 50 μm to about 100 μm. In some embodiments, In some embodiments, the amorphous solid dispersion has a D50 value of about 50 μm to about 100 μm. In some embodiments, In some embodiments, the amorphous solid dispersion has a D50 value of about 10 μm to about 450 μm. In some embodiments, In some embodiments, the amorphous solid dispersion has a D50 value of about 10 μm to about 400 μm. In some embodiments, In some embodiments, the amorphous solid dispersion has a D50 value of about 10 μm to about 350 μm. In some embodiments, In some embodiments, the amorphous solid dispersion has a D50 value of about 10 μm to about 300 μm. In some embodiments, In some embodiments, the amorphous solid dispersion has a D50 value of about 10 μm to about 250 μm. In some embodiments, In some embodiments, the amorphous solid dispersion has a D50 value of about 10 μm to about 200 μm. In some embodiments, In some embodiments, the amorphous solid dispersion has a D50 value of about 10 μm to about 150 μm. In some embodiments, In some embodiments, the amorphous solid dispersion has a D50 value of about 10 μm to about 100 μm. In some embodiments, In some embodiments, the amorphous solid dispersion has a D50 value of about 10 μm to about 100 μm

In some embodiments, the amorphous solid dispersion has a D50 value of about 10 μm, about 15 μm, about 20 μm, about 25 μm, about 30 μm, about 35 μm, about 40 μm, about 45 μm, about 50 μm, about 55 μm, about 60 μm, about 65 μm, about 70 μm, about 75 μm, about 80 μm, about 85 μm, about 90 μm, about 95 μm, about 100 μm, about 105 μm, about 110 μm, about 115 μm, about 120 μm, about 125 μm, about 130 μm, about 135 μm, about 140 μm, about 145 μm, about 150 μm, about 155 μm, about 160 μm, about 165 μm, about 170 μm, about 175 μm, about 180 μm, about 185 μm, about 190 μm, about 195 μm, about 200 μm, about 205 μm, about 210 μm, about 220 μm, about 220 μm, about 230 μm, about 240 μm, about 250 μm, about 260 μm, about 270 μm, about 280 μm, about 290 μm, about 300 μm, about 310 μm, about 320 μm, about 330 μm, about 340 μm, about 350 μm, about 360 μm, about 370 μm, about 380 μm, about 390 μm, about 400 μm, about 410 μm, about 420 μm, about 430 μm, about 440 μm, about 450 μm, about 460 μm, about 470 μm, about 480 μm, about 490 μm, or about 500 μm. In some embodiment, the solid dispersion has a D50 value of at least 10 μm, at least 15 μm, at least 20 μm, at least 25 μm, at least 30 μm, at least 35 μm, at least 40 μm, at least 45 μm, at least 50 μm, at least 75 μm, at least 80 μm, at least 85 μm, at least 90 μm, at least 95 μm, at least 100 μm, at least 110 μm, at least 120 μm, at least 125 μm, at least 150 μm, at least 175 μm, at least 200 μm, at least 250 μm, at least 300 μm, at least 350 μm, at least 360 μm, at least 370 μm, at least 380 μm, at least 390 μm, at least 400 μm, at least 410 μm, at least 420 μm, at least 430 μm, at least 440 μm, at least 450 μm, at least 460 μm, at least 470 μm, at least 480 μm, at least 490 μm, or at least 500 μm. In some embodiments, the solid dispersion has a D50 value of at most 10 μm, at most 15 μm, at most 20 μm, at most 25 μm, at most 30 μm, at most 35 μm, at most 40 μm, at most 45 μm, at most 50 μm, at most 75 μm, at most 80 μm, at most 85 μm, at most 90 μm, at most 95 μm, at most 100 μm, at most 110 μm, at most 120 μm, at most 125 μm, at most 150 μm, at most 175 μm, at most 200 μm, at most 250 μm, at most 300 μm, at most 350 μm, at most 360 μm, at most 370 μm, at most 380 μm, at most 390 μm, at most 400 μm, at most 410 μm, at most 420 μm, at most 430 μm, at most 440 μm, at most 450 μm, at most 460 μm, at most 470 μm, at most 480 μm, at most 490 μm, or at most 500 μm. In some embodiments, the D50 is determined by sieving particle size analysis.

An amorphous solid dispersion can be produced by any suitable methods known in the art. In some embodiments, the amorphous solid dispersion of the present disclosure is produced by hot melt extrusion. In some embodiments, the amorphous solid dispersion of the present disclosure is produced by spray-drying. In some embodiments, the amorphous solid dispersion of the present disclosure is produced by dissolving Compound A or a salt thereof and a polymer in a solution and then removing the solvent.

### Amorphous Solid Dispersion—Polymers

In one aspect, described herein is an amorphous solid dispersion that comprises Compound A or a pharmaceutically acceptable salt thereof. In some embodiments, the amorphous solid dispersion comprises one or more polymers. In some embodiments, the one or more polymers comprise polyvinylpyrrolidone, vinylpyrrolidone-vinyl acetate copolymer (PVP-VA), cross linked polyvinyl N-pyrrolidone, polyvinyl alcohol (PVA), polysaccharide, hydroxypropyl methylcellulose (HPMC or Hypromellose), hydroxyethyl cellulose (HEC), hydroxypropyl cellulose (HPC), polyethylene oxide, hydroxypropyl-β-cyclodextrin (HP-β-CD), sulfobutylether-β-cyclodextrin (Captisol), cyclodextrin (e.g., γ-cyclodextrin), hydroxypropyl methylcellulose acetate succinate (HPMCAS), polyethylene glycol (PEG), polyvinyl caprolactam-polyvinyl acetate-polyethylene glycol graft copolymer (PVAc-PVCap-PEG), polysaccharide, poly(methacrylic acid-co-methyl methacrylate) (Eudragit), poloxamers, silica gel, aluminosilicate, or a combination thereof. In some embodiments, the one or more polymers comprise vinylpyrrolidone-vinyl acetate copolymer (PVP-VA) or HPMCAS. In some embodiments, the one or more polymers comprise HPMCAS. In some embodiments, the one or more polymers comprise vinylpyrrolidone-vinyl acetate copolymer (PVP-VA).

In some embodiments, the amorphous solid dispersion comprises about 10 wt % to about 90 wt % of the one or more polymers. In some embodiments, the amorphous solid dispersion comprises about 10 wt % to about 80 wt % of the one or more polymers. In some embodiments, the amorphous solid dispersion comprises about 10 wt % to about 70 wt % of the one or more polymers. In some embodiments, the amorphous solid dispersion comprises about 10 wt % to about 60 wt % of the one or more polymers. In some embodiments, the amorphous solid dispersion comprises about 10 wt % to about 50 wt % of the one or more polymers. In some embodiments, the amorphous solid dispersion comprises about 10 wt % to about 40 wt % of the one or more polymers. In some embodiments, the amorphous solid dispersion comprises about 10 wt % to about 30 wt % of the one or more polymers. In some embodiments, the amorphous solid dispersion comprises about 10 wt % to about 20 wt % of the one or more polymers. In some embodiments, the amorphous solid dispersion comprises about 20 wt % to about 90 wt % of the one or more polymers. In some embodiments, the amorphous solid dispersion comprises about 20 wt % to about 80 wt % of the one or more polymers. In some embodiments, the amorphous solid dispersion comprises about 20 wt % to about 70 wt % of the one or more polymers. In some embodiments, the amorphous solid dispersion comprises about 20 wt % to about 60 wt % of the one or more polymers. In some embodiments, the amorphous solid dispersion comprises about 20 wt % to about 50 wt % of the one or more polymers. In some embodiments, the amorphous solid dispersion comprises about 20 wt % to about 40 wt % of the one or more polymers. In some embodiments, the amorphous solid dispersion comprises about 20 wt % to about 30 wt % of the one or more polymers. In some embodiments, the amorphous solid dispersion comprises about 30 wt % to about 90 wt % of the one or more polymers. In some embodiments, the amorphous solid dispersion comprises about 40 wt % to about 80 wt % of the one or more polymers. In some embodiments, the amorphous solid dispersion comprises about 50 wt % to about 70 wt % of the one or more polymers. In some embodiments, the amorphous solid dispersion comprises about 40 wt % to about 70 wt % of the one or more polymers. In some embodiments, the amorphous solid dispersion comprises about 40 wt % to about 60 wt % of the one or more polymers. In some embodiments, the amorphous solid dispersion comprises about 20 wt % to about 90 wt % of the one or more polymers.

In some embodiments, the amorphous solid dispersion comprises about 5 wt %, about 10 wt %, about 15 wt %, about 20 wt %, about 25 wt %, about 30 wt %, about 35 wt %, about 40 wt %, about 45 wt %, about 50 wt %, about 55 wt %, about 60 wt %, about 65 wt %, about 70 wt %, about 75 wt %, about 80 wt %, about 85 wt %, about 90 wt %, or about 95 wt % of the one or more polymers. In some embodiments, the amorphous solid dispersion comprises at least 5 wt %, at least 10 wt %, at least 15 wt %, at least 20 wt %, at least 25 wt %, at least 30 wt %, at least 35 wt %, at least 40 wt %, at least 45 wt %, at least 50 wt %, at least 55 wt %, at least 60 wt %, at least 65 wt %, at least 70 wt %, at least 75 wt %, at least 80 wt %, at least 85 wt %, at least 90 wt %, or at least 95 wt % of the one or more polymers. at most 5 wt %, at most 10 wt %, at most 15 wt %, at most 20 wt %, at most 25 wt %, at most 30 wt %, at most 35 wt %, at most 40 wt %, at most 45 wt %, at most 50 wt %, at most 55 wt %, at most 60 wt %, at most 65 wt %, at most 70 wt %, at most 75 wt %, at most 80 wt %, at most 85 wt %, at most 90 wt %, or at most 95 wt % of the one or more polymers.

In some embodiments, a suspension described herein comprises one or more polymers as disclosed herein. In some embodiments, a concentration of the one or more polymers in the suspension is about 10 mg/mL to about 200 mg/mL. In some embodiments, a concentration of the one or more polymers in the suspension is about 10 mg/mL to about 150 mg/mL. In some embodiments, a concentration of the one or more polymers in the suspension is about 10 mg/mL to about 125 mg/mL. In some embodiments, a concentration of the one or more polymers in the suspension is about 10 mg/mL to about 100 mg/mL. In some embodiments, a concentration of the one or more polymers in the suspension is about 10 mg/mL to about 75 mg/mL. In some embodiments, a concentration of the one or more polymers in the suspension is about 10 mg/mL to about 50 mg/mL. In some embodiments, a concentration of the one or more polymers in the suspension is about 10 mg/mL to about 25 mg/mL. In some embodiments, a concentration of the one or more polymers in the suspension is about 20 mg/mL to about 200 mg/mL. In some embodiments, a concentration of the one or more polymers in the suspension is about 20 mg/mL to about 150 mg/mL. In some embodiments, a concentration of the one or more polymers in the suspension is about 20 mg/mL to about 125 mg/mL. In some embodiments, a concentration of the one or more polymers in the suspension is about 20 mg/mL to about 100 mg/mL. In some embodiments, a concentration of the one or more polymers in the suspension is about 20 mg/mL to about 75 mg/mL. In some embodiments, a concentration of the one or more polymers in the suspension is about 20 mg/mL to about 50 mg/mL. In some embodiments, a concentration of the one or more polymers in the suspension is about 20 mg/mL to about 25 mg/mL. In some embodiments, the concentration of the one or more polymers in the suspension is about 30 mg/mL to about 125 mg/mL. In some embodiments, the concentration of the one or more polymers in the suspension is about 40 mg/mL to about 125 mg/mL. In some embodiments, the concentration of the one or more polymers in the suspension is about 40 mg/mL to about 125 mg/mL. In some embodiments, the concentration of the one or more polymers in the suspension is about 50 mg/mL to about 125 mg/mL. In some embodiments, the concentration of the one or more polymers in the suspension is about 60 mg/mL to about 125 mg/mL. In some embodiments, the concentration of the one or more polymers in the suspension is about 70 mg/mL to about 125 mg/mL. In some embodiments, the concentration of the one or more polymers in the suspension is about 80 mg/mL to about 125 mg/mL. In some embodiments, the concentration of the one or more polymers in the suspension is about 90 mg/mL to about 125 mg/mL. In some embodiments, the concentration of the one or more polymers in the suspension is about 100 mg/mL to about 125 mg/mL. In some embodiments, the concentration of the one or more polymers in the suspension is about 30 mg/mL to about 50 mg/mL. In some embodiments, the concentration of the one or more polymers in the suspension is about 30 mg/mL to about 60 mg/mL. In some embodiments, the concentration of the one or more polymers in the suspension is about 30 mg/mL to about 70 mg/mL. In some embodiments, the concentration of the one or more polymers in the suspension is about 30 mg/mL to about 80 mg/mL. In some embodiments, the concentration of the one or more polymers in the suspension is about 30 mg/mL to about 90 mg/mL. In some embodiments, the concentration of the one or more polymers in the suspension is about 30 mg/mL to about 100 mg/mL. In some embodiments, the concentration of the one or more polymers in the suspension is about 30 mg/mL to about 110 mg/mL. In some embodiments, the concentration of the one or more polymers in the suspension is about 30 mg/mL to about 120 mg/mL. In some embodiments, the concentration of the one or more polymers in the suspension is about 30 mg/mL to about 125 mg/mL. In some embodiments, the concentration of the one or more polymers in the suspension is about 50 mg/mL to about 60 mg/mL. In some embodiments, the concentration of the one or more polymers in the suspension is about 50 mg/mL to about 70 mg/mL. In some embodiments, the concentration of the one or more polymers in the suspension is about 50 mg/mL to about 80 mg/mL. In some embodiments, the concentration of the one or more polymers in the suspension is about 50 mg/mL to about 90 mg/mL. In some embodiments, the concentration of the one or more polymers in the suspension is about 50 mg/mL to about 100 mg/mL. In some embodiments, the concentration of the one or more polymers in the suspension is about 50 mg/mL to about 110 mg/mL. In some embodiments, the concentration of the one or more polymers in the suspension is about 50 mg/mL to about 120 mg/mL. In some embodiments, the concentration of the one or more polymers in the suspension is about 50 mg/mL to about 125 mg/mL. In some embodiments, the concentration of the one or more polymers in the suspension is about 60 mg/mL to about 70 mg/mL. In some embodiments, the concentration of the one or more polymers in the suspension is about 60 mg/mL to about 80 mg/mL. In some embodiments, the concentration of the one or more polymers in the suspension is about 60 mg/mL to about 90 mg/mL. In some embodiments, the concentration of the one or more polymers in the suspension is about 60 mg/mL to about 100 mg/mL. In some embodiments, the concentration of the one or more polymers in the suspension is about 60 mg/mL to about 110 mg/mL. In some embodiments, the concentration of the one or more polymers in the suspension is about 60 mg/mL to about 125 mg/mL.

In some embodiments, the concentration of the one or more polymers in the suspension is about 10 mg/mL, about 15 mg/mL, about 20 mg/mL, about 25 mg/mL, about 25 mg/mL, about 30 mg/mL, about 35 mg/mL, about 40 mg/mL, about 45 mg/mL, about 50 mg/mL, about 55 mg/mL, about 60 mg/mL, about 65 mg/mL, about 70 mg/mL, about 75 mg/mL, about 80 mg/mL, about 85 mg/mL, about 90 mg/mL, about 95 mg/mL, about 100 mg/mL, about 105 mg/mL, about 100 mg/mL, about 115 mg/mL, about 120 mg/mL or about 125 mg/mL. In some embodiments, the concentration of the one or more polymers in the suspension is at least 10 mg/mL, at least 15 mg/mL, at least 20 mg/mL, at least 25 mg/mL, at least 25 mg/mL, at least 30 mg/mL, at least 35 mg/mL, at least 40 mg/mL, at least 45 mg/mL, at least 50 mg/mL, at least 55 mg/mL, at least 60 mg/mL, at least 65 mg/mL, at least 70 mg/mL, at least 75 mg/mL, at least 80 mg/mL, at least 85 mg/mL, at least 90 mg/mL, at least 95 mg/mL, at least 100 mg/mL, at least 105 mg/mL, at least 100 mg/mL, at least 115 mg/mL, at least 120 mg/mL or at least 125 mg/mL. In some embodiments, the concentration of the one or more polymers in the suspension is at most 10 mg/mL, at most 15 mg/mL, at most 20 mg/mL, at most 25 mg/mL, at most 25 mg/mL, at most 30 mg/mL, at most 35 mg/mL, at most 40 mg/mL, at most 45 mg/mL, at most 50 mg/mL, at most 55 mg/mL, at most 60 mg/mL, at most 65 mg/mL, at most 70 mg/mL, at most 75 mg/mL, at most 80 mg/mL, at most 85 mg/mL, at most 90 mg/mL, at most 95 mg/mL, at most 100 mg/mL, at most 105 mg/mL, at most 100 mg/mL, at most 115 mg/mL, at most 120 mg/mL or at most 125 mg/mL.

### Amorphous Solid Dispersion—Compound A

In some embodiments, an amorphous solid dispersion disclosed herein comprises Compound A. In some embodiments, the amorphous solid dispersion comprises (R)-2-(1-(6-amino-5-chloropyrimidine-4-carboxamido)ethyl)-N-(5-chloro-4-(trifluoromethyl)pyridin-2-yl)thiazole-5-carboxamide. In some embodiments, the amorphous solid dispersion comprises

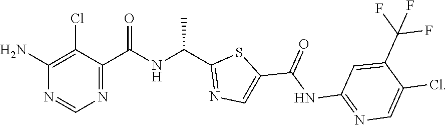 [SMILES: C[C@@H](NC(=O)c1ncnc(N)c1Cl)c1ncc(C(=O)Nc2cc(C(F)(F)F)c(Cl)cn2)s1]

In some embodiments, the amorphous solid dispersion is a hot melt extrudate (HME). In some embodiments, the amorphous solid dispersion is prepared by dissolving the Compound A or a pharmaceutically acceptable salt thereof in a solvent and then removing at least a part of the solvent. In some embodiments, Compound A or a pharmaceutically acceptable salt thereof is amorphous.

In some embodiments, the amorphous solid dispersion comprises about 10 wt % to about 90 wt % of the Compound A or a pharmaceutically acceptable salt thereof. In some embodiments, the amorphous solid dispersion comprises about 10 wt % to about 80 wt % of the Compound A or a pharmaceutically acceptable salt thereof. In some embodiments, the amorphous solid dispersion comprises about 10 wt % to about 70 wt % of the Compound A or a pharmaceutically acceptable salt thereof. In some embodiments, the amorphous solid dispersion comprises about 10 wt % to about 60 wt % of the Compound A or a pharmaceutically acceptable salt thereof. In some embodiments, the amorphous solid dispersion comprises about 10 wt % to about 50 wt % of the Compound A or a pharmaceutically acceptable salt thereof. In some embodiments, the amorphous solid dispersion comprises about 10 wt % to about 40 wt % of the Compound A or a pharmaceutically acceptable salt thereof. In some embodiments, the amorphous solid dispersion comprises about 10 wt % to about 30 wt % of the Compound A or a pharmaceutically acceptable salt thereof. In some embodiments, the amorphous solid dispersion comprises about 10 wt % to about 20 wt % of the Compound A or a pharmaceutically acceptable salt thereof. In some embodiments, the amorphous solid dispersion comprises about 20 wt % to about 90 wt % of the Compound A or a pharmaceutically acceptable salt thereof. In some embodiments, the amorphous solid dispersion comprises about 20 wt % to about 80 wt % of the Compound A or a pharmaceutically acceptable salt thereof. In some embodiments, the amorphous solid dispersion comprises about 20 wt % to about 70 wt % of the Compound A or a pharmaceutically acceptable salt thereof. In some embodiments, the amorphous solid dispersion comprises about 20 wt % to about 60 wt % of the Compound A or a pharmaceutically acceptable salt thereof. In some embodiments, the amorphous solid dispersion comprises about 20 wt % to about 50 wt % of the Compound A or a pharmaceutically acceptable salt thereof. In some embodiments, the amorphous solid dispersion comprises about 20 wt % to about 40 wt % of the Compound A or a pharmaceutically acceptable salt thereof. In some embodiments, the amorphous solid dispersion comprises about 20 wt % to about 30 wt % of the Compound A or a pharmaceutically acceptable salt thereof. In some embodiments, the amorphous solid dispersion comprises about 35 wt % to about 45 wt % of the Compound A or a pharmaceutically acceptable salt thereof. In some embodiments, the amorphous solid dispersion comprises about 30 wt % to about 90 wt % of the Compound A or a pharmaceutically acceptable salt thereof. In some embodiments, the amorphous solid dispersion comprises about 40 wt % to about 80 wt % of the Compound A or a pharmaceutically acceptable salt thereof. In some embodiments, the amorphous solid dispersion comprises about 50 wt % to about 70 wt % of the Compound A or a pharmaceutically acceptable salt thereof. In some embodiments, the amorphous solid dispersion comprises about 40 wt % to about 70 wt % of the Compound A or a pharmaceutically acceptable salt thereof. In some embodiments, the amorphous solid dispersion comprises about 40 wt % to about 60 wt % of the Compound A or a pharmaceutically acceptable salt thereof. In some embodiments, the amorphous solid dispersion comprises about 20 wt % to about 90 wt % of the Compound A or a pharmaceutically acceptable salt thereof.

In some embodiments, the amorphous solid dispersion comprises about 10 wt %, about 20 wt %, about 30 wt %, about 40 wt %, about 50 wt %, about 60 wt %, about 70 wt %, about 80 wt %, about 90 wt %, or about 95 wt % of the Compound A or a pharmaceutically acceptable salt thereof. In some embodiments, the amorphous solid dispersion comprises at least 10 wt %, at least 20 wt %, at least 30 wt %, at least 40 wt %, at least 50 wt %, at least 60 wt %, at least 70 wt %, at least 80 wt %, at least 90 wt %, at least 95 wt % of the Compound A or a pharmaceutically acceptable salt thereof. In some embodiments, the amorphous solid dispersion comprises at most 10 wt %, at most 20 wt %, at most 30 wt %, at most 40 wt %, at most 50 wt %, at most 60 wt %, at most 70 wt %, at most 80 wt %, at most 90 wt %, or at most 95 wt % of the Compound A or a pharmaceutically acceptable salt thereof.

In some embodiments, a weight ratio of the Compound A or a pharmaceutically acceptable salt thereof to the one or more polymers is from 10:1 to 1:10 or 5:1 to 1:5. In some embodiments, a weight ratio of the Compound A or a pharmaceutically acceptable salt thereof to the one or more polymers is from 2:1 to 1:2. In some embodiments, a weight ratio of the Compound A or a pharmaceutically acceptable salt thereof to the one or more polymers is about 1:1 to 1:2. In some embodiments, the weight ratio of the Compound A or a pharmaceutically acceptable salt thereof to the one or more polymers is from 2 to 0.5. In some embodiments, the weight ratio of the Compound A or a pharmaceutically acceptable salt thereof to the one or more polymers is from 2 to 0.5. In some embodiments, the weight ratio of the Compound A or a pharmaceutically acceptable salt thereof to the one or more polymers is from 2 to 0.75. In some embodiments, the weight ratio of the Compound A or a pharmaceutically acceptable salt thereof to the one or more polymers is from 2 to 1. In some embodiments, the weight ratio of the Compound A or a pharmaceutically acceptable salt thereof to the one or more polymers is from 2 to 1.25. In some embodiments, the weight ratio of the Compound A or a pharmaceutically acceptable salt thereof to the one or more polymers is from 2 to 1.5. In some embodiments, the weight ratio of the Compound A or a pharmaceutically acceptable salt thereof to the one or more polymers is from 2 to 1.75. In some embodiments, the weight ratio of the Compound A or a pharmaceutically acceptable salt thereof to the one or more polymers is from 1.75 to 0.5. In some embodiments, the weight ratio of the Compound A or a pharmaceutically acceptable salt thereof to the one or more polymers is from 1.50 to 0.5. In some embodiments, the weight ratio of the Compound A or a pharmaceutically acceptable salt thereof to the one or more polymers is from 1.25 to 0.5. In some embodiments, the weight ratio of the Compound A or a pharmaceutically acceptable salt thereof to the one or more polymers is from 1 to 0.5. In some embodiments, the weight ratio of the Compound A or a pharmaceutically acceptable salt thereof to the one or more polymers is from 0.75 to 0.5. In some embodiments, the weight ratio of the Compound A or a pharmaceutically acceptable salt thereof to the one or more polymers is from 1 to 1.

In some embodiments, the weight ratio of the Compound A or a pharmaceutically acceptable salt thereof to the one or more polymers is about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, or about 2.0. In some embodiments, the weight ratio of the Compound A or a pharmaceutically acceptable salt thereof to the one or more polymers is at least 0.5, at least 0.6, at least 0.7, at least 0.8, at least 0.9, at least 1.0, at least 1.1, at least 1.2, at least 1.3, at least 1.4, at least 1.5, at least 1.6, at least 1.7, at least 1.8, at least 1.9, or at least 2.0. In some embodiments, the weight ratio of the Compound A or a pharmaceutically acceptable salt thereof to the one or more polymers is at most 0.5, at most 0.6, at most 0.7, at most 0.8, at most 0.9, at most 1.0, at most 1.1, at most 1.2, at most 1.3, at most 1.4, at most 1.5, at most 1.6, at most 1.7, at most 1.8, at most 1.9, or at most 2.0.

In some embodiments, Compound A or a pharmaceutically acceptable salt thereof is in a form of an amorphous solid dispersion. In some embodiments, the concentration of Compound A or a pharmaceutically acceptable salt thereof is about 10 mg/mL to about 125 mg/mL in an oral liquid suspension described herein. In some embodiments, the concentration of Compound A or a pharmaceutically acceptable salt thereof is about 10 mg/mL to about 100 mg/mL in the oral liquid suspension. In some embodiments, the concentration of Compound A or a pharmaceutically acceptable salt thereof is about 10 mg/mL to about 75 mg/mL in the oral liquid suspension. In some embodiments, the concentration of Compound A or a pharmaceutically acceptable salt thereof is about 10 to about 50 mg/mL in the oral liquid suspension. In some embodiments, the concentration of Compound A or a pharmaceutically acceptable salt thereof is about 10 to about 30 mg/mL in the oral liquid suspension. In some embodiments, the concentration of Compound A or a pharmaceutically acceptable salt thereof is about 10 to about 35 mg/mL in the oral liquid suspension. In some embodiments, the concentration of Compound A or a pharmaceutically acceptable salt thereof is about 10 to about 40 mg/mL in the oral liquid suspension. In some embodiments, the concentration of Compound A or a pharmaceutically acceptable salt thereof is about 10 to about 45 mg/mL in the oral liquid suspension. In some embodiments, the concentration of Compound A or a pharmaceutically acceptable salt thereof is about 25 mg/mL to about 100 mg/mL in the oral liquid suspension. In some embodiments, the concentration of Compound A or a pharmaceutically acceptable salt thereof is about 25 mg/mL to about 75 mg/mL in the oral liquid suspension. In some embodiments, the concentration of Compound A or a pharmaceutically acceptable salt thereof is about 25 to about 50 mg/mL in the oral liquid suspension. In some embodiments, the concentration of Compound A or a pharmaceutically acceptable salt thereof is about 25 to about 30 mg/mL in the oral liquid suspension. In some embodiments, the concentration of Compound A or a pharmaceutically acceptable salt thereof is about 25 to about 35 mg/mL in the oral liquid suspension. In some embodiments, the concentration of Compound A or a pharmaceutically acceptable salt thereof is about 25 to about 40 mg/mL in the oral liquid suspension. In some embodiments, the concentration of Compound A or a pharmaceutically acceptable salt thereof is about 25 to about 45 mg/mL in the oral liquid suspension. In some embodiments, the concentration of Compound A or a pharmaceutically acceptable salt thereof is about 50 to about 125 mg/mL in the oral liquid suspension. In some embodiments, the concentration of Compound A or a pharmaceutically acceptable salt thereof is about 50 to about 100 mg/mL in the oral liquid suspension. In some embodiments, the concentration of Compound A or a pharmaceutically acceptable salt thereof is about 50 to about 75 mg/mL in the oral liquid suspension.

In some embodiments, the concentration of Compound A or a pharmaceutically acceptable salt thereof is about 10 mg/mL, about 15 mg/mL, about 20 mg/mL, about 25 mg/mL, about 30 mg/mL, about 35 mg/mL, about 40 mg/mL, about 45 mg/mL, about 50 mg/mL, about 55 mg/mL, about 65 mg/mL, about 70 mg/mL, about 75 mg/mL, about 80 mg/mL, about 85 mg/mL, about 90 mg/mL, about 95 mg/mL, about 100 mg/mL, about 105 mg/mL, about 110 mg/mL, about 115 mg/mL, about 120 mg/mL, or about 125 mg/mL in the oral liquid suspension. In some embodiments, the concentration of Compound A or a pharmaceutically acceptable salt thereof is about 10 mg/mL in the oral liquid suspension. In some embodiments, the concentration of Compound A or a pharmaceutically acceptable salt thereof is about 25 mg/mL in the oral liquid suspension. In some embodiments, the concentration of Compound A or a pharmaceutically acceptable salt thereof is about 50 mg/mL in the oral liquid suspension. In some embodiments, the concentration of Compound A or a pharmaceutically acceptable salt thereof is at least 10 mg/mL, at least 15 mg/mL, at least 20 mg/mL, at least 25 mg/mL, at least 30 mg/mL, at least 35 mg/mL, at least t 40 mg/mL, at least 45 mg/mL, at least 50 mg/mL, at least 55 mg/mL, at least 65 mg/mL, at least 70 mg/mL, at least 75 mg/mL, at least 80 mg/mL, at least 85 mg/mL, at least 90 mg/mL, at least 95 mg/mL, at least 100 mg/mL, at least 105 mg/mL, at least 110 mg/mL, at least 115 mg/mL, at least 120 mg/mL, or at least 125 mg/mL in the oral liquid suspension. In some embodiments, the concentration of Compound A or a pharmaceutically acceptable salt thereof is at most 10 mg/mL, at most 15 mg/mL, at most 20 mg/mL, at most 25 mg/mL, at most 30 mg/mL, at most 35 mg/mL, at most 40 mg/mL, at most 45 mg/mL, at most 50 mg/mL, at most 55 mg/mL, at most 65 mg/mL, at most 70 mg/mL, at most 75 mg/mL, at most 80 mg/mL, at most 85 mg/mL, at most 90 mg/mL, at most 95 mg/mL, at most 100 mg/mL, at most 105 mg/mL, at most 110 mg/mL, at most 115 mg/mL, at most 120 mg/mL, or at most 125 mg/mL in the oral liquid suspension.

### Pharmaceutically Acceptable Excipients

Pharmaceutical compositions described herein, e.g., solid form pharmaceutical compositions (such as pharmaceutical powders) and suspensions, can comprise one or more pharmaceutically acceptable excipients. In some embodiments, the one or more pharmaceutically acceptable excipients are in a mixture with the amorphous solid dispersion in the pharmaceutical powder. In some embodiments, the pharmaceutical powder comprises about 5 wt % to about 90 wt % of the one or more pharmaceutically acceptable excipients. In some embodiments, the pharmaceutical powder comprises about 5 wt % to about 80 wt % of the one or more pharmaceutically acceptable excipients. In some embodiments, the pharmaceutical powder comprises about 5 wt % to about 70 wt % of the one or more pharmaceutically acceptable excipients. In some embodiments, the pharmaceutical powder comprises about 5 wt % to about 60 wt % of the one or more pharmaceutically acceptable excipients. In some embodiments, the pharmaceutical powder comprises about 5 wt % to about 50 wt % of the one or more pharmaceutically acceptable excipients. In some embodiments, the pharmaceutical powder comprises about 5 wt % to about 40 wt % of the one or more pharmaceutically acceptable excipients. In some embodiments, the pharmaceutical powder comprises about 5 wt % to about 30 wt % of the one or more pharmaceutically acceptable excipients. In some embodiments, the pharmaceutical powder comprises about 5 wt % to about 20 wt % of the one or more pharmaceutically acceptable excipients. In some embodiments, the pharmaceutical powder comprises about 10 wt % to about 90 wt % of the one or more pharmaceutically acceptable excipients. In some embodiments, the pharmaceutical powder comprises about 10 wt % to about 80 wt % of the one or more pharmaceutically acceptable excipients. In some embodiments, the pharmaceutical powder comprises about 10 wt % to about 70 wt % of the one or more pharmaceutically acceptable excipients. In some embodiments, the pharmaceutical powder comprises about 10 wt % to about 60 wt % of the one or more pharmaceutically acceptable excipients. In some embodiments, the pharmaceutical powder comprises about 10 wt % to about 50 wt % of the one or more pharmaceutically acceptable excipients. In some embodiments, the pharmaceutical powder comprises about 10 wt % to about 40 wt % of the one or more pharmaceutically acceptable excipients. In some embodiments, the pharmaceutical powder comprises about 10 wt % to about 30 wt % of the one or more pharmaceutically acceptable excipients. In some embodiments, the pharmaceutical powder comprises about 40 wt % to about 80 wt % of the one or more pharmaceutically acceptable excipients. In some embodiments, the pharmaceutical powder comprises about 50 wt % to about 70 wt % of the one or more pharmaceutically acceptable excipients. In some embodiments, the pharmaceutical powder comprises about 40 wt % to about 70 wt % of the one or more pharmaceutically acceptable excipients. In some embodiments, the pharmaceutical powder comprises about 40 wt % to about 60 wt % of the one or more pharmaceutically acceptable excipients. In some embodiments, the pharmaceutical powder comprises about 20 wt % to about 90 wt % of the one or more pharmaceutically acceptable excipients. In some embodiments, the pharmaceutical powder comprises about 5 wt % to about 25 wt % of the one or more pharmaceutically acceptable excipients. In some embodiments, the pharmaceutical powder comprises about 5 wt % to about 20 wt % of the one or more pharmaceutically acceptable excipients. In some embodiments, the pharmaceutical powder comprises about 5 wt % to about 15 wt % of the one or more pharmaceutically acceptable excipients. In some embodiments, the pharmaceutical powder comprises about 5 wt % to about 10 wt % of the one or more pharmaceutically acceptable excipients.

In some embodiments, the pharmaceutical powder comprises about 5 wt %, about 10 wt %, about 15 wt %, about 20 wt %, about 25 wt %, about 30 wt %, about 35 wt %, about 40 wt %, about 45 wt %, about 50 wt %, about 55 wt %, about 60 wt %, about 65 wt %, about 70 wt %, about 75 wt %, about 80 wt %, about 85 wt %, about 90 wt %, or about 95 wt % of the one or more pharmaceutically acceptable excipients. In some embodiments, the pharmaceutical powder comprises at least 10 wt %, at least 20 wt %, at least 30 wt %, at least 40 wt %, at least 50 wt %, at least 60 wt %, at least 70 wt %, at least 80 wt %, at least 90 wt %, at least 95 wt % of the one or more pharmaceutically acceptable excipients. In some embodiments, the pharmaceutical powder comprises at most 5 wt %, at most 10 wt %, at most 15 wt %, at most 20 wt %, at most 25 wt %, at most 30 wt %, at most 35 wt %, at most 40 wt %, at most 45 wt %, at most 50 wt %, at most 55 wt %, at most 60 wt %, at most 65 wt %, at most 70 wt %, at most 75 wt %, at most 80 wt %, at most 85 wt %, at most 90 wt %, or at most 95 wt % of the one or more pharmaceutically acceptable excipients.

In some embodiments, a solid formulation described herein comprises one or more pharmaceutically acceptable excipients and an amorphous solid dispersion. In some embodiments, the solid formulation comprises about 5 wt % to about 90 wt % of the one or more pharmaceutically acceptable excipients (i.e., the one or more pharmaceutically acceptable excipients are present in the solid formulation at about 5 wt % to about 90 wt %). In some embodiments, the solid formulation comprises about 5 wt % to about 80 wt % of the one or more pharmaceutically acceptable excipients. In some embodiments, the solid formulation comprises about 5 wt % to about 70 wt % of the one or more pharmaceutically acceptable excipients. In some embodiments, the solid formulation comprises about 5 wt % to about 60 wt % of the one or more pharmaceutically acceptable excipients. In some embodiments, the solid formulation comprises about 5 wt % to about 50 wt % of the one or more pharmaceutically acceptable excipients. In some embodiments, the solid formulation comprises about 5 wt % to about 40 wt % of the one or more pharmaceutically acceptable excipients. In some embodiments, the solid formulation comprises about 5 wt % to about 30 wt % of the one or more pharmaceutically acceptable excipients. In some embodiments, the solid formulation comprises about 5 wt % to about 20 wt % of the one or more pharmaceutically acceptable excipients. In some embodiments, the solid formulation comprises about 10 wt % to about 90 wt % of the one or more pharmaceutically acceptable excipients. In some embodiments, the solid formulation comprises about 10 wt % to about 80 wt % of the one or more pharmaceutically acceptable excipients. In some embodiments, the solid formulation comprises about 10 wt % to about 70 wt % of the one or more pharmaceutically acceptable excipients. In some embodiments, the solid formulation comprises about 10 wt % to about 60 wt % of the one or more pharmaceutically acceptable excipients. In some embodiments, the solid formulation comprises about 10 wt % to about 50 wt % of the one or more pharmaceutically acceptable excipients. In some embodiments, the solid formulation comprises about 10 wt % to about 40 wt % of the one or more pharmaceutically acceptable excipients. In some embodiments, the solid formulation comprises about 10 wt % to about 30 wt % of the one or more pharmaceutically acceptable excipients. In some embodiments, the solid formulation comprises about 40 wt % to about 80 wt % of the one or more pharmaceutically acceptable excipients. In some embodiments, the solid formulation comprises about 50 wt % to about 70 wt % of the one or more pharmaceutically acceptable excipients. In some embodiments, the solid formulation comprises about 40 wt % to about 70 wt % of the one or more pharmaceutically acceptable excipients. In some embodiments, the solid formulation comprises about 40 wt % to about 60 wt % of the one or more pharmaceutically acceptable excipients. In some embodiments, the solid formulation comprises about 20 wt % to about 90 wt % of the one or more pharmaceutically acceptable excipients. In some embodiments, the solid formulation comprises about 5 wt % to about 25 wt % of the one or more pharmaceutically acceptable excipients. In some embodiments, the solid formulation comprises about 5 wt % to about 20 wt % of the one or more pharmaceutically acceptable excipients. In some embodiments, the solid formulation comprises about 5 wt % to about 15 wt % of the one or more pharmaceutically acceptable excipients. In some embodiments, the solid formulation comprises about 5 wt % to about 10 wt % of the one or more pharmaceutically acceptable excipients. In some embodiments, the solid formulation comprises about 5 wt %, about 10 wt %, about 15 wt %, about 20 wt %, about 25 wt %, about 30 wt %, about 35 wt %, about 40 wt %, about 45 wt %, about 50 wt %, about 55 wt %, about 60 wt %, about 65 wt %, about 70 wt %, about 75 wt %, about 80 wt %, about 85 wt %, about 90 wt %, or about 95 wt % of the one or more pharmaceutically acceptable excipients. In some embodiments, the solid formulation comprises at least 10 wt %, at least 20 wt %, at least 30 wt %, at least 40 wt %, at least 50 wt %, at least 60 wt %, at least 70 wt %, at least 80 wt %, at least 90 wt %, at least 95 wt % of the one or more pharmaceutically acceptable excipients. In some embodiments, the solid formulation comprises at most 5 wt %, at most 10 wt %, at most 15 wt %, at most 20 wt %, at most 25 wt %, at most 30 wt %, at most 35 wt %, at most 40 wt %, at most 45 wt %, at most 50 wt %, at most 55 wt %, at most 60 wt %, at most 65 wt %, at most 70 wt %, at most 75 wt %, at most 80 wt %, at most 85 wt %, at most 90 wt %, or at most 95 wt % of the one or more pharmaceutically acceptable excipients. In some embodiments, the solid formulation is a powder. In some embodiments, the solid formulation is in a form of granules or pellets.

In some embodiments, the one or more pharmaceutically acceptable excipients comprise an antifoam, a filler, a colorant, a preservative, a flavoring agent, a sweetener, or a combination thereof. In some embodiments, the one or more pharmaceutically acceptable excipients comprise a colorant, a preservative, a flavoring agent, a sweetener, or a combination thereof. In some embodiments, the one or more pharmaceutically acceptable excipients comprise a colorant. In some embodiments, the one or more pharmaceutically acceptable excipients comprises preservative. In some embodiments, the one or more pharmaceutically acceptable excipients comprises a flavoring agent. In some embodiments, the one or more pharmaceutically acceptable excipients comprises a sweetener.

Some pharmaceutical acceptable excipients are well known in the pharmaceutical art and described, for example, in Rowe et al., Handbook of Pharmaceutical Excipients: A Comprehensive Guide to Uses, Properties, and Safety, 5^{th} Ed., 2006, and in Remington: The Science and Practice of Pharmacy (Gennaro, 21st Ed. Mack Pub. Co., Easton, PA (2005)). Exemplary pharmaceutically acceptable excipients include sterile saline and phosphate buffered saline at physiological pH. Preservatives, stabilizers, dyes, buffers, and the like may be provided in the pharmaceutical composition. In addition, antioxidants and suspending agents may also be used. In general, the type of excipient is selected based on the mode of administration, as well as the chemical composition of the active ingredient(s). Alternatively, compositions described herein may be formulated as a lyophilizate. A composition described herein may be lyophilized or otherwise formulated as a lyophilized product using one or more appropriate excipient solutions for solubilizing and/or diluting the pharmaceutical agent(s) of the composition upon administration. In other embodiments, the pharmaceutical agent may be encapsulated within liposomes using technology known and practiced in the art. In certain particular embodiments, a pharmaceutical agent is not formulated within liposomes for application to a stent that is used for treating highly, though not totally, occluded arteries. Pharmaceutical compositions may be formulated for any appropriate manner of administration described herein and in the art.

Antifoam

In some embodiments, the one or more pharmaceutically acceptable excipients described herein comprise an antifoam. In some embodiments, the antifoam comprises simethicone or dimethicone. In some embodiments, the antifoam comprises simethicone. The antifoam can be in the form of powder on a solid carrier (e.g., antifoam and maltodextrin such as 30% Simethione/70% Maltodextrin). In some embodiments, the antifoam is carried by a solid carrier. In some embodiments, the solid carrier is maltodextrin, microcrystalline cellulose, or calcium carbonate. In some embodiments, the antifoam is in a liquid form. In some embodiments, the antifoam is a suspension.

In some embodiments, the solid formulation such as a pharmaceutical powder comprises about 0.1 wt % to about 15 wt % of the antifoam. In some embodiments, the solid formulation comprises about 0.1 wt % to about 14 wt % of the antifoam. In some embodiments, the solid formulation comprises about 0.1 wt % to about 13 wt % of the antifoam. In some embodiments, the solid formulation comprises about 0.1 wt % to about 12 wt % of the antifoam. In some embodiments, the solid formulation comprises about 0.1 wt % to about 11 wt % of the antifoam. In some embodiments, the solid formulation comprises about 0.1 wt % to about 10 wt % of the antifoam. In some embodiments, the solid formulation comprises about 0.1 wt % to about 9 wt % of the antifoam. In some embodiments, the solid formulation comprises about 0.1 wt % to about 8 wt % of the antifoam. In some embodiments, the solid formulation comprises about 5 wt % to about 8 wt % of the antifoam. In some embodiments, the solid formulation comprises about 0.1 wt % to about 7 wt % of the antifoam. In some embodiments, the solid formulation comprises about 0.1 wt % to about 6 wt % of the antifoam. In some embodiments, the solid formulation comprises about 0.1 wt % to about 5 wt % of the antifoam. In some embodiments, the solid formulation comprises about 0.1 wt % to about 4 wt % of the antifoam. In some embodiments, the solid formulation comprises about 0.1 wt % to about 3 wt % of the antifoam. In some embodiments, the solid formulation comprises about 0.1 wt % to about 2 wt % of the antifoam. In some embodiments, the solid formulation comprises about 0.1 wt % to about 1 wt % of the antifoam. In some embodiments, the solid formulation comprises about 0.1 wt % to about 0.9 wt % of the antifoam. In some embodiments, the solid formulation comprises about 0.1 wt % to about 0.8 wt % of the antifoam. In some embodiments, the solid formulation comprises about 0.1 wt % to about 0.7 wt % of the antifoam. In some embodiments, the solid formulation comprises about 0.1 wt % to about 0.6 wt % of the antifoam. In some embodiments, the solid formulation comprises about 0.1 wt % to about 0.5 wt % of the antifoam. In some embodiments, the solid formulation comprises about 0.1 wt % to about 0.4 wt % of the antifoam. In some embodiments, the solid formulation comprises about 0.1 wt % to about 0.3 wt % of the antifoam. In some embodiments, the solid formulation comprises about 0.1 wt % to about 0.25 wt % of the antifoam. In some embodiments, the solid formulation is a powder. In some embodiments, the solid formulation is in a form of flake, granules or pellets. In some embodiments, the antifoam comprises simethicone and a carrier such as maltodextrin. In some embodiments, the carrier is a starch. In some embodiments, the antifoam comprises simethicone and maltodextrin. In some embodiments, the antifoam comprises simethicone (about 30 wt %) and maltodextrin (about 70 wt %). In some embodiments, the antifoam comprises simethicone (30 wt %) and maltodextrin (70 wt %). In some embodiments, the antifoam comprises simethicone (20-40 wt %) and maltodextrin (60-80 wt %).

In some embodiments, the solid formulation such as pharmaceutical powder comprises about 0.1 wt % to about 0.2 wt % of the antifoam. In some embodiments, the solid formulation comprises about 0.1 wt % to about 0.15 wt % of the antifoam. In some embodiments, the solid formulation comprises about 0.15 wt % to about 15 wt % of the antifoam. In some embodiments, the solid formulation comprises about 0.2 wt % to about 15 wt % of the antifoam. In some embodiments, the solid formulation comprises about 0.25 wt % to about 15 wt % of the antifoam. In some embodiments, the solid formulation comprises about 0.3 wt % to about 15 wt % of the antifoam. In some embodiments, the solid formulation comprises about 0.4 wt % to about 15 wt % of the antifoam. In some embodiments, the solid formulation comprises about 0.5 wt % to about 15 wt % of the antifoam. In some embodiments, the solid formulation comprises about 0.6 wt % to about 15 wt % of the antifoam. In some embodiments, the solid formulation comprises about 0.7 wt % to about 15 wt % of the antifoam. In some embodiments, the solid formulation comprises about 0.8 wt % to about 15 wt % of the antifoam. In some embodiments, the solid formulation comprises about 0.9 wt % to about 15 wt % of the antifoam. In some embodiments, the solid formulation comprises about 1 wt % to about 15 wt % of the antifoam. In some embodiments, the solid formulation comprises about 2 wt % to about 15 wt % of the antifoam. In some embodiments, the solid formulation comprises about 3 wt % to about 15 wt % of the antifoam. In some embodiments, the solid formulation comprises about 4 wt % to about 15 wt % of the antifoam. In some embodiments, the solid formulation comprises about 5 wt % to about 15 wt % of the antifoam. In some embodiments, the solid formulation comprises about 6 wt % to about 15 wt % of the antifoam. In some embodiments, the solid formulation comprises about 7 wt % to about 15 wt % of the antifoam. In some embodiments, the solid formulation comprises about 8 wt % to about 15 wt % of the antifoam. In some embodiments, the solid formulation comprises about 9 wt % to about 15 wt % of the antifoam. In some embodiments, the solid formulation comprises about 10 wt % to about 15 wt % of the antifoam. In some embodiments, the solid formulation comprises about 11 wt % to about 15 wt % of the antifoam. In some embodiments, the solid formulation comprises about 12 wt % to about 15 wt % of the antifoam. In some embodiments, the solid formulation comprises about 13 wt % to about 15 wt % of the antifoam. In some embodiments, the solid formulation comprises about 14 wt % to about 15 wt % of the antifoam. In some embodiments, the solid formulation comprises about 2 wt % to about 10 wt % of the antifoam. In some embodiments, the solid formulation comprises about 3 wt % to about 10 wt % of the antifoam. In some embodiments, the solid formulation comprises about 3 wt % to about 9 wt % of the antifoam. In some embodiments, the solid formulation comprises about 3 wt % to about 8 wt % of the antifoam. In some embodiments, the solid formulation comprises about 0.1 wt % to about 15 wt % of simethicone. In some embodiments, the solid formulation comprises about 3 wt % to about 8 wt % of simethicone. In some embodiments, the solid formulation comprises about 0.5 wt % to about 3 wt % of simethicone. In some embodiments, the solid formulation is a powder. In some embodiments, the solid formulation is in a form of flake, granules or pellets.

In some embodiments, the solid formulation such as pharmaceutical powder comprises about 0.1 wt %, about 0.2 wt %, about 0.3 wt %, about 0.4 wt %, about 0.5 wt %, about 0.6 wt %, about 0.7 wt %, about 0.8 wt %, about 0.9 wt %, about 1 wt %, about 1.1 wt %, about 1.2 wt %, about 1.3 wt %, about 1.4 wt %, about 1.5 wt %, about 2 wt %, about 2.5 wt %, about 3 wt %, about 3.5 wt %, about 4 wt %, about 4.5 wt %, about 5 wt %, about 6 wt %, about 7 wt %, about 8 wt %, about 9 wt %, about 10 wt %, about 11 wt %, about 12 wt %, about 13 wt %, about 14 wt %, or about 15 wt % of the antifoam. In some embodiments, the solid formulation comprises from at least 0.1 wt %, at least 0.2 wt %, at least 0.3 wt %, at least 0.4 wt %, at least 0.5 wt %, at least 0.6 wt %, at least 0.7 wt %, at least 0.8 wt %, at least 0.9 wt %, at least 1 wt %, at least 1.1 wt %, at least 1.2 wt %, at least 1.3 wt %, at least 1.4 wt %, at least 1.5 wt %, at least 2 wt %, at least 2.5 wt %, at least 3 wt %, at least 3.5 wt %, at least 4 wt %, at least 4.5 wt %, at least 5 wt %, at least 6 wt %, at least 7 wt %, at least 8 wt %, at least 9 wt %, at least 10 wt %, at least 11 wt %, at least 12 wt %, at least 13 wt %, at least 14 wt %, or at least 15 wt % of the antifoam. In some embodiments, the solid formulation comprises at most 0.1 wt %, at most 0.2 wt %, at most 0.3 wt %, at most 0.4 wt %, at most 0.5 wt %, at most 0.6 wt %, at most 0.7 wt %, at most 0.8 wt %, at most 0.9 wt %, at most 1 wt %, at most 1.1 wt %, at most 1.2 wt %, at most 1.3 wt %, at most 1.4 wt %, at most 1.5 wt %, at most 2 wt %, at most 2.5 wt %, at most 3 wt %, at most 3.5 wt %, at most 4 wt %, at most 4.5 wt %, at most 5 wt %, at most 6 wt %, at most 7 wt %, at most 8 wt %, at most 9 wt %, at most 10 wt %, at most 11 wt %, at most 12 wt %, at most 13 wt %, at most 14 wt %, or at most 15 wt % of the antifoam. In some embodiments, the solid formulation is a powder. In some embodiments, the solid formulation is in a form of flake, granules or pellets.

In some embodiments, a suspension described herein comprises one or more pharmaceutically acceptable excipients as disclosed herein. In some embodiments, the one or more pharmaceutically acceptable excipients comprises an antifoam. In some embodiments, a concentration of the antifoam in the suspension is about 1 mg/mL to about 30 mg/mL. In some embodiments, a concentration of the antifoam in the suspension is about 1 mg/mL to about 25 mg/mL. In some embodiments, a concentration of the antifoam in the suspension is about 1 mg/mL to about 20 mg/mL. In some embodiments, a concentration of the antifoam in the suspension is about 1 mg/mL to about 15 mg/mL. In some embodiments, a concentration of the antifoam in the suspension is about 1 mg/mL to about 10 mg/mL. In some embodiments, a concentration of the antifoam in the suspension is about 1 mg/mL to about 9 mg/mL. In some embodiments, a concentration of the antifoam in the suspension is about 1 mg/mL to about 8 mg/mL. In some embodiments, a concentration of the antifoam in the suspension is about 1 mg/mL to about 7 mg/mL. In some embodiments, a concentration of the antifoam in the suspension is about 1 mg/mL to about 6 mg/mL. In some embodiments, a concentration of the antifoam in the suspension is about 1 mg/mL to about 5 mg/mL. In some embodiments, a concentration of the antifoam in the suspension is about 1 mg/mL to about 4 mg/mL. In some embodiments, a concentration of the antifoam in the suspension is about 1 mg/mL to about 3 mg/mL. In some embodiments, a concentration of the antifoam in the suspension is about 1 mg/mL to about 2 mg/mL. In some embodiments, a concentration of the antifoam in the suspension is about 5 mg/mL to about 30 mg/mL. In some embodiments, a concentration of the antifoam in the suspension is about 10 mg/mL to about 30 mg/mL. In some embodiments, a concentration of the antifoam in the suspension is about 15 mg/mL to about 30 mg/mL. In some embodiments, a concentration of the antifoam in the suspension is about 20 mg/mL to about 30 mg/mL. In some embodiments, a concentration of the antifoam in the suspension is about 25 mg/mL to about 30 mg/mL. In some embodiments, the concentration of the antifoam in the suspension is about 1 mg/mL to about 10 mg/mL. In some embodiments, the concentration of the antifoam in the suspension is about 3 mg/mL to about 8 mg/mL. In some embodiments, the concentration of the antifoam in the suspension is about 1 mg/mL, about 2 mg/mL, about 3 mg/mL, about 4 mg/mL, about 5 mg/mL, about 10 mg/mL, about 15 mg/m, about 20 mg/mL, about 25 mg/mL, or about 30 mg/mL. In some embodiments, the concentration of the antifoam in the suspension is at least 1 mg/mL, at least 2 mg/mL, at least 3 mg/mL, at least 4 mg/mL, at least 5 mg/mL, at least 10 mg/mL, at least 15 mg/mL, at least 20 mg/mL, at least 25 mg/mL, or at least 30 mg/mL. In some embodiments, the concentration of the antifoam in the suspension is at most 1 mg/mL, at most 2 mg/mL, at most 3 mg/mL, at most 4 mg/mL, at most 5 mg/mL, at most 10 mg/mL, at most 15 mg/mL, at most 20 mg/mL, at most 25 mg/mL, or at most 30 mg/mL.

Filler

In some embodiments, the one or more pharmaceutically acceptable excipients described herein comprise a filler. In some embodiments, the filler comprises a polysaccharide, a sugar or a derivative thereof, or both. In some embodiments, the filler comprises a polysaccharide. In some embodiments, the filler is a sugar. In some embodiments, the filler comprises cellulose, starch, a synthetic soluble fiber, a sugar alcohol, or a combination thereof. In some embodiments, the filler comprises cellulose. In some embodiments, the filler comprises starch. In some embodiments, the filler comprises maltodextrin. In some embodiments, the filler comprises HPMC. In some embodiments, the filler comprises dicalcium phosphate dihydrate (DCP). In some embodiments, the filler comprises a synthetic soluble fiber. In some embodiments, the filler comprises a sugar alcohol. In some embodiments, the filler comprises mannitol. In some embodiments, the filler comprises microcrystalline cellulose, polydextrose, sodium carboxymethyl cellulose (sodium CMC), or a combination thereof. In some embodiments, the filler comprises microcrystalline cellulose. In some embodiments, the filler comprises polydextrose. In some embodiments, the filler comprises sodium carboxymethyl cellulose.

In some embodiments, a solid formulation such as a pharmaceutical powder described herein comprises about 5 wt % to about 90 wt % of the filler (i.e., the filler is present in the solid formulation at about 5 wt % to about 90 wt %). In some embodiments, the solid formulation comprises about 5 wt % to about 80 wt % of the filler. In some embodiments, the solid formulation comprises about 5 wt % to about 70 wt % of the filler. In some embodiments, the solid formulation comprises about 5 wt % to about 60 wt % of the filler. In some embodiments, the solid formulation comprises about 5 wt % to about 50 wt % of the filler. In some embodiments, the solid formulation comprises about 5 wt % to about 40 wt % of the filler. In some embodiments, the solid formulation comprises about 5 wt % to about 30 wt % of the filler. In some embodiments, the solid formulation comprises about 5 wt % to about 20 wt % of the filler. In some embodiments, the solid formulation comprises about 10 wt % to about 90 wt % of the filler. In some embodiments, the solid formulation comprises about 10 wt % to about 80 wt % of the filler. In some embodiments, the solid formulation comprises about 10 wt % to about 70 wt % of the filler. In some embodiments, the solid formulation comprises about 10 wt % to about 60 wt % of the filler. In some embodiments, the solid formulation comprises about 10 wt % to about 50 wt % of the filler. In some embodiments, the solid formulation comprises about 10 wt % to about 40 wt % of the filler. In some embodiments, the solid formulation comprises about 10 wt % to about 30 wt % of the filler. In some embodiments, the solid formulation comprises about 40 wt % to about 80 wt % of the filler. In some embodiments, the solid formulation comprises about 50 wt % to about 70 wt % of the filler. In some embodiments, the solid formulation comprises about 40 wt % to about 70 wt % of the filler. In some embodiments, the solid formulation comprises about 40 wt % to about 60 wt % of the filler. In some embodiments, the solid formulation comprises about 20 wt % to about 90 wt % of the filler. In some embodiments, the solid formulation comprises about 5 wt % to about 25 wt % of the filler. In some embodiments, the solid formulation comprises about 5 wt % to about 20 wt % of the filler. In some embodiments, the solid formulation comprises about 5 wt % to about 15 wt % of the filler. In some embodiments, the solid formulation comprises about 5 wt % to about 10 wt % of the filler. In some embodiments, the solid formulation is a powder. In some embodiments, the solid formulation is in a form of flake, granules or pellets.

In some embodiments, the solid formulation such as pharmaceutical powder comprises about 5 wt %, about 10 wt %, about 15 wt %, about 20 wt %, about 25 wt %, about 30 wt %, about 35 wt %, about 40 wt %, about 45 wt %, about 50 wt %, about 55 wt %, about 60 wt %, about 65 wt %, about 70 wt %, about 75 wt %, about 80 wt %, about 85 wt %, about 90 wt %, or about 95 wt % of the filler. In some embodiments, the solid formulation comprises at least 5 wt %, at least 10 wt %, at least 15 wt %, at least 20 wt %, at least 25 wt %, at least 30 wt %, at least 35 wt %, at least 40 wt %, at least 45 wt %, at least 50 wt %, at least 55 wt %, at least 60 wt %, at least 65 wt %, at least 70 wt %, at least 75 wt %, at least 80 wt %, at least 85 wt %, at least 90 wt %, or at least 95 wt % of the filler. In some embodiments, the solid formulation comprises at most 5 wt %, at most 10 wt %, at most 15 wt %, at most 20 wt %, at most 25 wt %, at most 30 wt %, at most 35 wt %, at most 40 wt %, at most 45 wt %, at most 50 wt %, at most 55 wt %, at most 60 wt %, at most 65 wt %, at most 70 wt %, at most 75 wt %, at most 80 wt %, at most 85 wt %, at most 90 wt %, or at most 95 wt % of the filler. In some embodiments, the solid formulation is a powder. In some embodiments, the solid formulation is in a form of flake, granules or pellets.

In some embodiments, the suspension comprises one or more pharmaceutically acceptable excipients as disclosed herein.

In some embodiments, a concentration of the filler in the suspension is about 30 mg/mL to about 500 mg/mL. In some embodiments, a concentration of the filler in the suspension is about 40 mg/mL to about 500 mg/mL. In some embodiments, a concentration of the filler in the suspension is about 50 mg/mL to about 500 mg/mL. In some embodiments, a concentration of the filler in the suspension is about 60 mg/mL to about 500 mg/mL. In some embodiments, a concentration of the filler in the suspension is about 70 mg/mL to about 500 mg/mL. In some embodiments, a concentration of the filler in the suspension is about 80 mg/mL to about 500 mg/mL. In some embodiments, a concentration of the filler in the suspension is about 90 mg/mL to about 500 mg/mL. In some embodiments, a concentration of the filler in the suspension is about 100 mg/mL to about 500 mg/mL. In some embodiments, a concentration of the filler in the suspension is about 125 mg/mL to about 500 mg/mL. In some embodiments, a concentration of the filler in the suspension is about 150 mg/mL to about 500 mg/mL. In some embodiments, a concentration of the filler in the suspension is about 175 mg/mL to about 500 mg/mL. In some embodiments, a concentration of the filler in the suspension is about 200 mg/mL to about 500 mg/mL. In some embodiments, a concentration of the filler in the suspension is about 250 mg/mL to about 500 mg/mL. In some embodiments, a concentration of the filler in the suspension is about 300 mg/mL to about 500 mg/mL. In some embodiments, a concentration of the filler in the suspension is about 350 mg/mL to about 500 mg/mL. In some embodiments, a concentration of the filler in the suspension is about 400 mg/mL to about 500 mg/mL. In some embodiments, a concentration of the filler in the suspension is about 450 mg/mL to about 500 mg/mL. In some embodiments, a concentration of the filler in the suspension is about 50 mg/mL to about 450 mg/mL. In some embodiments, a concentration of the filler in the suspension is about 50 mg/mL to about 400 mg/mL. In some embodiments, a concentration of the filler in the suspension is about 50 mg/mL to about 350 mg/mL. In some embodiments, a concentration of the filler in the suspension is about 50 mg/mL to about 300 mg/mL. In some embodiments, a concentration of the filler in the suspension is about 50 mg/mL to about 250 mg/mL. In some embodiments, a concentration of the filler in the suspension is about 50 mg/mL to about 200 mg/mL. In some embodiments, a concentration of the filler in the suspension is about 50 mg/mL to about 150 mg/mL. In some embodiments, a concentration of the filler in the suspension is about 50 mg/mL to about 100 mg/mL. In some embodiments, the concentration of the filler in the suspension is about 100 mg/mL to about 250 mg/mL. In some embodiments, the concentration of the filler in the suspension is about 125 mg/mL to about 200 mg/mL. In some embodiments, the concentration of the filler in the suspension is about 50 mg/mL to about 150 mg/mL. In some embodiments, the concentration of the filler in the suspension is about 50 mg/mL, about 75 mg/mL, about 100 mg/mL, about 125 mg/mL, about 150 mg/mL, about 175 mg/mL, about 200 mg/mL, about 250 mg/mL, about 300 mg/mL, about 350 mg/mL, about 400 mg/mL, about 450 mg/mL, or about 500 mg/mL. In some embodiments, the concentration of the filler in the suspension is at least 50 mg/mL, at least 75 mg/mL, at least 100 mg/mL, at least 125 mg/mL, at least 150 mg/mL, at least 175 mg/mL, at least 200 mg/mL, at least 250 mg/mL, at least 300 mg/mL, at least 350 mg/mL, at least 400 mg/mL, at least 450 mg/mL, or at least 500 mg/mL. In some embodiments, the concentration of the filler in the suspension is at most 50 mg/mL, at most 75 mg/mL, at most 100 mg/mL, at most 125 mg/mL, at most 150 mg/mL, at most 175 mg/mL, at most 200 mg/mL, at most 250 mg/mL, at most 300 mg/mL, at most 350 mg/mL, at most 400 mg/mL, at most 450 mg/mL, or at most 500 mg/mL.

Flow-Aid

In some embodiments, one or more pharmaceutically acceptable excipients described herein comprise a flow-aid. In some embodiments, the flow-aid is a glidant. In some embodiments, the flow-aid is selected from silicon dioxide, magnesium stearate, talc, starch, magnesium silicate, hydrated sodium sulfoaluminate, and a combination thereof. In some embodiments, the flow-aid is selected from magnesium stearate, talc, starch, magnesium silicate, hydrated sodium sulfoaluminate, and a combination thereof. In some embodiments, the flow-aid is silicon dioxide. In some embodiments, the silicon dioxide comprises fumed silica, colloidal silicon dioxide (CSD), or both. In some embodiments, the flow-aid is porous silica. In some embodiments, the flow-aid is non-porous silica.

In some embodiments, a herein-described solid formulation such as pharmaceutical powder comprises about 0.25 wt % to about 10 wt % of the flow-aid (i.e., the flow-aid is present in the solid formulation at about 0.25 wt % to about 10 wt %). In some embodiments, the solid formulation comprises about 0.5 wt % to about 10 wt % of the flow-aid. In some embodiments, the solid formulation comprises about 1 wt % to about 10 wt % of the flow-aid. In some embodiments, the solid formulation comprises about 1.5 wt % to about 10 wt % of the flow-aid. In some embodiments, the solid formulation comprises about 2 wt % to about 10 wt % of the flow-aid. In some embodiments, the solid formulation comprises about 2.5 wt % to about 10 wt % of the flow-aid. In some embodiments, the solid formulation comprises about 3 wt % to about 10 wt % of the flow-aid. In some embodiments, the solid formulation comprises about 3.5 wt % to about 10 wt % of the flow-aid. In some embodiments, the solid formulation comprises about 4 wt % to about 10 wt % of the flow-aid. In some embodiments, the solid formulation comprises about 4.5 wt % to about 10 wt % of the flow-aid. In some embodiments, the solid formulation comprises about 5 wt % to about 10 wt % of the flow-aid. In some embodiments, the solid formulation comprises about 6 wt % to about 10 wt % of the flow-aid. In some embodiments, the solid formulation comprises about 7 wt % to about 10 wt % of the flow-aid. In some embodiments, the solid formulation comprises about 8 wt % to about 10 wt % of the flow-aid. In some embodiments, the solid formulation comprises about 9 wt % to about 10 wt % of the flow-aid. In some embodiments, the solid formulation comprises about 0.5 wt % to about 9 wt % of the flow-aid. In some embodiments, the solid formulation comprises about 0.5 wt % to about 8 wt % of the flow-aid. In some embodiments, the solid formulation comprises about 0.5 wt % to about 7 wt % of the flow-aid. In some embodiments, the solid formulation comprises about 0.5 wt % to about 6 wt % of the flow-aid. In some embodiments, the solid formulation comprises about 0.5 wt % to about 5 wt % of the flow-aid. In some embodiments, the solid formulation comprises about 0.5 wt % to about 4.5 wt % of the flow-aid. In some embodiments, the solid formulation comprises about 0.5 wt % to about 4 wt % of the flow-aid. In some embodiments, the solid formulation comprises about 0.5 wt % to about 3.5 wt % of the flow-aid. In some embodiments, the solid formulation comprises about 0.5 wt % to about 3 wt % of the flow-aid. In some embodiments, the solid formulation comprises about 0.5 wt % to about 2.5 wt % of the flow-aid. In some embodiments, the solid formulation comprises about 0.5 wt % to about 2 wt % of the flow-aid. In some embodiments, the solid formulation comprises about 0.5 wt % to about 1.5 wt % of the flow-aid. In some embodiments, the solid formulation comprises about 0.5 wt % to about 1 wt % of the flow-aid. In some embodiments, the solid formulation is a powder. In some embodiments, the solid formulation is in a form of flake, granules or pellets.

In some embodiments, a herein-described solid formulation such as a pharmaceutical powder comprises about 0.5 wt %, about 0.6 wt %, about 0.7 wt %, about 0.8 wt %, about 0.9 wt %, about 1 wt %, about 1.1 wt %, about 1.2 wt %, about 1.3 wt %, about 1.4 wt %, about 1.5 wt %, about 2 wt %, about 2.5 wt %, about 3 wt %, about 3.5 wt %, about 4 wt %, about 4.5 wt %, about 5 wt %, about 6 wt %, about 7 wt %, about 8 wt %, about 9 wt %, or about 10 wt % of the flow-aid. In some embodiments, the solid formulation comprises %, at least 0.5 wt %, at least 0.6 wt %, at least 0.7 wt %, at least 0.8 wt %, at least 0.9 wt %, at least 1 wt %, at least 1.1 wt %, at least 1.2 wt %, at least 1.3 wt %, at least 1.4 wt %, at least 1.5 wt %, at least 2 wt %, at least 2.5 wt %, at least 3 wt %, at least 3.5 wt %, at least 4 wt %, at least 4.5 wt %, at least 5 wt %, at least 6 wt %, at least 7 wt %, at least 8 wt %, at least 9 wt %, or at least 10 wt % of the flow-aid. In some embodiments, the pharmaceutical composition comprises at most 0.5 wt %, at most 0.6 wt %, at most 0.7 wt %, at most 0.8 wt %, at most 0.9 wt %, at most 1 wt %, at most 1.1 wt %, at most 1.2 wt %, at most 1.3 wt %, at most 1.4 wt %, at most 1.5 wt %, at most 2 wt %, at most 2.5 wt %, at most 3 wt %, at most 3.5 wt %, at most 4 wt %, at most 4.5 wt %, at most 5 wt %, at most 6 wt %, at most 7 wt %, at most 8 wt %, at most 9 wt %, or at most 10 wt % of the flow-aid.

In some embodiments, a suspension described herein comprises one or more pharmaceutically acceptable excipients as disclosed herein. In some embodiments, the one or more pharmaceutically acceptable excipients comprises a flow-aid. In some embodiments, a concentration of the flow-aid in the suspension is about 3 mg/mL to about 30 mg/mL. In some embodiments, a concentration of the flow-aid in the suspension is about 3 mg/mL to about 25 mg/mL. In some embodiments, a concentration of the flow-aid in the suspension is about 3 mg/mL to about 20 mg/mL. In some embodiments, a concentration of the flow-aid in the suspension is about 3 mg/mL to about 15 mg/mL. In some embodiments, a concentration of the flow-aid in the suspension is about 3 mg/mL to about 10 mg/mL. In some embodiments, a concentration of the flow-aid in the suspension is about 3 mg/mL to about 5 mg/mL. In some embodiments, a concentration of the flow-aid in the suspension is about 5 mg/mL to about 30 mg/mL. In some embodiments, a concentration of the flow-aid in the suspension is about 10 mg/mL to about 30 mg/mL. In some embodiments, a concentration of the flow-aid in the suspension is about 15 mg/mL to about 30 mg/mL. In some embodiments, a concentration of the flow-aid in the suspension is about 20 mg/mL to about 30 mg/mL. In some embodiments, a concentration of the flow-aid in the suspension is about 25 mg/mL to about 30 mg/mL. In some embodiments, the concentration of the flow-aid in the suspension is about 5 mg/mL to about 15 mg/mL. In some embodiments, the concentration of the flow-aid in the suspension is about 10 mg/mL to about 15 mg/mL. In some embodiments, the concentration of the flow-aid in the suspension is about 3 mg/mL, about 5 mg/mL, about 10 mg/mL, about 15 mg/mL, about 20 mg/mL, about 25 mg/mL, or about 30 mg/mL. In some embodiments, the concentration of the flow-aid in the suspension is at least 3 mg/mL, at least 5 mg/mL, at least 10 mg/mL, at least 15 mg/mL, at least 20 mg/mL, at least 25 mg/mL, or at least 30 mg/mL. In some embodiments, the concentration of the flow-aid in the suspension is at most 3 mg/mL, at most 5 mg/mL, at most 10 mg/mL, at most 15 mg/mL, at most 20 mg/mL, at most 25 mg/mL, or at most 30 mg/mL.

Surfactant

Solid formulations and/or liquid formulations described herein can comprise one or more surfactants. In some embodiments, one or more pharmaceutically acceptable excipients described herein comprise a surfactant. In some embodiments, the surfactant comprises cationic surfactant, anionic surfactant, non-ionic surfactant, or a combination thereof. In some embodiments, the surfactant is a cationic surfactant. In some embodiments, the surfactant is an anionic surfactant. In some embodiments, the surfactant is non-ionic. A non-ionic surfactant has no charged groups in its head. Exemplary nonionic surfactants include, without limitation, fatty alcohols, cetyl alcohol, stearyl alcohol, cetostearyl alcohol, and oleyl alcohol. Exemplary nonionic surfactants include, but are not limited to, polyethylene glycol alkyl ethers (such as octaethylene glycol monododecyl ether, pentaethylene glycol monododecyl ether), polypropylene glycol alkyl ethers, glucoside alkyl ethers (such as decyl glucoside, lauryl glucoside, octyl glucoside), polyethylene glycol octylphenyl ethers (such as Triton X-100), polyethylene glycol alkylphenyl ethers (such as nonoxynol-9), glycerol alkyl esters (such as glyceryl laurate), polyoxyethylene glycol sorbitan alkyl esters (such as polysorbate), sorbitan alkyl esters (such as Spans), cocamide MEA, cocamide DEA, dodecyldimethylamine oxide, Tocofersolan (TPGS), block copolymers of polyethylene glycol and polypropylene glycol (such as poloxamers), and polyethoxylated tallow amine (POEA). In some embodiments, the surfactant is a non-ionic surfactant that comprises polyethylene glycol. In some embodiments, the surfactant is a block copolymer of polyethylene glycol and polypropylene glycol. In some embodiments, the surfactant comprises poloxamer.

In some embodiments, the non-ionic surfactant has a number average molecular weight of from about from about 1000 to about 100,000 Da, 2000 to about 20,000 Da, from about 4000 to about 15,000 Da, from about 6000 to about 12,000 Da, or from about 7000 to about 10,000 Da. In some embodiments, the non-ionic surfactant has a number average molecular weight of from about 7000 to about 10,000 Da. In some embodiments, the non-ionic surfactant has an ethylene glycol content of from about 30 wt % to about 99 wt %, from about 50 wt % to about 95 wt %, from about 60 wt % to about 95 wt %, from about 75 wt % to about 90 wt %, or from about 80 wt % to about 85 wt %. In some embodiments, the non-ionic surfactant has an ethylene glycol content of from about 80 wt % to about 85 wt %.

The surfactant used in the present disclosure can comprise a cationic surfactant. Cationic surfactants include pH-dependent primary, secondary, or tertiary amines such as octenidine dihydrochloride; and permanently charged quaternary ammonium salts such as cetrimonium bromide (CTAB), cetylpyridinium chloride (CPC), benzalkonium chloride (BAC), benzethonium chloride (BZT), dimethyldioctadecylammonium chloride, and dioctadecyldimethylammonium bromide (DODAB).

The surfactant used in the present disclosure can comprise an anionic surfactant. Anionic surfactants contain anionic functional groups at their head, such as sulfate, sulfonate, phosphate, and carboxylates. Exemplary anionic surfactants include, but are not limited to, ammonium lauryl sulfate, sodium lauryl sulfate (sodium dodecyl sulfate, SLS, or SDS), and the related alkyl-ether sulfates sodium laureth sulfate (sodium lauryl ether sulfate or SLES), sodium myreth sulfate, docusate (dioctyl sodium sulfosuccinate), perfluorooctanesulfonate (PFOS), perfluorobutanesulfonate, docusate sodium, alkyl-aryl ether phosphates, and alkyl ether phosphates. In some embodiments, the surfactant comprises sodium lauryl sulfate (SLS).

The surfactant used in the present disclosure can be a zwitterionic surfactant. Zwitterionic (amphoteric) surfactants refer to those having cationic and anionic centers attached to the same molecule. Exemplary zwitterionic surfactants include, without limitation, phospholipids phosphatidylserine, phosphatidylethanolamine, phosphatidylcholine, and sphingomyelins.

In some embodiments, the surfactant comprises cationic surfactant, anionic surfactant, non-ionic surfactant, or a combination thereof. In some embodiments, the surfactant comprises sodium lauryl sulfate (SLS). In some embodiments, the surfactant comprises poloxamer.

In some embodiments, one or more pharmaceutically acceptable excipients described herein comprise CTAB. In some embodiments, one or more pharmaceutically acceptable excipients described herein comprise SLS. In some embodiments, one or more pharmaceutically acceptable excipients described herein comprise sodium docussate. In some embodiments, one or more pharmaceutically acceptable excipients described herein comprise Tween80. In some embodiments, one or more pharmaceutically acceptable excipients described herein comprise Tween 20. In some embodiments, one or more pharmaceutically acceptable excipients described herein comprise a Poloxamer.

In some embodiments, a herein-described solid formulation comprises about 0.01 wt % to about 10 wt % of surfactant. In some embodiments, the solid formulation comprises about 0.02 wt % to about 10 wt % of the surfactant. In some embodiments, the solid formulation comprises about 0.03 wt % to about 10 wt % of surfactant. In some embodiments, the solid formulation comprises about 0.04 wt % to about 10 wt % of surfactant. In some embodiments, the solid formulation comprises about 0.05 wt % to about 10 wt % of surfactant. In some embodiments, the solid formulation comprises about 0.1 wt % to about 10 wt % of surfactant. In some embodiments, the solid formulation comprises about 0.25 wt % to about 10 wt % of surfactant. In some embodiments, the solid formulation comprises about 0.5 wt % to about 10 wt % of surfactant. In some embodiments, the solid formulation comprises about 1 wt % to about 10 wt % of surfactant. In some embodiments, the solid formulation comprises about 2 wt % to about 10 wt % of surfactant. In some embodiments, the solid formulation comprises about 3 wt % to about 10 wt % of surfactant. In some embodiments, the solid formulation comprises about 4 wt % to about 10 wt % of surfactant. In some embodiments, the solid formulation comprises about 5 wt % to about 10 wt % of surfactant. In some embodiments, the solid formulation comprises about 6 wt % to about 10 wt % of surfactant. In some embodiments, the solid formulation comprises about 7 wt % to about 10 wt % of surfactant. In some embodiments, the solid formulation comprises about 8 wt % to about 10 wt % of surfactant. In some embodiments, the solid formulation comprises about 9 wt % to about 10 wt % of surfactant. In some embodiments, the solid formulation comprises about 0.01 wt % to about 9 wt % of surfactant. In some embodiments, the solid formulation comprises about 0.01 wt % to about 8 wt % of surfactant. In some embodiments, the solid formulation comprises about 0.01 wt % to about 7 wt % of surfactant. In some embodiments, the solid formulation comprises about 0.01 wt % to about 6 wt % of surfactant. In some embodiments, the solid formulation comprises about 0.01 wt % to about 5 wt % of surfactant. In some embodiments, the solid formulation comprises about 0.01 wt % to about 4 wt % of surfactant. In some embodiments, the solid formulation comprises about 0.01 wt % to about 3 wt % of surfactant. In some embodiments, the solid formulation comprises about 0.01 wt % to about 2 wt % of surfactant. In some embodiments, the solid formulation comprises about 0.01 wt % to about 1 wt % of surfactant. In some embodiments, the solid formulation comprises about 0.01 wt % to about 0.5 wt % of surfactant. In some embodiments, the solid formulation comprises about 0.01 wt % to about 0.25 wt % of surfactant. In some embodiments, the solid formulation comprises about 0.01 wt % to about 0.1 wt % of surfactant. In some embodiments, the solid formulation is a powder. In some embodiments, the solid formulation is in a form of flake, granules or pellets.

In some embodiments, the solid formulation such as a pharmaceutical powder comprises about 0.01 wt %, about 0.02 wt %, about 0.03 wt %, about 0.04 wt %, about 0.05 wt %, 0.1 wt %, about 0.2 wt %, about 0.3 wt %, about 0.4 wt %, about 0.5 wt %, about 0.6 wt %, about 0.7 wt %, about 0.8 wt %, about 0.9 wt %, about 1 wt %, about 1.1 wt %, about 1.2 wt %, about 1.3 wt %, about 1.4 wt %, about 1.5 wt %, about 2 wt %, about 2.5 wt %, about 3 wt %, about 3.5 wt %, about 4 wt %, about 4.5 wt %, about 5 wt %, about 6 wt %, about 7 wt %, about 8 wt %, about 9 wt %, or about 10 wt % of surfactant. In some embodiments, the solid formulation comprises at least 0.01 wt %, at least 0.02 wt %, at least 0.03 wt %, at least 0.04 wt %, at least 0.05 wt %, at least 0.1 wt %, at least 0.2 wt %, at least 0.3 wt %, at least 0.4 wt %, at least 0.5 wt %, at least 0.6 wt %, at least 0.7 wt %, at least 0.8 wt %, at least 0.9 wt %, at least 1 wt %, at least 1.1 wt %, at least 1.2 wt %, at least 1.3 wt %, at least 1.4 wt %, at least 1.5 wt %, at least 2 wt %, at least 2.5 wt %, at least 3 wt %, at least 3.5 wt %, at least 4 wt %, at least 4.5 wt %, at least 5 wt %, at least 6 wt %, at least 7 wt %, at least 8 wt %, at least 9 wt %, or at least 10 wt % of surfactant. In some embodiments, the solid formulation comprises at most 0.01 wt %, at most 0.02 wt %, at most 0.03 wt %, at most 0.04 wt %, at most 0.05 wt %, at most 0.1 wt %, at most 0.2 wt %, at most 0.3 wt %, at most 0.4 wt %, at most 0.5 wt %, at most 0.6 wt %, at most 0.7 wt %, at most 0.8 wt %, at most 0.9 wt %, at most 1 wt %, at most 1.1 wt %, at most 1.2 wt %, at most 1.3 wt %, at most 1.4 wt %, at most 1.5 wt %, at most 2 wt %, at most 2.5 wt %, at most 3 wt %, at most 3.5 wt %, at most 4 wt %, at most 4.5 wt %, at most 5 wt %, at most 6 wt %, at most 7 wt %, at most 8 wt %, at most 9 wt %, or at most 10 wt % of surfactant. In some embodiments, the solid formulation is a powder. In some embodiments, the solid formulation is in a form of flake, granules or pellets.

In some embodiments, a suspension described herein comprises one or more pharmaceutically acceptable excipients as disclosed herein. In some embodiments, the one or more pharmaceutically acceptable excipients comprises a surfactant. In some embodiments, a concentration of the surfactant in the suspension is about 0.5 mg/mL to about 10 mg/mL. In some embodiments, a concentration of the surfactant in the suspension is about 1 mg/mL to about 10 mg/mL. In some embodiments, a concentration of the surfactant in the suspension is about 1.5 mg/mL to about 10 mg/mL. In some embodiments, a concentration of the surfactant in the suspension is about 2 mg/mL to about 10 mg/mL. In some embodiments, a concentration of the surfactant in the suspension is about 2.5 mg/mL to about 10 mg/mL. In some embodiments, a concentration of the surfactant in the suspension is about 3 mg/mL to about 10 mg/mL. In some embodiments, a concentration of the surfactant in the suspension is about 3.5 mg/mL to about 10 mg/mL. In some embodiments, a concentration of the surfactant in the suspension is about 4 mg/mL to about 10 mg/mL. In some embodiments, a concentration of the surfactant in the suspension is about 4.5 mg/mL to about 10 mg/mL. In some embodiments, a concentration of the surfactant in the suspension is about 5 mg/mL to about 10 mg/mL. In some embodiments, a concentration of the surfactant in the suspension is about 6 mg/mL to about 10 mg/mL. In some embodiments, a concentration of the surfactant in the suspension is about 7 mg/mL to about 10 mg/mL. In some embodiments, a concentration of the surfactant in the suspension is about 8 mg/mL to about 10 mg/mL. In some embodiments, a concentration of the surfactant in the suspension is about 9 mg/mL to about 10 mg/mL. In some embodiments, a concentration of the surfactant in the suspension is about 0.5 mg/mL to about 10 mg/mL. In some embodiments, a concentration of the surfactant in the suspension is about 0.5 mg/mL to about 9 mg/mL. In some embodiments, a concentration of the surfactant in the suspension is about 0.5 mg/mL to about 8 mg/mL. In some embodiments, a concentration of the surfactant in the suspension is about 0.5 mg/mL to about 7 mg/mL. In some embodiments, a concentration of the surfactant in the suspension is about 0.5 mg/mL to about 6 mg/mL. In some embodiments, a concentration of the surfactant in the suspension is about 0.5 mg/mL to about 5 mg/mL. In some embodiments, a concentration of the surfactant in the suspension is about 0.5 mg/mL to about 4.5 mg/mL. In some embodiments, a concentration of the surfactant in the suspension is about 0.5 mg/mL to about 4 mg/mL. In some embodiments, a concentration of the surfactant in the suspension is about 3.5 mg/mL to about 10 mg/mL. In some embodiments, a concentration of the surfactant in the suspension is about 0.5 mg/mL to about 3 mg/mL. In some embodiments, the concentration of the surfactant in the suspension is about 1 mg/mL to about 2.5 mg/mL. In some embodiments, a concentration of the surfactant in the suspension is about 0.5 mg/mL to about 2.5 mg/mL. In some embodiments, a concentration of the surfactant in the suspension is about 0.5 mg/mL to about 2 mg/mL. In some embodiments, a concentration of the surfactant in the suspension is about 0.5 mg/mL to about 1 mg/mL. In some embodiments, a concentration of the surfactant in the suspension is about 0.5 mg/mL, about 1 mg/mL, about 1.5 mg/mL, about 2 mg/mL, about 2.5 mg/mL, about 3 mg/mL, about 3.5 mg/mL, about 4 mg/mL, about 4.5 mg/mL, about 5 mg/mL, about 6 mg/mL, about 7 mg/mL, about 8 mg/mL, about 9 mg/mL, or about 10 mg/mL. In some embodiments, a concentration of the surfactant in the suspension is at least 0.5 mg/mL, at least 1 mg/mL, at least 1.5 mg/mL, at least 2 mg/mL, at least 2.5 mg/mL, at least 3 mg/mL, at least 3.5 mg/mL, at least 4 mg/mL, at least 4.5 mg/mL, at least 5 mg/mL, at least 6 mg/mL, at least 7 mg/mL, at least 8 mg/mL, at least 9 mg/mL, or at least 10 mg/mL. In some embodiments, a concentration of the surfactant in the suspension is at most 0.5 mg/mL, at most 1 mg/mL, at most 1.5 mg/mL, at most 2 mg/mL, at most 2.5 mg/mL, at most 3 mg/mL, at most 3.5 mg/mL, at most 4 mg/mL, at most 4.5 mg/mL, at most 5 mg/mL, at most 6 mg/mL, at most 7 mg/mL, at most 8 mg/mL, at most 9 mg/mL, or at most 10 mg/mL.

In some embodiments, a pharmaceutical powder of the present disclosure comprises:
about 10 wt % to about 60 wt % of an amorphous solid dispersion comprising (i) about 40 wt % to about 60 wt % of Compound A and (ii) about 40 wt % to about 60 wt % of PVP-VA, wherein the amorphous solid dispersion is a hot melt extrudate;
about 30 wt % to about 70 wt % of a filler, wherein the filler comprises microcrystalline cellulose and mannitol;
about 0.1 wt % to 5 wt % of a surfactant, wherein the surfactant is SLS;
about 0.25 wt % to 6 wt % of a flow-aid, wherein the flow-aid is CSD; and
about 0.5 wt % to 5 wt % of an antifoam, wherein the antifoam is simethicone or dimethicone.

In some embodiments, the solid formulation comprises:
(a) an amorphous solid dispersion comprising about 40 wt % to about 60 wt % of Compound A and about 40 wt % to about 60 wt % of PVP-VA, wherein the amorphous solid dispersion is a hot melt extrudate and is present in the solid formulation at about 40 wt % to about 60 wt %;
(b) about 30 wt % to 70 wt % of a filler, wherein the filler comprises microcrystalline cellulose and mannitol;
(c) about 0.1 wt % to 5 wt % of a surfactant, wherein the surfactant is SLS;
(d) about 0.25 wt % to 6 wt % of a flow-aid, wherein the flow-aid is CSD; and
(e) about 0.5 wt % to 5 wt % of an antifoam, wherein the antifoam is simethicone.

Sweeteners and Flavoring Agents

Solid formulations and/or liquid formulations described herein can comprise one or more sweeteners, one or more flavoring agents, or both. In some embodiments, one or more pharmaceutically acceptable excipients described herein comprise a sweetener. In some embodiments, one or more pharmaceutically acceptable excipients described herein comprise a flavoring agent.

Sweeteners or sweetening agents may include any compounds that provide a sweet taste to enhance the palatability of the formulation, including natural and synthetic sugars and natural and synthetic non-sugar sweeteners. These could include glucose, fructose, sucrose, or other pharmaceutically acceptable monosaccharides and disaccharides or sugar alcohols, such as xylitol. Also, sweeteners may include maltodextrin, polydextrose and the like. Other sweeteners may include glycerin, inulin, maltol, salts of acesulfame, alitame, aspartame, neotame, cyclamate salts, sucralose, sorbitol solution, saccharin and its salts, and other artificial and naturally-occurring agents providing sweetness either singly or in combination.

Exemplary sweeteners include, but are not limited to, glucose, fructose, sucrose, xylitol, tagatose, sucralose, maltitol, isomaltulose, hydrogenated isomaltulose sold under the trademark Isomalt™, lactitol, sorbitol, erythritol, trehalose, maltodextrin, polydextrose, and the like. Other sweetening agents illustratively include glycerin, inulin, maltol, acesulfame and salts thereof, e.g., acesulfame potassium, alitame, aspartame, neotame, sodium cyclamate, saccharin and salts thereof, e.g., saccharin sodium or saccharin calcium, neohesperidin dihydrochalcone, stevioside, thaumatin, and the like. Sweetening agents can be used in the form of crude or refined products such as hydrogenated starch hydrolysates, maltitol syrup, high fructose corn syrup, etc., and as branded products, e.g., a combination of propylene glycol, ethyl alcohol, and proprietary artificial flavor sold under the trademark Sweet Am™ liquid by Flavors of North America, a combination of maltodextrin, sorbitol, and fructose sold under the trademark Sweet Am™ powder with Product Code 918.005, a combination of water, propylene glycol, sorbitol, fructose, and proprietary natural and artificial flavor sold under the trademark Sweet Am™ powder with Product Code 918.010 by Flavors of North America, a combination of 1-10% proprietary plant/vegetable extract and 90-99% dextrose sold under the trademark ProSweet™ by Virginia Dare, a maltitol solution sold under the trademark Maltisweet™ by Ingredion, a sorbitol and sorbitol/xylitol solution sold under the trademark Sorbo™ by SPI Polyols, a high fructose corn syrup sold under the trademark Invertose™ by Ingredion, a combination of sucralose and maltodextrin sold under the trademark Rebalance M60 and X60 by Tate and Lyle, and a sugar containing and sugar-free flavored syrups sold under the trademarks Ora-Sweet® and Ora-Sweet-SF®, respectively, by Paddock Laboratories, Inc. Sweetening agents can be used singly or in combinations of two or more. Suitable concentrations of different sweetening agents can be selected based on published information, manufacturers' data sheets and by routine testing.

In some embodiments, a herein-described solid formulation such as a pharmaceutical powder comprises about 0.01 wt % to about 10 wt % of sweetener(s). In some embodiments, a herein-described solid formulation such as a pharmaceutical powder optionally comprises about 0.01 wt % to about 10 wt % of sweetener(s). In some embodiments, the solid formulation comprises about 0.01 wt % to about 5 wt % of sweetener. In some embodiments, the solid formulation comprises about 0.01 wt % to about 1 wt % of sweetener. In some embodiments, the solid formulation comprises about 0.1 wt % to about 0.5 wt % of sweetener. In some embodiments, the solid formulation comprises about 0.2 wt % to about 0.8 wt % of sweetener. In some embodiments, the solid formulation comprises about 0.5 wt % to about 0.6 wt % of sweetener. In some embodiments, the solid formulation comprises about 0.6 wt % of sweetener.

In some embodiments, a herein-described solid formulation such as a pharmaceutical powder comprises about 0.01 wt % to about 2 wt % of flavoring agent(s). In some embodiments, a herein-described solid formulation such as a pharmaceutical powder optionally comprises about 0.01 wt % to about 2 wt % of flavoring agent(s). In some embodiments, the solid formulation comprises about 0.01 wt % to about 2 wt % of flavoring agent. In some embodiments, the solid formulation comprises about 0.001 wt % to about 1 wt % of flavoring agent. In some embodiments, the solid formulation comprises about 0.01 wt % to about 0.5 wt % of flavoring agent.

In some embodiments of the present disclosure, pharmaceutical compositions described herein, e.g., pharmaceutical powders and suspensions, comprise one or more sweeteners (e.g., sucralose and sorbitol solution). In some embodiments, a suspension described herein comprises a sweetener present from about 0.01% w/v to about 30% w/v of the liquid formulation. In some embodiments, the sweetener comprises a mixture of two or more sweetening agents. In some embodiments, the sweetener comprises sucralose. In some embodiments, the sweetener comprises sorbitol. In some embodiments, the sweetener comprises sucrose. In some embodiments, a suspension described herein comprises 0.1%-14%, 0.2%-7%, 0.3%-5%, 0.35%-0.5%, 0.35%-1.0%, 0.35%-1.5%, 0.35%-2.0%, 0.35%-2.5%, 0.35%-3.0%, 0.35%-3.5%, 0.5%-1.0%, 0.5%-1.5%, 0.5%-2.0%, 0.5%-2.5%, 0.5%-3.0%, 0.5%-3.5%, 1.0%-1.5%, 1.0%-2.0%, 1.0%-2.5%, 1.0%-3.0%, 1.0%-3.5%, 1.5%-2.0%, 1.5%-2.5%, 1.5%-3.0%, 1.5%-3.5%, 1.5%-4.0%, 2.0%-2.5%, 2.0%-3.0%, 2.0%-3.5%, 2.0%-4.0%, 2.5%-3.0%, 2.5%-3.5%, 2.5%-4.0%, 3.0%-3.5%, 3.0%-4.0%, 3.5%-4.0% w/v of sweetener or any ranges or values contemplated therein. In some embodiments, a suspension described herein comprises 0.1%-1% w/v of sweetener. In some embodiments, a suspension described herein comprises 0.05%-0.5% w/v of sweetener. In some embodiments, a suspension described herein comprises 0.2%-0.8% w/v of sweetener. In some embodiments, the sweetener or the combination of sweeteners is present from about 0.35% w/v to about 3.5% w/v. In some embodiments, the sweetener or the combination of sweeteners is present from about 0.35% w/v to about 3.5% w/v, from about 0.2% w/v to about 7.0% w/v, from about 0.10% w/v to about 10% w/v, from about 1% w/v to about 10% w/v, from about 1% w/v to about 15% w/v, from about 0.5% w/v to about 12% w/v, or from about 0.1% w/v to about 15% w/v in the suspension.

In some embodiments, a suspension described herein comprises 0.1%-14% w/v of sweetener. In some embodiments, a suspension described herein comprises 0.2%-7% w/v of sweetener. In some embodiments, a suspension described herein comprises 0.35%-3.5% w/v of sweetener. In some embodiments, a suspension described herein comprises 0.05%-0.5% w/v of sweetener. In some embodiments, a suspension described herein comprises 0.15%-0.35% w/v of sweetener. In some embodiments, a suspension described herein comprises 0.3%-5% w/v of sweetener. In some embodiments, a suspension described herein comprises 0.35%-3.5% w/v of sweetener. In some embodiments, a suspension described herein comprises 0.35%-3.0% w/v of sweetener. In some embodiments, a suspension described herein comprises 0.5%-1.0% w/v of sweetener. In some embodiments, a suspension described herein comprises 1.0%-2.5% w/v of sweetener. In some embodiments, a suspension described herein comprises 1.0%-2.5% w/v of sweetener. In some embodiments, a suspension described herein comprises 2.0%-4.0% w/v of sweetener. In some embodiments, a suspension described herein comprises 0.1%-5% w/v of sweetener. In some embodiments, a suspension described herein comprises 0.1%-15% w/v of sweetener. In some embodiments, a suspension described herein comprises 0.1%-10% w/v of sweetener. In some embodiments, a suspension described herein comprises 5%-15% w/v of sweetener. In some embodiments, a suspension described herein comprises 5%-25% w/v of sweetener. In some embodiments, the sweetener comprises sucralose. In some embodiments, the sweetener comprises sorbitol. In some embodiments, the sweetener comprises sucrose. In some embodiments, the sweetener comprises glycyrrhizin.

In some embodiments, pharmaceutical compositions described herein, e.g., pharmaceutical powders and suspensions, comprise a flavoring agent or flavorant to enhance the flavor or aroma of the dose, and to improve general palatability of the dose, thus helping to mask the flavor of the active ingredient which patients may find unpleasant. This can provide an improved experience for patients, and better compliance with the drug regimen desired by clinicians. Suitable natural or artificial flavors can be selected from pharmaceutically acceptable options as described in standard pharmacy references which are known to those skilled in the art. Suitable natural or synthetic flavoring agents can be selected from standard reference books, such as Remington: The Science and Practice of Pharmacy (2000) and Fenaroli's Handbook of Flavor Ingredients (1994).

Non-limiting examples of suitable natural flavors, some of which can be readily simulated with synthetic agents or combinations thereof, include almond, anise, apple, apricot, banana, blackberry, blackcurrant, blueberry, caramel, cherry, chocolate, cinnamon, cranberry, grape, lemon, lime, orange, peppermint, pineapple, raspberry, spearmint, strawberry, vanilla, etc. In some embodiments, the flavors include which can be readily simulated with synthetic agents or combinations thereof include fat, poultry, fish, beef, and other meats. In some embodiments, vanilla flavor is used. In some embodiments, mint flavor is used. In some embodiments, strawberry flavor (e.g., Strawberry Flavor CW08) is used. In other embodiments, other pharmaceutically acceptable flavors can be used to mask the flavor of other ingredients, for example other APIs, and to enhance palatability and thus compliance in a range of patient populations. In some embodiments, the natural flavor may be natural or synthetic. In some embodiments, the synthetic flavor is an artificial flavor, for example artificial strawberry flavor. Natural and synthetic flavors can be used and adapted to the palate of diverse patient populations, including but not limited to, age- and culturally-related flavor preferences (for example bubble gum flavor for pediatric patients).

In some embodiments, a herein-described solid formulation such as a pharmaceutical powder comprises about 0.01 wt % to about 10 wt % of flavoring agent. In some embodiments, a herein-described solid formulation such as a pharmaceutical powder optionally comprises about 0.01 wt % to about 10 wt % of flavoring agent. In some embodiments, the solid formulation comprises about 0.01 wt % to about 5 wt % of flavoring agent. In some embodiments, the solid formulation comprises about 0.01 wt % to about 1 wt % of flavoring agent. In some embodiments, the solid formulation comprises about 0.1 wt % to about 0.5 wt % of flavoring agent. In some embodiments, the solid formulation comprises about 1 wt % to about 2 wt % of flavoring agent. In some embodiments, the solid formulation comprises about 0.5 wt % to about 2.5 wt % of flavoring agent. In some embodiments, the solid formulation comprises about 0.01 wt % to about 2 wt % of flavoring agent. In some embodiments, the solid formulation comprises about 0.001 wt % to about 1 wt % of flavoring agent. In some embodiments, the solid formulation comprises about 0.01 wt % to about 0.5 wt % of flavoring agent.

In some embodiments of the present disclosure, pharmaceutical compositions described herein, e.g., pharmaceutical powders and suspensions, comprise one or more flavoring agents. In some embodiments, a suspension described herein comprises a flavoring agent present from about 0.01% w/v to about 30% w/v of the liquid formulation. In some embodiments, a suspension described herein comprises 0.1%-14%, 0.2%-7%, 0.3%-5%, 0.35%-0.5%, 0.35%-1.0%, 0.35%-1.5%, 0.35%-2.0%, 0.35%-2.5%, 0.35%-3.0%, 0.35%-3.5%, 0.5%-1.0%, 0.5%-1.5%, 0.5%-2.0%, 0.5%-2.5%, 0.5%-3.0%, 0.5%-3.5%, 1.0%-1.5%, 1.0%-2.0%, 1.0%-2.5%, 1.0%-3.0%, 1.0%-3.5%, 1.5%-2.0%, 1.5%-2.5%, 1.5%-3.0%, 1.5%-3.5%, 1.5%-4.0%, 2.0%-2.5%, 2.0%-3.0%, 2.0%-3.5%, 2.0%-4.0%, 2.5%-3.0%, 2.5%-3.5%, 2.5%-4.0%, 3.0%-3.5%, 3.0%-4.0%, 3.5%-4.0% w/v of flavoring agent or any ranges or values contemplated therein. In some embodiments, a suspension described herein comprises 0.1%-1% w/v of flavoring agent. In some embodiments, a suspension described herein comprises 0.05%-0.5% w/v of flavoring agent. In some embodiments, a suspension described herein comprises 0.2%-2% w/v of flavoring agent. In some embodiments, the flavoring agent or the combination of flavoring agents is present from about 0.35% w/v to about 3.5% w/v. In some embodiments, the flavoring agent or the combination of flavoring agents is present from about 0.35% w/v to about 3.5% w/v, from about 0.2% w/v to about 7.0% w/v, from about 0.1% w/v to about 10% w/v, from about 1% w/v to about 10% w/v, from about 1% w/v to about 15% w/v, from about 0.5% w/v to about 12% w/v, or from about 0.1% w/v to about 15% w/v in the suspension.

Coloring Agents

Solid formulations and/or liquid formulations described herein can comprise one or more coloring agents. Many such agents are approved for use by the U.S. Food and Drug Administration, and are well known to those skilled in the art of compounding pharmacy. Suitable coloring agents approved by the U.S. Food and Drug Administration (FDA) include FD&C Red No. 3, FD&C Red No. 20, FD&C Red No. 40, FD&C Yellow No. 6, FD&C Blue No. 2, D&C Green No. 5, D&C Orange No. 5, D&C Yellow No. 10, caramel, ferric oxide and mixtures thereof.

The use of color can enhance the aesthetic appearance of the dose as well as providing confirmation of the identity of the drug in a context where more than one oral formulation is being prepared, stored, transported, or used. Enhancing the aesthetic appearance of the dose increases the overall palatability of the dose, which can provide benefits to patients and clinicians in terms of improved patient experience and improved compliance with the drug regimen. The ability to unambiguously identify the medication in the pharmacy, clinical, and patient context provides benefits to the patient by reducing the scope for errors in the preparation, storage, handling, transport, and use of the medication.

In some embodiments, a herein-described solid formulation such as a pharmaceutical powder comprises about 0.0001 wt % to about 1 wt % of coloring agent. In some embodiments, a herein-described solid formulation such as a pharmaceutical powder optionally comprises about 0.0001 wt % to about 1 wt % of coloring agent.

In some embodiments of the present disclosure, the coloring agent comprises FD&C Red No. 40. However, any FDA-approved coloring agent is contemplated herein. In some embodiments, the concentration of coloring agent is 0.001%-0.1% w/v or any range or value therein in a suspension. In some embodiments, the concentration of coloring agent in a suspension is 0.001%-1%, 0.010%-2%, 0.002%-0.005%, 0.002%-0.004%, 0.002%-0.003% w/v or any range or value therein. In some embodiments, the concentration of coloring agent in a suspension or pharmaceutical powder is less than 0.002%. In some embodiments, the concentration of coloring agent in a suspension or pharmaceutical powder is greater than 0.005%. In some embodiments, a liquid suspension described herein comprises about 0.001%-1% w/v coloring agent. In some embodiments, a liquid suspension described herein comprises about 0.01%-2% w/v coloring agent. In some embodiments, a liquid suspension described herein comprises about 0.002%-0.005% w/v coloring agent. In some embodiments, a liquid suspension described herein comprises about 0.002%-0.003% w/v coloring agent. In some embodiments, a liquid suspension described herein comprises about 0.002%-0.004% w/v coloring agent.

In some embodiments, a herein-described solid formulation does not contain any coloring agent.

In some embodiments, the pH value of a liquid suspension described herein is about 2 to 7.

In certain aspects the present disclosure provides an oral liquid suspension comprising: (a) (R)-2-(1-(6-amino-5-chloropyrimidine-4-carboxamido)ethyl)-N-(5-chloro-4-(trifluoromethyl)pyridin-2-yl)thiazole-5-carboxamide (Compound A) or a pharmaceutically acceptable salt thereof; (b) one or more pharmaceutically acceptable excipients; and (c) water.

In some embodiments, the suspension comprises, based on the weight of the solids:
(a) about 10 wt % to about 50 wt % of an amorphous solid dispersion comprising: (i) about 40 wt % to about 60 wt % of Compound A and
   (ii) about 40 wt % to about 60 wt % of PVP-VA, wherein the amorphous solid dispersion is a hot melt extrudate;
   (b) about 40 wt % to about 70 wt % of a filler, wherein the filler comprises microcrystalline cellulose and mannitol;
(c) about 0.25 wt % to about 1 wt % of a surfactant, wherein the surfactant is SLS; (d) about 1 wt % to about 6 wt % of a flow-aid, wherein the flow-aid is colloidal silicon dioxide (CSD);
(e) about 1 wt % to about 10 wt % of an antifoam (e.g., about 1 wt % to about 5 wt % of an antifoam), wherein the antifoam comprises or is simethicone; and
(f) optionally a preservative, a flavoring agent, a sweetener, or a combination thereof.
In some embodiments, the antifoam comprises simethicone and maltodextrin. In some embodiments, the antifoam comprises simethicone (about 30 wt %) and maltodextrin (about 70 wt %).

In some embodiments, the suspension comprises a flavoring agent. In some embodiments, the suspension comprises strawberry flavor.

In some embodiments, the suspension comprises a sweetener. In some embodiments, the suspension comprises sucralose.

In some embodiments, the suspension comprises about 25 mg/mL of Compound A or a salt thereof. In some embodiments, the suspension comprises about 25 mg/mL of Compound A.

In some embodiments, the suspension comprises, based on the weight of the solids:
(a) about 10 wt % to 30 wt % (e.g., about 25 wt %) of an amorphous solid dispersion comprising:
   (i) about 40 wt % of Compound A; and
   (ii) about 60 wt % of copovidone, wherein the amorphous solid dispersion is a hot melt extrudate;
(b) about 30 wt % to 32 wt % mannitol;
(c) about 30 wt % to 32 wt % microcrystalline cellulose;
(d) about 0.5 wt % to 1 wt % SLS;
(e) about 4 wt % to 5 wt % CSD;
(f) about 2 wt % to 3 wt % of defoamer;
optionally a carrier of the defoamer;
and optionally a preservative, a flavoring agent, a sweetener, or a combination thereof.

In some embodiments, the suspension comprises, based on the weight of the solids:
(a) about 10 wt % to 30 wt % (e.g., about 25 wt %) of an amorphous solid dispersion comprising:
   (i) about 40 wt % of Compound A; and
   (ii) about 60 wt % of copovidone, wherein the amorphous solid dispersion is a hot melt extrudate;
(b) about 30 wt % to 32 wt % mannitol;
(c) about 30 wt % to 32 wt % microcrystalline cellulose;
(d) about 0.5 wt % to 1 wt % SLS;
(e) about 4 wt % to 5 wt % CSD;
(f) about 2 wt % to 3 wt % simethicone;
optionally a carrier of the defoamer;
and optionally a preservative, a flavoring agent, a sweetener, or a combination thereof.

In some embodiments, the suspension comprises one or more of the following components, based on the weight of the solids:
(a) about 10 wt % to 30 wt % (e.g., about 25 wt %) of an amorphous solid dispersion comprising:
   (i) about 40 wt % of Compound A; and
   (ii) about 60 wt % of copovidone;
(b) about 20 wt % to 40 wt % mannitol;
(c) about 20 wt % to 40 wt % microcrystalline cellulose;
(d) about 0.1 wt % to 2 wt % SLS;
(e) about 1 wt % to 10 wt % CSD;
(f) about 1 wt % to 5 wt % simethicone;
(g) about 1 wt % to 10 wt % maltodextrin;
and optionally a preservative, a flavoring agent, a sweetener, or a combination thereof.

In some embodiments, the suspension comprises one or more of the following components, based on the weight of the solids:
(a) about 10 wt % to 30 wt % (e.g., about 25 wt %) of an amorphous solid dispersion comprising:
   (i) about 40 wt % of Compound A; and
   (ii) about 60 wt % of copovidone;
(b) about 20 wt % to 40 wt % mannitol;
(c) about 20 wt % to 40 wt % microcrystalline cellulose;
(d) about 0.1 wt % to 2 wt % SLS;
(e) about 1 wt % to 10 wt % CSD;
(f) about 1 wt % to 5 wt % simethicone;
(g) about 1 wt % to 10 wt % maltodextrin;
(h) about 0.1 wt % to 5 wt % of a flavoring agent; and
(i) about 0.1 wt % to 5 wt % of, a sweetener.

In some embodiments, the suspension comprises, based on the weight of the solids:
(a) about 10 wt % to 30 wt % (e.g., about 25 wt %) of an amorphous solid dispersion comprising: (i) about 40 wt % of Compound A; and
   (ii) about 60 wt % of copovidone;
   (b) about 30 wt % to 32 wt % mannitol;
(c) about 30 wt % to 32 wt % microcrystalline cellulose;
(d) about 0.5 wt % to 1 wt % SLS;
(e) about 4 wt % to 5 wt % CSD;
(f) about 2 wt % to 3 wt % simethicone;
(g) about 3 wt % to 8 wt % maltodextrin;
and optionally a preservative, a flavoring agent, a sweetener, or a combination thereof.

In some embodiments, the oral liquid suspension comprises a carrier for the defoamer. In some embodiments, the oral liquid suspension comprises a carrier for simethicone. In some embodiments, the carrier is a starch. In some embodiments, the carrier is a filler. In some embodiments, the carrier is Maltodextrin. In some embodiments, the carrier is present, based on the weight of the solids, at about 1-15 wt %. In some embodiments, the carrier is present, based on the weight of the solids, at about 1-10 wt %. In some embodiments, the carrier is present, based on the weight of the solids, at about 2-8 wt %. In some embodiments, the carrier is present, based on the weight of the solids, at about 4-6 wt %. In some embodiments, the carrier is present, based on the weight of the solids, at about 5 wt %. In some embodiments, the weight ratio of the carrier to the defoamer is about 1:9 to 9:1. In some embodiments, the weight ratio of the carrier to the defoamer is about 1:4 to 4:1. In some embodiments, the weight ratio of the carrier to the defoamer is about 1:3 to 3:1. In some embodiments, the weight ratio of the carrier to the defoamer is about 8:3 to 6:3. In some embodiments, the weight ratio of the carrier to the defoamer is about 7:3.

In some embodiments, the oral liquid suspension is bioequivalent to a tablet formulation of Compound A or a pharmaceutically acceptable salt thereof, wherein the tablet composition comprises an amorphous solid dispersion comprising (i) about 40 wt % of Compound A and (ii) about 60 wt % of copovidone, wherein the amorphous solid dispersion is a hot melt extrudate; and one or more pharmaceutically acceptable excipients as disclosed herein.

Pharmaceutical compositions (including the pharmaceutical powders and oral formulations therefrom) may be formulated using one or more physiologically acceptable carriers including excipients and auxiliaries which facilitate processing of the pharmaceutical agent into preparations which are used pharmaceutically. Proper formulation is dependent upon the route of administration chosen. A summary of pharmaceutical compositions is found, for example, in Remington: The Science and Practice of Pharmacy, Nineteenth Ed (Easton, Pa., Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins, 1999).

A pharmaceutically acceptable excipient can contain physiologically acceptable agents that act, for example, to stabilize, increase solubility or to increase the absorption of a compound such as a pharmaceutical agent. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable excipient, including a physiologically acceptable agent, depends, for example, on the route of administration of the composition. The preparation or pharmaceutical composition can be a self emulsifying drug delivery system or a self microemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

A pharmaceutical composition (including the pharmaceutical powders and oral formulations therefrom) may be a sterile aqueous or non-aqueous solution, suspension or emulsion, e.g., a microemulsion. The excipients described herein are examples and are in no way limiting. An effective amount or therapeutically effective amount refers to an amount of the one or more pharmaceutical agents administered to a subject, either as a single dose or as part of a series of doses, which is effective to produce a desired therapeutic effect.

The dose of a pharmaceutical powder as described herein and oral formulations therefrom for treating a disease or disorder may depend upon the subject's condition, that is, stage of the disease, severity of symptoms caused by the disease, general health status, as well as age, gender, and weight, and other factors apparent to a person skilled in the medical art. Pharmaceutical compositions may be administered in a manner appropriate to the disease to be treated as determined by persons skilled in the medical arts. In addition to the factors described herein and above related to use of pharmaceutical agent for treating a disease or disorder, suitable duration and frequency of administration of the pharmaceutical agent may also be determined or adjusted by such factors as the condition of the patient, the type and severity of the patient's disease, the particular form of the active ingredient, and the method of administration. Optimal doses of an agent may generally be determined using experimental models and/or clinical trials. The optimal dose may depend upon the body mass, weight, or blood volume of the subject. The use of the minimum dose that is sufficient to provide effective therapy is usually preferred. Design and execution of pre-clinical and clinical studies for a pharmaceutical agent, including when administered for prophylactic benefit, described herein are well within the skill of a person skilled in the relevant art. When two or more pharmaceutical agents are administered to treat a disease or disorder, the optimal dose of each pharmaceutical agent may be different, such as less than when either agent is administered alone as a single agent therapy. In certain particular embodiments, two pharmaceutical agents in combination may act synergistically or additively, and either agent may be used in a lesser amount than if administered alone. An amount of a pharmaceutical agent that may be administered per day may be, for example, between about 0.01 mg/kg and 100 mg/kg, e.g., between about 0.1 to 1 mg/kg, between about 1 to 10 mg/kg, between about 10-50 mg/kg, between about 50-100 mg/kg body weight. In other embodiments, the amount of a pharmaceutical agent that may be administered per day is between about 0.01 mg/kg and 1000 mg/kg, between about 100-500 mg/kg, or between about 500-1000 mg/kg body weight. The optimal dose, per day or per course of treatment, may be different for the disease or disorder to be treated and may also vary with the administrative route and therapeutic regimen.

### In-Use and Long Term Stability

In one aspect, provided herein are oral liquid suspensions that are stable during reconstitution and use. In some case, oral liquid suspensions comprising an amorphous solid dispersion of Compound A with PVP-VA or a salt thereof tend to agglomerate and/or form a viscous gel immediately after reconstitution, resulting in the difficulties for a patient to accurately measure and administer the liquid formulation. It is surprisingly discovered that certain pharmaceutical excipients and combinations thereof reduce and slow down the gel formation, thereby improving the in-use stability of the oral liquid suspension.

In some embodiments, the suspension retains syringeability for at least 15 minutes. In some embodiments, the suspension retains syringeability for at least 30 minutes. In some embodiments, the suspension retains syringeability for at least 5 minutes. In some embodiments, the suspension retains syringeability for at least 10 minutes. In some embodiments, the suspension retains syringeability for at least 20 minutes. In some embodiments, the suspension retains syringeability for at least 25 minutes. In some embodiments, the syringeability is tested at room temperature.

In one aspect, provided herein are solid form pharmaceutical formulations (e.g., pharmaceutical powders) that are stable after long-term storage. In some embodiments, a solid form pharmaceutical formulation described herein (e.g., a pharmaceutical powder) retains at least 90% of the initial amount of Compound A or a pharmaceutically acceptable salt thereof after storing for 3 months at 20±5° C. In some embodiments, a solid form pharmaceutical formulation described herein (e.g., a pharmaceutical powder) retains at least 90% of the initial amount of Compound A or a pharmaceutically acceptable salt thereof after storing for 6 months at 20±5° C. In some embodiments, a solid form pharmaceutical formulation described herein (e.g., a pharmaceutical powder) retains at least 90% of the initial amount of Compound A or a pharmaceutically acceptable salt thereof after storing for 12 months at 20±5° C. In some embodiments, a solid form pharmaceutical formulation described herein (e.g., a pharmaceutical powder) retains at least 90% of the initial amount of Compound A or a pharmaceutically acceptable salt thereof after storing for 18 months at 20±5° C. In some embodiments, a solid form pharmaceutical formulation described herein (e.g., a pharmaceutical powder) retains at least 90% of the initial amount of Compound A or a pharmaceutically acceptable salt thereof after storing for 24 months at 20±5° C. In some embodiments, the amount of Compound A or a pharmaceutically acceptable salt thereof in the solid form pharmaceutical formulation is determined by HPLC.

In some embodiments, a solid form pharmaceutical formulation described herein (e.g., a pharmaceutical powder) retains at least 90% of the initial amount of Compound A or a pharmaceutically acceptable salt thereof after storing for 3 months at 5±3° C. In some embodiments, a solid form pharmaceutical formulation described herein (e.g., a pharmaceutical powder) retains at least 90% of the initial amount of Compound A or a pharmaceutically acceptable salt thereof after storing for 6 months at 5±3° C. In some embodiments, a solid form pharmaceutical formulation described herein (e.g., a pharmaceutical powder) retains at least 90% of the initial amount of Compound A or a pharmaceutically acceptable salt thereof after storing for 12 months at 5±3° C. In some embodiments, a solid form pharmaceutical formulation described herein (e.g., a pharmaceutical powder) retains at least 90% of the initial amount of Compound A or a pharmaceutically acceptable salt thereof after storing for 18 months at 5±3° C. In some embodiments, a solid form pharmaceutical formulation described herein (e.g., a pharmaceutical powder) retains at least 90% of the initial amount of Compound A or a pharmaceutically acceptable salt thereof after storing for 24 months at 5±3° C.

In some embodiments, a solid form pharmaceutical formulation described herein (e.g., a pharmaceutical powder) retains at least 90% of the initial amount of Compound A or a pharmaceutically acceptable salt thereof after storing for 3 months at about 40° C. In some embodiments, a solid form pharmaceutical formulation described herein (e.g., a pharmaceutical powder) retains at least 90% of the initial amount of Compound A or a pharmaceutically acceptable salt thereof after storing for 6 months at about 40° C. In some embodiments, a solid form pharmaceutical formulation described herein (e.g., a pharmaceutical powder) retains at least 90% of the initial amount of Compound A or a pharmaceutically acceptable salt thereof after storing for 12 months at about 40° C. In some embodiments, a solid form pharmaceutical formulation described herein (e.g., a pharmaceutical powder) retains at least 90% of the initial amount of Compound A or a pharmaceutically acceptable salt thereof after storing for 18 months at about 40° C. In some embodiments, a solid form pharmaceutical formulation described herein (e.g., a pharmaceutical powder) retains at least 90% of the initial amount of Compound A or a pharmaceutically acceptable salt thereof after storing for 24 months at about 40° C.

In some embodiments, a solid form pharmaceutical formulation described herein (e.g., a pharmaceutical powder) retains at least 95% of the initial amount of Compound A or a pharmaceutically acceptable salt thereof after storing for 3 months at 20±5° C. In some embodiments, a solid form pharmaceutical formulation described herein (e.g., a pharmaceutical powder) retains at least 95% of the initial amount of Compound A or a pharmaceutically acceptable salt thereof after storing for 6 months at 20±5° C. In some embodiments, a solid form pharmaceutical formulation described herein (e.g., a pharmaceutical powder) retains at least 95% of the initial amount of Compound A or a pharmaceutically acceptable salt thereof after storing for 12 months at 20±5° C. In some embodiments, a solid form pharmaceutical formulation described herein (e.g., a pharmaceutical powder) retains at least 95% of the initial amount of Compound A or a pharmaceutically acceptable salt thereof after storing for 18 months at 20±5° C. In some embodiments, a solid form pharmaceutical formulation described herein (e.g., a pharmaceutical powder) retains at least 95% of the initial amount of Compound A or a pharmaceutically acceptable salt thereof after storing for 24 months at 20±5° C.

In some embodiments, a solid form pharmaceutical formulation described herein (e.g., a pharmaceutical powder) retains at least 95% of the initial amount of Compound A or a pharmaceutically acceptable salt thereof after storing for 3 months at 5±3° C. In some embodiments, a solid form pharmaceutical formulation described herein (e.g., a pharmaceutical powder) retains at least 95% of the initial amount of Compound A or a pharmaceutically acceptable salt thereof after storing for 6 months at 5±3° C. In some embodiments, a solid form pharmaceutical formulation described herein (e.g., a pharmaceutical powder) retains at least 95% of the initial amount of Compound A or a pharmaceutically acceptable salt thereof after storing for 12 months at 5±3° C. In some embodiments, a solid form pharmaceutical formulation described herein (e.g., a pharmaceutical powder) retains at least 95% of the initial amount of Compound A or a pharmaceutically acceptable salt thereof after storing for 18 months at 5±3° C. In some embodiments, a solid form pharmaceutical formulation described herein (e.g., a pharmaceutical powder) retains at least 95% of the initial amount of Compound A or a pharmaceutically acceptable salt thereof after storing for 24 months at 5±3° C.

In some embodiments, a solid form pharmaceutical formulation described herein (e.g., a pharmaceutical powder) retains at least 95% of the initial amount of Compound A or a pharmaceutically acceptable salt thereof after storing for 3 months at about 40° C. In some embodiments, a solid form pharmaceutical formulation described herein (e.g., a pharmaceutical powder) retains at least 95% of the initial amount of Compound A or a pharmaceutically acceptable salt thereof after storing for 6 months at about 40° C. In some embodiments, a solid form pharmaceutical formulation described herein (e.g., a pharmaceutical powder) retains at least 95% of the initial amount of Compound A or a pharmaceutically acceptable salt thereof after storing for 12 months at about 40° C. In some embodiments, a solid form pharmaceutical formulation described herein (e.g., a pharmaceutical powder) retains at least 95% of the initial amount of Compound A or a pharmaceutically acceptable salt thereof after storing for 18 months at about 40° C. In some embodiments, a solid form pharmaceutical formulation described herein (e.g., a pharmaceutical powder) retains at least 95% of the initial amount of Compound A or a pharmaceutically acceptable salt thereof after storing for 24 months at about 40° C.

### Method of Preparing Oral Formulations

In certain aspects the present disclosure provides methods of preparing an aqueous formulation comprising:
(a) providing a solid formulation of an amorphous solid dispersion, wherein the amorphous solid dispersion comprises:
   (i) (R)-2-(1-(6-amino-5-chloropyrimidine-4-carboxamido)ethyl)-N-(5-chloro-4-(trifluoromethyl)pyridin-2-yl)thiazole-5-carboxamide (Compound A) or a pharmaceutically acceptable salt thereof and
   (ii) one or more polymers; and
(b) contacting the solid formulation with an aqueous solution

In some embodiments, the solid formulation is a pharmaceutical powder as disclosed herein. In some embodiments, the contacting comprises mixing the solid formulation with an aqueous solution to form a suspension. In some embodiments, the mixing comprises shaking the solid formulation and the aqueous solution to form a suspension. In some embodiments, the mixing comprises adding the solid formulation to a container that comprises the aqueous solution and then shaking the container to form a suspension. In some embodiments, the container is a container as described herein. In some embodiments, the container is shaken in all directions. In some embodiments, wherein the mixing or shaking continues until all of the solid formulation is dispersed in the aqueous solution.

In some embodiments, the mixing lasts about 10 seconds to about 240 seconds. the mixing lasts about 10 seconds to about 60 seconds, about 60 seconds to about 120 seconds, about 40 seconds to about 80 seconds, or about 50 seconds to about 70 seconds In some embodiments, the mixing lasts about 10 seconds, about 15 seconds, about 20 seconds, about 25 seconds, about 30 seconds, about 35 seconds, about 40 seconds, about 45 seconds, about 50 seconds, about 55 seconds, about 60 seconds, about 65 seconds, about 70 seconds, about 75 seconds, about 80 seconds, about 85 seconds, about 90 seconds, about 95 seconds, about 100 seconds, about 105 seconds, about 110 seconds, about 115 seconds, or about 120 seconds. In some embodiments, the mixing lasts at least 10 seconds, at least 15 seconds, at least 20 seconds, at least 25 seconds, at least 30 seconds, at least 35 seconds, at least 40 seconds, at least 45 seconds, at least 50 seconds, at least 55 seconds, at least 60 seconds, at least 65 seconds, at least 70 seconds, at least 75 seconds, at least 80 seconds, at least 85 seconds, at least 90 seconds, at least 95 seconds, at least 100 seconds, at least 105 seconds, at least 110 seconds, at least 115 seconds, or at least 120 seconds. In some embodiments, the mixing lasts at most 10 seconds, at most 15 seconds, at most 20 seconds, at most 25 seconds, at most 30 seconds, at most 35 seconds, at most 40 seconds, at most 45 seconds, at most 50 seconds, at most 55 seconds, at most 60 seconds, at most 65 seconds, at most 70 seconds, at most 75 seconds, at most 80 seconds, at most 85 seconds, at most 90 seconds, at most 95 seconds, at most 100 seconds, at most 105 seconds, at most 110 seconds, at most 115 seconds, or most 120 seconds. In some embodiments, the mixing lasts 60 seconds. In some embodiments, the mixing does not last more than about 120 seconds.

In some embodiments, the shaking lasts about 10 seconds to about 240 seconds. the mixing lasts about 10 seconds to about 60 seconds, about 60 seconds to about 120 seconds, about 40 seconds to about 80 seconds, or about 50 seconds to about 70 seconds In some embodiments, the shaking lasts about 10 seconds, about 15 seconds, about 20 seconds, about 25 seconds, about 30 seconds, about 35 seconds, about 40 seconds, about 45 seconds, about 50 seconds, about 55 seconds, about 60 seconds, about 65 seconds, about 70 seconds, about 75 seconds, about 80 seconds, about 85 seconds, about 90 seconds, about 95 seconds, about 100 seconds, about 105 seconds, about 110 seconds, about 115 seconds, or about 120 seconds. In some embodiments, the mixing lasts at least 10 seconds, at least 15 seconds, at least 20 seconds, at least 25 seconds, at least 30 seconds, at least 35 seconds, at least 40 seconds, at least 45 seconds, at least 50 seconds, at least 55 seconds, at least 60 seconds, at least 65 seconds, at least 70 seconds, at least 75 seconds, at least 80 seconds, at least 85 seconds, at least 90 seconds, at least 95 seconds, at least 100 seconds, at least 105 seconds, at least 110 seconds, at least 115 seconds, or at least 120 seconds. In some embodiments, the shaking lasts at most 10 seconds, at most 15 seconds, at most 20 seconds, at most 25 seconds, at most 30 seconds, at most 35 seconds, at most 40 seconds, at most 45 seconds, at most 50 seconds, at most 55 seconds, at most 60 seconds, at most 65 seconds, at most 70 seconds, at most 75 seconds, at most 80 seconds, at most 85 seconds, at most 90 seconds, at most 95 seconds, at most 100 seconds, at most 105 seconds, at most 110 seconds, at most 115 seconds, or most 120 seconds. In some embodiments, the shaking lasts 60 seconds. In some embodiments, the shaking does not last more than about 120 seconds.

In some embodiments, the mixing or shaking is done in increments. In some embodiments, the mixing or shaking occurs in 5-second, 10-second, 15-second, 20-second, 25-second, or 30-second increments. In some embodiments, the mixing or shaking occurs in about 5-second, about 10-second, about 15-second, about 20-second, about 25-second, or about 30-second increments. In some embodiments, the mixing or shaking occurs in 15-second increments.

In some embodiments, the method further comprises comprising allow the container to sit for a period time without mixing or shaking. In some embodiments, the period of time is about 1 minute, about 2 minutes, about 3 minutes, about 4 minutes, about 5 minutes, about 10 minutes, about 15 minutes, about 20 minutes, about 25 minutes, or about 30 minutes.

In some embodiments, the method further comprises, inverting the container and continuing to mix the suspension. In some embodiments, comprising inverting the container and swirling for about 10 seconds to about 60 seconds, about 20 seconds to about 40 seconds, about 40 seconds to about 80 seconds, or about 15 seconds to about 45 seconds. In some embodiments, the swirling is for about 10 seconds, about 15 seconds, about 20 seconds, about 25 seconds, about 30 seconds, about 35 seconds, about 40 seconds, about 45 seconds, about 50 seconds, about 55 seconds, about 60 seconds, about 65 seconds, about 70 seconds, about 75 seconds, or about 80 seconds.

In some embodiments, the aqueous solution is at an elevated temperature or near room temperature. In some embodiments, the aqueous solution is near room temperature. In some embodiments, the aqueous solution has a temperature of about 15° C. to about 80° C., about 20° C. to about 60° C., or about 20° C. to about 50° C. In some embodiments, the aqueous solution has a temperature of about 15° C., about 20° C., about 25° C., about 30° C., about 35° C., about 40° C., about 45° C., about 50° C., about 55° C., about 60° C., about 65° C., about 70° C., about 75° C., about 80° C., or about 85° C. In some embodiments, the aqueous solution has a temperature of at least 15° C., at least 20° C., at least 25° C., at least 30° C., at least 35° C., at least 40° C., at least 45° C., at least 50° C., at least 55°, C, at least 60° C., at least 65° C., at least 70° C., at least 75° C., at least 80° C., or at least 85° C. In some embodiments, the aqueous solution has a temperature of at most 15° C., at most 20° C., at most 25° C., at most 30° C., at most 35° C., at most 40° C., at most 45° C., at most 50° C., at most 55°, C, at most 60° C., at most 65° C., at most 70° C., at most 75° C., at most 80° C., or at most 85° C. In some embodiments, the aqueous solution is warm or room temperature water. In some embodiments, the aqueous solution is warm water. In some embodiments, aqueous solution is room temperature water.

In some embodiments, a volume of the aqueous solution is about 5 mL to about 200 mL, about 5 mL to 100 mL, about 5 mL to 50 mL, about 5 mL to 25 mL, about 5 mL to 20 mL, about 10 mL to 50 mL, about 10 mL to 25 mL, about 25 mL to 50 mL, about 25 mL to 150 mL, or about 10 mL to about 20 mL. In some embodiments, the volume of the aqueous solution is about 5 mL, about 10 mL, about 15 mL, about 20 mL, about 25 mL, about 30 mL, about 35 mL, about 40 mL, about 45 mL, about 50 mL, about 60 mL, about 70 mL, about 80 mL, about 90 mL, about 100 mL, about 125 mL, about 150 mL, about 175 mL, or about 200 mL. In some embodiments, a volume of the aqueous solution is about 10 mL, about 11 mL, about 12 mL, about 13 mL, about 14 mL, about 15 mL, about 16 mL, about 17 mL, about 18 mL, about 19 mL, or about 20 mL. In some embodiments, a volume of the aqueous solution is about 14 mL.

In some embodiments, the method further comprising withdrawing the suspension from the container with a syringe. In some embodiments, the suspension administered to a subject directly from the syringe.

In some embodiments, the aqueous formulation as disclosed herein is administered with food, before consuming food, or after consuming food. In some embodiments, the aqueous formulation is administered with food. In some embodiments, the aqueous formulation is administered before the subject consumes food. In some embodiments, the aqueous formulation is administered after the subject consumes food.

### Kits

In certain aspects the present disclosure provides a kit comprising a pharmaceutical powder or pharmaceutical compositions as disclosed herein for reconstitution. In some embodiments, a pharmaceutical powder or pharmaceutical composition in the kit is reconstituted using preparation methods as disclosed herein.

In certain aspects, the present disclosure provides a method of preparing an oral liquid suspension of Compound A or a salt thereof, comprising reconstituting the solid formulation in a kit as disclosed herein in an aqueous solution.

In certain aspects the present disclosure provides a method of preparing an oral liquid suspension of Compound A or a salt thereof, comprising reconstituting a pharmaceutical powder as disclosed herein in an aqueous solution.

In another aspect, the present disclosure provides methods of treating cancer in a subject comprising reconstituting a solid formulation in a kit as disclosed herein and administering the reconstituted formulation to the subject in need thereof. For the compositions of Compound A or a pharmaceutically acceptable salt thereof including powder and liquid compositions described herein, kits and articles of manufacture are also described. Such kits can comprise a carrier, package, or container that is compartmentalized to receive one or more containers such as vials, tubes, bottles, and the like, each of the container(s) comprising one of the separate elements to be used in a method described herein including a solid formulation or liquid formulation. Suitable containers include, for example, bottles, vials, syringes, and test tubes. The containers can be formed from a variety of materials such as glass or plastic (e.g., HDPE).

In some embodiments, the kit comprises one or more containers that optionally comprise markings denoting the measurement of volume. In some embodiments, the kit comprises one container that optionally comprises markings denoting the measurement of volume. In some embodiments, the kit comprises 1-10 containers that optionally comprise markings denoting the measurement of volume. In some embodiments, the kit comprises 1, 2, 3, 4, 5, 6, 7, 8, 9, or 10 containers that optionally comprise markings denoting the measurement of volume. In some embodiments, the container is reusable after each use. In some embodiments, the container is disposable after use.

A kit can comprise one or more additional containers, each with one or more of various materials (such as reagents, optionally in concentrated form, and/or devices) desirable from a commercial and user standpoint for a solid formulation or liquid formulation described herein.

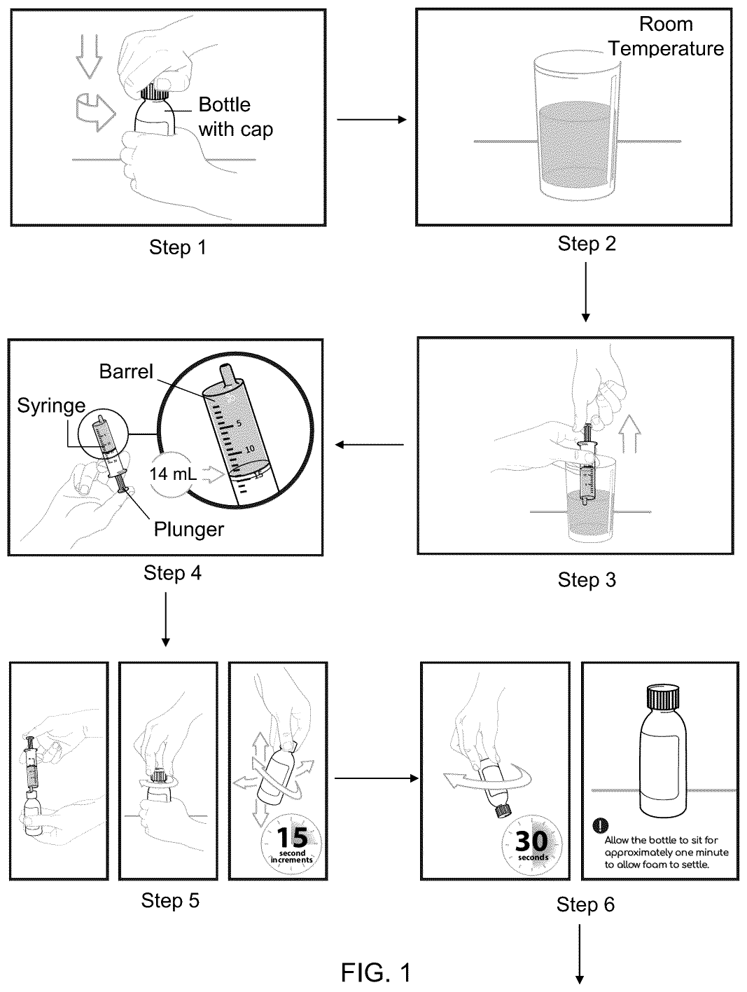

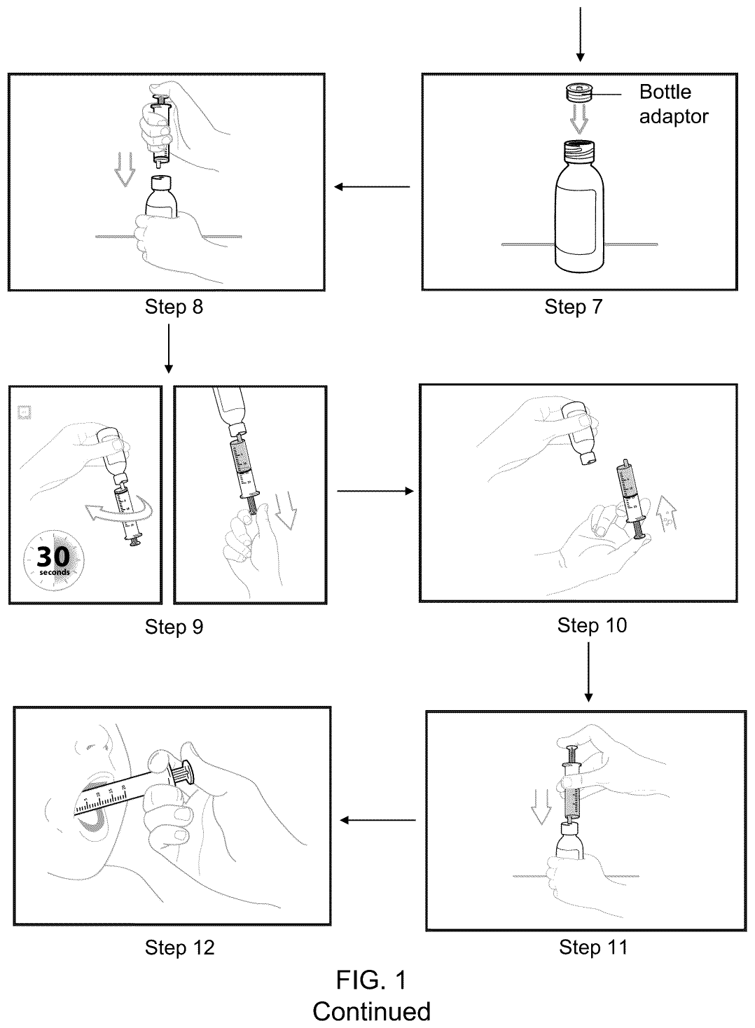

Non-limiting examples of such materials include, but not limited to, buffers, diluents, filters, needles, syringes; carrier, package, container, vial and/or tube labels listing contents and/or instructions for use, and package inserts with instructions for use associated with a solid formulation or liquid formulation. A set of instructions can also be included. In some embodiments, the kit comprises a set of instructions as shown in FIG. 1. In some embodiments, a kit can comprise a dosing syringe and/or a dispensing syringe. In some embodiments, a kit comprises a bottle adapter.

A label can be on or associated with the container. A label can be on a container when letters, numbers or other characters forming the label are attached, molded or etched into the container itself; a label can be associated with a container when it is present within a receptacle or carrier that also holds the container, e.g., as a package insert. A label can be used to indicate that the contents are to be used for a specific therapeutic application. The label can also indicate directions for use of the contents, such as in the methods described herein.

In some embodiments, the syringe in a kit is used to transfer a predetermined amount of the liquid formulation comprising Compound A or a pharmaceutically acceptable salt thereof from a container to form a suspension. The suspension can be prepared by dispersing Compound A or a pharmaceutically acceptable salt thereof in powder form into a predefined volume of reconstitution media for single use or in a bottle for multi-use. The patient individual dose can be applied by using the dosing syringe. The suspension in the syringe can be directly given into a child's mouth. In certain aspects the present disclosure provides a kit comprising (a) a solid formulation of an amorphous solid dispersion of (R)-2-(1-(6-amino-5-chloropyrimidine-4-carboxamido)ethyl)-N-(5-chloro-4-(trifluoromethyl)pyridin-2-yl)thiazole-5-carboxamide (Compound A) or a pharmaceutically acceptable salt thereof, wherein the solid formulation comprises one or more pharmaceutically acceptable excipients; and (b) instructions for aqueous reconstitution of the solid formulation. An example of the instruction is shown in FIG. 1. In some embodiments, the instructions for aqueous reconstitution are in the form of a reference that refers to the instructions.

In some embodiments, the solid formulation is in a powder, granular, or pellet form. In some embodiments, the solid formulation is in a powder form. In some embodiments, the solid formulation is in a granular form. In some embodiments, the solid formulation is in pellet form. In some embodiments, the solid formulation is a tablet wherein the tablet is orange, film-coated, and oval and debossed with “100” on one side and “D101” on the opposite side. In some embodiments, the solid formulation is in a power form wherein the powder is white to off white and is further reconstituted with water to form a uniform, flavored, white suspension with a concentration of 25 mg/mL.

In some embodiments, a kit of the present disclosure comprises one or more of the following: a solid formulation of Compound A or a pharmaceutically acceptable salt thereof, instructions for aqueous reconstitution of the solid formulation, a bottle, a bottle cap, a bottle adaptor, and/or a syringe. In some embodiments, the kit comprises a solid formulation of Compound A or a pharmaceutically acceptable salt thereof, instructions for aqueous reconstitution of the solid formulation, a bottle, a bottle cap, a bottle adaptor, and a syringe. The syringe can be a dosing syringe with markings indicating the volume of the liquid therein. The syringe can optionally be individually wrapped, e.g., in a plastic bag. In some embodiments, the solid formulation of Compound A or a pharmaceutically acceptable salt thereof is contained in the bottle, which can be sealed by the bottle cap and/or a seal. In some embodiments, the bottle is configured to contain a liquid volume of at least 25 mL, at least 50 mL, at least 75 mL, at least 100 mL, at least 125 mL, at least 150 mL, or least 200 mL. In some embodiments, the bottle is configured to contain a liquid volume of about 25 mL to about 75 mL. In some embodiments, the bottle is configured to contain a liquid volume of about 15 mL to about 50 mL. In some embodiments, the bottle is configured to contain a liquid volume of about 50 mL to about 150 mL. In some embodiments, the bottle is configured to contain a liquid volume of about 100 mL to about 200 mL. In some embodiments, the bottle adaptor is configured to fit into the bottle neck and allow the tip of the syringe to be inserted into a hole of the bottle adaptor. In some embodiments, the bottle adaptor is configured to allow a syringe to draw a liquid from the bottle without spilling or leaking. In some embodiments, a kit of the present disclosure comprises 1 mg to 5000 mg of a solid formulation (e.g., a pharmaceutical powder) of Compound A or a pharmaceutically acceptable salt thereof. In some embodiments, the kit comprises about 100 mg to about 1000 mg of a solid formulation (e.g., a pharmaceutical powder) comprising Compound A. In some embodiments, the kit comprises about 300 mg to about 600 mg of a solid formulation (e.g., a pharmaceutical powder) comprising Compound A. In some embodiments, the kit comprises about 100 mg to about 200 mg of a solid formulation (e.g., a pharmaceutical powder) comprising Compound A. In some embodiments, the kit comprises about 500 mg to about 1500 mg of a solid formulation (e.g., a pharmaceutical powder) comprising Compound A. In some embodiments, the kit comprises about 200 mg to about 800 mg of a solid formulation (e.g., a pharmaceutical powder) comprising Compound A. In some embodiments, the kit comprises about 400 mg to about 450 mg of a solid formulation (e.g., a pharmaceutical powder) comprising Compound A. In some embodiments, the kit comprises about 400 mg to about 500 mg of a solid formulation (e.g., a pharmaceutical powder) comprising Compound A. In some embodiments, the kit comprises about 430 mg of a solid formulation (e.g., a pharmaceutical powder) comprising Compound A. In some embodiments, the kit comprises about 300 mg, about 350 mg, about 400 mg, about 450 mg, about 500 mg, about 550 mg, about 600 mg, or about 650 mg of a solid formulation (e.g., a pharmaceutical powder) comprising Compound A.

### Dosing

Kits with unit doses of one or more of the agents described herein, usually in oral or injectable doses, are provided. Such kits may include a container containing the unit dose, an informational package insert describing the use and attendant benefits of the drugs in treating disease, and optionally an appliance or device for delivery of the composition.

Described herein are solid and liquid formulations of Compound A or a pharmaceutically acceptable salt thereof. In some embodiments, the solid and liquid formulations of Compound A or a pharmaceutically acceptable salts thereof, are used as unit dosing.

The present disclosure provides kits with a unit dose or doses. A unit dose comprises an amount of a solid formulation (pharmaceutical powders) as disclosed herein. In some embodiments, the kit comprises a plurality of unit doses each of which comprise compound A or a pharmaceutically acceptable salt thereof. In some embodiments, the kit comprises one unit dose which comprises compound A or a pharmaceutically acceptable salt thereof. In some embodiments, the kit comprises a single dose that comprises compound A or a pharmaceutically acceptable salt thereof. In some embodiments, the kit comprises two or more unit doses, which comprise compound A or a pharmaceutically acceptable salt thereof. In some embodiments, the kit comprises 1-10 unit doses, each of which comprise compound A or a pharmaceutically acceptable salt thereof. In some embodiments, the kit comprises 1-5 unit doses, each of which comprise compound A or a pharmaceutically acceptable salt thereof. In some embodiments, the kit comprises 1, 2, 3, 4, 5, 6, 7, 8, 9, or 10 unit doses, each of which comprise compound A or a pharmaceutically acceptable salt thereof. In some embodiments, the unit dose is a pediatric dose.

In some embodiments, each unit dose comprises about 25 mg to about 1000 mg of compound A or a pharmaceutically acceptable salt thereof. In some embodiments, each unit dose comprises about 25 mg to about 900 mg of compound A or a pharmaceutically acceptable salt thereof. In some embodiments, each unit dose comprises about 25 mg to about 800 mg of compound A or a pharmaceutically acceptable salt thereof. In some embodiments, each unit dose comprises about 25 mg to about 700 mg of compound A or a pharmaceutically acceptable salt thereof. In some embodiments, each unit dose comprises about 25 mg to about 600 mg of compound A or a pharmaceutically acceptable salt thereof. In some embodiments, each unit dose comprises about 25 mg to about 500 mg of compound A or a pharmaceutically acceptable salt thereof. In some embodiments, each unit dose comprises about 25 mg to about 400 mg of compound A or a pharmaceutically acceptable salt thereof. In some embodiments, each unit dose comprises about 25 mg to about 300 mg of compound A or a pharmaceutically acceptable salt thereof. In some embodiments, each unit dose comprises about 25 mg to about 200 mg of compound A or a pharmaceutically acceptable salt thereof. In some embodiments, each unit dose comprises about 25 mg to about 100 mg of compound A or a pharmaceutically acceptable salt thereof. In some embodiments, each unit dose comprises about 100 mg to about 1000 mg of compound A or a pharmaceutically acceptable salt thereof. In some embodiments, each unit dose comprises about 200 mg to about 1000 mg of compound A or a pharmaceutically acceptable salt thereof. In some embodiments, each unit dose comprises about 300 mg to about 1000 mg of compound A or a pharmaceutically acceptable salt thereof. In some embodiments, each unit dose comprises about 400 mg to about 1000 mg of compound A or a pharmaceutically acceptable salt thereof. In some embodiments, each unit dose comprises about 500 mg to about 1000 mg of compound A or a pharmaceutically acceptable salt thereof. In some embodiments, each unit dose comprises about 600 mg to about 1000 mg of compound A or a pharmaceutically acceptable salt thereof. In some embodiments, each unit dose comprises about 700 mg to about 1000 mg of compound A or a pharmaceutically acceptable salt thereof. In some embodiments, each unit dose comprises about 800 mg to about 1000 mg of compound A or a pharmaceutically acceptable salt thereof. In some embodiments, each unit dose comprises about 900 mg to about 1000 mg of compound A or a pharmaceutically acceptable salt thereof. In some embodiments, each unit dose comprises about 200 mg to about 900 mg of compound A or a pharmaceutically acceptable salt thereof. In some embodiments, each unit dose comprises about 300 mg to about 800 mg of compound A or a pharmaceutically acceptable salt thereof. In some embodiments, each unit dose comprises about 400 mg to about 700 mg of compound A or a pharmaceutically acceptable salt thereof. In some embodiments, each unit dose comprises about 500 mg to about 600 mg of compound A or a pharmaceutically acceptable salt thereof. In some embodiments, each unit dose comprises about 400 mg to about 600 mg of compound A or a pharmaceutically acceptable salt thereof. In some embodiments, each unit dose comprises about 300 mg to about 600 mg of compound A or a pharmaceutically acceptable salt thereof.

In some embodiments, each unit dose comprises about 25 mg, about 50 mg, about 100 mg, about 150 mg, about 200 mg, about 250 mg, about 300 mg, about 350 mg, about 400 mg, about 450 mg, about 500 mg, about 600 mg, about 700 mg, about 800 mg, about 900 mg, or about 1000 mg of compound A or a pharmaceutically acceptable salt thereof. In some embodiments, each unit dose comprises at least 25, at least 50 mg, at least 100 mg, at least 150 mg, at least 200 mg, at least 250 mg, at least 300 mg, at least 350 mg, at least 400 mg, at least 450 mg, at least 500 mg, at least 600 mg, at least 700 mg, at least 800 mg, at least 900 mg, or at least 1000 mg of compound A or a pharmaceutically acceptable salt thereof. In some embodiments, each unit dose comprises at most 25 mg, at most 50 mg, at most 100 mg, at most 150 mg, at most 200 mg, at most 250 mg, at most 300 mg, at most 350 mg, at most 400 mg, at most 450 mg, at most 500 mg, at most 600 mg, at most 700 mg, at most 800 mg, at most 900 mg, or at most 1000 mg of compound A or a pharmaceutically acceptable salt thereof.

In some embodiments, if a weekly dose is missed by 3 days or less, the missed dose should be taken as soon as possible and the next dose should be taken on the usual day. In some embodiments, if a weekly dose is missed by more than 3 days, the missed dose should be skipped and the next dose should be taken on the usual day. In some embodiments, a minimum of four days should occur between doses.

In some embodiments, the dose is not co-administered with strong or moderate CYP2C8 inhibitors. In some embodiments, the dose is not co-administered with a strong or moderate CYP2C8 inducer. In some embodiments, the dose is not co-administered with sensitive CYP3A4 substrates. In some embodiments, the dose is not co-administered with a BCRP substrate. In some embodiments, the dose is not administered to pregnant subjects. In some embodiments, the dose is not administered to lactating subjects. In some embodiments, the dose is not administered to subjects with hepatic impairment.

### Oral Formulations for the Treatment of Cancer

In certain aspects the present disclosure provides methods of treating cancer in a subject comprising administering to the subject a pharmaceutical powder as disclosed herein or a reconstituted oral liquid formulation as disclosed herein.

In certain aspects, the present disclosure provides methods of treating cancer comprising administering to the subject an oral liquid suspension comprising (R)-2-(1-(6-amino-5-chloropyrimidine-4-carboxamido)ethyl)-N-(5-chloro-4-(trifluoromethyl)pyridin-2-yl)thiazole-5-carboxamide (Compound A), or a pharmaceutically acceptable salt thereof to achieve a prescribed pharmacokinetic profile. During or following the administration of the oral liquid suspension, plasma concentrations of (R)-2-(1-(6-amino-5-chloropyrimidine-4-carboxamido)ethyl)-N-(5-chloro-4-(trifluoromethyl)pyridin-2-yl)thiazole-5-carboxamide (Compound A) or a pharmaceutically acceptable salt thereof, can be determined with a validated bioanalytical assay. For example, the following pharmacokinetic (PK) parameters can be calculated where appropriate: maximum observed blood plasma concentration (Cmax) and area under the concentration versus time curve from time 0 to t (AUC₀₋ₜ).

In certain aspects the present disclosure provides methods of treating cancer comprising administering to the subject an oral liquid suspension comprising (R)-2-(1-(6-amino-5-chloropyrimidine-4-carboxamido)ethyl)-N-(5-chloro-4-(trifluoromethyl)pyridin-2-yl)thiazole-5-carboxamide (Compound A), or a pharmaceutically acceptable salt thereof in an amount sufficient to achieve in the subject a maximum observed blood plasma concentration (Cmax) of Compound A of at least 2000 ng/mL. In some embodiments, the oral liquid suspension is reconstituted from a solid formulation described herein, e.g., a pharmaceutical powder comprising Compound A or a pharmaceutically acceptable salt thereof.

### Pharmacokinetic Parameters

In some embodiments, the oral liquid suspension comprising Compound A or a pharmaceutically acceptable salt thereof is administered in an amount sufficient to achieve in the subject a maximum observed blood plasma concentration (Cmax) of Compound A of at least 100 ng/mL, at least 200 ng/mL, at least 300 ng/mL, at least 400 ng/mL, at least 500 ng/mL, at least 1000 ng/mL, at least 1500 ng/mL, at least 2000 ng/mL, at least 2500 ng/mL, at least 3000 ng/mL, at least 3500 ng/mL, at least 4000 ng/mL, at least 4500 ng/mL, at least 5000 ng/mL, at least 5500 ng/mL, at least 6000 ng/mL, at least 6500 ng/mL, at least 7000 ng/mL, at least 7500 ng/mL, or at least 8000 ng/mL. In some embodiments, the oral liquid suspension comprising Compound A or a pharmaceutically acceptable salt thereof is administered in an amount sufficient to achieve in the subject a maximum observed blood plasma concentration (Cmax) of Compound A of at most 100 ng/mL, at most 200 ng/mL, at most 300 ng/mL, at most 400 ng/mL, at most 500 ng/mL, at most 1000 ng/mL, at most 1500 ng/mL, at most 2000 ng/mL, at most 2500 ng/mL, at most 3000 ng/mL, at most 3500 ng/mL, at most 4000 ng/mL, at most 4500 ng/mL, at most 5000 ng/mL, at most 5500 ng/mL, at most 6000 ng/mL, at most 6500 ng/mL, at most 7000 ng/mL, at most 7500 ng/mL, or at most 8000 ng/mL.

In some embodiments, the oral liquid suspension comprising Compound A or a pharmaceutically acceptable salt thereof is administered in an amount sufficient to achieve in the subject a maximum observed blood plasma concentration (Cmax) of Compound A of about 100 ng/mL, about 200 ng/mL, about 300 ng/mL, about 400 ng/mL, about 500 ng/mL, about 1000 ng/mL, about 1500 ng/mL, about 2000 ng/mL, about 2500 ng/mL, about 3000 ng/mL, about 3500 ng/mL, about 4000 ng/mL, about 4500 ng/mL, about 5000 ng/mL, about 5500 ng/mL, about 6000 ng/mL, about 6500 ng/mL, about 7000 ng/mL, about 7500 ng/mL, or about 8000 ng/mL. In some embodiments, the oral liquid suspension comprising Compound A or a pharmaceutically acceptable salt thereof is administered in amount that is sufficient to achieve in the subject a Cmax of Compound A that is within a suitable range. In some embodiments, the Cmax is about 100 ng/mL to about 8,000 ng/mL. In some embodiments, the Cmax is about 100 ng/mL to about 2,000 ng/mL. In some embodiments, the Cmax is about 2,000 ng/mL to about 8,000 ng/mL. In some embodiments, the Cmax is at least about 100 ng/mL. In some embodiments, the Cmax is at least about 200 ng/mL. In some embodiments, the Cmax is at least about 300 ng/mL. In some embodiments, the Cmax is at least about 400 ng/mL. In some embodiments, the Cmax is at least about 500 ng/mL. In some embodiments, the Cmax is at least about 1000 ng/mL. In some embodiments, the Cmax is at least about 2,000 ng/mL. In some embodiments, the Cmax is at least about 3,000 ng/mL. In some embodiments, the Cmax is at least about 4,000 ng/mL. In some embodiments, the Cmax is at least about 5,000 ng/mL. In some embodiments, the Cmax is at least about 6,000 ng/mL. In some embodiments, the Cmax is at least about 7,000 ng/mL. In some embodiments, the Cmax is at most about 8,000 ng/mL.

In some embodiments, the oral liquid suspension comprising Compound A or a pharmaceutically acceptable salt thereof is administered in amount that is sufficient to achieve in the subject a Cmax of Compound A that is about 100 ng/mL to about 300 ng/mL, about 100 ng/mL to about 500 ng/mL, about 100 ng/mL to about 1,000 ng/mL, about 100 ng/mL to about 1,500 ng/mL, about 100 ng/mL to about 2,000 ng/mL, about 100 ng/mL to about 2,500 ng/mL, about 100 ng/mL to about 3,000 ng/mL, about 100 ng/mL to about 3,500 ng/mL, about 100 ng/mL to about 4,000 ng/mL, about 100 ng/mL to about 4,500 ng/mL, about 100 ng/mL to about 5,000 ng/mL, about 100 ng/mL to about 5,500 ng/mL, about 100 ng/mL to about 6,000 ng/mL, about 100 ng/mL to about 6,500 ng/mL, about 100 ng/mL to about 7,000 ng/mL, about 100 ng/mL to about 8,000 ng/mL, about 2,000 ng/mL to about 2,500 ng/mL, about 2,000 ng/mL to about 3,000 ng/mL, about 2,000 ng/mL to about 3,500 ng/mL, about 2,000 ng/mL to about 4,000 ng/mL, about 2,000 ng/mL to about 4,500 ng/mL, about 2,000 ng/mL to about 5,000 ng/mL, about 2,000 ng/mL to about 5,500 ng/mL, about 2,000 ng/mL to about 6,000 ng/mL, about 2,000 ng/mL to about 6,500 ng/mL, about 2,000 ng/mL to about 7,000 ng/mL, about 2,000 ng/mL to about 8,000 ng/mL, about 2,500 ng/mL to about 3,000 ng/mL, about 2,500 ng/mL to about 3,500 ng/mL, about 2,500 ng/mL to about 4,000 ng/mL, about 2,500 ng/mL to about 4,500 ng/mL, about 2,500 ng/mL to about 5,000 ng/mL, about 2,500 ng/mL to about 5,500 ng/mL, about 2,500 ng/mL to about 6,000 ng/mL, about 2,500 ng/mL to about 6,500 ng/mL, about 2,500 ng/mL to about 7,000 ng/mL, about 2,500 ng/mL to about 8,000 ng/mL, about 3,000 ng/mL to about 3,500 ng/mL, about 3,000 ng/mL to about 4,000 ng/mL, about 3,000 ng/mL to about 4,500 ng/mL, about 3,000 ng/mL to about 5,000 ng/mL, about 3,000 ng/mL to about 5,500 ng/mL, about 3,000 ng/mL to about 6,000 ng/mL, about 3,000 ng/mL to about 6,500 ng/mL, about 3,000 ng/mL to about 7,000 ng/mL, about 3,000 ng/mL to about 8,000 ng/mL, about 3,500 ng/mL to about 4,000 ng/mL, about 3,500 ng/mL to about 4,500 ng/mL, about 3,500 ng/mL to about 5,000 ng/mL, about 3,500 ng/mL to about 5,500 ng/mL, about 3,500 ng/mL to about 6,000 ng/mL, about 3,500 ng/mL to about 6,500 ng/mL, about 3,500 ng/mL to about 7,000 ng/mL, about 3,500 ng/mL to about 8,000 ng/mL, about 4,000 ng/mL to about 4,500 ng/mL, about 4,000 ng/mL to about 5,000 ng/mL, about 4,000 ng/mL to about 5,500 ng/mL, about 4,000 ng/mL to about 6,000 ng/mL, about 4,000 ng/mL to about 6,500 ng/mL, about 4,000 ng/mL to about 7,000 ng/mL, about 4,000 ng/mL to about 8,000 ng/mL, about 4,500 ng/mL to about 5,000 ng/mL, about 4,500 ng/mL to about 5,500 ng/mL, about 4,500 ng/mL to about 6,000 ng/mL, about 4,500 ng/mL to about 6,500 ng/mL, about 4,500 ng/mL to about 7,000 ng/mL, about 4,500 ng/mL to about 8,000 ng/mL, about 5,000 ng/mL to about 5,500 ng/mL, about 5,000 ng/mL to about 6,000 ng/mL, about 5,000 ng/mL to about 6,500 ng/mL, about 5,000 ng/mL to about 7,000 ng/mL, about 5,000 ng/mL to about 8,000 ng/mL, about 5,500 ng/mL to about 6,000 ng/mL, about 5,500 ng/mL to about 6,500 ng/mL, about 5,500 ng/mL to about 7,000 ng/mL, about 5,500 ng/mL to about 8,000 ng/mL, about 6,000 ng/mL to about 6,500 ng/mL, about 6,000 ng/mL to about 7,000 ng/mL, about 6,000 ng/mL to about 8,000 ng/mL, about 6,500 ng/mL to about 7,000 ng/mL, about 6,500 ng/mL to about 8,000 ng/mL, or about 7,000 ng/mL to about 8,000 ng/mL.

In some embodiments, the oral liquid suspension comprising Compound A or a pharmaceutically acceptable salt thereof is administered in amount that is sufficient to achieve a prescribed AUC level of Compound A. In some embodiments, the area under the concentration versus time curve from time 0 to t (AUC₀₋ₜ) or AUCss (steady-state AUC) is measured in a subject administered Compound A, or a pharmaceutically acceptable salt thereof. In some embodiments, AUC₀₋ₜ is AUC₀₋₁₂ (or AUC₀₋₁₂ hr), AUC₀₋₂₄ (or AUC₀₋₂₄ hr), or AUC₀₋₄₈ (or AUC₀₋₄₈ hr). In some embodiments, the AUC₀₋ₜ is AUC₀₋₂₄. In some embodiments, the AUC is AUCss.

In some embodiments, the oral liquid suspension comprising Compound A or a pharmaceutically acceptable salt thereof is administered in an amount sufficient to achieve in the subject a prescribed steady-state AUC (AUCss). In some embodiments, the oral liquid suspension comprising Compound A or a pharmaceutically acceptable salt thereof is administered in an amount that is sufficient to achieve in the subject an AUCss for Compound A that is at least about 100,000 ng*h/mL. In some embodiments, the AUCss is at least about 200,000 ng*h/mL. In some embodiments, the AUCss is at least about 300,000 ng*h/mL. In some embodiments, the oral liquid suspension comprising Compound A or a pharmaceutically acceptable salt thereof is administered in an amount that is sufficient to achieve in the subject an AUCss for Compound A that is at least about 400,000 ng*h/mL. In some embodiments, the AUCss is at least about 500,000 ng*h/mL. In some embodiments, the AUCss is at least about 600,000 ng*h/mL. In some embodiments, the AUCss is at least about 400,000 ng*h/mL to at least about 800,000 ng*h/mL. In some embodiments, the AUCss is at least about 500,000 ng*h/mL to at least about 700,000 ng*h/mL. In some embodiments, the AUCss is at least about 300,000 ng*h/mL to at least about 800,000 ng*h/mL. In some embodiments, the AUCss is at least about 200,000 ng*h/mL to at least about 800,000 ng*h/mL. In some embodiments, the AUCss is about 100,000 ng*h/mL to about 800,000 ng*h/mL. In some embodiments, the AUCss is at most about 600,000 ng*h/mL. In some embodiments, the AUCss is at most about 800,000 ng*h/mL. In some embodiments, the AUCss is at most about 1,000,000 ng*h/mL. In some embodiments, the AUCss is at most about 1,200,000 ng*h/mL. In some embodiments, the AUCss is at most about 1,600,000 ng*h/mL.

In some embodiments, the oral liquid suspension comprising Compound A or a pharmaceutically acceptable salt thereof is administered in an amount that is sufficient to achieve in the subject an AUCss for Compound A that is about 100,000 ng*h/mL to about 1,600,000 ng*h/mL, about 100,000 ng*h/mL to about 1,000,000 ng*h/mL, about 100,000 ng*h/mL to about 800,000 ng*h/mL, about 100,000 ng*h/mL to about 600,000 ng*h/mL, 200,000 ng*h/mL to about 1,600,000 ng*h/mL, about 200,000 ng*h/mL to about 1,000,000 ng*h/mL, about 200,000 ng*h/mL to about 800,000 ng*h/mL, about 200,000 ng*h/mL to about 600,000 ng*h/mL, 300,000 ng*h/mL to about 1,600,000 ng*h/mL, about 300,000 ng*h/mL to about 1,000,000 ng*h/mL, about 300,000 ng*h/mL to about 800,000 ng*h/mL, about 300,000 ng*h/mL to about 600,000 ng*h/mL, 400,000 ng*h/mL to about 1,600,000 ng*h/mL, about 400,000 ng*h/mL to about 1,000,000 ng*h/mL, about 400,000 ng*h/mL to about 800,000 ng*h/mL, about 400,000 ng*h/mL to about 600,000 ng*h/mL, 500,000 ng*h/mL to about 1,600,000 ng*h/mL, about 500,000 ng*h/mL to about 1,000,000 ng*h/mL, about 500,000 ng*h/mL to about 800,000 ng*h/mL, about 500,000 ng*h/mL to about 600,000 ng*h/mL, 600,000 ng*h/mL to about 1,600,000 ng*h/mL, about 600,000 ng*h/mL to about 1,000,000 ng*h/mL, or about 600,000 ng*h/mL to about 800,000 ng*h/mL.

In some embodiments, the oral liquid suspension comprising Compound A or a pharmaceutically acceptable salt thereof is administered in an amount that is sufficient to achieve in the subject an AUCss for Compound A that is about 300,000 ng*h/mL to about 450,000 ng*h/mL, about 300,000 ng*h/mL to about 500,000 ng*h/mL, about 300,000 ng*h/mL to about 550,000 ng*h/mL, about 300,000 ng*h/mL to about 650,000 ng*h/mL, about 350,000 ng*h/mL to about 750,000 ng*h/mL, about 400,000 ng*h/mL to about 650,000 ng*h/mL, about 400,000 ng*h/mL to about 750,000 ng*h/mL, about 400,000 ng*h/mL to about 850,000 ng*h/mL, about 400,000 ng*h/mL to about 950,000 ng*h/mL, or about 400,000 ng*h/mL to about 1,000,000 ng*h/mL.

In some embodiments, the oral liquid suspension comprising Compound A or a pharmaceutically acceptable salt thereof is administered in an amount that is sufficient to achieve in the subject a prescribed AUC₀₋ₜ. In some embodiments, the AUC₀₋ₜ is AUC₀₋₂₄. In some embodiments, the oral liquid suspension comprising Compound A or a pharmaceutically acceptable salt thereof is administered in an amount that is sufficient to achieve in the subject an AUC₀₋₂₄ for Compound A that is at least about 10,000 ng*h/mL. In some embodiments, the oral liquid suspension comprising Compound A or a pharmaceutically acceptable salt thereof is administered in an amount that is sufficient to achieve in the subject an AUC₀₋₂₄ of Compound A that is at least about 50,000 ng*h/mL. In some embodiments, the oral liquid suspension comprising Compound A or a pharmaceutically acceptable salt thereof is administered in an amount that is sufficient to achieve in the subject an AUC₀₋₂₄ of Compound A that is at least about 100,000 ng*h/mL. In some embodiments, the AUC₀₋₂₄ is at least about 100,000 ng*h/mL to at least about 600,000 ng*h/mL. In some embodiments, the AUC₀₋₂₄ is about 100,000 ng*h/mL to about 600,000 ng*h/mL. In some embodiments, the AUC₀₋₂₄ is at least about 100,000 ng*h/mL. In some embodiments, the AUC₀₋₂₄ is at most about 600,000 ng*h/mL.

In some embodiments, the oral liquid suspension comprising Compound A or a pharmaceutically acceptable salt thereof is administered in an amount that is sufficient to achieve in the subject an AUC₀₋₂₄ of Compound A that is about 100,000 ng*h/mL to about 150,000 ng*h/mL, about 100,000 ng*h/mL to about 200,000 ng*h/mL, about 100,000 ng*h/mL to about 250,000 ng*h/mL, about 100,000 ng*h/mL to about 300,000 ng*h/mL, about 100,000 ng*h/mL to about 350,000 ng*h/mL, about 100,000 ng*h/mL to about 400,000 ng*h/mL, about 100,000 ng*h/mL to about 450,000 ng*h/mL, about 100,000 ng*h/mL to about 500,000 ng*h/mL, about 100,000 ng*h/mL to about 550,000 ng*h/mL, about 100,000 ng*h/mL to about 600,000 ng*h/mL, about 150,000 ng*h/mL to about 200,000 ng*h/mL, about 150,000 ng*h/mL to about 250,000 ng*h/mL, about 150,000 ng*h/mL to about 300,000 ng*h/mL, about 150,000 ng*h/mL to about 350,000 ng*h/mL, about 150,000 ng*h/mL to about 400,000 ng*h/mL, about 150,000 ng*h/mL to about 450,000 ng*h/mL, about 150,000 ng*h/mL to about 500,000 ng*h/mL, about 150,000 ng*h/mL to about 550,000 ng*h/mL, about 150,000 ng*h/mL to about 600,000 ng*h/mL, about 200,000 ng*h/mL to about 250,000 ng*h/mL, about 200,000 ng*h/mL to about 300,000 ng*h/mL, about 200,000 ng*h/mL to about 350,000 ng*h/mL, about 200,000 ng*h/mL to about 400,000 ng*h/mL, about 200,000 ng*h/mL to about 450,000 ng*h/mL, about 200,000 ng*h/mL to about 500,000 ng*h/mL, about 200,000 ng*h/mL to about 550,000 ng*h/mL, about 200,000 ng*h/mL to about 600,000 ng*h/mL, about 250,000 ng*h/mL to about 300,000 ng*h/mL, about 250,000 ng*h/mL to about 350,000 ng*h/mL, about 250,000 ng*h/mL to about 400,000 ng*h/mL, about 250,000 ng*h/mL to about 450,000 ng*h/mL, about 250,000 ng*h/mL to about 500,000 ng*h/mL, about 250,000 ng*h/mL to about 550,000 ng*h/mL, about 250,000 ng*h/mL to about 600,000 ng*h/mL, about 300,000 ng*h/mL to about 350,000 ng*h/mL, about 300,000 ng*h/mL to about 400,000 ng*h/mL, about 300,000 ng*h/mL to about 450,000 ng*h/mL, about 300,000 ng*h/mL to about 500,000 ng*h/mL, about 300,000 ng*h/mL to about 550,000 ng*h/mL, about 300,000 ng*h/mL to about 600,000 ng*h/mL, about 350,000 ng*h/mL to about 400,000 ng*h/mL, about 350,000 ng*h/mL to about 450,000 ng*h/mL, about 350,000 ng*h/mL to about 500,000 ng*h/mL, about 350,000 ng*h/mL to about 550,000 ng*h/mL, about 350,000 ng*h/mL to about 600,000 ng*h/mL, about 400,000 ng*h/mL to about 450,000 ng*h/mL, about 400,000 ng*h/mL to about 500,000 ng*h/mL, about 400,000 ng*h/mL to about 550,000 ng*h/mL, about 400,000 ng*h/mL to about 600,000 ng*h/mL, about 450,000 ng*h/mL to about 500,000 ng*h/mL, about 450,000 ng*h/mL to about 550,000 ng*h/mL, about 450,000 ng*h/mL to about 600,000 ng*h/mL, about 500,000 ng*h/mL to about 550,000 ng*h/mL, about 500,000 ng*h/mL to about 600,000 ng*h/mL, or about 550,000 ng*h/mL to about 600,000 ng*h/mL.

In some embodiments, the oral liquid suspension comprising Compound A or a pharmaceutically acceptable salt thereof is administered in an amount that is sufficient to achieve in the subject a prescribed AUC_{0-∞}. In some embodiments, the AUC_{0-∞} of Compound A comprises about 250 μg·hr/L to about 1,600 μg·hr/L. In some embodiments, the AUC_{0-∞} of Compound A comprises at least about 250 μg·hr/L. In some embodiments, the AUC_{0-∞} of Compound A comprises at most about 1,600 μg·hr/L. In some embodiments, the AUC_{0-∞} of Compound A comprises about 250 μg·hr/L to about 350 μg·hr/L, about 250 μg·hr/L to about 450 μg·hr/L, about 250 μg·hr/L to about 550 μg·hr/L, about 250 μg·hr/L to about 650 μg·hr/L, about 250 μg·hr/L to about 750 μg·hr/L, about 250 μg·hr/L to about 850 μg·hr/L, about 250 μg·hr/L to about 950 μg·hr/L, about 250 μg·hr/L to about 1,000 μg·hr/L, about 250 μg·hr/L to about 1,250 μg·hr/L, about 250 μg·hr/L to about 1,500 μg·hr/L, about 250 μg·hr/L to about 1,600 μg·hr/L, about 350 μg·hr/L to about 450 μg·hr/L, about 350 μg·hr/L to about 550 μg·hr/L, about 350 μg·hr/L to about 650 μg·hr/L, about 350 μg·hr/L to about 750 μg·hr/L, about 350 μg·hr/L to about 850 μg·hr/L, about 350 μg·hr/L to about 950 μg·hr/L, about 350 μg·hr/L to about 1,000 μg·hr/L, about 350 μg·hr/L to about 1,250 μg·hr/L, about 350 μg·hr/L to about 1,500 μg·hr/L, about 350 μg·hr/L to about 1,600 μg·hr/L, about 450 μg·hr/L to about 550 μg·hr/L, about 450 μg·hr/L to about 650 μg·hr/L, about 450 μg·hr/L to about 750 μg·hr/L, about 450 μg·hr/L to about 850 μg·hr/L, about 450 μg·hr/L to about 950 μg·hr/L, about 450 μg·hr/L to about 1,000 μg·hr/L, about 450 μg·hr/L to about 1,250 μg·hr/L, about 450 μg·hr/L to about 1,500 μg·hr/L, about 450 μg·hr/L to about 1,600 μg·hr/L, about 550 μg·hr/L to about 650 μg·hr/L, about 550 μg·hr/L to about 750 μg·hr/L, about 550 μg·hr/L to about 850 μg·hr/L, about 550 μg·hr/L to about 950 μg·hr/L, about 550 μg·hr/L to about 1,000 μg·hr/L, about 550 μg·hr/L to about 1,250 μg·hr/L, about 550 μg·hr/L to about 1,500 μg·hr/L, about 550 μg·hr/L to about 1,600 μg·hr/L, about 650 μg·hr/L to about 750 μg·hr/L, about 650 μg·hr/L to about 850 μg·hr/L, about 650 μg·hr/L to about 950 μg·hr/L, about 650 μg·hr/L to about 1,000 μg·hr/L, about 650 μg·hr/L to about 1,250 μg·hr/L, about 650 μg·hr/L to about 1,500 μg·hr/L, about 650 μg·hr/L to about 1,600 μg·hr/L, about 750 μg·hr/L to about 850 μg·hr/L, about 750 μg·hr/L to about 950 μg·hr/L, about 750 μg·hr/L to about 1,000 μg·hr/L, about 750 μg·hr/L to about 1,250 μg·hr/L, about 750 μg·hr/L to about 1,500 μg·hr/L, about 750 μg·hr/L to about 1,600 μg·hr/L, about 850 μg·hr/L to about 950 μg·hr/L, about 850 μg·hr/L to about 1,000 μg·hr/L, about 850 μg·hr/L to about 1,250 μg·hr/L, about 850 μg·hr/L to about 1,500 μg·hr/L, about 850 μg·hr/L to about 1,600 μg·hr/L, about 950 μg·hr/L to about 1,000 μg·hr/L, about 950 μg·hr/L to about 1,250 μg·hr/L, about 950 μg·hr/L to about 1,500 μg·hr/L, about 950 μg·hr/L to about 1,600 μg·hr/L, about 1,000 μg·hr/L to about 1,250 μg·hr/L, about 1,000 μg·hr/L to about 1,500 μg·hr/L, about 1,000 μg·hr/L to about 1,600 μg·hr/L, about 1,250 μg·hr/L to about 1,500 μg·hr/L, about 1,250 μg·hr/L to about 1,600 μg·hr/L, or about 1,500 μg·hr/L to about 1,600 μg·hr/L.

In some embodiments, the oral liquid suspension, when administered to a human subject in an amount equivalent to about 100 mg of Compound A, is sufficient to achieve in the subject a maximum observed blood plasma concentration (Cmax) of Compound A of at least 100 ng/mL. In some embodiments, the oral liquid suspension, when administered to a human subject in an amount equivalent to about 100 mg of Compound A, is sufficient to achieve in the subject a maximum observed blood plasma concentration (Cmax) of Compound A of at least 100 ng/mL, at least 200 ng/mL, at least 300 ng/mL, at least 400 ng/mL, at least 500 ng/mL, at least 1000 ng/mL, at least 1,500 ng/mL, at least 2000 ng/mL, at least 2500 ng/mL, at least 3000 ng/mL, at least 3500 ng/mL, at least 4000 ng/mL, at least 4500 ng/mL, at least 5000 ng/mL, at least 5500 ng/mL, at least 6000 ng/mL, at least 6500 ng/mL, at least 7000 ng/mL, at least 7500 ng/mL, or at least 8000 ng/mL. In some embodiments, the oral liquid suspension, when administered to a human subject in an amount equivalent to about 100 mg of Compound A, is sufficient to achieve in the subject a maximum observed blood plasma concentration (Cmax) of Compound A of at most 100 ng/mL, at most 200 ng/mL, at most 300 ng/mL, at most 400 ng/mL, at most 500 ng/mL, at most 1000 ng/mL, at most 1,500 ng/mL, at most 2000 ng/mL, at most 2500 ng/mL, at most 3000 ng/mL, at most 3500 ng/mL, at most 4000 ng/mL, at most 4500 ng/mL, at most 5000 ng/mL, at most 5500 ng/mL, at most 6000 ng/mL, at most 6500 ng/mL, at most 7000 ng/mL, at most 7500 ng/mL, or at most 8000 ng/mL. In some embodiments, the oral liquid suspension, when administered to a human subject in an amount equivalent to about 100 mg of Compound A, is sufficient to achieve in the subject a maximum observed blood plasma concentration (Cmax) of Compound A of about 100 ng/mL, about 200 ng/mL, about 300 ng/mL, about 400 ng/mL, about 500 ng/mL, about 1000 ng/mL, about 1,500 ng/mL, about 2000 ng/mL, about 2500 ng/mL, about 3000 ng/mL, about 3500 ng/mL, about 4000 ng/mL, about 4500 ng/mL, about 5000 ng/mL, about 5500 ng/mL, about 6000 ng/mL, about 6500 ng/mL, about 7000 ng/mL, about 7500 ng/mL, or about 8000 ng/mL.

In some embodiments, the oral liquid suspension, when administered to a human subject in an amount equivalent to about 75 mg of Compound A, is sufficient to achieve in the subject a maximum observed blood plasma concentration (Cmax) of Compound A of at least 100 ng/mL. In some embodiments, the oral liquid suspension, when administered to a human subject in an amount equivalent to about 75 mg of Compound A, is sufficient to achieve in the subject a maximum observed blood plasma concentration (Cmax) of Compound A of at least 100 ng/mL, at least 200 ng/mL, at least 300 ng/mL, at least 400 ng/mL, at least 500 ng/mL, at least 1000 ng/mL, at least 1,500 ng/mL, at least 2000 ng/mL, at least 2500 ng/mL, at least 3000 ng/mL, at least 3500 ng/mL, at least 4000 ng/mL, at least 4500 ng/mL, at least 5000 ng/mL, at least 5500 ng/mL, at least 6000 ng/mL, at least 6500 ng/mL, at least 7000 ng/mL, at least 7500 ng/mL, or at least 8000 ng/mL. In some embodiments, the oral liquid suspension, when administered to a human subject in an amount equivalent to about 75 mg of Compound A, is sufficient to achieve in the subject a maximum observed blood plasma concentration (Cmax) of Compound A of at most 100 ng/mL, at most 200 ng/mL, at most 300 ng/mL, at most 400 ng/mL, at most 500 ng/mL, at most 1000 ng/mL, at most 1,500 ng/mL, at most 2000 ng/mL, at most 2500 ng/mL, at most 3000 ng/mL, at most 3500 ng/mL, at most 4000 ng/mL, at most 4500 ng/mL, at most 5000 ng/mL, at most 5500 ng/mL, at most 6000 ng/mL, at most 6500 ng/mL, at most 7000 ng/mL, at most 7500 ng/mL, or at most 8000 ng/mL. In some embodiments, the oral liquid suspension, when administered to a human subject in an amount equivalent to about 75 mg of Compound A, is sufficient to achieve in the subject a maximum observed blood plasma concentration (Cmax) of Compound A of about 100 ng/mL, about 200 ng/mL, about 300 ng/mL, about 400 ng/mL, about 500 ng/mL, about 1000 ng/mL, about 1,500 ng/mL, about 2000 ng/mL, about 2500 ng/mL, about 3000 ng/mL, about 3500 ng/mL, about 4000 ng/mL, about 4500 ng/mL, about 5000 ng/mL, about 5500 ng/mL, about 6000 ng/mL, about 6500 ng/mL, about 7000 ng/mL, about 7500 ng/mL, or about 8000 ng/mL.

In some embodiments, the oral liquid suspension, when administered to a human subject in an amount equivalent to about 50 mg of Compound A, is sufficient to achieve in the subject a maximum observed blood plasma concentration (Cmax) of Compound A of at least about at least 2000 ng/mL. In some embodiments, the oral liquid suspension, when administered to a human subject in an amount equivalent to about 50 mg of Compound A, is sufficient to achieve in the subject a maximum observed blood plasma concentration (Cmax) of Compound A of at least 100 ng/mL, at least 200 ng/mL, at least 300 ng/mL, at least 400 ng/mL, at least 500 ng/mL, at least 1000 ng/mL, at least 1,500 ng/mL, at least 2000 ng/mL, at least 2500 ng/mL, at least 3000 ng/mL, at least 3500 ng/mL, at least 4000 ng/mL, at least 4500 ng/mL, at least 5000 ng/mL, at least 5500 ng/mL, at least 6000 ng/mL, at least 6500 ng/mL, at least 7000 ng/mL, at least 7500 ng/mL, or at least 8000 ng/mL. In some embodiments, the oral liquid suspension, when administered to a human subject in an amount equivalent to about 50 mg of Compound A, is sufficient to achieve in the subject a maximum observed blood plasma concentration (Cmax) of Compound A of at most 100 ng/mL, at most 200 ng/mL, at most 300 ng/mL, at most 400 ng/mL, at most 500 ng/mL, at most 1000 ng/mL, at most 1,500 ng/mL, at most 2000 ng/mL, at most 2500 ng/mL, at most 3000 ng/mL, at most 3500 ng/mL, at most 4000 ng/mL, at most 4500 ng/mL, at most 5000 ng/mL, at most 5500 ng/mL, at most 6000 ng/mL, at most 6500 ng/mL, at most 7000 ng/mL, at most 7500 ng/mL, or at most 8000 ng/mL. In some embodiments, the oral liquid suspension, when administered to a human subject in an amount equivalent to about 50 mg of Compound A, is sufficient to achieve in the subject a maximum observed blood plasma concentration (Cmax) of Compound A of about 100 ng/mL, about 200 ng/mL, about 300 ng/mL, about 400 ng/mL, about 500 ng/mL, about 1000 ng/mL, about 1,500 ng/mL, about 2000 ng/mL, about 2500 ng/mL, about 3000 ng/mL, about 3500 ng/mL, about 4000 ng/mL, about 4500 ng/mL, about 5000 ng/mL, about 5500 ng/mL, about 6000 ng/mL, about 6500 ng/mL, about 7000 ng/mL, about 7500 ng/mL, or about 8000 ng/mL.

In some embodiments, the oral liquid suspension, when administered to a human subject in an amount equivalent to about 25 mg of Compound A, is sufficient to achieve in the subject a maximum observed blood plasma concentration (Cmax) of Compound A of at least about at least 100 ng/mL. In some embodiments, the oral liquid suspension, when administered to a human subject in an amount equivalent to about 25 mg of Compound A, is sufficient to achieve in the subject a maximum observed blood plasma concentration (Cmax) of Compound A of at least 100 ng/mL, at least 200 ng/mL, at least 300 ng/mL, at least 400 ng/mL, at least 500 ng/mL, at least 1000 ng/mL, at least 1,500 ng/mL, at least 2000 ng/mL, at least 2500 ng/mL, at least 3000 ng/mL, at least 3500 ng/mL, at least 4000 ng/mL, at least 4500 ng/mL, at least 5000 ng/mL, at least 5500 ng/mL, at least 6000 ng/mL, at least 6500 ng/mL, at least 7000 ng/mL, at least 7500 ng/mL, or at least 8000 ng/mL. In some embodiments, the oral liquid suspension, when administered to a human subject in an amount equivalent to about 25 mg of Compound A, is sufficient to achieve in the subject a maximum observed blood plasma concentration (Cmax) of Compound A of at most 100 ng/mL, at most 200 ng/mL, at most 300 ng/mL, at most 400 ng/mL, at most 500 ng/mL, at most 1000 ng/mL, at most 1,500 ng/mL, at most 2000 ng/mL, at most 2500 ng/mL, at most 3000 ng/mL, at most 3500 ng/mL, at most 4000 ng/mL, at most 4500 ng/mL, at most 5000 ng/mL, at most 5500 ng/mL, at most 6000 ng/mL, at most 6500 ng/mL, at most 7000 ng/mL, at most 7500 ng/mL, or at most 8000 ng/mL. In some embodiments, the oral liquid suspension, when administered to a human subject in an amount equivalent to about 25 mg of Compound A, is sufficient to achieve in the subject a maximum observed blood plasma concentration (Cmax) of Compound A of about 100 ng/mL, about 200 ng/mL, about 300 ng/mL, about 400 ng/mL, about 500 ng/mL, about 1000 ng/mL, about 1,500 ng/mL, about 2000 ng/mL, about 2500 ng/mL, about 3000 ng/mL, about 3500 ng/mL, about 4000 ng/mL, about 4500 ng/mL, about 5000 ng/mL, about 5500 ng/mL, about 6000 ng/mL, about 6500 ng/mL, about 7000 ng/mL, about 7500 ng/mL, or about 8000 ng/mL.

Subjects can be, for example, mammal, humans, pregnant women, elderly adults, adults, adolescents, pre-adolescents, children, toddlers, infants, newborn, or neonates. A subject can be a patient. In some cases, a subject can be a human. In some cases, a subject can be a child (e.g., a young human being below the age of puberty). In some cases, a subject can be an infant. In some cases, the subject can be a formula-fed infant. In some cases, a subject can be an individual enrolled in a clinical study. In some cases, a subject can be a laboratory animal, for example, a mammal, or a rodent. In some cases, the subject can be a mouse. In some cases, the subject can be an obese or overweight subject.

### Age of Subject

In some embodiments, a subject described herein is younger than 18 years old. In some embodiments, a subject described herein is from one week to 6 months old. In some embodiments, a subject described herein is from 28 days to 6 months old. In some embodiments, a subject described herein is at least 6 months old. In some embodiments, a subject described herein is at most 6 months old. In some embodiments, a subject described herein is at least 28 days, 1 month, 2 months, 3 months, 4 months, 5 months, or 6 months old. In some embodiments, a subject described herein is at most 28 days, 1 month, 2 months, 3 months, 4 months, 5 months, or 6 months old. In some embodiments, a subject described herein is at least 1 year, 2 years, 3 years, 4 years, 5 years, 6 years, 7 years, 8 years, 9 years, 10 years, 11 years, 12 years, 13 years, 14 years, 15 years, 16 years, 17 years, or 18 years of age. In some embodiments, a subject described herein is at least 1 year, 2 years, 3 years, 4 years, 5 years, 6 years, 7 years, 8 years, 9 years, 10 years, 11 years, or 12 years of age. In some embodiments, a subject described herein is at most 1 year, 2 years, 3 years, 4 years, 5 years, 6 years, 7 years, 8 years, 9 years, 10 years, 11 years, 12 years, 13 years, 14 years, 15 years, 16 years, 17 years, or 18 years of age. In some embodiments, a subject described herein is at most 1 year, 2 years, 3 years, 4 years, 5 years, 6 years, 7 years, 8 years, 9 years, 10 years, 11 years, or 12 years of age. In some embodiments, a subject described herein is at most 28 days, 1 month, 2 months, 3 months, 4 months, 5 months, 6 months, 7 months, 8 months, 9 months, 10 months, 11 months, 12 months, 24 months, 48 months, 60 months, 72 months, 84 months, 96 months, 108 months, 120 months, 132 months, 144 months, 156 months, 168 months, 180 months, 192 months, 204 months, or 216 months old. In some embodiments, a subject described herein is at most 28 days, 1 month, 2 months, 3 months, 4 months, 5 months, 6 months, 7 months, 8 months, 9 months, 10 months, 11 months, 12 months, 2 years old, 3 years, 4 years, 5 years, 6 years, 7 years, 8 years, 9 years, 10 years, 11 years, 12 years, 13 years, 14 years, 15 years, 16 years, 17 years, or 18 years old.

In some embodiments, the subject has previously been treated with one or more different cancer treatment modalities. In some embodiments, the subject has previously been treated with one or more of radiotherapy, chemotherapy, or immunotherapy. In some embodiments, the subject has been treated with one, two, three, four, or five lines of prior therapy. In some embodiments, the prior therapy is a cytotoxic therapy.

### Cancer and Tumors

In certain aspects, provided herein are methods of treating cancer comprising administering pharmaceutical powders or suspensions disclosed herein, comprising Compound A or a pharmaceutically acceptable salt thereof. In some embodiments, the cancer is a cancer selected from the group consisting of colorectal cancer, pancreatic cancer, lung cancer, ovarian cancer, liver cancer, breast cancer, kidney cancer, prostate cancer, gastrointestinal cancer, melanoma, cervical cancer, neuroendocrine cancer, bladder cancer, glioblastoma, and head and neck cancer. In certain embodiments, the cancer is pancreatic cancer. In certain embodiments, the cancer is ovarian cancer. In certain embodiments, the cancer is colorectal cancer. In certain embodiments, the cancer is breast cancer. In certain embodiments, the cancer is prostate cancer. In certain embodiments, the cancer is lung cancer. In certain embodiments, the cancer is melanoma. In some embodiments, the cancer is a solid cancer.

In some embodiments, the cancer is a hematologic cancer. In some embodiment, the cancer is selected from the group consisting of: acute myelogenous leukemia (AML), Hodgkin lymphoma, multiple myeloma, T cell acute lymphoblastic leukemia (T-ALL), chronic lymphocytic leukemia (CLL), hairy cell leukemia, chronic myelogenous leukemia (CML), non-Hodgkin lymphoma, diffuse large B-cell lymphoma (DLBCL), mantle cell lymphoma (MCL), and cutaneous T cell lymphoma (CTCL).

The methods and pharmaceutical compositions of Compound A or a salt thereof can be used to treat any suitable cancer known in the art. Non-limiting examples of cancers to be treated by the methods of the present disclosure can include melanoma (e.g., metastatic malignant melanoma), renal cancer (e.g., clear cell carcinoma), prostate cancer (e.g., hormone refractory prostate adenocarcinoma), pancreatic adenocarcinoma, breast cancer, colon cancer, lung cancer (e.g., non-small cell lung cancer), esophageal cancer, squamous cell carcinoma of the head and neck, liver cancer, ovarian cancer, cervical cancer, thyroid cancer, glioblastoma, glioma, leukemia, lymphoma, and other neoplastic malignancies. In some embodiments, the cancer is low grade glioma (LGG), e.g., pediatric low grade glioma (pLGG). In some embodiments, pharmaceutical compositions of Compound A or a salt thereof are used as front-line therapy for treating the cancer. In some embodiments, the subject has not previously received any standard-of-care therapies for treating cancer. In some embodiments, pharmaceutical compositions of Compound A or a salt thereof are used to treat a newly diagnosed cancer.

In some embodiments, the cancer is low grade glioma. In some embodiments, the cancer is pLGG. In some embodiments, the pLGG is identified as having one or more of the following mutations: RAS positive mutation, RAF positive mutation, MEK positive mutation, and ERK positive mutation. In some embodiments, the pLGG has a BRAF mutation. In some embodiments, the BRAF mutation is a non-V600 BRAF mutation. In some embodiments, the BRAF mutation is a V600 BRAF mutation. In some embodiments, the presence of BRAF fusion or rearrangement, or BRAF mutation, in a subject is confirmed prior to treatment. In some embodiments, the subject is identified having one or more of the following wild-type fusions: KIAA1549:BRAF, STARD3NL:BRAF, BCAS1:BRAF, KHDRBS2:BRAF, CCDC6:BRAF, FAM131B:BRAF, SRGAP:BRAF, CLCN6:BRAF, GNAI1:BRAF, MRKN1:BRAF, GIT2:BRAF, GTF21:BRAF, FXR1:BRAF, RNF130:BRAF, BRAF:MACF1, TMEM106B:BRAF, PPC1CC:BRAF, CUX1:BRAF, SRGAP3:RAF1, QK1:RAF1, FYCO:RAF1, ATG7:RAF1, and NFIA:RAF1. In some embodiments, the subject is identified having KIAA1549:BRAF wild-type fusion. In some embodiments, the pLGG has a gene fusion. In some embodiments, the cancer is pLGG with RIN2:BRAF gene fusion. In some embodiments, the subject has one or more of the following gene fusions: KIAA1549:BRAF, AGK:BRAF, STARD3NL:BRAF, BCAS1:BRAF, KHDRBS2:BRAF, CCDC6:BRAF, FAM131B:BRAF, SRGAP:BRAF, CLCN6:BRAF, GNAI1:BRAF, MRKN1:BRAF, GIT2:BRAF, GTF2I:BRAF, FXR1:BRAF, RNF130:BRAF, MACF1:BRAF, TMEM106B:BRAF, PPC1CC:BRAF, CUX1:BRAF, CCD6:BRAF, PPP1CC:BRAF, SEPT7:BRAF, PDE10A:BRAF, EPB41L2:BRAF, OSBP:BRAF, DAAM1:BRAF, TEX41:BRAF, FOXN3:BRAF, TRIPP1:BRAF, TOM1L2:BRAF, TMEM106B:BRAF, SRGAP3:RAF1, QK1:RAF1, FYCO:RAF1, ATG7:RAF1, NFIA-RAF1, TMF1:RAF1, GOLGA3:RAF1, SOX6:RAF1, BMPR1A:RAF1, PDZRN3:RAF1, SLMAP:RAF1, MAP4:RAF1, BCL6-RAF1, SEPT17:BRAF, ZNF767:BRAF, CCDC91:BRAF, DYNC1/2:BRAF, ZKSCAN1:BRAF, GTF2I:BRAF, MZTl:BRAF, RAD18:BRAF, CUX1:BRAF, CUX1-RAF1, CULl:BRAF, SLC12A7:BRAF, TRIM24:BRAF, AGAP3:BRAF, AKAP9:BRAF, TAX1BP1:BRAF, CDC27:BRAF, FKBP15:BRAF, SKAP2:BRAF, TARDBP:BRAF, SEPT3:BRAF, ARMC10:BRAF, PAPSSI:BRAF, FCHSD1:BRAF, ERC1:BRAF, CDK5RAP2:BRAF, TMEM178B:BRAF, BAIAP2L1:BRAF, CEP89:BRAF, CNTNAP2:BRAF, EML4:BRAF, KCTD7:BRAF, LSM14A:BRAF, NFIC:BRAF, NUDCD3:BRAF, PHTF2:BRAF, PLIN3:BRAF, RP2:BRAF, SOX5:BRAF, SOX6:BRAF, TLK2:BRAF, ZKSCAN5:BRAF, KLC1-RAF1, DAAM1-RAF1, ZNF444-RAF1, LRCH3-RAF1, GOLGA4-RAF1, CTDSPL-RAF1, PRKAR2A-RAF1, CTNNAl-RAF1, MKRN1-RAF1, DYNC1H1-RAF1, GPHN-RAF1, KLHL7:BRAF, TANK:BRAF, RBMS3:BRAF, FAM114A2:BRAF, AGGF1-RAF1, EPS15:BRAF, NUP214:BRAF, BTF3L4:BRAF, GHR:BRAF, DOCK4:BRAF, ZC3HAV1:BRAF, MKRN1:BRAF, MYRIP:BRAF, SND1:BRAF, TNS3:BRAF, ATG7:BRAF, NUB1:BRAF, STRN3:BRAF, STK35:BRAF, ETFA:BRAF, SVOPL:BRAF, JHDMlD:BRAF, PPFIBP2:BRAF, SCL45A3:BRAF, ESRP1-RAF1, AGTRAP:BRAF, SVIP:BRAF, NRF1:BRAF, RAF1-CCDC176, RAF1-TRAK1, ESYT2:BRAF, PCBP2:BRAF, and SALL2:BRAF. In some embodiments, the subject has TRIM33:BRAF gene fusion.

In some embodiments, the subject has one or more of the following gene fusions: AGK:BRAF, STARD3NL:BRAF, BCAS1:BRAF, KHDRBS2:BRAF, CCDC6:BRAF, FAM131B:BRAF, SRGAP:BRAF, CLCN6:BRAF, GNAI1:BRAF, MRKN1:BRAF, GIT2:BRAF, GTF2I:BRAF, FXR1:BRAF, RNF130:BRAF, MACF1:BRAF, TMEM106B:BRAF, PPC1CC:BRAF, CUX1:BRAF, CCD6:BRAF, PPP1CC:BRAF, SEPT7:BRAF, PDE10A:BRAF, EPB41L2:BRAF, OSBP:BRAF, DAAM1:BRAF, TEX41:BRAF, FOXN3:BRAF, TRIPP1:BRAF, TOM1L2:BRAF, TMEM106B:BRAF, QK1:RAF1, FYCO:RAF1, ATG7:RAF1, NFIA-RAF1, TMF1:RAF1, GOLGA3:RAF1, SOX6:RAF1, BMPR1A:RAF1, PDZRN3:RAF1, SLMAP:RAF1, MAP4:RAF1, BCL6-RAF1, SEPT17:BRAF, ZNF767:BRAF, CCDC91:BRAF, DYNC1/2:BRAF, ZKSCAN1:BRAF, GTF2I:BRAF, MZTl:BRAF, RAD18:BRAF, CUX1:BRAF, CUX1-RAF1, CUL1:BRAF, SLC12A7:BRAF, TRIM24:BRAF, AGAP3:BRAF, AKAP9:BRAF, TAX1BPl:BRAF, CDC27:BRAF, FKBP15:BRAF, SKAP2:BRAF, TARDBP:BRAF, SEPT3:BRAF, ARMC10:BRAF, PAPSSI:BRAF, FCHSD1:BRAF, ERC1:BRAF, CDK5RAP2:BRAF, TMEM178B:BRAF, BAIAP2L1:BRAF, CEP89:BRAF, CNTNAP2:BRAF, EML4:BRAF, KCTD7:BRAF, LSM14A:BRAF, NFIC:BRAF, NUDCD3:BRAF, PHTF2:BRAF, PLIN3:BRAF, RP2:BRAF, SOX5:BRAF, SOX6:BRAF, TLK2:BRAF, ZKSCAN5:BRAF, KLC1-RAF1, DAAM1-RAF1, ZNF444-RAF1, LRCH3-RAF1, GOLGA4-RAF1, CTDSPL-RAF1, PRKAR2A-RAF1, CTNNAl-RAF1, MKRN1-RAF1, DYNC1H1-RAF1, GPHN-RAF1, KLHL7:BRAF, TANK:BRAF, RBMS3:BRAF, FAM114A2:BRAF, AGGF1-RAF1, EPS15:BRAF, NUP214:BRAF, BTF3L4:BRAF, GHR:BRAF, DOCK4:BRAF, ZC3HAV1:BRAF, MKRN1:BRAF, MYRIP:BRAF, SND1:BRAF, TNS3:BRAF, ATG7:BRAF, NUB1:BRAF, STRN3:BRAF, STK35:BRAF, ETFA:BRAF, SVOPL:BRAF, JHDMlD:BRAF, PPFIBP2:BRAF, SCL45A3:BRAF, ESRP1-RAF1, AGTRAP:BRAF, SVIP:BRAF, NRF1:BRAF, RAF1-CCDC176, RAF1-TRAK1, ESYT2:BRAF, PCBP2:BRAF, and SALL2:BRAF.

In some embodiments, the gene fusion is a BRAF fusion. In some embodiments, the BRAF fusion is selected from: AGK:BRAF, STARD3NL:BRAF, BCAS1:BRAF, KHDRBS2:BRAF, CCDC6:BRAF, FAM131B:BRAF, SRGAP:BRAF, CLCN6:BRAF, GNAI1:BRAF, MRKN1:BRAF, GIT2:BRAF, GTF2I:BRAF, FXR1:BRAF, RNF130:BRAF, MACF1:BRAF, TMEM106B:BRAF, PPC1CC:BRAF, CUX1:BRAF, CCD6:BRAF, PPP1CC:BRAF, SEPT7:BRAF, PDE10A:BRAF, EPB41L2:BRAF, OSBP:BRAF, DAAM1:BRAF, TEX41:BRAF, FOXN3:BRAF, TRIPP1:BRAF, TOM1L2:BRAF, 5 BRAF fusions, TMEM106B:BRAF, SEPT17:BRAF, ZNF767:BRAF, CCDC91:BRAF, DYNC1/2:BRAF, ZKSCAN1:BRAF, GTF2I:BRAF, MZTl:BRAF, RAD18:BRAF, CUX1:BRAF, CUL1:BRAF, SLC12A7:BRAF, TRIM24:BRAF, AGAP3:BRAF, AKAP9:BRAF, TAX1BPl:BRAF, CDC27:BRAF, FKBP15:BRAF, SKAP2:BRAF, TARDBP:BRAF, SEPT3:BRAF, ARMC10:BRAF, PAPSS1:BRAF, FCHSD1:BRAF, ERC1:BRAF, CDK5RAP2:BRAF, TMEM178B:BRAF, BAIAP2L1:BRAF, CEP89:BRAF, CNTNAP2:BRAF, EML4:BRAF, KCTD7:BRAF, LSM14A:BRAF, NFIC:BRAF, NUDCD3:BRAF, PHTF2:BRAF, PLIN3:BRAF, RP2:BRAF, SOX5:BRAF, SOX6:BRAF, TLK2:BRAF, ZKSCAN5:BRAF, KLHL7:BRAF, TANK:BRAF, RBMS3:BRAF, FAM114A2:BRAF, EPS15:BRAF, NUP214:BRAF, BTF3L4:BRAF, GHR:BRAF, DOCK4:BRAF, ZC3HAV1:BRAF, MKRN1:BRAF, MYRIP:BRAF, SND1:BRAF, TNS3:BRAF, ATG7:BRAF, NUB1:BRAF, STRN3:BRAF, STK35:BRAF, ETFA:BRAF, SVOPL:BRAF, JHDMlD:BRAF, PPFIBP2:BRAF, SCL45A3:BRAF, AGTRAP:BRAF, SVIP:BRAF, NRF1:BRAF, ESYT2:BRAF, PCBP2:BRAF, and SALL2:BRAF.

In some embodiments, the gene fusion is a RAF1 fusion. In some embodiments, the RAF1 fusion is selected from: SRGAP3:RAF1, QK1:RAF1, FYCO:RAF1, ATG7:RAF1, NFIA:RAF1, TMF1:RAF1, GOLGA3:RAF1, SOX6:RAF1, BMPR1A:RAF1, PDZRN3:RAF1, SLMAP:RAF1, MAP4:RAF1, BCL6:RAF1, CUX1:RAF1, KLC1:RAF1, DAAM1:RAF1, ZNF444:RAF1, LRCH3:RAF1, GOLGA4:RAF1, CTDSPL:RAF1, PRKAR2A:RAF1, CTNNA1:RAF1, MKRN1:RAF1, DYNC1H1:RAF1, AGGF1:RAF1, ESRP1:RAF1, GPHN:RAF1, RAF1-CCDC176, and RAF1-TRAK1.

In some embodiments, a subject has a CRAF gene fusion. In some embodiments, the CRAF gene fusion is SRGAP3:RAF1, QK1:RAF1, FYCO:RAF1, ATG7:RAF1, NFIA:RAF1, TMF1:RAF1, GOLGA3:RAF1, SOX6:RAF1, BMPR1A:RAF1, PDZRN3:RAF1, SLMAP:RAF1, MAP4:RAF1, BCL6:RAF1, CUX1:RAF1, KLC1:RAF1, DAAM1:RAF1, ZNF444:RAF1, LRCH3:RAF1, GOLGA4:RAF1, CTDSPL:RAF1, PRKAR2A:RAF1, CTNNA1:RAF1, MKRN1:RAF1, DYNC1H1:RAF1, AGGF1:RAF1, ESRP1:RAF1, GPHN:RAF1, RAF1-CCDC176, or RAF1-TRAK1.

In some embodiments, the cancer has one more of the following gene fusions: KIAA1549:BRAF, AGK:BRAF, STARD3NL:BRAF, BCAS1:BRAF, KHDRBS2:BRAF, CCDC6:BRAF, FAM131B:BRAF, SRGAP:BRAF, CLCN6:BRAF, GNAI1:BRAF, MRKN1:BRAF, GIT2:BRAF, GTF2I:BRAF, FXR1:BRAF, RNF130:BRAF, MACF1:BRAF, TMEM106B:BRAF, PPC1CC:BRAF, CUX1:BRAF, CCD6:BRAF, PPP1CC:BRAF, SEPT7:BRAF, PDE10A:BRAF, EPB41L2:BRAF, OSBP:BRAF, DAAM1:BRAF, TEX41:BRAF, FOXN3:BRAF, TRIPP1:BRAF, TOM1L2:BRAF, TMEM106B:BRAF, SRGAP3:RAF1, QK1:RAF1, FYCO:RAF1, ATG7:RAF1, NFIA-RAF1, TMF1:RAF1, GOLGA3:RAF1, SOX6:RAF1, BMPR1A:RAF1, PDZRN3:RAF1, SLMAP:RAF1, MAP4:RAF1, BCL6-RAF1, SEPT17:BRAF, ZNF767:BRAF, CCDC91:BRAF, DYNC1/2:BRAF, ZKSCAN1:BRAF, GTF2I:BRAF, MZTl:BRAF, RAD18:BRAF, CUX1:BRAF, CUX1-RAF1, CUL1:BRAF, SLC12A7:BRAF, TRIM24:BRAF, AGAP3:BRAF, AKAP9:BRAF, TAX1BPl:BRAF, CDC27:BRAF, FKBP15:BRAF, SKAP2:BRAF, TARDBP:BRAF, SEPT3:BRAF, ARMC10:BRAF, PAPSS1:BRAF, FCHSD1:BRAF, ERC1:BRAF, CDK5RAP2:BRAF, TMEM178B:BRAF, BAIAP2L1:BRAF, CEP89:BRAF, CNTNAP2:BRAF, EML4:BRAF, KCTD7:BRAF, LSM14A:BRAF, NFIC:BRAF, NUDCD3:BRAF, PHTF2:BRAF, PLIN3:BRAF, RP2:BRAF, SOX5:BRAF, SOX6:BRAF, TLK2:BRAF, ZKSCAN5:BRAF, KLC1-RAF1, DAAM1-RAF1, ZNF444-RAF1, LRCH3-RAF1, GOLGA4-RAF1, CTDSPL-RAF1, PRKAR2A-RAF1, CTNNAl-RAF1, MKRN1-RAF1, DYNC1H1-RAF1, GPHN-RAF1, KLHL7:BRAF, TANK:BRAF, RBMS3:BRAF, FAM114A2:BRAF, AGGF1-RAF1, EPS15:BRAF, NUP214:BRAF, BTF3L4:BRAF, GHR:BRAF, DOCK4:BRAF, ZC3HAV1:BRAF, MKRN1:BRAF, MYRIP:BRAF, SND1:BRAF, TNS3:BRAF, ATG7:BRAF, NUB1:BRAF, STRN3:BRAF, STK35:BRAF, ETFA:BRAF, SVOPL:BRAF, JHDMlD:BRAF, PPFIBP2:BRAF, SCL45A3:BRAF, ESRP1-RAF1, AGTRAP:BRAF, SVIP:BRAF, NRF1:BRAF, RAF1-CCDC176, RAF1-TRAK1, ESYT2:BRAF, PCBP2:BRAF, or SALL2:BRAF.

In some aspects the present disclosure provides methods of treating a subject with pediatric low grade glioma (pLGG), comprising reconstituting an amorphous solid dispersion of Compound A or a salt thereof in an aqueous solution and administering a pharmaceutically acceptable dosage of the reconstituted Compound A or a salt thereof to the subject in need thereof.

Additionally, the disease or condition provided herein includes refractory or recurrent malignancies whose growth may be inhibited using the methods of treatment of the present disclosure. In some embodiments, a cancer to be treated by the methods of treatment of the present disclosure is selected from the group consisting of carcinoma, squamous carcinoma, adenocarcinoma, sarcomata, endometrial cancer, breast cancer, ovarian cancer, cervical cancer, fallopian tube cancer, primary peritoneal cancer, colon cancer, colorectal cancer, squamous cell carcinoma of the anogenital region, melanoma, renal cell carcinoma, lung cancer, non-small cell lung cancer, squamous cell carcinoma of the lung, stomach cancer, bladder cancer, gall bladder cancer, liver cancer, thyroid cancer, laryngeal cancer, salivary gland cancer, esophageal cancer, head and neck cancer, glioblastoma, glioma, squamous cell carcinoma of the head and neck, prostate cancer, pancreatic cancer, mesothelioma, sarcoma, hematological cancer, leukemia, lymphoma, neuroma, and combinations thereof. In some embodiments, a cancer to be treated by the methods of the present disclosure include, for example, carcinoma, squamous carcinoma (for example, cervical canal, eyelid, tunica conjunctiva, vagina, lung, oral cavity, skin, urinary bladder, tongue, larynx, and gullet), and adenocarcinoma (for example, prostate, small intestine, endometrium, cervical canal, large intestine, lung, pancreas, gullet, rectum, uterus, stomach, mammary gland, and ovary). In some embodiments, a cancer to be treated by the methods of the present disclosure further include sarcomata (for example, myogenic sarcoma), leukosis, neuroma, melanoma, and lymphoma. In some embodiments, a cancer to be treated by the methods of the present disclosure is breast cancer. In some embodiments, a cancer to be treated by the methods of treatment of the present disclosure is triple negative breast cancer (TNBC). In some embodiments, a cancer to be treated by the methods of treatment of the present disclosure is ovarian cancer. In some embodiments, a cancer to be treated by the methods of treatment of the present disclosure is colorectal cancer. In some embodiments, the cancer is Cutaneous melanoma non-Spitzoid. In some embodiments, the cancer is

Specific examples of cancers that can be prevented and/or treated in accordance with present disclosure include, but are not limited to, the following: renal cancer, kidney cancer, glioblastoma multiforme, metastatic breast cancer; breast carcinoma; breast sarcoma; neurofibroma; neurofibromatosis; pediatric tumors; neuroblastoma; malignant melanoma; carcinomas of the epidermis; leukemias such as but not limited to, acute leukemia, acute lymphocytic leukemia, acute myelocytic leukemias such as myeloblastic, promyelocytic, myelomonocytic, monocytic, erythroleukemia leukemias and myclodysplastic syndrome, chronic leukemias such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenstrom's macroglobulinemia; monoclonal gammopathy of undetermined significance; benign monoclonal gammopathy; heavy chain disease; bone cancer and connective tissue sarcomas such as but not limited to bone sarcoma, myeloma bone disease, multiple myeloma, cholesteatoma-induced bone osteosarcoma, Paget's disease of bone, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangio sarcoma, neurilemmoma, rhabdomyosarcoma, and synovial sarcoma; brain tumors such as but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, and primary brain lymphoma; breast cancer including but not limited to adenocarcinoma, lobular (small cell) carcinoma, intraductal carcinoma, medullary breast cancer, mucinous breast cancer, tubular breast cancer, papillary breast cancer, Paget's disease (including juvenile Paget's disease) and inflammatory breast cancer; adrenal cancer such as but not limited to pheochromocytom and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor, acromegaly, and diabetes insipius; eye cancers such as but not limited to ocular melanoma such as iris melanoma, choroidal melanoma, and cilliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, and stromal tumor; cervical carcinoma; esophageal cancers such as but not limited to, squamous cancer, adenocarcinoma, adenoid cyctic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fungating (polypoid), ulcerating, superficial spreading, diffusely spreading, malignant lymphoma, liposarcoma, fibrosarcoma, and carcinosarcoma; colon cancers; colorectal cancer, KRAS mutated colorectal cancer; colon carcinoma; rectal cancers; liver cancers such as but not limited to hepatocellular carcinoma and hepatoblastoma, gallbladder cancers such as adenocarcinoma; cholangiocarcinomas such as but not limited to pappillary, nodular, and diffuse; lung cancers such as KRAS-mutated non-small cell lung cancer, non-small cell lung cancer, squamous cell carcinoma (epidermoid carcinoma), adenocarcinoma, large-cell carcinoma and small-cell lung cancer; lung carcinoma; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, androgen-independent prostate cancer, androgen-dependent prostate cancer, adenocarcinoma, leiomyosarcoma, and rhabdomyosarcoma; penal cancers; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acrallentiginous melanoma; kidney cancers such as but not limited to renal cell cancer, adenocarcinoma, hypernephroma, fibrosarcoma, transitional cell cancer (renal pelvis and/or uterer); renal carcinoma; Wilms' tumor; bladder cancers such as but not limited to transitional cell carcinoma, squamous cell cancer, adenocarcinoma, carcinosarcoma. In addition, cancers include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas.

In some embodiments the cancer is a tumor. In some embodiments, the tumor is a solid tumor. In certain embodiments, the tumor is a tumor selected from the group consisting of: colorectal tumor, pancreatic tumor, lung tumor, ovarian tumor, liver tumor, breast tumor, kidney tumor, prostate tumor, neuroendocrine tumor, gastrointestinal tumor, melanoma, cervical tumor, bladder tumor, glioblastoma, and head and neck tumor. In certain embodiments, the tumor is a colorectal tumor. In certain embodiments, the tumor is an ovarian tumor. In some embodiments, the tumor is a breast tumor. In some embodiments, the tumor is a lung tumor. In certain embodiments, the tumor is a pancreatic tumor. In some embodiments, the tumor is a melanoma tumor. In some embodiments, the tumor comprises or is characterized as having a gene fusion. In some embodiments, the cancer is a cancerous tumor. In certain embodiments, the subject in need thereof has had a tumor at least partially removed.

In some embodiments, a patient or population of patients to be treated with a pharmaceutical composition of the present disclosure have a solid tumor. In some embodiments, a solid tumor is a melanoma, renal cell carcinoma, lung cancer, bladder cancer, breast cancer, cervical cancer, colon cancer, gall bladder cancer, laryngeal cancer, liver cancer, thyroid cancer, stomach cancer, salivary gland cancer, prostate cancer, pancreatic cancer, or Merkel cell carcinoma. In some embodiments, a patient or population of patients to be treated with a pharmaceutical composition of the present disclosure have a hematological cancer. In some embodiments, the patient has a hematological cancer such as Diffuse large B cell lymphoma (“DLBCL”), Hodgkin's lymphoma (“HL”), Non-Hodgkin's lymphoma (“NHL”), Follicular lymphoma (“FL”), acute myeloid leukemia (“AML”), or Multiple myeloma (“MM”). In some embodiments, a patient or population of patients to be treated having the cancer selected from the group consisting of ovarian cancer, lung cancer and melanoma.

In some embodiments, a patient or population of patients to be treated with a pharmaceutical composition of the present disclosure has not previously been treated with MEK inhibitor or MAPK inhibitor. In some embodiments, a pharmaceutical composition of the present disclosure is administered in combination with a MEK inhibitor or MAPK inhibitor. In some embodiments, the MEK inhibitor is selected from: cobimetinib, selumetinib, pimasertib, PD0325901, refametinib, binimetinib, BI-847325, trametinib, GDC-0623, G-573, CH5126766, CIP-137401 and a compound having a structure of

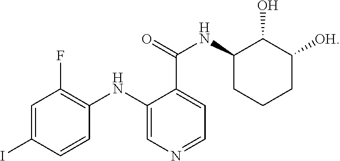 [SMILES: O=C(N[C@@H]1CCC[C@@H](O)[C@H]1O)c1ccncc1Nc1ccc(I)cc1F]

In some embodiments, the MEK inhibitor is selumetinib, binimetinib, or pimasertib. In some embodiments, the MEK inhibitor is pimasertib.

In some embodiments, the MEK inhibitor or a pharmaceutically acceptable salt thereof is selected from cobimetinib, selumetinib, pimasertib, PD0325901, refametinib, binimetinib, BI-847325, trametinib, GDC-0623, G-573, CH5126766, CI-1040, PD035901 and TAK-933. In some embodiments, the MEK inhibitor or a pharmaceutically acceptable salt thereof is selected from cobimetinib, selumetinib, pimasertib, PD0325901, refametinib, binimetinib, BI-847325, trametinib, GDC-0623, G-573, CH5126766, CI-1040, PD035901, TAK-933, and CIP-137401.

In some embodiments, the MEK inhibitor is a MEK inhibitor as described in U.S. Pat. Nos. 7,777,050, 8,178,693, 9,562,016, 7,425,637, 8,178,693, 9,156,795, 9,562,017, 7,378,423, 8,703,781, 9,290,468, each of which are hereby individually incorporated by reference in their entirety.

### EXAMPLES

The invention now being generally described, it will be more readily understood by reference to the following examples which are included merely for purposes of illustration of certain aspects and embodiments of the present invention, and are not intended to limit the invention in any way.

### Example 1. Patient Admixture Instructions (FIG. 1; Steps 1 Through 12)

In step 1, open the bottle containing a powder formulation and remove the seal. Do not throw away the child-resistant cap. Remove the oral syringe from the wrapper. In step 2, fill a cup with warm or room temperature water. Do not use cold water. In step 3, pull up on the plunger to draw water into the oral syringe above the 14 mL mark. In step 4, remove oral syringe from cup. Gently push the plunger to force all air bubbles out of the tip. Expel excess water so the plunger top is aligned with the 14 mL mark of the syringe. In step 5, Inject 14 mL of water to the bottle. Immediately replace the cap and vigorously shake the bottle for 60 seconds in all directions. Continue shaking in 15 sec increments until powder is fully dispersed. Do not shake for >2 min total time. Observe if the powder is fully dispersed into the liquid. In step 6, invert the bottle and swirl for 30 seconds. Remove the child-resistant cap and confirm that no solids are adhered in the bottle neck. If solids are present in the bottle neck, recap the bottle, invert, and swirl for an additional 15 seconds. Then, allow the bottle to sit for approximately one minute. In step 7, replace the cap with the bottle adaptor. Secure it to the bottle by pressing it into the bottle neck. In step 8, insert the tip of the oral syringe into the bottle adaptor. The tip of the syringe should fit snugly into the hole of the bottle of adaptor. In step 9, with syringe in place, swirl the suspension for 30 seconds, turn the bottle upside down and draw the prescribed volume plus 1 mL extra into the syringe. In step 10, remove the oral syringe from the bottle. Remove only air bubbles out of the tip by gently pushing up on the plunger. In step 11, reinsert syringe into the bottle and adjust to the prescribed volume. In step 12, gently remove syringe from the bottle. Place the tip of the syringe in the child's mouth against the inside of the cheek. Slowly squirt medicine into the mouth by pressing down on the plunger. Allow the child to swallow.

### Example 2. Part 1 Taste/Palatability Assessment

The objective of this study was to evaluate the taste and palatability attributes (smell, sweetness, bitterness, flavor, mouth feel/texture, and aftertaste) and overall acceptability of Compound A hot-melt extrusion (HME) powder for reconstitution (PfR) suspension formulations.

Part 1 was a single center, open-label, randomized, single period, 6-way crossover design to evaluate the taste and palatability attributes and overall palatability of several Compound A HME PfR suspension formulations with different flavors and sweetener levels using the sip and spit methodology in 12 volunteers. On the morning of Day 1, subjects were randomized to 1 of 6 sequences (ABFCED, BCADFE, CDBEAF, DECFBA, EFDACB, and FAEBDC), with 2 subjects assigned to each sequence. Each subject received the following regimens in a randomized manner. (Tables 1 and 2)

TABLE 1
Description of Regimens in Part 1

| | | | Route of |
|---|---|---|---|
| | Formulations^{a} | Volume | Administration |
| A | Compound A HME PfR suspension | 5 mL | Oral (sip and |
| | formulation 1 (reference; | | spit), Fasted |
| | no sweetener or flavoring) | | |
| B | Compound A HME PfR | 5 mL | Oral (sip and |
| | suspension formulation 2 | | spit), Fasted |
| C | Compound A HME PfR | 5 mL | Oral (sip and |
| | suspension formulation 3 | | spit), Fasted |
| D | Compound A HME PfR | 5 mL | Oral (sip and |
| | suspension formulation 4 | | spit), Fasted |
| E | Compound A HME PfR | 5 mL | Oral (sip and |
| | suspension formulation 5 | | spit), Fasted |
| F | Compound A HME PfR | 5 mL | Oral (sip and |
| | suspension formulation 6 | | spit), Fasted |

^{a} Regimen A contained 25 mg/mL Compound A and no sweetener or flavoring. Regimens B to F contained different flavors, sweetener levels and the same amount of Compound A.

The drug product of Compound A used in Part I has been formulated as a powder for reconstitution (PfR) dosage form. This PfR formulation was used herein.

The drug product is packaged as a 300 mg active strength (to deliver) in bottles equipped with child-resistant caps and an induction seal liner. It is reconstituted with water or an aqueous diluents containing a flavoring agent and/or sweetener in Table 2b to a 25 mg/mL active concentration and dosed with a syringe. In one example, the bottle is overfilled to 365 mg Compound A, to allow for reproducible dosing delivery of 300 mg Compound A (i.e. 12 mL of 25 mg/mL Compound A concentration). The dose composition of Compound A PfR is provided below in Table 2a as a base formulation. Formulation A of Table 1 is the same as the base formulation Table 2a, and formulations B-F in Table 1 contain the base formulation as shown Table 2a and the corresponding flavoring agent and/or sweetener illustrated in Table 2b.

The flavoring agent/sweetener are pharmacy compounded with water as described below in Table 2b.

TABLE 2a
Composition of Compound A Powder for
Reconstitution, 300 mg Bottles

| | | Compendial | | mg/ |
|---|---|---|---|---|
| Ingredients | Function | Status | Wt. % | bottle |
| Compound A | Active | — | 10..0 | 365.9 |
| Copovidone | Solubilizer | USP-NF/Ph. Eur | 15.0 | 548.8 |
| Microcrystalline | Filler | USP-NF/Ph. Eur | 31.1 | 1138.9 |
| Cellulose | | | | |
| Mannitol | Filler | USP-NF/Ph. Eur | 31.1 | 1138.9 |
| Sodium Lauryl Sulfate | Solubilizer | USP-NF/Ph. Eur | 0.75 | 27.4 |
| Simethicone GS | Anti-foam | Manufacturer's | 7.5 | 274.4 |
| | agent | CoA¹ | | |
| Colloidal Silicon | Dispersant | USP-NF/Ph. Eur | 4.5 | 164.6 |
| Dioxide | | | | |
| Total | | | 100 | 3659.0 |

¹Tested to USP monograph for Simethicone, USP for Assay and Identification.

TABLE 2b
Description of Flavor and Sweetener Levels For Formulation

| | | Level | | Level |
|---|---|---|---|---|
| Regimen | Flavoring | (% wt/vol) | Sweetener | (% wt/vol) |
| A | None | 0 | None | 0 |
| B | None | 0 | Sucralose | 0.15% |
| C | Strawberry | 0.4% | Sucralose | 0.15% |
| D | Strawberry | 0.4% | Sucralose | 0.3% |
| E | Vanilla | 0.4% | Sucralose | 0.3% |
| F | Vanilla | 0.4% | Sucralose | 0.15% |

### Example 3. Part 2 Relative Bioavailability and Food Effect

The objective of this study was (1) to determine the relative bioavailability of the Compound A HME PfR suspension formulation compared to that of the Compound A HME tablet formulation in healthy subjects; (2) to evaluate the PK profiles of Compound A HME tablet and Compound A HME PfR suspension formulations in healthy subjects; (3) to assess the effect of food on the PK profiles of Compound A following a single dose of the Compound A HME tablet formulation in healthy subjects in the fed state; and (4) to provide information on the safety and tolerability of Compound A after oral administration.

Part 2 was a single center, open-label, randomized, 3-period crossover design to assess the relative bioavailability of the selected Compound A HME PfR suspension formulation compared to the HME tablet reference formulation in 12 volunteers. Effected of food on the Compound A HME tablet exposure was also investigated. In Period 1, subjects were randomized prior to the first dose of Compound A to 1 of 3 regimen sequences (GHI, HIG, and IGH), with 4 subjects assigned to each regimen sequence. On Day 1 of each study period, subjects received the following regimens in Table 2.

TABLE 2
Formulations Used in Part 2

| | | Dose Period | Dose Period | Route of |
|---|---|---|---|---|
| Regimen | Formulation | 1^{a} | 2 and 3^{a} | Administration |
| G | Compound A HME | 300 mg | 100 mg | Oral, Fasted |
| | Tablet Formulation | (Regimen G1) | (Regimen G2) | |
| | (reference) | | | |
| H | Compound A selected | 300 mg | 100 mg | Oral, Fasted |
| | HME PfR suspension | (Regimen H1) | (Regimen H2) | |
| | formulation | | | |
| I | Compound A HME | 300 mg | 100 mg | Oral, Fed |
| | Tablet Formulation | (Regimen I1) | (Regimen I2) | |

^{a} Due to the musculoskeletal AEs experienced in Period 1 following administration of 300 mg, a reduced dose of 100 mg was administered in Period 2 and Period 3. In order to distinguish between the 300 mg and 100 mg doses, Period 1 regimens were identified as G1, H1, and I1, and Period 2/Period 3 regimens were identified as G2, H2, and I2.

Compound A drug product was formulated as a powder for reconstitution (PfR) dosage form for use in clinical studies. The drug product was packaged as 300 mg (active strength, to deliver) in glass bottles equipped with child-resistant caps and an induction seal liner. It is reconstituted with water to a 25 mg/mL active concentration and dosed with a syringe. The bottle is overfilled to 430 mg Compound A and reconstituted with 14 mL of water, to allow for reproducible dosing delivery of 300 mg Compound A (i.e. 12 mL of 25 mg/mL Compound A concentration). The 100 mg dose utilized the same bottle and reconstitution procedure as the 300 mg dose. A 4 mL aliquot at 25 mg/mL was utilized to provide the 100 mg dose. The dose composition of Compound A PfR is provided below in Table 4.

TABLE 4
Composition of Compound A Powder for Reconstitution Flavored,
100 mg (H2 Regimen) and 300 mg Bottles (Regimen H1)

| | | Compendial | | 100 mg Dose | 300 mg Dose |
|---|---|---|---|---|---|
| Ingredients | Function | Status | Wt. % | (mg/bottle) | (mg/bottle) |
| Compound A | Active | — | 9.738 | 143.24 | 429.72 |
| Copovidone | Solubilizer | USP-NF/Ph. Eur | 14.675 | 214.86 | 644.58 |
| Microcrystalline Cellulose | Filler | USP-NF/Ph. Eur | 30.457 | 445.90 | 1337.7 |
| Mannitol | Filler | USP-NF/Ph. Eur | 30.457 | 445.90 | 1337.7 |
| Sodium Lauryl Sulfate | Solubilizer | USP-NF/Ph. Eur | 0.734 | 10.73 | 32.2 |
| Simethicone GS | Anti-foam agent | Manufacturer's CoA¹ | 7.338 | 107.43 | 322.3 |
| Colloidal Silicon Dioxide | Dispersant | USP-NF/Ph. Eur | 4.403 | 64.47 | 193.4 |
| Sucralose Powder | Sweetener | USP-NF/Ph. Eur | 0.587 | 8.60 | 25.8 |
| Artificial Strawberry Flavor | Flavoring | Manufacturer COA | 1.565 | 22.93 | 68.8 |
| Total | | | 100 | 1464.07 | 4392.2 |

¹The 100 mg dose was supplied from the 300 mg bottle. After reconstitution to 25 mg/mL, a 4 mL aliquot for the 100 mg dose was dosed instead of the 12 mL dose as for the 300 mg dose.

Subjects underwent preliminary screening procedures to determine their eligibility for Part 2 of the study at the screening visit (Day −28 to Day −2). Each period followed the same study design. Subjects admitted in the morning on the day before dosing (Day −1) for all regimens.

Subjects received a single dose of Compound A in the morning of Day 1 following an overnight fast of a minimum of 10 h (Regimens G and H, fasted state) or 30 min after the start of a standard high-fat breakfast (Regimen I, fed state). Blood samples were collected at regular intervals for PK analysis from pre-dose to 120 h post-dose. After administration of Regimen H (selected Compound A HME PfR suspension formulation in the fasted state), subjects completed a taste/palatability questionnaire individually and privately.

Subjects remained in the clinical unit until 96 h post-dose (Day 5). Subjects returned to the clinic at 120 h post-dose (Day 6) for a final PK sample.

There was a minimum 14-day washout between each Compound A dose. A follow-up call took place 7 to 10 days after the final dose to ensure the ongoing wellbeing of the subjects.

### Example 4—Taste/Palatability and Pharmacokinetic Evaluation

Taste/Palatability Results (Part 1 and Part 2 Regimen H Only)

Taste was assessed on palatability attributes (smell, sweetness, bitterness, flavor, mouth feel and aftertaste) and overall, on a 9-grade system. The median (min-max) scores for each taste/palatability attribute for Part 1 and Part 2 Regimen H are summarized in Table 5.

TABLE 5
Median (Min-Max) Scores of Different Taste/Palatability Attributes for the Assessment
of Compound A HME PfR Suspension Formulation Administered in Different Regimens:
Taste/Palatability Analysis Set (Part 1 and Part 2 Regimen H only)

| | Test Treatment (0.4% Flavoring/Sucralose %) | | | | | | |
|---|---|---|---|---|---|---|---|
| | Regimen A | Regimen B | Regimen C | Regimen D | Regimen E | Regimen F | Regimen H |
| | (NA/ | (NA/ | (Strawberry/ | (Strawberry/ | (Vanilla/ | (Vanilla/ | (Strawberry/ |
| | NA) | 0.15%) | 0.15%) | 0.3%) | 0.3%) | 0.15%) | 0.15%) |
| | Median | Median | Median | Median | Median | Median | Median |
| Taste | Score | Score | Score | Score | Score | Score | Score |
| Attribute | (Min-Max) | (Min-Max) | (Min-Max) | (Min-Max) | (Min-Max) | (Min-Max) | (Min-Max) |
| Smell | 5.0 (1-6) | 5.0 (4-7) | 7.0 (3-8) | 7.0 (6-8) | 7.0 (6-8) | 7.0 (3-9) | 7.0 (5-9) |
| Sweetness | 4.0 (1-6) | 6.0 (1-7) | 7.0 (4-8) | 7.0 (3-8) | 7.0 (4-8) | 6.0 (3-9) | 7.0 (4-8) |
| Bitterness | 4.0 (1-7) | 5.0 (3-7) | 5.5 (4-7) | 5.0 (4-8) | 5.5 (5-7) | 5.0 (2-7) | 4.5 (2-8) |
| Flavour | 3.0 (1-6) | 5.5 (3-7) | 7.0 (3-8) | 6.5 (3-8) | 6.5 (5-8) | 5.5 (3-8) | 6.5 (4-8) |
| Mouth feel/ | 3.0 (1-7) | 4.0 (1-7) | 6.0 (1-8) | 5.5 (2-8) | 6.0 (2-8) | 5.0 (2-8) | 6.0 (1-8) |
| Texture | | | | | | | |
| Aftertaste | 3.0 (1-6) | 4.0 (1-7) | 6.0 (2-8) | 7.0 (4-8) | 7.0 (2-8) | 5.0 (3-7) | 4.0 (2-8) |
| Overall | 3.0 (1-6) | 5.0 (1-7) | 7.0 (3-8) | 6.0 (3-8) | 6.5 (4-8) | 5.5 (3-8) | 7.0 (3-8) |

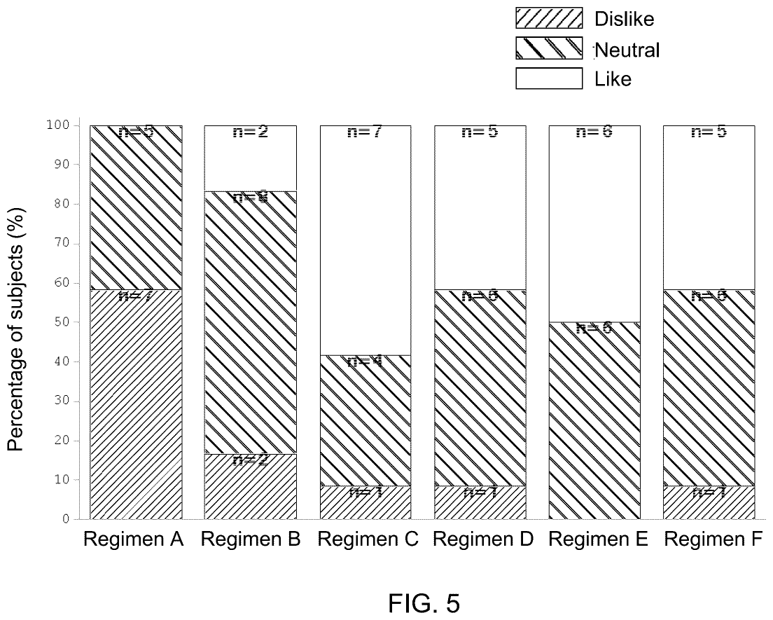

The overall taste/palatability assessments are presented on a stacked bar chart in FIG. 5.

Taste/Palatability Analysis (Part 1)

Median scores for the reference (Regimen A) ranged from 3.0 to 5.0 for all taste attributes including overall acceptability, indicating subjects had a moderate dislike to neutral view on the product. When a sweetener was added palatability was improved with median scores ranging from 4.0 to 6.0, indicating a largely neutral view on the product. Flavoring paired with different sweetener levels increased median scores from 5.0 to 7.0 showing acceptability ranged from neutral to a moderate liking.

Based on the grouping (Grade 1-3: Dislike, Grade 4-6: Neutral, Grade 7-9: Like), 7 subjects indicated a dislike for the reference product based on overall acceptability. This was reduced to 2 subjects for the sweetened product and was no more than 1 subject for each flavored regimen. The regimen with the highest proportion of subjects scoring as ‘Like’ was Regimen C with 7 subjects selecting this rating.

The addition of a sweetener alone to the Compound A HME PfR suspension formulation provided modest improvements vs. the reference product, however these were not significant in all cases. Each of the Compound A formulations including a flavoring provided a more robust improvement over the reference product for each taste attribute with the exception of bitterness. There did not appear to be a clear preference between flavored regimens, however, Regimen F (vanilla, low sucralose) tended to show the smallest improvements.

With the exception of bitterness, the Friedman's test was statistically significant at the 5% significance level for each taste aspect including overall acceptability, indicating that at least one of the flavor/sweetener levels had a significantly different taste score to the other regimens (p=<0.001, 0.013, <0.001, <0.001, 0.022 and 0.033 for overall acceptability, smell, sweetness, flavor, mouth feel/texture and aftertaste). For bitterness, the p-value was greater than 0.05 (p=0.650), suggesting there was no significant difference between at least 2 of the regimens for bitterness scores.

For all pairwise comparisons, the median of the paired differences was positive, indicating that each flavor/sweetener combination showed an improved acceptability for each taste aspect analyzed and overall acceptability when compared to the reference with no sweetener or flavoring.

Statistical analysis of the taste/palatability assessments using the Wilcoxon signed rank test for taste/palatability is presented in Table 6.

TABLE 6
Taste/Palatability Assessment Results from the Wilcoxon Signed-
Rank Test: Taste/Palatability Analysis Set (Part 1)

| | Test Treatment (0.4% Flavoring/Sucralose %) | | | | | | | | | |
|---|---|---|---|---|---|---|---|---|---|---|
| | Regimen B | | Regimen C | | Regimen D | | Regimen E | | Regimen F | |
| | (NA/ | | (Strawberry/ | | (Strawberry/ | | (Vanilla/ | | (Vanilla/ | |
| | 0.15%) | | 0.15%) | | 0.3%) | | 0.3%) | | 0.15%) | |
| Taste | Diff | P-val | Diff | P-val | Diff | P-val | Diff | P-val | Diff | P-val |
| Attribute | (1) | (2) | (1) | (2) | (1) | (2) | (1) | (2) | (1) | (2) |
| Smell | 0.5 | 0.50 | 3.0 | 0.031 | 3.0 | 0.031 | 2.5 | 0.031 | 3.0 | 0.19 |
| Sweetness | 2.0 | 0.094 | 3.5 | 0.004 | 2.0 | 0.004 | 3.0 | 0.002 | 2.0 | 0.047 |
| Flavour | 2.0 | 0.004 | 3.0 | 0.002 | 3.0 | 0.004 | 4.0 | 0.004 | 3.0 | 0.016 |
| Mouth feel/ | 0.5 | 0.055 | 2.0 | 0.025 | 2.0 | 0.008 | 2.0 | 0.008 | 1.5 | 0.004 |
| Texture | | | | | | | | | | |
| Aftertaste | 3.0 | 0.037 | 3.0 | 0.021 | 3.0 | 0.012 | 3.0 | 0.006 | 2.5 | 0.012 |
| Overall | 1.0 | 0.066 | 2.0 | <0.001 | 3.0 | <0.001 | 3.0 | 0.002 | 1.5 | 0.006 |

(1) Median of the paired difference (Test Regimen − Regimen A) for each subject
(2) P-value from Wilcoxon Signed Rank Test under null hypothesis that median difference is equal to 0 Subjects tasted 5 mL Compound A HME PfR oral suspension, 6 different formulations (Regimen A-F = Formulation 1-6) Regimen A = 25 mg/mL Compound A (no sweetener/flavouring), Regimens B to F = different flavours, sweetener levels concentrations (mg/mL)

In general, the smallest improvements from Regimen A (no flavoring or sucralose) were observed for Regimen B (0.15% sucralose) across the taste aspects with the exception of aftertaste, where the improvement equaled those regimens with flavoring. Regimen C (strawberry flavoring and 0.15% sucralose), Regimen D (strawberry flavoring and 0.3% sucralose), and Regimen E (vanilla flavoring and 0.3% sucralose), all provided similar improvements across the taste aspects and overall acceptability, when compared to reference Regimen A, with these improvements generally greater than Regimen F (vanilla flavoring and 0.15% sucralose). Regimen F had the smallest improvement in aftertaste compared to Regimen A across regimens, and the joint smallest improvement in sweetness with unflavored Regimen B.

Taste/Palatability Analysis (Part 2)

The overall taste profile scores for Regimen H were comparable to those for Regimen C in Part 1, which used the same flavor and sweetener (Strawberry/0.15%).

Taste/Palatability Statistical Issues

For the Friedman's test, a subject can only be included within the comparison of all regimens if they had a numeric score for each regimen. As some of the subjects scored a taste aspect with “NA” (not applicable) for at least 1 regimen, they therefore were not included and hence the number of subjects included dropped for the taste aspects (number of subjects included in the analysis): smell (4), bitterness (4), sweetness (9), flavor (11) and aftertaste (9). Some caution should be used when interpreting the result for regimens with significant missing data.

Taste/Palatability Conclusions

For overall acceptability and all taste aspects, the taste scores were significantly improved for the Compound A HME PfR suspension formulation with addition of sweetener sucralose alone or additionally including flavoring (strawberry or vanilla), with greater improvement seen with addition of both sweetener and flavoring

Pharmacokinetic Results and Analysis (Part 2)

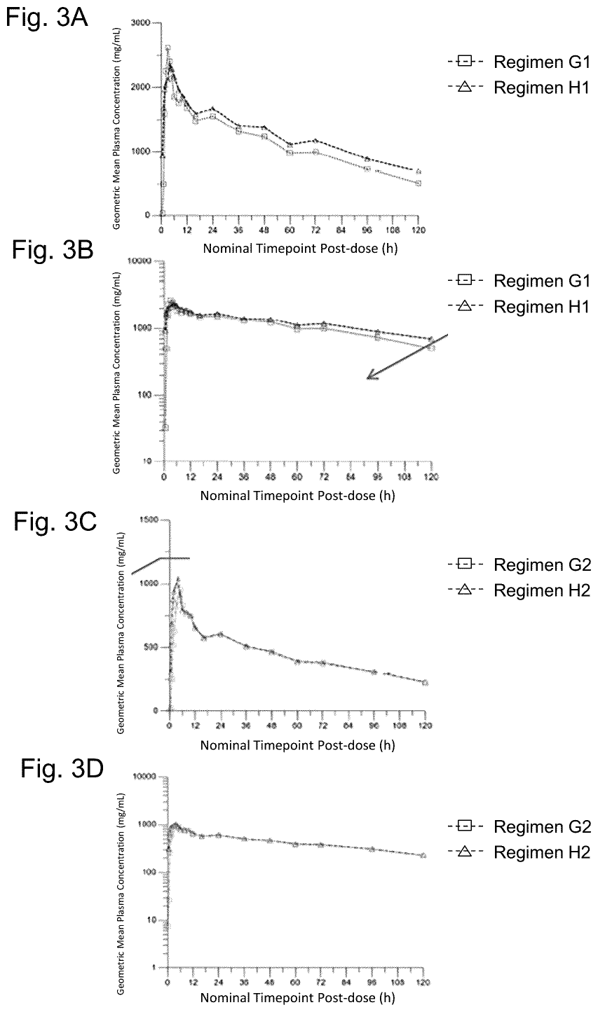

In FIGS. 3(A)-3(D) and Table, following administration of Compound A as the reference HME tablet in the fasted state at 300 mg and 100 mg dose levels, Tₘₐₓ was reached at 3.00 h post dose for all subjects in Regimen G1, and between 2.00 and 5.00 h post dose for subjects in Regimen G2, with a median Tₘₐₓ of 3.5 h. Geometric mean T_{1/2} was 67.4 h and 73.5 h for the 300 mg and 100 mg dose levels, respectively. When Compound A was administered in the PfR suspension (Regimen H1 and H2) formulation at the 300 mg and 100 mg dose levels, median Tₘₐₓ occurred at 3.000 h for both regimens. Geometric mean T_{1/2} estimates were long at 83.177 h and 65.363 h for the 300 mg and 100 mg dose levels, respectively.

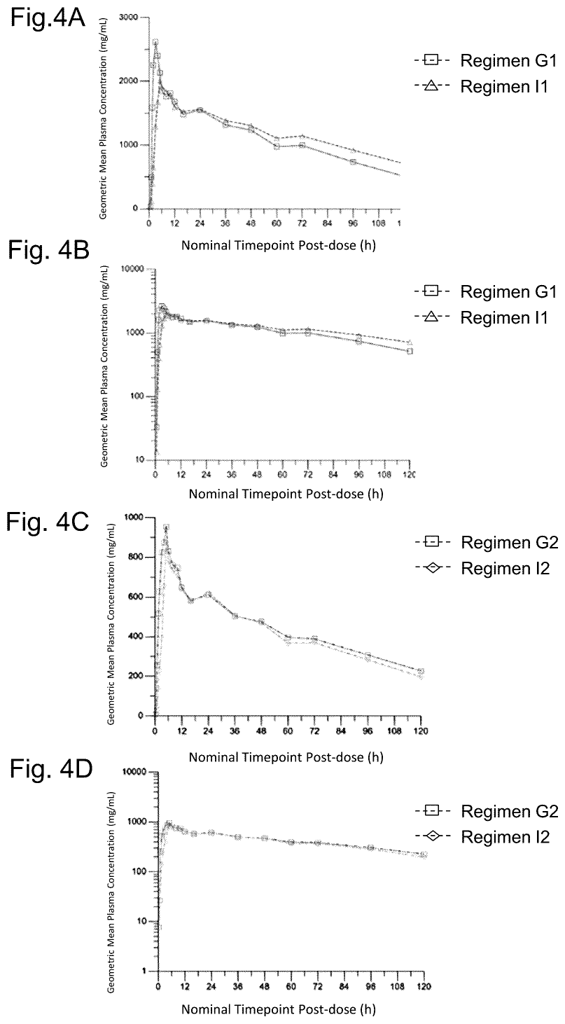

In FIGS. 4(A)-4(D) and Table 7, following administration of Compound A as the HME tablet in the fed state at 300 mg (Regimen H1) and 100 mg (Regimen H2) dose levels, median Tₘₐₓ was 6.5 h and 5.0 for Regimen I1 and 12, respectively. Geometric mean T_{1/2} estimates were long at 85.4 h and 61.7 h for Regimen I1 and 12, respectively. The inter-subject variability for Cmax and AUC(0-last) was low to moderate for the 300 mg tablet administered in the fed state (Regimen I1), with CVs of 26.5% and 11.7%, respectively, and CVs of 23.4% and 20.7% for the 100 mg tablet administered in the fed state (Regimen I2), respectively.

TABLE 7
Geometric Mean (Geometric CV %) Plasma Pharmacokinetic Parameters for Compound
A Following Single Oral Doses of 100 mg and 300 mg Compound A to Healthy Male
and Female Volunteers Administered as Tablet and Suspension Formulations

| Regimen | G1 | H1 | I1 | G2 | H2 | I2 |
|---|---|---|---|---|---|---|
| Dose Level (mg) | 300 mg | 300 mg | 300 mg | 100 mg | 100 mg | 100 mg |
| Treatment | HME | HME PfR | HME | HME | HME PfR | HME PfR |
| | Tablet | suspension | Tablet | Tablet | suspension | suspension |
| Prandial State | Fasted | Fasted | Fasted | Fasted | Fasted | Fed |
| N | N = 4 | N = 4 | N = 4 | N = 7 | N = 8 | N = 8 |
| Parameter (units) | | | | | | |
| Tₘₐₓ^{a} (h) | 3.0 | 3.0 | 6.5 | 3.5 | 3.0 | 5.0 |
| | (3.00-3.00) | (1.50-4.00) | (4.00-24.00) | (2.00-5.00) | (1.50-4.00) | (4.0-24.0) |
| | | | | [n = 6] | [n = 7] | |
| Cₘₐₓ | 2620 | 2470 | 2870 | 1180 | 1110 | 910 |
| (ng/mL) | (24.3) | (11.7) | (26.5) | (21.6) | (22.0) | (23.4) |
| | | | | [n = 6] | [n = 7] | |
| Cₘₐₓ/D | 8.73 | 8.24 | 7.42 | 11.8 | 11.1 | 9.31 |
| (ng/mL/mg) | (24.3) | (11.7) | (1.91) | (21.6) | (22.0) | (2.19) |
| | | | | [n = 6] | [n = 7] | |
| AUC(0-24) | 40200 | 43100 | 36900 | 16400 | 16600 | 15300 |
| (ng · h/mL) | (26.4) | (18.8) | (11.7) | (14.3) | (20.2) | (21.4) |
| | | | | [n = 6] | [n = 7] | |
| AUC(0-24)/D | 134 | 144 | 123 | 164 | 166 | 149 |
| (ng · h/mL/mg) | (26.4) | (18.8) | (13.0) | (14.3) | (20.2) | (29.6) |
| | | | | [n = 6] | [n = 7] | |
| AUC(0-120) | 134000 | 151000 | 147000 | 53500 | 52400 | 52800 |
| (ng · h/mL) | (29.3) | (18.0) | (11.6) | (15.2) | (22.3) | (20.8) |
| | | | | [n = 6] | [n = 7] | |
| AUC(0-120)/D | 445 | 503 | 482 | 535 | 524 | 514 |
| (ng · h/mL/mg) | (29.3) | (18.0) | (54.9) | (15.2) | (22.3) | (100) |
| | | | | [n = 6] | [n = 7] | |
| AUC(0-last) | 133000 | 151000 | 146000 | 53500 | 52400 | 52800 |
| (ng · h/mL) | (29.4) | (17.9) | (11.6) | (15.0) | (22.3) | (20.7) |
| | | | | [n = 6] | [n = 7] | |
| AUC(0-last)/D | 445 | 503 | 479 | 535 | 524 | 513 |
| (ng · h/mL/mg) | (29.4) | (17.9) | (53.6) | (15.0) | (22.3) | (99.6) |
| | | | | [n = 6] | [n = 7] | |
| AUC(0-inf) | NC | NC | NC | NC | NC | NC |
| (ng · h/mL) | (NC) | (NC) | (NC) | (NC) | (NC) | (NC) |
| AUC(0-inf)/D | NC | NC | NC | NC | NC | NC |
| (ng · h/mL/mg) | (NC) | (NC) | (NC) | (NC) | (NC) | (NC) |
| T_{1/2} (h) | 67.4 | 83.2 | 85.4 | 73.5 | 65.4 | 60.0 |
| | (17.2) | (22.0) | (39.2) | (26.0) | (13.2) | (13.6) |
| | | | | [n = 6] | [n = 6] | |

As a result of the long T_{1/2} relative to the sampling schedule, the area over which the AUC(0-inf) was extrapolated was large (>20%) and hence AUC(0 inf) was not reliably calculated for any subject.

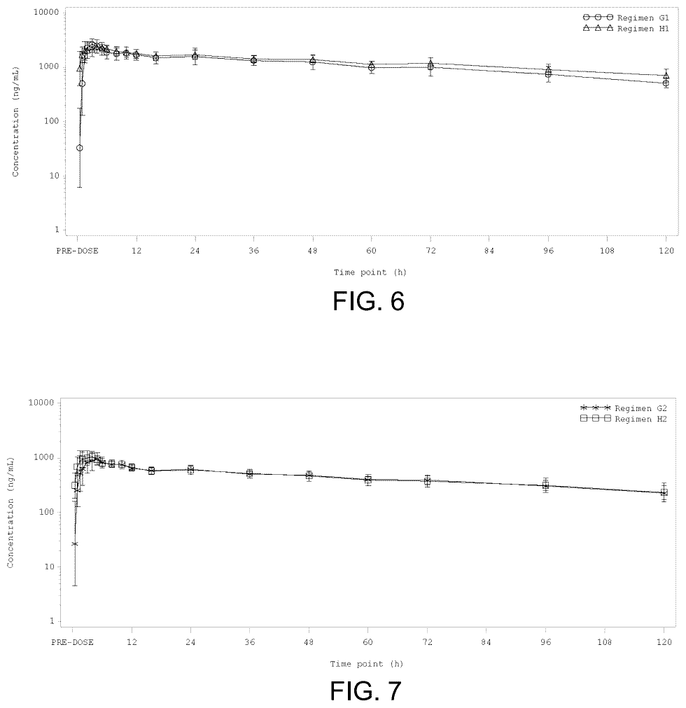

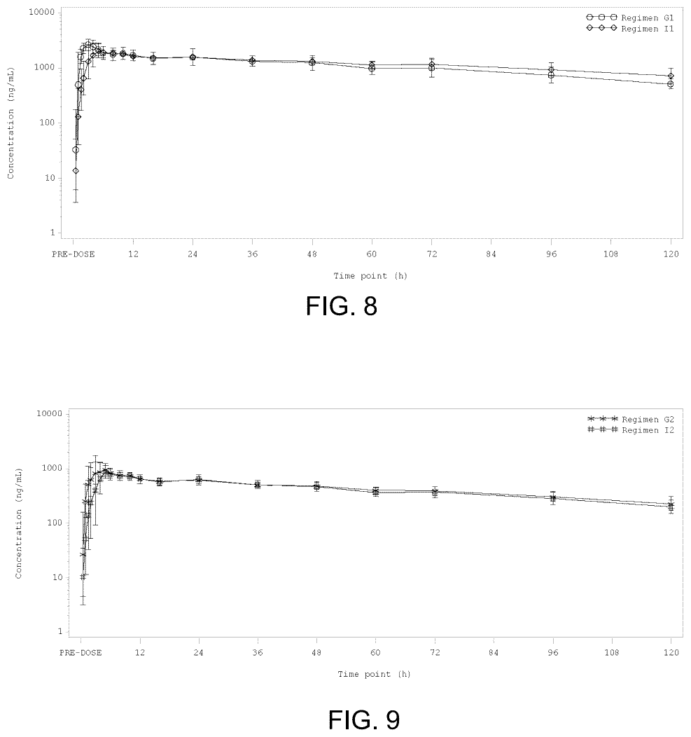

The mean plasma concentration vs. time profiles of Compound A are illustrated for regimen (Regimens H1 and G2, Regimens H2 and G2, Regimens I1 and G1 and Regimens I2 and G2) on a log₁₀/linear scale in FIGS. 6-9, respectively.

### Regimen G1 (300 mg HME Tablet, Fasted) and Regimen G2 (100 mg HME Tablet, Fasted)

Following a single oral administration of Compound A to healthy male and female subjects as the reference HME tablet in the fasted state at 300 mg (Regimen G1) and 100 mg (Regimen G2) dose levels, quantifiable plasma concentrations of Compound A were observed from the first time-point (0.5 h post-dose) for Regimen G1 and were observed at pre-dose (due to carryover from the previous period) for all subjects for Regimen G2. Subject 2005 was excluded from the Regimen G2 summary statistics and statistical analysis as their pre-dose value (36.4 ng/mL) represented more than 5% of their corresponding Cₘₐₓ value (724 ng/mL). Maximum plasma concentrations (Cₘₐₓ) of Compound A were reached at 3.00 h post-dose for all subjects in Regimen G1 and between 2.00 h and 5.00 h post-dose for all subjects for Regimen G2, with a median Tₘₐₓ of 3.50 h for Regimen G2.

Following Cₘₐₓ, plasma concentrations declined in a multiphasic manner and remained quantifiable until the last sampling time-point (120 h post-dose) in all subjects for both Regimen G1 and G2. At the 300 mg dose level (Regimen G1), T_{1/2} was reliably calculated for all 4 subjects, ranging between 56.7 h and 82.6 h, with a geometric mean of 67.4 h. At the 100 mg dose level (Regimen G2), T_{1/2} was reliably calculated for all 6 subjects included in the summary statistics and ranged between 58.3 h and 113 h with a geometric mean of 73.5 h.

At the 300 mg dose level (Regimen G1), Cmax ranged from 2040 ng/mL to 3240 ng/mL, with a geometric mean (geometric CV %) of 2620 ng/mL (24.3%). AUC(0-last) ranged from 103000 ng·h/mL to 183000 ng·h/mL, with a geometric mean (geometric mean CV %) of 133000 ng·h/mL (29.4%). At the 100 mg dose level (Regimen G2), Cmax ranged from 1010 ng/mL to 1670 ng/mL, with a geometric mean (geometric mean CV %) of 1180 ng/mL (21.6%). AUC(0-last) ranged from 44000 ng·h/mL to 66900 ng·h/mL, with a geometric mean (geometric mean CV %) of 53500 ng·h/mL (15.0%). In all subjects for both regimens, AUC(0-inf) was not able to be reliably calculated due to the extrapolated portion of the curve representing >20% of the total area.

### Regimen H1 (300 mg HME PfR Suspension, Fasted) and Regimen H2 (100 mg HME PfR Suspension, Fasted)

Following a single oral administration of Compound A to healthy male and female subjects as the HME PfR suspension in the fasted state at 300 mg (Regimen H1) and 100 mg (Regimen H2) dose levels, quantifiable plasma concentrations of Compound A were observed from the first time-point (0.5 h post-dose) for Regimen H1 and were observed at pre-dose (due to carryover from the previous period) for 7 out of 8 subjects for Regimen H2, with concentrations quantifiable from the first time-point of 0.5 h post-dose in the remaining subject. Subject 2010 was excluded from the Regimen H2 summary statistics and statistical analysis as their pre-dose value (86.6 ng/mL) represented more than 5% of the corresponding Cₘₐₓ value (1200 ng/mL). Maximum plasma concentrations of Compound A were reached between 1.50 h and 4.00 h post-dose for all subjects in Regimens H1 and H2, with a median Tₘₐₓ of 3.00 h for both regimens.

Following Cₘₐₓ, plasma concentrations declined in a multi-phasic manner and remained quantifiable until the last sampling time-point (120 h post-dose) in all subjects for both regimens. At the 300 mg dose level (Regimen H1), T_{1/2} was reliably calculated for all 4 subjects, ranging between 71.9 h and 114 h, with a geometric mean of 83.2 h. At the 100 mg dose level (Regimen H2), T_{1/2} was reliably calculated for 6 subjects and ranged between 57.3 h and 77.5 h with a geometric mean of 65.4 h. Where terminal slopes could not be reliably determined, this was a result of an unacceptable coefficient of determination (i.e., R²<0.9).

At the 300 mg dose level (Regimen H1), Cₘₐₓ ranged from 2300 ng/mL to 2940 ng/mL, with a geometric mean (geometric CV %) of 2470 ng/mL (11.7%). AUC(0-last) ranged from 127000 ng·h/mL to 189000 ng·h/mL, with a geometric mean (geometric CV %) of 151000 ng·h/mL (17.9%). At the 100 mg dose level (Regimen H2), Cₘₐₓ ranged from 884 ng/mL to 1580 ng/mL, with a geometric mean (geometric mean CV %) of 1110 ng/mL (22.0%). AUC(0-last) ranged from 42500 ng·h/mL to 72500 ng·h/mL, with a geometric mean (geometric CV %) of 52400 ng·h/mL (22.3%). Where AUC(0-inf) was not able to be reliably calculated, this was due to the extrapolated portion of the curve representing >20% of the total area and the result of an unacceptable coefficient of determination (i.e., R²<0.9).

### Regimen I1 (300 mg HME Tablet, Fed) and Regimen I2 (100 mg HME Tablet, Fed)

Following a single oral administration of Compound A to healthy male and female subjects as the HME tablet in the fed state at 300 mg (Regimen I1) and 100 mg (Regimen I2) dose levels, quantifiable plasma concentrations of Compound A were observed from the first timepoint (0.5 h post-dose) for Regimen I1 and were observed at pre-dose (due to carryover from the previous period) for 7 out of 8 subjects for Regimen I2, with concentrations quantifiable from 0.5 h in the remaining subject. Maximum plasma concentrations of Compound A were reached between 4.00 h and 24.00 h post-dose for both regimens, with median Tₘₐₓ of 6.50 h and 5.00 h for Regimens I1 and I2, respectively.

Following Cₘₐₓ, plasma concentrations declined in a multi-phasic manner and remained quantifiable until the last sampling time-point (120 h post-dose) in all subjects for both regimens. At the 300 mg dose level (Regimen I1), T_{1/2} was reliably calculated for 3 out of 4 subjects, ranging between 58.9 h and 125 h, with a geometric mean of 85.4 h. At the 100 mg dose level (Regimen I2), T½ was reliably calculated for 7 out of 8 subjects and ranged between 51.3 h and 77.2 h with a geometric mean of 61.7 h. Where terminal slopes could not be reliably determined, this was a result of an unacceptable coefficient of determination (i.e., R²<0.9).

At the 300 mg dose level (Regimen I1), Cₘₐₓ ranged from 1650 ng/mL to 2870 ng/mL, with a geometric mean (geometric CV %) of 2170 ng/mL (26.5%). AUC(0-last) ranged from 124000 ng·h/mL to 159000 ng·h/mL, with a geometric mean (geometric CV %) of 143000 ng·h/mL (11.4%). At the 100 mg dose level (Regimen I2), Cₘₐₓ ranged from 667 ng/mL to 1250 ng/mL, with a geometric mean (geometric CV %) of 910 ng/mL (23.4%). AUC(0-last) ranged from 34600 ng·h/mL to 66000 ng·h/mL, with a geometric mean (geometric CV %) of 50400 ng·h/mL (20.7%). Due to the extrapolated portion of the curve representing >20% of the total area and the result of an unacceptable coefficient of determination (i.e., R²<0.9), AUC(0-inf) was only able to be reliably calculated for Subject 2008 in Regimen I2, with a value of 57300 ng·h/mL.

Statistical Results

Analysis of Dose Proportionality

The results from the formal statistical analyses of the PK parameters Cₘₐₓ/D and AUC(0 last)/D for the assessment of dose proportionality are presented in Table 8.

Generally, a sub-proportional increase in exposure was seen from 100 mg to 300 mg dose levels, particularly for peak exposure levels (i.e., Cₘₐₓ) and as such conclusions are presented for each dose level separately and not for both dose levels combined.

TABLE 8
Dose proportionality (GMR 90% CIs)

| | G1 vs G2 | H1 vs H2 | I1 vs I2 |
|---|---|---|---|
| Analyte | HME Tablet | HME PfR suspension | HME Tablet Fed |
| Cₘₐₓ (%) | 73.79 (56.43, 96.50) | 75.71 (58.59, 97.84) | 79.48 (59.42, 106.30) |
| AUC(0-last) (%) | 83.13 (64.45, 107.22) | 99.66 (75.60, 131.37) | 95.78 (76.99, 119.15) |

Subjects received Compound A HME formulation fasted (Regimens G1/G2 and Regimen H1/H2) or fed (Regimen I1/I2). Regimens G and I = Tablet formulations, Regimen H = PfR suspension formulation. G1/H1/I1 = 300 mg, G2/H2/I2 = 100 mg. Results obtained from log-transformed dose-corrected PK parameters using an ANOVA model. Separate models were fitted for each formulation and included a term for dose fitted as fixed effect. CI = confidence interval for ratio of adj geo means; Adj geo mean = adjusted geometric mean from model; Ratio of adj geo means for Test/Reference.

The results indicate that the peak and overall exposure of Compound A, as measured by Cₘₐₓ/D and AUC(0-last)/D of 300 mg Compound A HME tablet formulation (Regimen G1, reference) were 73.79% and 83.13% of those for 100 mg Compound A HME tablet formulation (Regimen G2, reference) for the respective PK parameter. The 90% CIs associated with this estimate were (56.43%, 96.50%) and (64.45%, 107.22%) for Cₘₐₓ/D and AUC(0-last), respectively.

The results indicate that the peak and overall exposure of Compound A, as measured by Cₘₐₓ/D and AUC(0-last)/D of 300 mg Compound A selected HME PfR suspension formulation (Regimen H1) were 75.71% and 99.66% of those for 100 mg Compound A selected HME PfR suspension formulation (Regimen H2) for the respective PK parameter. The 90% CIs associated with this estimate were (58.59%, 97.84%) and (75.60%, 131.37%) for Cₘₐₓ/D and AUC(0-last)/D, respectively.

The results indicate that the peak and overall exposure of Compound A, as measured by Cₘₐₓ/D and AUC(0-last)/D of 300 mg Compound A HME tablet formulation fed (Regimen I1) were 79.48% and 95.78% of 100 mg Compound A HME tablet formulation fed (Regimen I2) for the respective PK parameter. The 90% CIs associated with this estimate were (59.42%, 106.30%) and (76.99%, 119.15%) for Cₘₐₓ/D and AUC(0-last)/D, respectively.

Dose Corrected Analysis

The results from the formal statistical analyses of the PK parameters Cₘₐₓ/D and AUC(0-last)/D for the assessment of relative bioavailability and food effect for Compound A are presented in Table 9.

TABLE 9
Dose Corrected Plasma Pharmacokinetic Parameters for Compound A - Assessment
of Relative Bioavailability: Pharmacokinetic Analysis Subset (Part 2)

| | Test | | Reference | | Ratio | 90% | P- | |
|---|---|---|---|---|---|---|---|---|
| | | Mean | | Mean | % | CI | Value | CVw |
| Compare Parameter | n | (1) | n | (1) | (2) | (3) | (4) | (5) |
| H vs G Cmax/ | 10 | 10.3 | 10 | 10.7 | 96.04 | (82.97, 111.17) | 0.64 | 18.40 |
| D (ng/mL/mg) | | | | | | | | |
| AUC(0-last)/ | 10 | 529 | 10 | 508 | 104.07 | (94.56, 114.54) | 0.48 | 12.00 |
| D(ng · h/mL/mg) | | | | | | | | |
| I vs G Cmax/ | 11 | 8.43 | 10 | 10.7 | 78.80 | (68.31, 90.91) | 0.010 | 18.40 |
| D(ng/mL/mg) | | | | | | | | |
| AUC(0-last)/ | 11 | 494 | 10 | 508 | 97.25 | (88.57, 106.79) | 0.61 | 12.00 |
| D(ng · h/mL/mg) | | | | | | | | |

Subjects received Compound A HME formulation fasted (Regimens G1/G2 and Regimen H1/H2) or fed (Regimen I1/I2). Regimens G and I = Tablet formulations, Regimen H = PfR suspension formulation. G1/H1/I1 = 300 mg, G2/H2/I2 = 100 mg. Results obtained from log-transformed dose-corrected PK parameters using an ANOVA model. Separate models were fitted for each formulation and included a term for dose fitted as fixed effect.
(1) Mean = adjusted geometric mean from model,
(2) Ratio of adj geo means for Test/Reference,
(3) CI = confidence interval for ratio of adj geo means,
(4) p-value from two-sided test with null hypothesis that ratio is equal to 100%,
(5) CVw = intra-subject variability

### Relative Bioavailability

The results indicate that the peak and overall exposure of Compound A, as measured by Cₘₐₓ/D and AUC(0-last)/D of Compound A HME PfR suspension (Regimen H) were 96.04% and 104.07% of those for Compound A HME tablet formulation (Regimen G) for the respective PK parameter. The 90% CIs suggest that the true measure is unlikely to be less than 82.97% or greater than 111.17% for Cₘₐₓ/D and unlikely to be less than 94.56% or greater than 114.54% for AUC(0-last)/D. The difference between treatments in peak exposure was not statistically significant (p=0.64) nor overall exposure (p=0.48).

### Food Effect

The results indicate that the peak and overall exposure of Compound A, as measured by Cₘₐₓ/D and AUC(0-last)/D of Compound A HME tablet formulation fed (Regimen I) were 78.80% and 97.25% of those for Compound A HME tablet formulation fasted (Regimen G) for the respective PK parameter. The 90% CIs suggest that the true measure is unlikely to be less than 68.31% or greater than 90.91% for Cₘₐₓ/D and unlikely to be less than 88.57% or greater than 106.79% for AUC(0-last)/D. The difference between treatments in peak exposure was statistically significant (p=0.010) but not for overall exposure (p=0.61).

Analysis Per Dose Level

300 mg Dose Level

The results from the formal statistical analyses of the PK parameters Cₘₐₓ and AUC(0-last) for the assessment of relative bioavailability for the 300 mg dose level of Compound A are presented in Table 10.

TABLE 10
Plasma Pharmacokinetic Parameters for Compound A - Assessment of Relative
Bioavailability 300 mg Dose Level: Pharmacokinetic Analysis Subset (Part 2)

| | Test | | Reference | | Ratio | 90% | P- |
|---|---|---|---|---|---|---|---|
| | | Mean | | Mean | % | CI | Value |
| Compare Parameter | n | (1) | n | (1) | (2) | (3) | (4) |
| H1 vs G1 | 3 | 2520 | 4 | 2620 | 96.42 | (69.87, 133.05) | 0.84 |
| Cmax (ng/mL) | | | | | | | |
| AUC(0-last) | 3 | 157000 | 4 | 133000 | 117.52 | (86.74, 159.23) | 0.35 |
| (ng · h/mL) | | | | | | | |
| I1 vs G1 | 4 | 2170 | 4 | 2620 | 82.94 | (61.56, 111.75) | 0.28 |
| Cmax (ng/mL) | | | | | | | |
| AUC(0-last) | 4 | 143000 | 4 | 133000 | 107.3 | (81.00, 142.15) | 0.65 |
| (ng · h/mL) | | | | | | | |

Subjects received Compound A HME formulation fasted (Regimens G1/G2 and Regimen H1/H2) or fed (Regimen I1/I2). Regimens G and I = Tablet formulations, Regimen H = PfR suspension formulation. G1/H1/I1 = 300 mg, G2/H2/I2 = 100 mg. Results obtained from log-transformed dose-corrected PK parameters using an ANOVA model. Separate models were fitted for each formulation and included a term for dose fitted as fixed effect.
(1) Mean = adjusted geometric mean from model,
(2) Ratio of adj geo means for Test/Reference,
(3) CI = confidence interval for ratio of adj geo means,
(4) p-value from two-sided test with null hypothesis that ratio is equal to 100%

The results indicate that the peak and overall exposure of Compound A, as measured by Cₘₐₓ and AUC(0-last) of 300 mg Compound A selected HME PfR suspension formulation (Regimen H1) were 96.42% and 117.52% of those for 300 mg Compound A HME tablet formulation (Regimen G1) for the respective PK parameter.

The results indicate that the peak and overall exposure of Compound A, as measured by Cₘₐₓ and AUC(0-last) of 300 mg Compound A HME tablet formulation fed (Regimen I1) were 82.94% and 107.30% of those for 300 mg Compound A HME tablet formulation fasted (Regimen G1) for the respective PK parameter.

100 mg Dose Level

The results from the formal statistical analyses of the PK parameters Cₘₐₓ and AUC(0-last) for the assessment of relative bioavailability for the 100 mg dose level of Compound A are presented in Table 11.

TABLE 11
Plasma Pharmacokinetic Parameters for Compound A - Assessment of Relative
Bioavailability 100 mg Dose Level: Pharmacokinetic Analysis Subset (Part 2)

| | Test | | Reference | | Ratio | 90% | P- | |
|---|---|---|---|---|---|---|---|---|
| | | Mean | | Mean | % | CI | Value | CVw |
| Compare Parameter | n | (1) | n | (1) | (2) | (3) | (4) | (5) |
| H2 vs G2 | 7 | 1130 | 6 | 1190 | 95.17 | (82.56, 109.71) | 0.53 | 10.80 |
| Cmax (ng/mL) | | | | | | | | |
| AUC(0-last) | 7 | 54300 | 6 | 52000 | 104.40 | (89.14, 122.26) | 0.62 | 12.30 |
| (ng · h/mL) | | | | | | | | |
| I2 vs G2 | 7 | 943 | 6 | 1190 | 79.37 | (67.76, 92.96) | 0.029 | 10.80 |
| Cmax (ng/mL) | | | | | | | | |
| AUC(0-last) | 7 | 51500 | 6 | 52000 | 99.07 | (83.14, 118.06) | 0.92 | 12.30 |
| (ng · h/mL) | | | | | | | | |

Subjects received Compound A HME formulation fasted (Regimens G1/G2 and Regimen H1/H2) or fed (Regimen I1/I2). Regimens G and I = Tablet formulations, Regimen H = PfR suspension formulation. G1/H1/I1 = 300 mg, G2/H2/I2 = 100 mg. Results obtained from log-transformed dose-corrected PK parameters using an ANOVA model. Separate models were fitted for each formulation and included a term for dose fitted as fixed effect.
(1) mean = adjusted geometric mean from model,
(2) Ratio of adj geo means for Test/Reference,
(3) CI = confidence interval for ratio of adj geo means,
(4) p-value from two-sided test with null hypothesis that ratio is equal to 100%,
(5) CVw = intra-subject variability

The results indicate that the peak and overall exposure of Compound A, as measured by Cₘₐₓ and AUC(0-last) of 100 mg Compound A selected HME PfR suspension formulation (Regimen H2), were on average 95% and 104% of those for 100 mg Compound A HME tablet formulation (Regimen G2, reference) for the respective PK parameter.

The results indicate that the peak and overall exposure of Compound A, as measured by Cₘₐₓ and AUC(0-last) of 100 mg Compound A HME tablet formulation fed (Regimen I2) were on average 79% and 99% of those for 100 mg Compound A HME tablet formulation fasted (Regimen G2) for the respective PK parameter.

Pharmacokinetic Conclusions

Following single oral administration of Compound A as the HME tablet formulation at the 100 mg and 300 mg doses in the fasted state (Regimen G) absorption of Compound A was similar with median Tₘₐₓ of 3.50 h and 3.00 h, respectively.

Elimination of Compound A was long for the 100 mg and 300 mg dose levels for Regimen G with geometric mean T½ of 73.5 h and 67.4 h, respectively.

The inter-subject variability associated with exposure (Cₘₐₓ and AUC(0-last)) was low to moderate for both dose levels (Regimen G), ranging from 15.0% to 29.4%.

When administered as the PfR suspension (test) formulation (Regimen H) at the 100 mg and 300 mg doses, absorption of Compound A was similar with median Tₘₐₓ of 3.00 h for both regimens.

Elimination of Compound A was long for the 100 mg and 300 mg dose levels for Regimen H with geometric mean T_{1/2} of 65.4 h and 83.2 h, respectively.

When administered as the HME tablet formulation in the fed state (Regimen I) at the 100 mg and 300 mg dose level, median Tₘₐₓ for Compound A occurred later than when administered in the fasted state, with median Tₘₐₓ of 5.00 h and 6.50 h, respectively.

Elimination of Compound A was long for the 100 mg and 300 mg dose for Regimen I levels with geometric mean T^{1/2} of 61.7 h and 85.4 h, respectively.

Generally, a sub-proportional increase in exposure was seen from 100 mg to 300 mg dose levels, particularly for peak exposure levels. As such, proportional increases in exposure between the dose levels could not be concluded for each of the Regimens G, H, and I.

At the 300 mg dose level, both peak and overall exposure levels of Compound A were similar between Compound A selected HME PfR suspension formulation and Compound A HME tablet formulation both in the fasted state (suspension formulation levels were 96.42% and 117.52% of those for the tablet formulation respectively) but results should be viewed with caution due to small number of subjects.

At the 300 mg dose level, both peak and overall exposure levels of Compound A were also similar between Compound A HME tablet formulation in the fed state compared to the fasted state (fed levels were 82.94% and 107.30% of those for the fasted levels respectively); again, results should be treated with caution due to small number of subjects.

At the 100 mg dose level, both peak and overall exposure levels of Compound A were similar between Compound A selected HME PfR suspension formulation and Compound A HME tablet formulation both in the fasted state (suspension formulation levels were 95.17% and 104.40% of those for the tablet formulation).

At the 100 mg dose level, the peak exposure levels of Compound A were significantly lower for Compound A HME tablet formulation in the fed state compared to the fasted state (fed levels were 79.37% of those for the fasted levels), whereas overall exposure levels were similar between the two tablet formulations (fed levels were 99.07% of those for the fasted levels).

### Example 4. —Stability of a Prototype Batch of Compound a, Powder for Reconsitutiion (PfR)

Stability of a prototype batch of Compound A PfR was under study with data collected for 3 months to date. This study supported stability of the initial Clinical Trial Batches and continued for at least the duration of the proposed clinical trial (QSC205140).

The prototype batch was manufactured using the same process, except for the inclusion of sucralose and strawberry flavoring. It was packaged in 2-ounce PET bottles and also 2-ounce amber glass bottles. The bottle size is considered worst-case (i.e., least protective packaging and largest headspace) when compared to the planned clinical packaging in 1 oz glass bottles. The Compound A active fill for the prototype was 600-mg versus the 430-mg (300 mg to deliver) planned for the Clinical Trial Material and is also considered to represent ‘worst case’ conditions from a stability perspective.

### Example 5. Pharmacokinetics of Compound a Following Administration to Male Cynomolgus Monkeys

The purpose of this study was to provide plasma samples to investigate the pharmacokinetics of Compound A in tablet and multiple dose formulations following oral administration to male Cambodian cynomolgus monkeys. (Tables 12.1-12.5).

TABLE 12.1
Group 1 Formulation: Composition of Compound
A Tablets, 100 mg (Film Coated HME Tablet)

| | | Compendial | mg/ |
|---|---|---|---|
| Ingredients | Function | Status | tablet |
| Compound A | Active | — | 100.00 |
| Copovidone | Solubilizer | USP-NF/Ph. Eur | 150.00 |
| Microcrystalline Cellulose | Filler | USP-NF/Ph. Eur | 318.75 |
| Croscarmellose Sodium | Disintegrant | USP-NF/Ph. Eur | 50.00 |
| Colloidal Silicon Dioxide | Glidant | USP-NF/Ph. Eur | 3.125 |
| Magnesium Stearate | Lubricant | USP-NF/Ph. Eur | 3.125 |
| Sub-total | | | 625.00 |
| Opadry ® Red 03F45081 | Film coat | Vendor | 7.0 |
| Opadry ® Yellow 03F42240 | Film coat | Vendor | 14.0 |
| Purified Water ^{(d)} | Solvent | USP-NF/Ph. Eur | q.s |
| | Total | | 646.00 |

(a) The theoretical weight gain after coating is 4.2%.
(b) The theoretical weight gain after coating is 3.3%.
(c) The total theoretical weight gain after coating is 3.4%.
^{(d)} Removed during drying of film coating.
q.s quantity sufficient.

TABLE 12.2
Group 3 Formulation: Composition of Pediatric Suspension
Formulation Containing Polaxamer, 100 mg

| Ingredients | Function | % Composition | mg/dose |
|---|---|---|---|
| Compound A | Active | 20% | 100.0 |
| Copovidone | Solubilizer | 30% | 150.0 |
| NaCMC | Filler | 1.00% | 5.0 |
| Mannitol (Partek 100) | Filler | 40.00% | 200.0 |
| Colloidal Silicon Dioxide | Glidant | 3.00% | 15.0 |
| Poloxamer 407 Micro | Lubricant | 1.00% | 5.0 |
| Nusil MED-342 | Anti-foaming | 5.00% | 25.0 |
| Total | | 100% | 500.00 mg |

TABLE 12.3
Groups 2 and 4 Formulations: Composition of Pediatric
Suspensions with HME in 75-150 micron range for Formulation
3 and Formulation 4 Utilizing a Larger HME particle
size with >100 micron particle size, 100 mg

| Ingredients | Function | % Composition | mg/dose |
|---|---|---|---|
| Compound A | Active | 20% | 100.00 |
| Copovidone | Solubilizer | 30% | 150.00 |
| Microcrystalline Cellulose | Filler | 20.75% | 103.75 |
| (PH102) | | | |
| Mannitol (Partek 100) | Filler | 20.75% | 103.75 |
| Colloidal Silicon Dioxide | Glidant | 3.00% | 15.00 |
| Sodium Lauryl Sulfate | Lubricant | 0.50% | 2.50 |
| Nusil MED-342 | Anti-foaming | 5.00% | 25.00 |
| Total | | 100.00% | 500.0 mg |

TABLE 12.4
Groups 5 Formulation: Composition of the Diluted
Lead Pediatric Suspension from Group 2 Diluted
from 50 mg/mL to 25 mg/mL Compound A, 100 mg

| Ingredients | Function | % Composition | mg/dose |
|---|---|---|---|
| Compound A | Active | 10% | 100.00 |
| Copovidone | Solubilizer | 15% | 150.00 |
| Microcrystalline Cellulose | Filler | 31.13% | 313.30 |
| (PH102) | | | |
| Mannitol (Partek 100) | Filler | 31.13% | 313.30 |
| Colloidal Silicon Dioxide | Glidant | 4.50% | 45.00 |
| Sodium Lauryl Sulfate | Lubricant | 0.75% | 7.50 |
| Nusil MED-342 | Anti-foaming | 7.5% | 75.00 |
| Total | | 100.00% | 1004.1 |

TABLE 12.5
Group 6 Formulation: Composition of the Pediatric Suspension
5 with Reduced Nusil versus the Group 5 formulation, 100 mg

| Ingredients | Function | % Composition | mg/dose |
|---|---|---|---|
| Compound A | Active | 10% | 100.00 |
| Copovidone | Solubilizer | 15% | 150.00 |
| Microcrystalline Cellulose | Filler | 32.75% | 327.50 |
| (PH102) | | | |
| Mannitol (Partek 100) | Filler | 32.75% | 327.50 |
| Colloidal Silicon Dioxide | Glidant | 4.25% | 42.50 |
| Sodium Lauryl Sulfate | Lubricant | 0.75% | 7.50 |
| Nusil MED-342 | Anti-foaming | 3.0% | 45.00 |
| Total | | 100.00% | 1000.0 mg |

The final formulations for Groups 2-6 were diluted with Reverse Osmosis De-Ionized (RODI) water to produce white viscous suspensions for oral dosing at the target concentrations of 50 mg/mL for Groups 2-4; and 25 mg/mL for Groups 5 and 6.

To decrease viscosity, the oral dose formulations for Groups 2-6 were prepared within 30 minutes of administration. Dose formulation samples (1 set of 3, top, mid, and bottom, 0.5 mL each) were collected from each dose formulation before dose administration. The dose formulation samples were stored at −20° C. until shipment for analysis. It was practiced loading the dose syringes once the DF suspension was homogeneous and the DF samples taken (usually between 10-15 min after the start of DF preparation). All DFs started to gel between 25 min and 30 min after the start of DF preparation.

A total of seven male non-naïve Cambodian cynomolgus monkeys (6±1 spare) were selected from the colony. All animals were approximately 2.5-3.5 kg at the time of dosing. All animals were fasted overnight before dosing and food was returned after the 4 hours after each test article dose. The final study design is presented in Table 13.

TABLE 13
Animal Testing Study Design

| | | No. of | | Dose | Dose | Dose | TA | Post- |
|---|---|---|---|---|---|---|---|---|
| | | Males ^{B} | Test | Level | Conc. | Volume | Dose | Dose |
| Grp | Session | * | Articles | (mg/animal) | (mg/mL) | (mL/animal) | Route | Flush (mL) |
| 1 | 1 | 6 | Compound A | 100 mg | NA | NA | PO | 10 |
| | | | | (API) | | | (tablet) | |
| 2 | 2 | | Compound A | 100 mg | 50 | 2 | PO | 10 |
| | | | HME^{A} | (API) | | | | |
| 3 | 3 | | Compound A | 100 mg | 50 | 2 | PO | 10 |
| | | | HME^{A} | (API) | | | | |
| 4 | 4 | | Compound A | 100 mg | 50 | 2 | PO | 10 |
| | | | HME^{A} | (API) | | | | |
| 5 | 5 | | Compound A | 100 mg | 25 | 4 | PO | 10 |
| | | | HME^{A} | (API) | | | | |
| 6 | 6 | | Compound A | 100 mg | 25 | 4 | PO | 10 |
| | | | HME^{A} | (API) | | | | |

Each animal was pre-treated with 6 ug/kg IM of Pentagastrin (0.12 mg/mL at 0.05 mL/kg) one hour before Test Article administration. For Group 1, each animal received a tablet via a tablet gun, followed by 10 mL of water through a syringe to facilitate swallowing. For the remaining groups, each animal received a PO dose formulation, followed by 10 mL of water by oral gavage. The amount of each dose delivered was 100 mg/animal. There was a washout period of at least 7 days between 6 dose sessions. All doses were completed without incident, except for animal 6001 which had a reflux (1-2 mL of white foamy liquid) right after gavage tube was pulled out post-dose.

All blood samples (0.25 mL each, K2EDTA anticoagulant) were collected by way of femoral venipuncture. For Groups 1-6, blood samples were collected from each animal at pre-dose, 0.5, 1, 2, 4, 6, 8, 12, 24, 36, 48, and 72 hours after oral dosing. All whole blood samples were placed on wet ice immediately after collection until centrifugation at 2200×g for 10 minutes at 4° C. to isolate plasma within 30 minutes of collection. The resulting plasma was transferred into cryo tube and immediately placed on dry ice until storage at nominally −80° C. before shipment for analysis (RGA 2).

Dose administration and body weight data are presented in Table 14Table. Following dosing and at each sample collection time point the animals were observed for any clinically relevant abnormalities and all animals appeared normal at the time of each observation.

TABLE 14
Mean PK Parameter in NHP following PO Dose of Compound A at 100 mg/animal

| | T_{1/2} | Tₘₐₓ | Cₘₐₓ | Cₘₐₓ/D | AUCₗₐₛₜ | AUC_{INF} | AUC_{INF}/D | AUC % |
|---|---|---|---|---|---|---|---|---|
| Group | (hr) | (hr) | (ng/mL) | (ng/mL/mg) | (ng · hr/mL) | (ng · hr/mL) | (hr · kg/mL/mg) | Extrap (%) |
| Group 1 | 17.0 | 4.33 | 16600 | 474.0 | 472000 | 509000 | 14700 | 6.8 |
| SD | 6.17 | 1.51 | 1970 | 40.1 | 56400 | 80800 | 2640 | 5.4 |
| Group 2 | 14.8 | 5.33 | 10500 | 295 | 335000 | 351000 | 9930 | 4.4 |
| SD | 2.59 | 3.27 | 2030 | 58.5 | 68700 | 72500 | 2200 | 1.5 |
| Group 3 | 17.6 | 4.33 | 8020 | 237 | 244000 | 261000 | 7710 | 7.3 |
| SD | 4.23 | 1.97 | 2430 | 77.0 | 54300 | 51500 | 1510 | 3.7 |
| Group 4 | 16.8 | 10.7 | 6810 | 194 | 285000 | 311000 | 8860 | 8.4 |
| SD | 2.96 | 6.89 | 2810 | 84.8 | 88400 | 94300 | 2940 | 3.4 |
| Group 5 | 17.6 | 4.00 | 20200 | 615 | 551000 | 596000 | 18300 | 7.5 |
| SD | 4.64 | 1.26 | 3340 | 88.1 | 44800 | 53400 | 2280 | 4.4 |
| Group 6 | 17.0 | 5.33 | 12300 | 377 | 395000 | 417000 | 12900 | 5.8 |
| SD | 2.80 | 1.03 | 2380 | 72.9 | 50800 | 56400 | 1490 | 2.3 |

Compound A was detectable in all post-dose plasma samples following all doses. It was also detectable in pre-dose samples for Groups 2, 3, and 4, but not for Groups 1, 5, and 6.

Noncompartmental PK Analysis (NCA) using WinNonlin software was performed with relevant PK parameters (Cₘₐₓ, Tₘₐₓ, T_{1/2}, AUC, etc.). It seems that the Group 5 (Diluted Active at 25 mg/mL active and Increased Solids:MCC/Mannitol/SLS/HME/Nusil/CSD Suspension) and Group 6 (Group 5 Formulation at 25 mg/mL active with Lower Nusil: MCC/Mannitol/SLS/HME/Nusil/CSD Suspension) yield similar exposure profile to Group 1 (Clinical Film Coated HME Tablet).

Compound A in tablet or variable dose formulations were well tolerated. The highest exposure of Compound A was observed in Group 5, followed by Groups 1, 6, and 2; which are seemingly higher than those in Groups 3 and 4.

In-Use Stability

The Compound A PfR drug product is reconstituted with water and dosed by syringe. Due to the properties of copovidone used in the hot melt extrusion, the formulation viscosity increases such that the product must be dosed using the supplied syringe within 30 minutes of reconstitution. The combination of the container and syringe was qualified for use by reconstituting Compound A PfR according to patient labeling instructions and then withdrawing and dispensing either a 5-mL or a 12-mL aliquot. Assay for the 125 mg dose was confirmed at 101.8±3.9% theoretical and that for the 300 mg dose was confirmed at 97.8±2.2% theoretical via HPLC. Stability of the reconstituted solution was studied. Assay was measured at the 20 minute time point, and confirmed results within 2% of time zero. The material was still syringe-able at 30 minutes.

### Example 6. Exemplary Formulations

TABLE 15
Formulation Ex-5.1

| Component | Wt % |
|---|---|
| HME solid dispersion of Compound | 50 |
| A and copovidone (2:3 by weight) | |
| MCC (PH102) | 20.75 |
| Mannitol | 20.75 |
| Kolliphor 188/407 micro | 1.0 |
| CSD | 3.0 |
| Nusil MED-342 | 5.0 |

TABLE 16
Example Formulations Prepared and Tested

| | NHP PK Study Formulations | | | |
|---|---|---|---|---|
| | | | Group 3 and 4 | |
| | Group 5 | Group 6 | Formulation | Group 2 |
| | Formulation | Formulation A3 | D1 and D2 | Formulation A |
| Blend Component | wt % | | | |
| 40:60 Compound | 25.00% | 25.00% | 50.00% | 50.00% |
| A:PVP-VA HME | | | | |
| MCC (PH102) | 31.10% | 33.50% | — | 20.75% |
| Mannitol (Partek 100) | 31.10% | 33.50% | 40.00% | 20.75% |
| SLS | 0.75% | 0.75% | — | 0.50% |
| CSD | 4.50% | 4.25% | 3.00% | 3.00% |
| Nusil MED-342 | 7.50% | 3.00% | 5.00% | 5.00% |
| NaCMC | — | — | 1.00% | — |
| Poloxamer Micro 407 | — | — | 1.00% | — |

### Example 7. Exemplary Formulations

A formulation DOE on the PfR evaluated the multivariate interactions of different concentrations of SLS [0.25-0.75%], Nusil [1-7.5%], and CSD [1-4.5%] as the Formulation Factors.

The measured outputs were on the reconstituted suspension at 25 mg/mL active. The following attributes were measured: suspension flowability, foaming, adhesion to the bottle walls, syringeability, density and dose recovery. These formulations are provided in Table 17.

TABLE 17
Exemplary DOE formulations

| | DoE Formulations | | | | | | | | | |
|---|---|---|---|---|---|---|---|---|---|---|
| | 1 | 2 | 3 | 4 | 5 | 6 | 7 | 8 | 9 | 10 |
| Blend Component | WT % | | | | | | | | | |
| 40:60 Compound | 25.00% | 25.00% | 25.00% | 25.00% | 25.00% | 25.00% | 25.00% | 25.00% | 25.00% | 25.00% |
| A:PVP-VA HME | | | | | | | | | | |
| MCC (PH102) | 31.38% | 33.75% | 36.38% | 32.88% | 33.13% | 31.13% | 34.38% | 36.13% | 34.63% | 31.13% |
| Mannitol | 31.38% | 33.75% | 36.38% | 32.88% | 33.13% | 31.13% | 34.38% | 36.13% | 34.63% | 31.13% |
| (Partek 100) | | | | | | | | | | |
| SLS | 0.25% | 0.50% | 0.25% | 0.75% | 0.25% | 0.75% | 0.75% | 0.75% | 0.25% | 0.75% |
| CSD | 4.50% | 2.75% | 1.00% | 1.00% | 1.00% | 4.50% | 4.50% | 1.00% | 4.50% | 4.50% |
| Nusil MED-342 | 7.50% | 4.25% | 1.00% | 7.50% | 7.50% | 7.50% | 1.00% | 1.00% | 1.00% | 7.50% |

DOE study results are provided in Tables 18-20.

TABLE 18
Ranking of the Flow, Foaming, and Adhesion properties of
the DoE Formulations after Reconstitution at 25 mg A/mL

| | Flow | Foam | Adhesion | |
|---|---|---|---|---|
| ID | (1-3) | (1-3) | (1-3) | Syringeability* |
| 1 | 1 | 1 | 2 | >30 minutes |
| 2 | 1 | 2 | 2 | >30 minutes |
| 3 | 1 | 2 | 2 | >30 minutes |
| 4 | 1 | 1 | 2 | >30 minutes |
| 5 | 1 | 1 | 2 | >30 minutes |
| 6 | 1 | 1 | 1-2 | >30 minutes |
| 7 | 1 | 2 | 2 | >30 minutes |
| 8 | 2 | 2-3 | 1-2 | >30 minutes |
| 9 | 1 | 2 | 1-2 | >30 minutes |
| 10 | 1 | 2 | 2 | >30 minutes |

*Syringable up to <15 minutes, <30 minutes, >30 minutes
*1—Not viscous, No foam, or No agglomerates adhered to sides.
*2—Slightly more viscous but still flowable, some agglomerates, or slightly foamy (top layer of foam, less than ½ of volume.
*3—Thick viscous (still flowable), Substantial agglomerate adhesion, substantial foam more than 50% of volume.

TABLE 19
The Dose Recovery of the Reconstituted Formulation Results from
the DoE Formulations after Reconstitution at 25 mg A/mL

| | Dose | Dose | Dose | | |
|---|---|---|---|---|---|
| | Recovery | Recovery | Recovery | | |
| ID | 1* (%) | 2 (%) | 3 (%) | Average | % RSD |
| 1 | NA | 106 | 106 | 106 | 0.0 |
| 2 | NA | 108 | 104 | 106 | 2.8 |
| 3 | NA | 109 | 106 | 108 | 2.1 |
| 4 | NA | 111 | 110 | 111 | 0.7 |
| 5 | 101 | 81 | 107 | 104 | 4.2 |
| 6 | 112 | 119 | 109 | 113 | 5.1 |
| 7 | 110 | 106 | 104 | 107 | 3.1 |
| 8 | 110 | 96 | 104 | 103 | 7.0 |
| 9 | 106 | 103 | 106 | 105 | 1.7 |
| 10 | 109 | 107 | 102 | 106 | 3.6 |

*125 mg A [5 mL, 25 mg A/mL] delivered from 300 mgA [12 mL] preparation. Dose recovery based on target and not corrected for actual weight of suspension dosed.

TABLE 20
The Density Measurements of the DoE Formulations
after Reconstitution at 25 mg A/mL

| | Measured | Measured | Measured | | |
|---|---|---|---|---|---|
| | Density | Density | Density | | |
| | Replicate | Replicate | Replicate | | |
| ID | 1 | 2 | 3 | Average | % RSD |
| 1 | 1.12 | 1.14 | 1.08 | 1.11 | 0.03 |
| 2 | 1.12 | 1.10 | 1.10 | 1.11 | 0.01 |
| 3 | 1.11 | 1.08 | 1.08 | 1.09 | 0.02 |
| 4 | 1.12 | 1.08 | 1.10 | 1.10 | 0.02 |
| 5 | 1.12 | 1.10 | 1.08 | 1.10 | 0.02 |
| 6 | 1.13 | 1.12 | 1.10 | 1.12 | 0.02 |
| 7 | 1.13 | 1.11 | 1.12 | 1.12 | 0.01 |
| 8 | 1.17 | 1.13 | 1.10 | 1.13 | 0.04 |
| 9 | 1.10 | 1.11 | 1.08 | 1.10 | 0.02 |
| 10 | 1.10 | 1.10 | 1.10 | 1.10 | 0.00 |

All formulations tested were syringable for up to 30 minutes

Density was on average 1.1 g/mL; SLS content and the interaction factor between SLS & Nusil had a statistically significant impact on measured density.

In some cases, in order to achieve an accurate dose, it can be critical to adjust the suspension recipe and concentration to account for density. Within the design space evaluated in Table 17, the density was not practically impacted by formulation parameters and an adjustment to the suspension recipe to account for a density of 1.1 g/mL could be applied for accurate dosing within this design space.

### Example 8—Exemplary Suspension Formulations

### Impact of Simethicone Content on Foaming:

Simethicone levels were tested at 1%, 2% and 5% wt/wt in the powder formulations below. The formulations were reconstituted with water to a 50 mg/mL active suspensions. Foaming was evaluated visually in the reconstituted suspension. With 5% simethicone, no significant foaming was observed, with 2% simethicone minor foaming was observed and with 1% simethicone significant foaming was observed. The formulations used for this study are provided in Table 21.

TABLE 21
Formulations of Blends A1-A3

| | Blend A1 | Blend A2 | Blend A3 |
|---|---|---|---|
| Component | (wt %) | (wt %) | (wt %) |
| Compound A* | 50.00% | 50.00% | 50.00% |
| MCC (PH 102) | 20.75% | 22.25% | 22.75% |
| Mannitol | 20.75% | 22.25% | 22.75% |
| SLS | 0.50% | 0.50% | 0.50% |
| CSD | 3.00% | 3.00% | 3.00% |
| Nusil MED-342 | 5.00% | 2.00% | 1.00% |

*Compound A refers to a 40:60 HME solid dispersion of Compound A:PVP-VA.

### Assessment of Impact of HME Particle Size Performance, Sink Dissolution on Suspension Blend A1.

The suspension was prepared with 3 different HME particle size ranges. The powder physical properties and visual suspension properties were evaluated. All suspensions were reconstituted and tested by Type II dissolution and were fully dissolved in 15 minutes using a volume of 900 mL 0.1 N HCl with 0.5% SLS (w/v) at 37° C. A particle size summary and formulation used for the study are provided in Tables 22 and 23.

TABLE 22
HME Particle Size Comparison

| HME Particle | Tap/Bulk | Angle of | Visual Assessment of |
|---|---|---|---|
| Size | Density | Repose | Suspension Homogeneity |
| A. Unmodified | 0.39 g/mL | 45° | No difference in visual |
| Starting Material | 0.50 g/mL | | appearance of suspension |
| B. HME | 0.42 g/mL | NA | homogeneity for Formulation |
| Particles <100 um | 0.55 g/mL | | A and B. |
| C. HME | 0.40 g/mL | NA | Potentially minor difference in |
| Particles >50 um | 0.55 g/mL | | suspension C viscosity. |

TABLE 23
Base formulation of Blend A1 for HME particle size effects

| Component | wt % |
|---|---|
| Compound A | 50.00% |
| MCC (PH 102) | 20.75% |
| Mannitol | 20.75% |
| SLS | 0.50% |
| CSD | 3.00% |
| Nusil MED-342 | 5.00% |

Formulation Screening Suspension Blend Formulation and Optimization. Suspensions with suitable colloidal properties can be prepared when reconstituted at 50 mg/mL with water, as illustrated in Table 24. Blends A to Blend D vary in the components used in the blends. Those difference are as follows: Blend A—MCC and SLS; Blend B—MCC and Poloxamer; Blend C—NaCMC and SLS; and Blend D—NaCMC and Poloxamer.

TABLE 24
Various Blend Formulations Used in
Formulation Optimization Studies.

| | Blend | Blend | Blend | Blend | Blend |
|---|---|---|---|---|---|
| Component | A | B | C1 | C2 | C3 |
| Compound A | 50.00% | 50.00% | 50.00% | 50.00% | 50.00% |
| MCC (PH 102) | 20.75% | 20.50% | — | — | — |
| Mannitol | 20.75% | 20.50% | 31.50% | 36.50% | 40.50% |
| SLS | 0.50% | — | 0.50% | 0.50% | 0.50% |
| CSD | 3.00% | 3.00% | 3.00% | 3.00% | 3.00% |
| Nusil MED-342 | 5.00% | 5.00% | 5.00% | 5.00% | 5.00% |
| Poloxamer Micro 407 | — | 1.00% | — | — | — |
| NaCMC | — | — | 10.00% | 5.00% | 1.00% |

### Example 9. Syringeability Evaluation of the Formulations

Formulations were prepared at 50 mg/mL by mixing and shaking with water. The formulations were drawn into 10 mL oral syringes and assessed using an Easy, Difficult, and Not syringible scale. Table 25 provides the results of the syringeability evaluation using various Blends. Table 26 provides the Blend formulations used for this study.

Qualitative Assessment of Syringeability

Easy—can be easily drawn into syringe, air bubbles can be easily removed from syringe and volume can be accurately adjusted. Suspension is easily dispensed.

Difficult—can be drawn into syringe, but may require multiple attempts, significant force, or may be dependent on draw speed. Air bubbles are difficult, but not impossible to dispense for an accurate volume. Suspension can be dispensed.

Not Syringable—cannot be drawn into syringe due to high viscosity or clumps, if drawn into syringe air bubbles cannot be dispensed for accurate adjustment of volume. Suspension may not be dispensable without significant force.

TABLE 25
Syringeability Evaluation of Various Compound
A Blends Over Time (0 mins to 60 mins)

| Formulation | T₀ | 30 minutes | 45 minutes | 60 minutes |
|---|---|---|---|---|
| A [MCC, SLS] | Easy | Easy | Not Syringable | |
| B [MCC, Poloxamer] | | Easy | | Not |
| | | | | Syringable |
| C3 [NaCMC, SLS] | | Easy | Difficult | Not |
| | | | | Syringable |
| D1 [NaCMC, | | Easy | | Not |
| Poloxamer] | | | | Syringable |

TABLE 26
Various Blend Formulations Used in Syringeability Studies

| | Blend | Blend | Blend | Blend | Blend |
|---|---|---|---|---|---|
| Component | A | B | C1 | C3 | D1 |
| Compound A | 50.00% | 50.00% | 50.00% | 50.00% | 50.00% |
| MCC (PH 102) | 20.75% | 20.50% | — | — | — |
| Mannitol | 20.75% | 20.50% | 31.50% | 40.50% | 40.00% |
| SLS | 0.50% | — | 0.50% | 0.50% | — |
| CSD | 3.00% | 3.00% | 3.00% | 3.00% | 3.00% |
| Nusil MED-342 | 5.00% | 5.00% | 5.00% | 5.00% | 5.00% |
| Poloxamer Micro 407 | — | 1.00% | — | — | 1.00% |
| NaCMC | — | — | 10.00% | 1.00% | 1.00% |

### Example 10. In-Use Stability of Formulation

An In-Use study was conducted to confirm that the drug product is stable following reconstitution but before withdrawal and dispensing. A 12 mL dose was considered to be ‘worst-case’ for this experiment, since any effect of gelation on foaming or ease of withdrawal would be maximized at the larger volume. Data show stability of the reconstituted PfR for at least 5 minutes and demonstrate that the drug product characteristics are therefore adequate for intended use.

A total of twelve bottles of Compound A PfR were reconstituted according to patient instructions, except that they were allowed to rest for 5 minutes prior to withdrawing the dose into the syringe. For each of the samples, appearance of the reconstituted suspension, pH, weight of dispensed suspension and the ease of syringeability were measured. Results show that Compound A PfR is stable for at least five minutes following reconstitution.

In addition, experiments were performed to characterize the robustness of the reconstitution procedure when modifying variations that could occur during ordinary use. Parameters assessed included variations to the shaking time and the temperature of the water used for reconstitution.

Results from these studies showed robustness of the formulation to water temperature (5° C. to 40° C.), and shaking time (50% to 200% of recommended time).

### Example 11. Optimization of PfR Preparation of Exemplary Formulation

The following factors were tested:

Water type: deionized vs. tap

Water Temperature: Cold water (5° C.) vs. Warm water (38° C.)

Rate of water addition: slow addition vs. bolus addition

Hold time after water addition and prior to mixing

Shaking duration & vigor

### Example 12. Long Term Stability of Formulation

Six (6) months of data at 25° C./60% RH and six (6) months of data at 40° C./75% RH are available for the PfR in PET and glass bottles. Three (3) months of data at 25° C./60% RH and three (3) months of data at 40° C./75% RH are available for the PfR in glass bottles.

Attributes tested included Appearance (Solid and Reconstituted), Assay, Related Substances, pH of reconstituted suspension, Reconstitution Time and Syringeability, Enantiomer Content, Dissolution and Water Content Microbial Limits and confirmation of physical form by XRPD. In the representative stability studies, no new degradants were detected at either condition. Additionally, all test results met specified acceptance criteria for the product quality attributes tested.

### Example 13. Exemplary Formulation and PK Study

Amorphous solid dispersions of Compound A in aqueous suspension formulations were administered via oral gavage in male CD-1 mice to assess Compound A plasma exposure. Pharmacokinetics (PK) of Compound A crystalline drug substance dissolved in organic solvent (PEG400) were used to generate a solution formulation—also included as the comparator group in this study. The concentrations of Compound A in mouse plasma were quantified with a qualified LC-MS/MS method.

Study Design

Each oral dosing group consisted of 3 male CD-1 mice. At dosing, the animals weighted between 0.0297 to 0.0376 kg. Animals were not fasted prior to oral administration in this study. Compound A drug substances in corresponding dosing vehicles were administered by oral route using polypropylene gavage tube to CD-1 mice. Serial blood samples were collected at 0.25, 0.50, 1, 4, 8 and 24 hours post-dose. Serial blood samples were collected from a tail vein or another approved method at 0.25, 0.50, 1, 4, 8 hours post-dose. Terminal blood collection were collected via cardiac puncture at 24 hours post-dose.

Blood samples were collected into K₂EDTA tubes according to in-life study protocol and immediately placed on wet ice and then centrifuged for 10 minutes at 3500 rpm for plasma preparation. All plasma samples were placed in 96-well strip-tube rack and stored in freezer until plasma analysis. Immediately following terminal blood collection, the brain tissue was collected for each animal. Each tissue was collected into pre-weighed tube and the weight of the tissue sample was determined. Samples were flash frozen and kept on dry ice until stored at −70° C. prior to sample processing and analysis. Table 27 provides the study design for the oral administration of Compound A in mice and summary of mouse PK parameters.

TABLE 27
Single Oral Dose Mouse PK with Compound A (Male CD1 mice, CRL) (Original
protocol was n = 4; error by CRL, n = 3 dosed) and Mean T_{ma.x} (hr), Mean
Cₘₐₓ (ng/ml), and Mean AUCₗₐₛₜ (hr*ng/ml) for Groups 1-4

| | | | True | Dose | Dose | Mean | Mean | Mean |
|---|---|---|---|---|---|---|---|---|
| | | | dose | conc | vol | Tₘₐₓ | Cₘₐₓ | AUCₗₐₛₜ |
| Group | N* | Test Article | (mg/kg) | (mg/mL) | (mL/kg) | (hr) | (ng/ml) | (hr*ng/ml) |
| 1 | 3 | Compound A API | 50 | 10 | 5 | 8 | 18,300 | 299,520 |
| 2 | 3 | 25% Compound | 50 | 10 | 5 | 5.33 | 11,960 | 188,243 |
| | | A:75% HPMCAS | | | | | | |
| | | SDD | | | | | | |
| 3 | 3 | 40% Compound | 50 | 10 | 5 | 4 | 15,833 | 254,145 |
| | | A:60% HPMCAS | | | | | | |
| | | SDD | | | | | | |
| 4 | 3 | 40% Compound | 50 | 10 | 5 | 6.67 | 21,110 | 347,554 |
| | | A:60% PVPVA | | | | | | |
| | | HME | | | | | | |

*Animals not fasted in this study; serial bleeding for PK samples
API = active pharmaceutical ingredient;
PEG = Polyethylene glycol;
HPMC = hydroxypropyl methylcellulose;
PVP = polyvinylpyrrolidone;
SDD = spray dried dispersion;
HME = hot melt extrusion

Pharmacokinetics Studies

Non compartmental pharmacokinetic (NCA) analysis was performed on plasma concentration data using WinNonlin module in the Phoenix Platform (version 8.3.1 or above, Certara Inc., Princeton, NJ 08540). Calculations were performed using nominal sampling times in the pharmacokinetic analysis. All pharmacokinetic parameters and summary statistics are reported to 3 significant digits except for Tₘₐₓ, which is reported to 2 decimal places.
Cₘₐₓ: Observed peak blood/plasma concentration
Tₘₐₓ: Time to reach observed peak blood/plasma concentration
kₑₗ: Elimination rate constant determined by linear regression analysis of selected time points in the apparent terminal phase of the log plasma concentration vs. time curve (not reported)
t_{1/2}: Apparent terminal half-life calculated as ln(2)/kₑₗ
MRT: Mean time a drug molecule resides in body
AUC₀₋ₗₐₛₜ: Area under the plasma concentration vs. time curve from 0 to the last quantifiable concentration calculated by the linear trapezoidal method
AUC_{0-inf}: Area under the plasma concentration vs. time curve from 0 h to infinity, calculated as AUC₀₋ₗₐₛₜ+Cₗₐₛₜ/kₑₗ

The pharmacokinetics parameters of Compound A following oral doses of several amorphous Compound A solid dispersions in aqueous suspension vehicle are summarized in Table 28. Compound A oral absorption in the male CD-1 mice was not very rapid, with the mean Tₘₐₓ ranging from 4 to 8 hours post-dose. The plasma terminal elimination half-life (t_{1/2}) was similar among the 4 treatment groups, ranging from 9.54 to 12.7 hours. The plasma Cₘₐₓ was 18.3±1.51, 12.0±2.77, 15.8±1.63 and 21.1±1.38 μg/mL for treatment groups 1 to 4, respectively. Plasma AUC₀₋ₗₐₛₜ was 300±24.7, 188 44.4, 254±25.0, and 348±16.7 hr*μg/mL and for groups 1 to 4, respectively.

TABLE 28
Mean Plasma PK of DAY 101 Following Oral Administration of a Single Dose
at 50 mg/kg in Male CD-1 Mice (Mean ± SD, n = 3)

| Dosing Group | | | |
|---|---|---|---|
| Group 1 | Group 2 | Group 3 | Group 4 |
| Test Article | | | |
| | 25% API:75% | 40% API:60% | 40% API:60% |
| DAY 101 API | HPMCAS, SDD | HPMCAS, SDD | PVPVA, HME |
| Dosing Vehicle | | | |
| | | | 0.5% methylcellulose |
| 100% PEG400 | DI water | DI water | in water (w/v) |

PK Parameters

| | | | | |
|---|---|---|---|---|
| Cₘₐₓ (μg/mL) | 18.3 ± 1.51 | 12.0 ± 2.77 | 15.8 ± 1.63 | 21.1 ± 1.38 |
| Tₘₐₓ (hr) | 8.00 ± 0.00 | 5.33 ± 2.31 | 4.00 ± 0.00 | 6.67 ± 2.31 |
| t_{1/2} (hr) | 12.7 ± 2.30 | 12.2 ± 6.46 | 9.54 ± 0.327 | 10.9 ± 2.06 |
| AUC₀₋ₗₐₛₜ (μg · hr/mL) | 300 ± 24.7 | 188 ± 44.4 | 254 ± 25.0 | 348 ± 16.7 |
| AUC_{0-inf} (μg · hr/mL) | 407 ± 20.4 | 253 ± 61.0 | 309 ± 32.8 | 446 ± 48.0 |
| MRTₗₐₛₜ (hr) | 9.42 ± 0.573 | 8.87 ± 1.28 | 8.51 ± 0.15 | 9.01 ± 0.480 |

Among the three Compound A amorphous solid dispersions dosed in aqueous suspension formulation at 50 mg/kg, the highest plasma exposure (Cₘₐₓ and AUC₀₋ₗₐₛₜ) was achieved using group 4 drug substance (40% API:60% PVPVA, TIME) which was also higher than the comparator in group 1 solution formulation (100% API, PEG400). The lowest plasma exposure was observed with group 2 solid dispersion (25% API:75% HPMCAS, SDD). The overall exposure differences between groups are less than 2-fold.

### Example 14. Treatment Schedule, Inclusion and Exclusion Criteria for Treatment Using Compound A

This is a multicenter, open-label, phase 2 study evaluating the safety and efficacy of Compound A monotherapy in pediatric and young adult patients with RAF-altered recurrent or progressive LGG or advanced solid tumors.

The study includes three arms. Arm 1: Compound A treatment for patients with LGG with activating BRAF alterations. Arm 2: Compound A treatment for patients with LGG harboring an activating RAF alteration after completion of enrollment to Arm 1, and prior to Compound A regulatory approval. Arm 3: Compound A treatment for patients with advanced solid tumors harboring an activating RAF fusion.

Compound A (available in tablet or liquid formulations) will be administered at the dose of 420 mg/m² (not to exceed 600 mg), orally, once weekly (days 1, 8, 15, and 22 of a 28-day cycle), in the absence of disease progression or unacceptable toxicity. Patients will be treated for a planned period of 26 cycles, after which they may continue on Compound A or, at any point, opt to enter a drug holiday discontinuation period.

Clinical assessments will be conducted days (D)1 and 15 of cycle (C)1, D1 of C2-26, then every third cycle to end of study (EOS). Radiological assessments will be conducted every 3 cycles to EOS for Arms 1 and 2, and every 2 cycles for 12 months, then every 3 cycles to EOS for Arm 3. Patients with radiographic evidence of disease progression may be allowed to continue treatment with Compound A if they are deriving clinical benefit from continuing treatment. A planned sample size of 60 evaluable patients in Arm 1 provides 88% power to reject the null overall response rate (ORR) of 21%, assuming that the true underlying ORR of Compound A is 40% based on a test at the 2-sided 0.05 level. A result of at least 20 out of 60 (0.33) will be statistically significant.

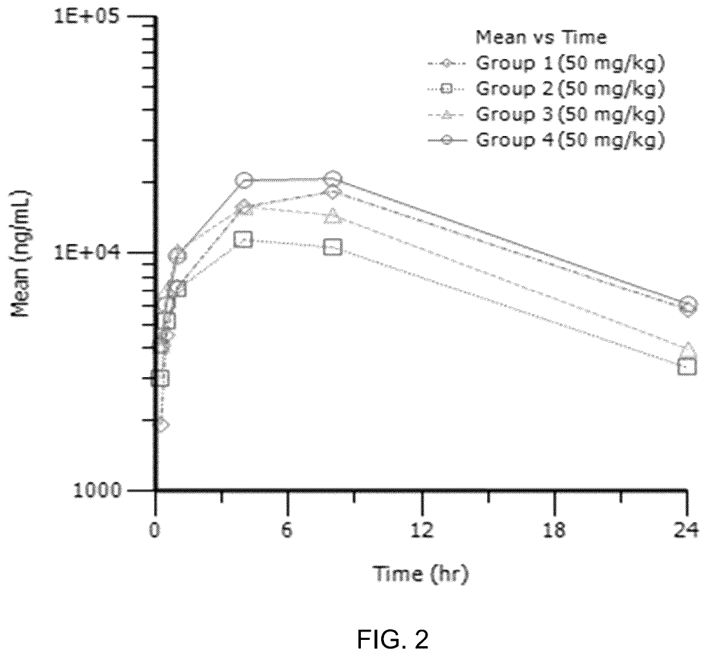

Inclusion Criteria can include: (a) Aged 6 months to 25 years with a RAF-altered LGG (Arms 1/2) or advanced solid tumor (Arm 3) histopathologically verified at either original diagnosis or relapse (per criteria defined in FIG. 2); (b) At least one line of prior systemic therapy and documented evidence of radiographic progression; (c) Evaluable and/or measurable disease (imaging performed within 28 days of initiation of treatment): Arm 1 (LGG): at least one measurable lesion as defined by Response Assessment in Neuro-Oncology (RANO) criteria; Arm 2 (LGG extension): evaluable (either unidimensionally measurable lesions, masses with margins not clearly defined, or lesions with maximal perpendicular diameters less than 10 mm) and/or measurable disease as defined by RANO criteria; and Arm 3 (advanced solid tumor): at least one measurable lesion as defined by Response Evaluation Criteria in Solid Tumors (RECIST) v1.1; (d) Karnofsky (aged >16 years) or Lansky (aged <16 years) performance score of at least 50; (e) Fully recovered from any prior surgery and the acute toxic effects of prior anticancer chemotherapy, and have undergone defined washout periods; (f) Chronic toxicities from prior anticancer therapy must be stable; (g) Available archival tumor tissue sample or fresh biopsy; and/or (h) Adequate organ function.

Exclusion Criteria can include: (a) Additional previously known or expected to be activating molecular alteration, including histone mutation, IDH1/2 mutation, FGFR mutation or fusion, MYBL alteration, NF1 somatic or germline mutation; (b) Symptoms of clinical progression without radiographically recurrent or radiographically progressive disease; (c) Known or suspected diagnosis of neurofibromatosis type 1; (d) History of any major disease, other than the primary malignancy under study, that might interfere with safe protocol participation; (e) Central serous retinopathy or retinal vein occlusion, or ophthalmopathy present at baseline that would be considered a risk factor for either; (f) Major surgery within 14 days prior to C1D1; (g) Clinically significant active cardiovascular disease; (h) Enrolled in any other investigational treatment study; (i) Active systemic bacterial, viral, or fungal infection; (j) Nausea and vomiting ≥National Cancer Institute Common Terminology Criteria for Adverse Events v5.0 grade 2, malabsorption requiring supplementation, or significant bowel or stomach resection that would preclude adequate absorption of Compound A; (k) Neurological instability despite adequate treatment; (1) Current treatment with a strong CYP2C8 inhibitor or inducer (other than those specified as allowed). Medications that are substrates of CYP2C8 are allowed but should be used with caution; (m) Pregnant or lactating; (n) Current treatment with sensitive CYP3A4 substrates (including hormonal contraceptives). Medications that are substrates of CYP3A4 are allowed but should be used with caution; and/or (o) Current treatment with BCRP substrates. Medications that are substrates of BCRP are allowed but should be used with caution.

Objectives of Arm 1 can include one or more of the following: (a) evaluate the efficacy of tovorafenib in patients with a relapsed or progressive LGG harboring a known activating BRAF alteration; (b) assess the safety and tolerability of tovorafenib in patients with LGG; (c) determine the relationship between PK and drug effects, including efficacy and safety; (d) evaluate the effect of tovorafenib on the QT interval and ECG parameters; (e) assess ORR based on the treating investigator's response assessment; (f) assess ORR based on RAPNO-LGG criteria as determined by an IRC; (g) evaluate the duration of PFS as determined by an IRC, based on RANO and RAPNO criteria, and by investigators, based on RANO criteria; (h) evaluate the DOR as determined by an IRC (RANO and RAPNO), and investigators (RANO only); (i) evaluate TTR as determined by an IRC (RANO and RAPNO) and investigators (RANO only): (j) evaluate the clinical benefit rate as determined by an IRC (RANO and RAPNO) and investigators (RANO only); (k) evaluate changes in BCVA outcomes; and/or (1) evaluate the concordance of prior local laboratory BRAF molecular profiling with a central BRAF alteration assay being evaluated.

Endpoints of Arm 1 can include one or more of the following: (a) ORR, as determined by an IRC, according to RANO criteria; (b) type, frequency, and severity of AEs and laboratory abnormalities; (c) PK profile of tovorafenib; (d) change from baseline (Δ)QTcF; ΔPR; ΔQRS; ΔHR; ECG waveform morphology; (e) ORR by RANO criteria; (f) ORR by RAPNO-LGG criteria; (g) time following initiation of tovorafenib to progression or death in treated patients; (h) length of response in patients with a confirmed response by RANO and RAPNO criteria; (i) time to first response by RANO and RAPNO criteria; (j) proportion of patients with BOR of CR, PR, or SD lasting 12 months or more, following initiation of tovorafenib; (k) change from baseline in BCVA (converted as logMAR) for each eye, and/or (1) molecular analysis of cells obtained from archival tissue.

Objectives of Arm 2 can include one or more of the following: (a) assess the safety and tolerability of tovorafenib in patients with LGG; (b) determine the ORR per RANO and RAPNO-LGG criteria as determined by an TRC and investigators (RANO only), and (c) evaluate PFS, DOR, TTR, clinical benefit rate, the relationship between PK and drug effects, and the effect of tovorafenib on the QT interval and ECG parameters, as described for Arm 1

Endpoints of Arm 2 can include one or more of the following: (a) Type, frequency, and severity of AEs and laboratory abnormalities; (b) ORR by RANO and RAPNO-LGG criteria; and/or (c) As described for Arm 1.

Objectives of Arm 3 can include one or more of the following: (a) evaluate the preliminary efficacy of tovorafenib in patients with a relapsed or progressive advanced solid tumor harboring a known or expected to be activating RAF fusion; (b) assess the safety and tolerability of tovorafenib in pediatric patients with advanced solid tumors; (c) evaluate the relationship between PK and drug effects, and the effect of tovorafenib on the QT interval and ECG parameters; (d) determine the ORR based on investigator assessment; (e) evaluate PFS, DOR, TTR, and clinical benefit rate; and/or (f) evaluate the concordance of prior local laboratory RAF molecular profiling with a central RAF alteration assay being evaluated.

Endpoints of Arm 3 can include one or more of the following: (a) ORR, as determined by an IRC, according to RECIST v1.1; (b) type, frequency, and severity of AEs and laboratory abnormalities; (c) as described for Arm 1; (d) ORR, as assessed by investigators, according to RECIST v1.1; (e) molecular analysis of cells obtained from archival tissue.

AEs, adverse events; BCVA, best corrected visual acuity; BOR, best overall response; CR, complete response; DOR, duration of response; ECG, electrocardiogram; HR, heart rate; IRC, independent radiology review committee; LGG, low-grade glioma; ORR, overall response rate, PFS, progression-free survival; PK, pharmacokinetics; PR, partial response; QTcF, QT interval corrected for heart rate by Fridericia's formula; RANG, Response Assessment in Neuro-Oncology; RAPNO, Response Assessment in Pediatric Neuro-Oncology; RECIST, Response Evaluation Criteria in Solid Tumors; SD, stable disease; TTR, time to response

### Example 15. Exemplary Formulation

An exemplary unit dose composition of the disclosed powder formulation of Compound A is illustrated in Table 29A below. The exemplary formulation of Table 29A was reconstituted in water, e.g., resulting a liquid suspension containing Compound A at about 25 mg/mL.

TABLE 29A
Exemplary Powder Formulation

| | 300 mg Bottle | | Pharmaceutical |
|---|---|---|---|
| | mg per bottle | % w/w | Function |
| Compound A | 429.72 | 9.784 | Active |
| Copovidone | 644.57 | 14.675 | Physical |
| (Kollidon VA 64) | | | Stability/Solubilizer |
| Microcrystalline | 1337.73 | 30.457 | Filler |
| Cellulose | | | |
| (Avicel PH-101) | | | |
| Mannitol | 1337.73 | 30.457 | Filler |
| Sodium Lauryl Sulfate | 32.24 | 0.734 | Solubilizer |
| Simethicone (Liveo | 96.67 | 2.201 | Anti-foam agent |
| Q7-2243 LVA) | | | |
| Maltodextrin | 225.63 | 5.137 | Carrier for |
| (Maltrin QD M500) | | | Simethicone |
| Colloidal Silicon Dioxide | 193.39 | 4.403 | Glidant |
| (Aerosil 200) | | | |
| Sucralose Powder | 25.78 | 0.587 | Sweetener |
| Artificial Strawberry | 68.74 | 1.565 | Flavoring |
| Flavor | | | |
| Total | 4392.2 | 100 | |

Unit dose compositions of the disclosed powder formulation of Compound A are illustrated in Tables 29B and 29C below. The formulations of Table 29B and Table 29C may be reconstituted in water, e.g., resulting a liquid suspension containing Compound A at about 25 mg/mL.

TABLE 29B
Exemplary Powder Formulations

| | F-29B-1 | F-29B-2 | F-29B-3 | F-29B-3 | F-29B-3 |
|---|---|---|---|---|---|
| Component | % w/w | % w/w | % w/w | % w/w | % w/w |
| Compound A | 5-15 | 5-15 | 5-15 | 5-15 | 5-15 |
| Copovidone | 5-20 | 5-10 | 10-20 | 5-15 | 15-20 |
| (Kollidon VA 64) | | | | | |
| Microcrystalline | 25-35 | 25-30 | 20-30 | 20-35 | 15-30 |
| Cellulose | | | | | |
| (Avicel PH-101) | | | | | |
| Mannitol | 25-35 | 25-30 | 20-30 | 20-25 | 25-35 |
| Sodium Lauryl Sulfate | 0.5-1.0 | 0.5-0.75 | 0.5-0.6 | 0.1-1.0 | 0.2-0.8 |
| Simethicone (Liveo | 0.5-2.0 | 1.0-2.0 | 0.5-1.0 | 0.25-0.75 | 0.5-1.0 |
| Q7-2243 LVA) | | | | | |
| Maltodextrin | 2.0-8.0 | 5.0-8.0 | 3.0-5.0 | 2.0-5.0 | 2.0-3.0 |
| (Maltrin QD M500) | | | | | |
| Colloidal Silicon Dioxide | 1.0-5.0 | 1.0-3.0 | 2.0-3.0 | 2.0-5.0 | 2.5-3.5 |
| (Aerosil 200) | | | | | |
| Sucralose Powder | 0.1-1.0 | 0.5-1.0 | 0.25-0.50 | 0.75-1.0 | 0.25-0.75 |
| Artificial Strawberry | 0.5-1.5 | 0.5-1.0 | 0.25-1.0 | 0.1-0.5 | 0.75-1.5 |
| Flavor | | | | | |

TABLE 29C
Exemplary Powder Formulations

| F-29C-1 | F-29C-2 | F-29C-3 | F-29C-4 | F-29C-5 |
|---|---|---|---|---|
| Component | Component | Component | Component | Component |
| (% w/w) | (% w/w) | (% w/w) | (% w/w) | (% w/w) |
| Compound A | Compound A | Compound A | Compound A | Compound A |
| (5-15) | (5-15) | (5-15) | (5-15) | (5-15) |
| vinylpyrrolidone- | vinylpyrrolidone- | vinylpyrrolidone- | vinylpyrrolidone- | vinylpyrrolidone- |
| vinyl acetate | vinyl acetate | vinyl acetate | vinyl acetate | vinyl acetate |
| copolymer | copolymer | copolymer | copolymer | copolymer |
| (10-20) | (10-20) | (10-20) | (10-20) | (10-20) |
| sodium | Microcrystalline | Microcrystalline | Microcrystalline | sodium |
| carboxymethyl | Cellulose | Cellulose | Cellulose | carboxymethyl |
| cellulose | (25-35) | (25-35) | (25-35) | cellulose |
| (25-35) | | | | (25-35) |
| Mannitol | Mannitol | Mannitol | Sodium | Mannitol |
| (25-35) | (25-35) | (25-35) | carboxymethyl | (25-35) |
| | | | cellulose | |
| | | | (25-35) | |
| Sodium Lauryl | Sodium dodecyl | Sodium Lauryl | Sodium Lauryl | poloxamer |
| Sulfate | sulfate | Sulfate | Sulfate | (0.5-4.0) |
| (0.5-1.0) | (0.5-1.0) | (0.5-1.0) | (0.5-1.0) | |
| Simethicone | Simethicone | Simethicone | Simethicone | Simethicone |
| (0.5-2.0) | (0.5-2.0) | (0.5-2.0) | (0.5-2.0) | (0.5-2.0) |
| Maltodextrin | Maltodextrin | Polydextrose | Maltodextrin | Maltodextrin |
| (2.0-8.0) | (2.0-8.0) | (2.0-8.0) | (2.0-8.0) | (2.0-8.0) |
| Colloidal Silicon | Silicon | Silicon | Silicon | Colloidal Silicon |
| Dioxide | Dioxide | Dioxide | Dioxide | Dioxide |
| (1.0-5.0) | (1.0-5.0) | (1.0-5.0) | (1.0-5.0) | (1.0-5.0) |
| Sucralose Powder | Sucralose Powder | Saccharin | Sucralose Powder | Sucralose Powder |
| (0.1-1.0) | (0.1-1.0) | (0.1-1.0) | (0.1-1.0) | (0.1-1.0) |
| Strawberry Flavor | Strawberry Flavor | Vanilla Flavor | Cherry Flavor | Cherry Flavor |
| (0.5-1.5) | (0.5-1.5) | (0.5-1.5) | (0.5-1.5) | (0.5-1.5) |

### Example 16. Exemplary Treatment Schedule

Exemplary dosage reductions for adverse reactions are provided in Table 30 (tablets) and Table 31 (liquid formulation/powder for oral suspension).

TABLE 30
Exemplary Dosage Reductions for Adverse Reactions (Tablets)

| | | |
|---|---|---|
| BSA | First Dose Reduction | Second Dose Reduction |
| (m²) | (mg) | (mg) |

| | |
|---|---|
| 0.3 | Oral suspension (see Table 31) |

| | | |
|---|---|---|
| 0.4 | | |
| 0.5 | | |
| 0.6 | | |
| 0.7 | | |
| 0.8 | | |
| 0.9-1.0 | 300 (3 × 100 mg) | 200 (2 × 100 mg) |
| 1.1-1.2 | 400 (4 × 100 mg) | 300 (3 × 100 mg) |
| 1.3-≥1.9 | 500 (5 × 100 mg) | 400 (4 × 100 mg) |

TABLE 31
Exemplary Dosage Reductions for Adverse Reactions
(Liquid Formulation/Powder for oral suspension)

| BSA (m²) | First Dose Reduction (mg) | Second Dose Reduction (mg) |
|---|---|---|
| 0.3 | 4 | 3 |
| 0.4 | 5 | 4 |
| 0.5 | 7 | 5 |
| 0.6 | 8 | 6 |
| 0.7-0.8 | 10 | 8 |
| 0.9 | 12 | 10 |
| 1.0 | 14 | 11 |
| 1.1 | 15 | 12 |
| 1.2 | 17 | 13 |
| 1.3 | 18 | 15 |
| 1.4-≥1.9 | 20 | 16 |

TABLE 32
Exemplary Dosage Modifications for Adverse Reactions

| | |
|---|---|
| Severity of ADR^{a} | Dose Modification |

Skin Toxicity [see Warnings and Precautions (5.1)]

| | |
|---|---|
| Grade 3 or 4 | Withhold until improvement. |
| | Resume at reduced dose. |

Photosensitivity [see Warnings and Precautions (5.2)]

| | |
|---|---|
| Intolerable Grade 2 | Withhold until improvement. |
| | Resume at reduced dose. |

ADRs [See Adverse Reactions (6.1)]

| | |
|---|---|
| Intolerable Grade 2 | Withhold until improvement. |
| Grade 3 | Resume at reduced dose. |
| Grade 4 | Withhold until improvement. |
| | Resume at reduced dose. |
| | Consider discontinuation. |

^{a}National Cancer Institute Common Terminology Criteria for Adverse Events (NCICTCAE) version 5.0.

### Example 16. Exemplary Administration Method

A powder formulation of Compound A is reconstituted with water and the resulting suspension is administered orally, or enterally via a nasal or gastric feeding tube with a dosing syringe.

Prior to preparing a dose of Compound A for the first time, refer to the Instructions for Use.

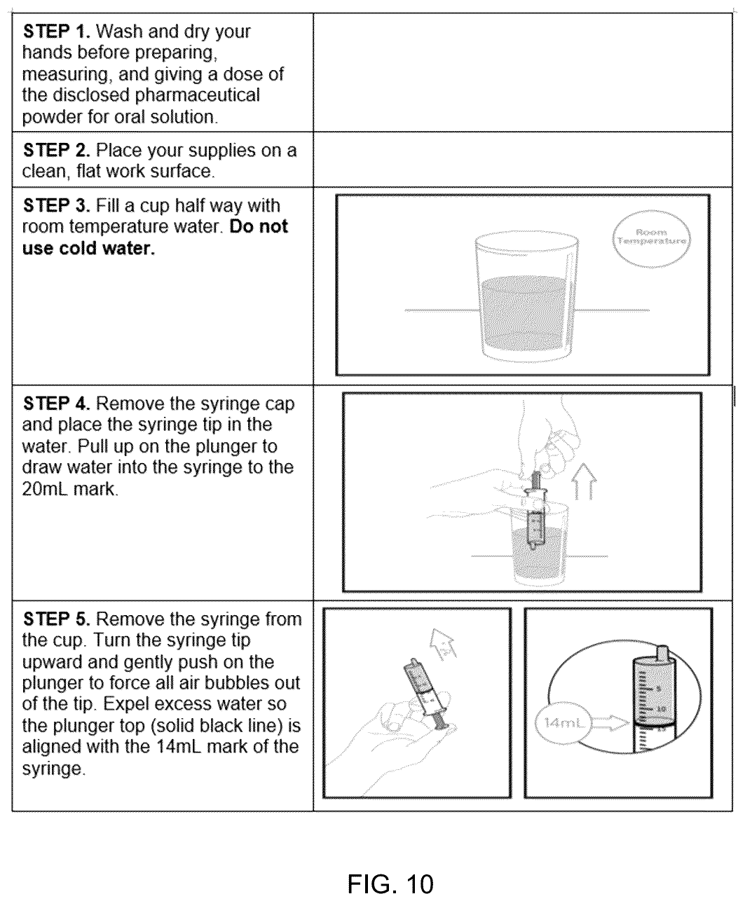

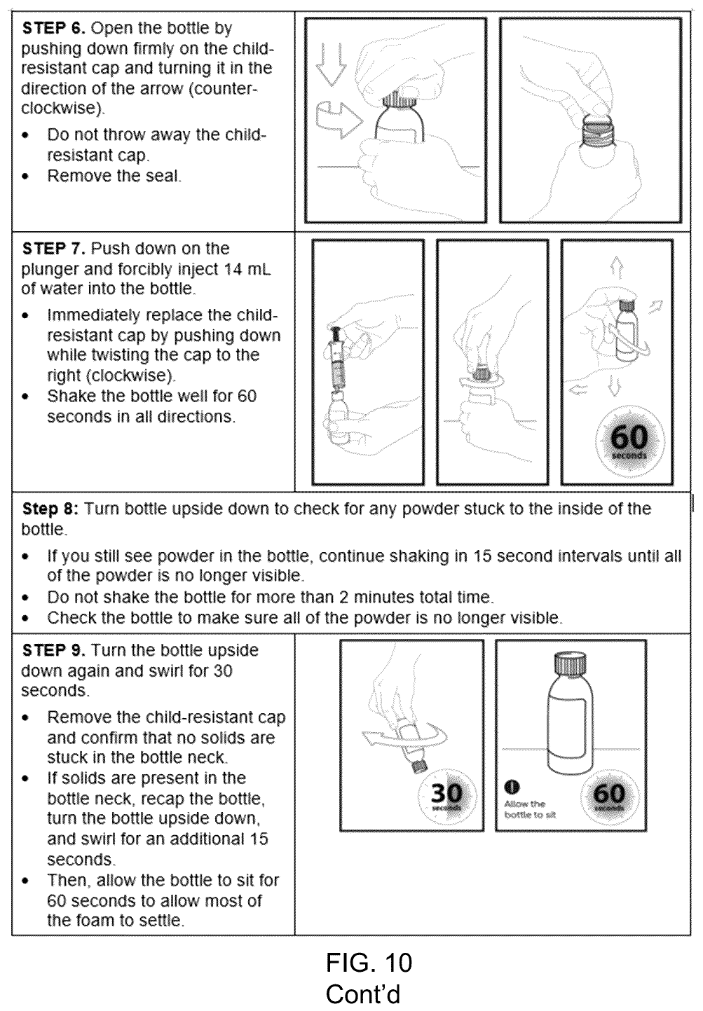

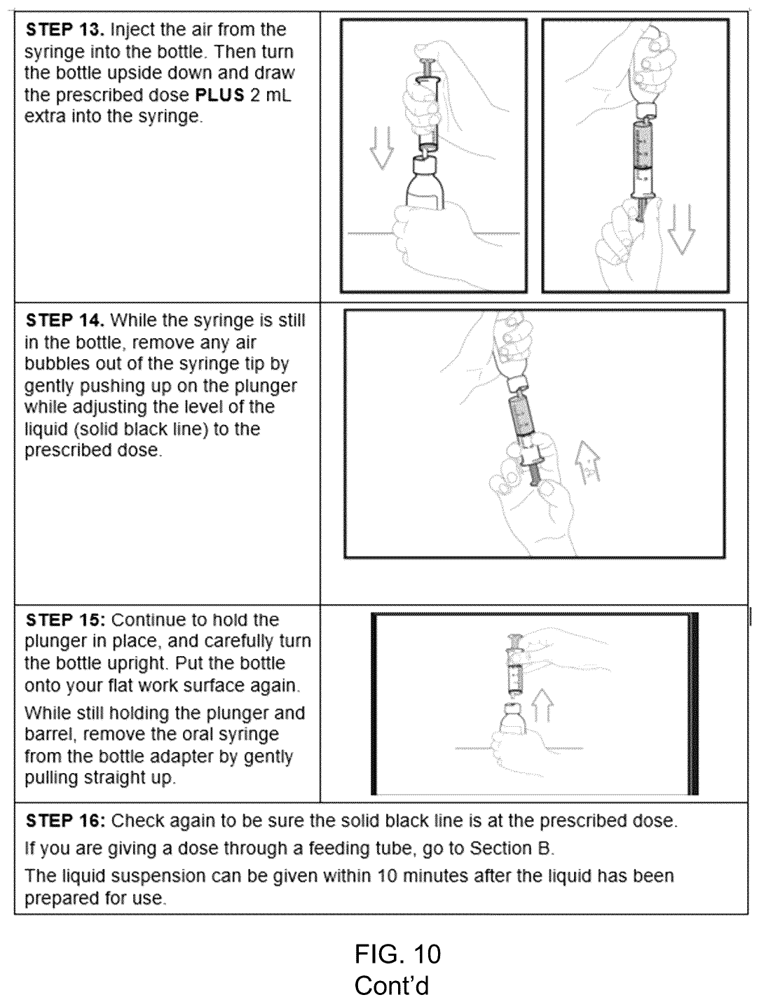

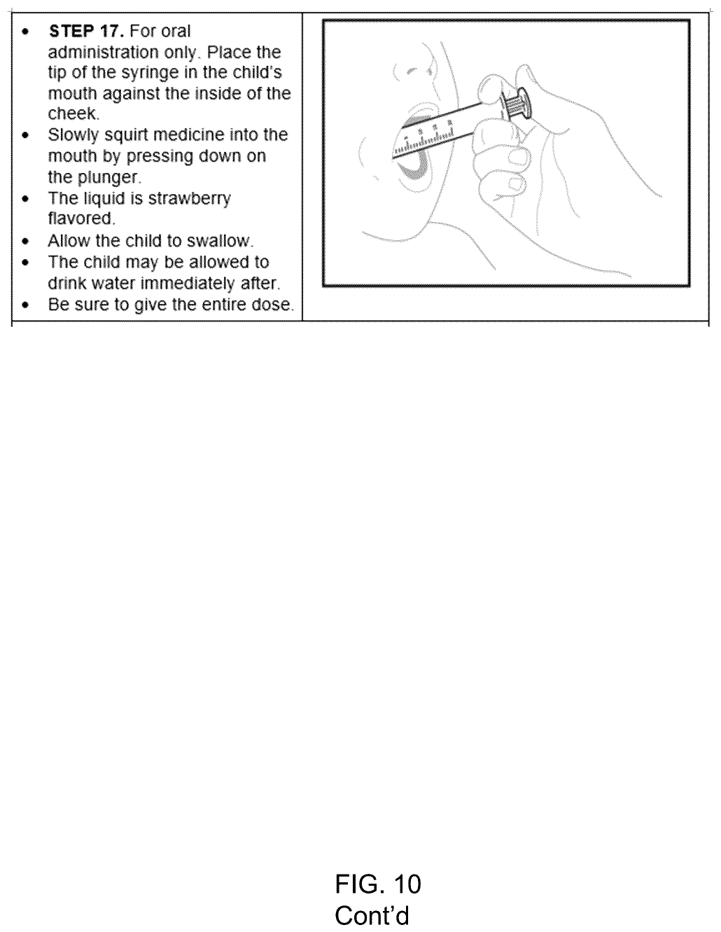

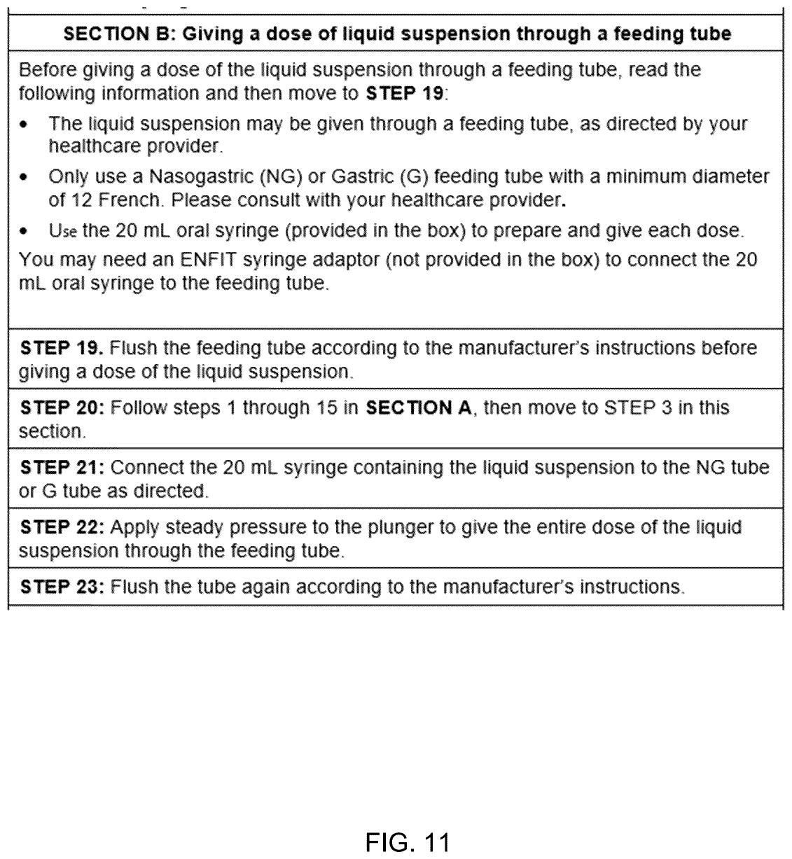

Further examples of admixture and administration are shown in FIGS. 10 and 11.

### Example 18. Suspension Stability

Suspension stability (or syringeability) of the reconstituted Powder for Reconstitution of Compound A was performed by allowing the sample to sit following reconstitution for iterative lengths of time. This was performed to determine at what length of time the reconstituted formulation could no longer be used correctly due to the PfR formulation propensity to ‘gel’ after a length of time.

When the sample dose becomes unable to be withdrawn from the sample bottle, it constitutes a failure at that time point.

The suspension stability of the herein disclosed formulation were tested at 10 min, 15 min and 20 min.

1. A kit comprising:
a) a solid formulation of an amorphous solid dispersion of (R)-2-(1-(6-amino-5-chloropyrimidine-4-carboxamido)ethyl)-N-(5-chloro-4-(trifluoromethyl)pyridin-2-yl)thiazole-5-carboxamide (Compound A) or a pharmaceutically acceptable salt thereof, wherein the solid formulation comprises one or more pharmaceutically acceptable excipients; and
b) instructions for aqueous reconstitution of the solid formulation.

2. The kit of claim 1, wherein the solid formulation is in a powder, granular, or pellet form.

3. (canceled)

4. (canceled)

5. The kit of claim 1, wherein the amorphous solid dispersion comprises one or more polymers, and wherein the one or more polymers comprise polyvinylpyrrolidone, polyvinylpyrrolidone-polyvinyl acetate copolymer (PVP-VA), cross linked polyvinyl N-pyrrolidone, polyvinyl alcohol (PVA), polysaccharide, hydroxypropyl methylcellulose (HPMC or Hypromellose), hydroxyethyl cellulose (HEC), hydroxypropyl cellulose (HPC), polyethylene oxide, hydroxypropyl-β-cyclodextrin (HP-β-CD), sulfobutylether-β-cyclodextrin (Captisol), cyclodextrin (e.g., γ-cyclodextrin), hydroxypropyl methylcellulose acetate succinate (HPMCAS), polyethylene glycol (PEG), polyvinyl caprolactam-polyvinyl acetate-polyethylene glycol graft copolymer (PVAc-PVCap-PEG), polysaccharide, poly(methacrylic acid-co-methyl methacrylate) (Eudragit), poloxamers, silica gel, aluminosilicate, or a combination thereof.

6-12. (canceled)

13. The kit of claim 5, wherein a weight ratio of the Compound A or a pharmaceutically acceptable salt thereof to the one or more polymers is 5:1 to 1:5.

14. (canceled)

15. The kit of claim 1, wherein the amorphous solid dispersion comprises Compound A.

16-26. (canceled)

27. The kit of claim 1, wherein the amorphous solid dispersion comprises one or more pharmaceutically acceptable excipients, and wherein the one or more pharmaceutically acceptable excipients are selected from an antifoam, a flow-aid, a surfactant, a filler, a colorant, a preservative, a flavoring agent, a sweetener, and a combination thereof.

28-49. (canceled)

50. The kit of claim 1, wherein the solid formulation comprises:
c) an amorphous solid dispersion comprising about 10 wt % to about 60 wt % of Compound A and about 40 wt % to about 60 wt % of PVP-VA, wherein the amorphous solid dispersion is a hot melt extrudate and is present in the solid formulation at about 10 wt % to about 30 wt %;
d) about 30 wt % to 70 wt % of a filler, wherein the filler comprises microcrystalline cellulose and mannitol;
e) about 0.1 wt % to 5 wt % of a surfactant, wherein the surfactant is SLS;
f) about 0.25 wt % to 6 wt % of a flow-aid, wherein the flow-aid is CSD;
g) about 0.5 wt % to 5 wt % of an antifoam, wherein the antifoam is simethicone.

51-59. (canceled)

60. A pharmaceutical powder comprising:
a) an amorphous solid dispersion that comprises (R)-2-(1-(6-amino-5-chloropyrimidine-4-carboxamido)ethyl)-N-(5-chloro-4-(trifluoromethyl)pyridin-2-yl)thiazole-5-carboxamide (Compound A) or a pharmaceutically acceptable salt thereof; and
b) one or more pharmaceutically acceptable excipients, wherein the excipients comprises a flow-aid and a surfactant.

61. The pharmaceutical powder of claim 60, wherein the pharmaceutical powder is configured to be reconstituted into an oral liquid suspension.

62. (canceled)

63. (canceled)

64. The pharmaceutical composition of claim 60, wherein the amorphous solid dispersion comprises one or more polymers and, wherein the one or more polymers comprise PVP-VA or HPMCAS.

65-84. (canceled)

85. The pharmaceutical powder of claim 60, wherein the flow-aid is selected from silicon dioxide, magnesium stearate, talc, starch, magnesium silicate, hydrated sodium sulfoaluminate, and a combination thereof.

86. The pharmaceutical powder of claim 85, wherein the silicon dioxide comprises fumed silica, colloidal silicon dioxide (CSD), or both.

87. (canceled)

88. (canceled)

89. The pharmaceutical powder of claim 60, wherein the surfactant comprises sodium lauryl sulfate (SLS) or poloxamer.

90. (canceled)

91. (canceled)

92. The pharmaceutical powder of claim 60, comprising:
a) about 10 wt % to about 60 wt % of an amorphous solid dispersion comprising (i) about 40 wt % to about 60 wt % of Compound A and (ii) about 40 wt % to about 60 wt % of PVP-VA, wherein the amorphous solid dispersion is a hot melt extrudate;
b) about 30 wt % to about 70 wt % of a filler, wherein the filler comprises microcrystalline cellulose and mannitol;
c) about 0.1 wt % to 5 wt % of a surfactant, wherein the surfactant is SLS;
d) about 0.25 wt % to 6 wt % of a flow-aid, wherein the flow-aid is CSD; and
e) about 0.5 wt % to 5 wt % of an antifoam, wherein the antifoam is simethicone or dimethicone.

93-95. (canceled)

96. An oral liquid suspension produced by contacting a pharmaceutical powder of claim 60 with an aqueous solution.

97. An oral liquid suspension, comprising:
a) (R)-2-(1-(6-amino-5-chloropyrimidine-4-carboxamido)ethyl)-N-(5-chloro-4-(trifluoromethyl)pyridin-2-yl)thiazole-5-carboxamide (Compound A) or a pharmaceutically acceptable salt thereof,
b) one or more pharmaceutically acceptable excipients; and
c) water.

98. The oral liquid suspension of claim 97, wherein Compound A or a pharmaceutically acceptable salt thereof is in a form of an amorphous solid dispersion.

99. (canceled)

100. The oral liquid suspension of claim 97, wherein the amorphous solid dispersion comprises one or more polymers, and wherein the one or more polymers comprise polyvinylpyrrolidone, vinylpyrrolidone-vinyl acetate copolymer (PVP-VA), cross linked polyvinyl N-pyrrolidone, polyvinyl alcohol (PVA), polysaccharide, hydroxypropyl methylcellulose (HPMC or Hypromellose), hydroxyethyl cellulose (HEC), hydroxypropyl cellulose (HPC), polyethylene oxide, hydroxypropyl-β-cyclodextrin (HP-β-CD), sulfobutylether-β-cyclodextrin (Captisol), γ-cyclodextrin, hydroxypropyl methylcellulose acetate succinate (HPMCAS), polyethylene glycol (PEG), polyvinyl caprolactam-polyvinyl acetate-polyethylene glycol graft copolymer (PVAc-PVCap-PEG), polysaccharide, poly(methacrylic acid-co-methyl methacrylate) (Eudragit), poloxamers, silica gel, aluminosilicate, or a combination thereof.

101. (canceled)

102. The oral liquid suspension of claim 97, wherein the oral liquid suspension is reconstituted.

103. (canceled)

104. The oral liquid suspension of claim 97, wherein a concentration of Compound A or a pharmaceutically acceptable salt thereof is about 10 to about 50 mg/mL in the oral liquid suspension.

105. (canceled)

106. (canceled)

107. The oral liquid suspension of claim 97, wherein the suspension retains syringeability for at least 20 minutes.

108-122. (canceled)

123. The oral liquid suspension of claim 97, wherein the suspension comprises, based on the weight of the solids:
a) about 10 wt % to about 50 wt % of an amorphous solid dispersion comprising (i) about 40 wt % to about 60 wt % of Compound A and (ii) about 40 wt % to about 60 wt % of PVP-VA, wherein the amorphous solid dispersion is a hot melt extrudate;
b) about 40 wt % to about 70 wt % of a filler, wherein the filler comprises microcrystalline cellulose and mannitol;
c) about 0.25 wt % to about 1 wt % of a surfactant, wherein the surfactant is SLS;
d) about 1 wt % to about 6 wt % of a flow-aid, wherein the flow-aid is colloidal silicon dioxide (CSD);
e) about 1 wt % to about 5 wt % of an antifoam, wherein the antifoam comprises simethicone; and
f) optionally a preservative, a flavoring agent, a sweetener, or a combination thereof.

124. The oral liquid suspension of claim 97, wherein the suspension comprises, based on the weight of the solids:
a) about 20-30 wt % of an amorphous solid dispersion comprising (i) about 40 wt % of Compound A and (ii) about 60 wt % of copovidone, wherein the amorphous solid dispersion is optionally a hot melt extrudate;
b) about 30 wt % to 32 wt % mannitol;
c) about 30 wt % to 32 wt % microcrystalline cellulose;
d) about 0.5 wt % to 1 wt % SLS;
e) about 4 wt % to 5 wt % CSD;
f) about 1 wt % to 3 wt % simethicone;
g) about 3 wt % to about 8% wt % of Maltodextrin;
h) optionally a preservative, a flavoring agent, a sweetener, or a combination thereof.

125. (canceled)

126. The oral liquid suspension of claim 97, wherein the oral liquid suspension is bioequivalent to a tablet formulation of Compound A, wherein the tablet composition comprises an amorphous solid dispersion comprising (i) about 40 wt % of Compound A and (ii) about 60 wt % of copovidone, wherein the amorphous solid dispersion is a hot melt extrudate; and one or more pharmaceutically acceptable excipients.

127. The oral liquid suspension of claim 97, wherein the oral liquid suspension, when administered to a human subject in an amount equivalent to about 100 mg of Compound A, is sufficient to achieve in the subject a maximum observed blood plasma concentration (Cmax) of Compound A of at least about 100 ng/mL.

128-130. (canceled)

131. A method of treating pediatric low grade glioma (pLGG) in a subject, comprising administering to the subject an oral liquid suspension comprising (R)-2-(1-(6-amino-5-chloropyrimidine-4-carboxamido)ethyl)-N-(5-chloro-4-(trifluoromethyl)pyridin-2-yl)thiazole-5-carboxamide (Compound A), or a pharmaceutically acceptable salt thereof.

132-134. (canceled)

135. A method of treating a subject with pediatric low grade glioma (pLGG), comprising reconstituting an amorphous solid dispersion of Compound A or a salt thereof in an aqueous solution and administering a pharmaceutically acceptable dosage of the reconstituted Compound A or a salt thereof to the subject in need thereof.

136-161. (canceled)

162. A method of preparing an oral liquid suspension of Compound A or a salt thereof, comprising reconstituting a pharmaceutical powder of claim 60 in an aqueous solution.

163-172. (canceled)
